SEQUENCE LISTING (71 sequences)

SEQ ID NO: 1 - protein, 1091 aa, Manihot esculenta
MNRNGNTRKRPPPTSQPTPSPPAPKHQATTPAVDDEFVDEDVFLDETLIAEEENLILRDLEERQALASRLAKWSRPSLSHEYTSQSRSIIFQQLEIDYVIGESHKELLPDRSGVAAIIRIFGVTREGHSVCCLVHGFEPYFYISCPPGMGPDDISRFQQILEGRMKEMNRNSQVPKFVRRIEMVQKRSIMYYQQQPSHPFLKIVVALPTMVASCRGILDKGIHIDGLGMKSFMTYESNVLFALRFMIDCNVVGGNWIEVPAGKYKKTSKNLSYGQLEFDCLYSELISHAPEGEFSKMAPFRILSFDIECAGRKGHFPEPTHDPVIQVANLVTMQGEDQPFIRNVMTLNSCSPIVGVDVMSFDTEREVLLAWRDFIREVDPDIIIGYNICKFDLPYLIERAETLGIAEFPVFGRVKNSRVRVKDTTFSSRQYGTRESKEVTLEGRVQFDLLQAMQRDYKLSSYSLNSVSAHFLSEQKEDVHHSIISDLQNGNAETRRRLAVYCLKDAYLPQRLLDKLMFIYNYVEMARVTGVPLSFLLSRGQSIKVLSQLLRKAKQKNLVIPNVKQAGSEQGTYEGATVLEARAGFYEKPIATLDFASLYPSIMMAYNLCYCTLVTPEDVRKLNIPPECVNKTPSGETFVKSNLQKGILPEILEELLAARRRAKADLKEAKDPLVKAVLDGRQLALKVSANSVYGFTGATIGQLPCIEISSSVTSYGRQMIEHTKKLVEEKFTITGGYEHNAEVIYGDTDSVMVQFGVPTVEEAMKLGREAAECISGTFIKPIKLEFEKVYYPYLLISKKRYAGLFWTNPNKFDKMDTKGIETVRRDNCLLVKNLVNECLHKILIDRDVPGAVQYVKNTISDLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNSDRTLCTHCKGREAELYCKTVSQVSELELLFGRLWTQCQECQGSLHQDVLCTSRDCPIFYRRKKAQKDMAEAKRQLDRWNF

SEQ ID NO: 2 - DNA, 22487 bp, Manihot esculenta
  source 1..22487: mol_type = genomic DNA
ctatctctcattctcactctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacaacccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgtccctctctcacgagtacacttcccaatctcgtagcattagtgagtaacttcggtgtttcctaattatgggtattattgttgtggattacttttattttcttttatctttcttttattgtgaaattcgatggatttattattttctttacggtcagtatttcagcagctggaaattgattatgtgattggagagagtcataaggagctgctgcctgataggtcaggggtcgctgcgattatcagaatttttggggtcacaagggaaggttaggtcattattgtaatttagaaaataatccctataaaactgagaacatgctatagaaaagtgaaattttttactttttttttttaataaatttgttaaatttgtggtgtagttgtattgtatatgtgtagaagcatacttttacttacactatctatgggttttttactcggtgaagcgaagggtgtagttttcaatttcaaaggtttctaattggcaaatttgataacatattcatggaattcattggtaaacgagtgtactaattatgttttatgcatgagtagttaaatatggcaatctattcatgtgcctggaaatgcaacatctatttattctgtaacattcttatttcagtatattttaatttggtttccaattatgctccatattttgcctctctcctctgatttccaaaccaaataaacaaaaatattgtacacatttgctttttcgactttggatcagtggtttcattctgatttcaacccagtttgtctatttcatcaaatttagatttgatagatacatgtaacacaatatccatattttttaaatttctttctctaattgacattggcacacacttgcactgatgaattctttgaataggtagatgattagtttctatatcatttaagattttttttttcttgttgaatttgaatcagcgacatattgctctttgagaatcacagttacttttttatcattttattctcttgccaatttcaacgttgtgttatataataaactttatagctatggatatggctaaagcatcttgaactacatacagtttgaaacttgtaattgtttctacctcacacaaatatatttacatactgactttttgttaggacatagtgtttgctgccttgttcacggatttgagccatatttttacatcagttgccctcctgggatgggtcctgatgatatttctcgttttcaacaaattcttgaggtatttcatctacgaatttctctgcctatatatttatagataactacatgcatacacacatatgtatatagagctaattgattgcttaattatctcaatatcatctttgttttgggtgtttaaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtaagcatcttcaattttttaatagatgtaactgtgatgcaaacatgtataatctcttttaagttgtttacagcataatgattttgatatctgtattgaattttgagtctcttgacaattcccaatcatttgcaggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtatccttatgcatgtcgatcattctttccttgagagaatacttcaattcttattgtccatacttggggctgttttcttgaaaacaattttctgtttttcattttttttttttttttggtaaggaaactctccattttgcattttctataaagtaagggaaaacgtggagatcccattatagaaaacaattttctaattcaattttttaaaatgttcatatcattcataataaaaaaaaaatcattataaattatatataaaattttgtttaacttttaaattttttaatatttgttattcatttaatgtataatagtatgattaatttttatcattgtaaattaatattttcctaaataattattcaattttagttatgacaaatttatggtgtaattttagttatagaaaagtcattattttctatgtcaaaaaatttggagacgaagattaaaaactaacttatcactattttcctaaccttagttaaattttcaaggcaaccggaagctagttttccaatccttcatttggaaaattggcattcgtgaatgaaactctgttttccagttctcttagctaacttttctagaaaactactccagttttccacagtgaacatttccgtagtgtttttcaagattaattccttgaccaatgttcataggtattcagaattgataagccatgccccagaaggggaattctcaaagatggctccattccgcattttaagttttgatattgaatgtgctggtcggaaaggtcattttcctgagcctacccatgatcctgttattcaggtttttccctagtttttatgcttgttttacttcagtcaagcagtctggttggtggatggatgataattcctttttcactgttgctgaaatcaaaatttccatttcattttaggtggcaaatctagttactatgcagggggaggaccaaccatttatccgtaatgttatgactttaaactcatgctctccaatagttggtgtagatgtgatgtcttttgatacggagagagaagtcctgctggcttggagggtatgtttgcttttctctatagttctcttttgctttgaagaaagctaggttgcatgtttataatttgaataactcttctatacaattattttaacatgttagatttggtgaggaaaatttctgtaagtaatataataaaaaactcataagataatagcaacctgagacttttgaatggcatagttgttctaaagcaaaaatattttagcaaatttaatagtatggtaatagatgcatctgtcatatactcctatcttcctgggtttgctgcttgaatccagtttgatctatagctgtagtggcccatgtgtctcctgcagttataatgttcatgtctttttggaaccaccatcttgaatgtgtctgtcaatggaagttggaagttgaagctttgagctatctgcatttgatcaatgggatggactttttttttttttttttatcttgtgaaatgccgtcttatttgcttgtttcagaaaataatggcttccacagttatttatttattactctggataaaaaaaatttcattcatagtgtttgattgaaaacctagcttaaagtacatacagccaggtgaaattgctcctctttatctctcactccatcctttatctcctcctcctcctttttatttatttatttctttatttttagatttcggtaaatctatcttattaaacaaaattctcagttcatagtcaacaatctcatatactgagcatgacctagaggtgtgcaaaatctgctttggcagttaaaaggattttggatatcaagaagaaaccacaatttacagtttgtgaatttctcaaaccaatttagactatctttgagtacagaaatcaaactaatcaaaatgttagttcacaattttgtttggcatggaaatcccttttggctgaagaagccattcttttagattctcctgtcacaactatgcttatcaaacacttggccatggttgcactaaaatccactttcttccatatatttgctgtgtaagcatgaatcttcatgcacccttagctttgccttctttgataccctaatctttgtgtagtgcttaaatataggactctagcttaacaatgtgtatcttaactgtactatcagttgatgggttgatcttgtattagttttatgatcaggcgctgcattaaggttattaataatttctcgtagtaggatatagtgcattttatgatgtagtaagcaggttttgcatgcaggattttatccgtgaagtggacccagatattataattggatataatatctgcaaatttgacttgccgtatctcattgaggtaattagaagtcttctatattctctttatgcatttttatagtgaagttttttttctttaattttgttttgcagtttaataaatttgaaatgctatttttcttggtttacagagagctgaaactttgggtatagcagagtttcctgtgttcggccgtgtcaagaatagcagggttcgggtcaaagacacaacattttcatcgaggtagtatttttgtttgttacaacaagtatcatgtctttcgtgtgcgtgtgtctttcgtgtgcgtgcttacatattcttgattggagggcattgctttataaactttttttttcttctctcgctttttataatcttcttgttgtagttgtatttggagctctttaggaggattccttgcttactatgctgtatttgttgagttctattcgattccttctgtaccttgttttattggtcaagttctgtagaagtattccattccatgtctaccatgtagcactctcctttctttctcaataaaatattttcgtatatactgaaaaaatattgatgccgtcataagctttaagttgaagattagaatacttgtgcacagtgcatgttatacttttgcttcctgtctaagatttttttttcccatgaaatttgatgagaagagttactgccttcccgagaatttcagagatttaatgagaagtttaatgagaagttcagagaatttcatggaatatgagtgatgtgcttttatttgtctataaacccaaaatgaacttttatgtccttttggtttcataaatttcagactctcaggtgaaatccctagtagtcgtaaagatagcaatataattgggaagatcgtattgtaatttgatttcaaaaatctgggtcatccttaatgtgagagtaagctttaaagaatgtgtgtatggtgttccttacctggatttagtgcttaaacattggcaccttaatccttgttgctgtttgcagacaatatggtacaagggaaagtaaagaagttacattggaaggaagggttcaatttgatttgcttcaggttttgtgcaataaaactttttctttccacaatctcatttttgtttctatgagataaaagttcatttaggcatgttgttatccttccatccaggcaatgcaacgagattataaattaagttcttactcattaaattctgtctcggcgcacttcctctctgagcaggtaagatatagaattgtttggctcaagtgtcatttcttcttgaagtttcagtacactatgattgacctgctttgttttttattatctaattgttaatatacaccatagaagtagtcttagctggatcaaaatatgagtaacttttcctccctaactttctccatttgctttctgtaattacttttgtactacgtctttgtaagtttctgccttgaattattgttcatactggtaatgaaagcattggttgcaataggaagttagcaattttctcagaaatttacgttgcacattaactattttggttctttttgggtctcaatcaatgtatgtgcagtttctctctctctctctctctctcagccattttctatttaaccttcttcattatggaaaaaagatatttggtgctgatgttgatgacatgttatttgtcttgttcagaaagaggatgtccaccattcaataatatctgatcttcagaatggtaatgcagaaactaggaggcggcttgccgtgtattgtttaaaggtgcatagttttgtttatcttctgtggccctttttcctaaaatttgccttactttgcttctctcttttgctggcaattctttcattattgaactatatttgatgcattaaaatttgacaatttatttcttctttgaaacaaggatttgcatcgactagtctatcttgatattatagattgtgaacttgatgtaatgaaatgtagtgagtctgctacctgtttataaatctttagtgttactggatattgattgaaatcgtgtggtactttacttttggaattgcaggatgcttatctcccacagcggctcttggacaagctgatgtttatttataattatgtagaaatggcccgtgtcacaggcgtccctttatctttccttctttccaggggacaaagtatcaaggttagtgacctgtcatcgcacatcagttgattgaatcttacttgatcaagctaatcaaatcataggatgttgacctcttgttttcaggtactttctcaactcctgaggaaggctaaacagaagaaccttgttattcccaatgtcaaacaggcaggatctgaacaaggaacatatgaaggagccactgtgagtaaattttatctgcatggtcatgtgtactgttttaagaattacaatggctttgagtcgatagttccatctactggtttagccttattttatgtaaatatatcttgctagaaagtaaaagtatatggcttgcaatgttatacatcagtttctgactattaatttgtagttagaactgcaagtgccactatttcaaaatgttacacatcagtttctgactattgatttatagtaagagctgcaagtaccactgcttcaaaatatttggtttttgcatatcctgcattgtttaggcatgtgggtaatctgtctcttataaagacttgagaaatcttcccccttccaaattcttaatggtaataataaaaaatttacatcacataactgattcactaacttgaatactaccatagttgatgaagttgttgtattctttcttttgtgagtacgtgtgttcatgtaggtgtggacatggatgtgagtcctccctcgtgtttctctcttctttcacctcttcttttgtgccccttttttgggttttttgtgagttgggggggtgggtttgggtgtggtttaagtcttgtatcttggtaattgtcttttacttccccctttacaaagctgaatcatctttgccaatgatttggatctaacaaaatttcaggttttggaggctagagcaggattctacgaaaagccaattgcaacactagattttgcatctttatatccttcaataatgatggcatataatctatgttactgcaccttggtaataatcttgaccatttattctttttgttggcataacgattatggcttgtaatattggcacaaactttgtttatttcaatgttaaacttttgtcttttgcttgtcaaggtgactcctgaagatgttcgtaaactcaatattccacctgaatgtgtcaacaaaactccgtctggtgaaacatttgtcaaatcaaatttacaaaaggttaaatcatgaatttttttataatcatttttattggtgtcatcatcatcttgattaccaggattaattagaattatctttcagggaattcttcctgaaattcttgaagaactattagctgctcgaagaagagcaaaagcagatttgaaggtaattatctattgttttggctgctttattaaatgtttgtatttattattcctttgtgcattgcttattattcctttgtgcgttgccaattttttactctgttgtaatgttaccgctaggtttctaggcctttgtgctttttatgattttatctaataattttaggaagctaaggatccgcttgtgaaggctgttctagatggtcgacaactggccttgaaggtaagcttatagatacattgatctgtcgatgctcatttttagtgttcacaatctatttactatctagtgaggccaaagcttatagaaagttatacgccatcaagtggatggcctcaaatgcttttaaaatattatctcttttctctttaaaattgattgaaatattgaatttttgttactaaaaaataaatataagtgattagttaaaatttaatgaatacgttgaatgaattatttcattatatcccagaatatctaactttgagttaaagctaacgtttgtacttcccatatttctttttgttaagattcaataattagaaaatatattttaaaaataatgttattcttagatcaactattattgcttgaaacttgtattctttactattttatattcatgaaagaatatttagcatttccataaagtgtatggtcttatcatcttggaaattatttatttttttgtgattaatataatattttatgaagtgtctttaaaattttccctcccttggcttccctttttggttatgttaatgtttaatgagaaaatgacttgtttatcattctgttatatacaagtaaatacttgttggtttttcagtaatccaaatatttaattttatgaattttattgataaatttttgtgtttatcaaagtttgactatgtatatttaacattttcaaactaattgttttatattatttagtgaacttagatcactcattttgtagacgatgcaaaaagtgcagtgagaagactagtataataagataaagaataattgggcaatttcagtgtgttttctctgttaaagcaagtgtgtatatatacagattacaagggcaatttctaagaattctttattaatcaattagcctatgattatataatctcctttaattatgtatagattatgttcacaatctaatactctcccttaagttggagcatagatgtttatcatgtccaacttgttagaaaaatattctattcaaggctcatttaaagttttggtgagtagatcacccagttgctctcctgtcttcacataactactcacccagttgctctcctgtcttcacataactactcacctagttgctctcctgtcttcacataactagtcacataaccagtggagattaaattttcttgaatcttctaatggatgaaatgacaatcaatttcaatatgcttagtccattcgtggtatacttgattgaaagcaatgtgaagagcagcctgattattacactaaaatttcacgggtgacgcaacatccataccaatttagaatctcacaagtgaatcattgagccatggctctgtattctgactaggcactgaatttggatatcatattttgtttcttactttttcaaaacactaggtttcctccaacaaatacacagtagtctatggtcgaccttttatcacttttgaatctcgcctaatcaacgtcaggaaaacttaagtgcagtatgcccatgatgactcaatcctttttagataatatagaatttgttctagagcagtataatgtttcaccataaatgcagacatgaactggcttacaatgcttactactaggggtgagcagtattcgtttcaaaccgaaaaaaccgaccgaaccgaattaatttcaaaattcggttcggttttttattcaattcggttcggtttgatttttaatttcagaaatttcggttatttcggttcggttttaatcagaaaaaaattaaaaaaaaccgaaccaaaccgaccgaatgctcatccctacttactgcaaaagcaatagctgactaagtcattaccgtaaagtaattcagctttccaaccaacttcctgtacctctttggatcatcacaagggtctccgtcatcctttgttagatatatatattaggaatcattggtgtgctagagagtttagctcctattttcccattttctgcaagtaagtcaaggatatatttcttttgagatagaaaaattctctttttactcctcaagacttcgactcctaagaaatatttaacttgactcaagtttttagattgaaaacttgcatgcaagtagtttttgagaggagatcttatactatcattctctataatgataatgtcattaacatacacaacaaggagaataatactagtatctgaatttttataaaagactaaatgatcacacttgctttttttcaatccaaatttttgaacaacttcactgaacttgtcaaaccatgcacggagattctgcttcaaaccatataaagattttttcaaatggctgacttttccatactcccttgagcaacaatggttgcttcatatacactttctcttggaagtcgccatgtaaaagtgcattcttgatatctaactggtgtaaaggacaatgctgagaagttgttagggagatgaacaaacaaattgagatcatttttgcctcaggcgaaaaagtgtcagaatagtcaacgccataggtttgtacataacctttagcgacatgaaggctatagaaccatctggattgactttgatgacaaaaatccatttagagttcacaacctttttgtttgagggtagattaactagttcgcaaatatgattttcatctagacccttaatctcctcaaacattgtatcatgccatccagaatgagtcagggcctctttgactgtcttaaacaaagaaatggaatctagagagacaattaaggagatagaaggagacaggtaattataagacagaaagttagcaatagagtatgtagactttcattgtcgtttacctttacaaaaactaatgtgaaggtcaaggtcactcggtggtgaatccgatggcaaagaagattctagtgcaggacatgtatcatcaggtacttggtctccgagagtaaacttcggtttaataggaggctaagtattaggaggaatgttacaatcatattgtacaacaaaaggcatactcaaagagttttcaccatcagatatgattttataaataagtcgcacatcatcctcctttcaactagtagaggtttgtgtaacaggataaaaaggaatttgctcgaaaaaggctatatctactgagactagatatttgttgaggtttggagagtaacaccgataccccttttgaaggggatcaagtttagtcacatgaggtttgacattccgaacatagcactaaaaagtcgtggtttcagagaaaataatagcttcttaaaaaagggcatgcattaatgagaaagcattcagtagagacaatatcggtccaaaattgcttagaaacttgcatttgaaacaacagagctcaggcagtcttaaggagatgtcgattcttcctttcagctatcccactttgagcgggtgtatcaacacatgactattggtaaagaatattgttgggtcatataagtctgaaatgattttgatatatattttttagcattgtcccttcgtaaaatttgcacaaaaacattaaattgagtcttgatttcagcataaaaggcaaaaatataagaaaacatctctgaatgacactttataaaataaatcaaagccgttctagagtaatcatccacaaaagtgataaaatatatgaatctggtcttagatccaaccggacatgggccctgaacttctgaatggactaattcaaaaacagactaagctggtttattaactctagtacttaaagagtttcaatgatgttttgtaaaatgacatgactcacattttagtgaagatacctcatgaaattgagggcatagcttcttcaaaactaaagaaggatgtcccaaccgacaatgtgctttgaacggagacacgacactagagcagacaacgcattaaggcacccatgcatccaaaatggaaagacccaagatacatgtcttttactaataatttgttttgttataagatcttgaatgagacaatgattaggaaaaaaaaaacacaataatttaggtctttggtaagtttacttacagagattagattaaaggaaaattaggtaagcttaacacacatgataggataataggagtaggtttaacagtctcagcctcagaatcctcaatattataggttgatctatcagctatagtaaaaagagaatgtgctttatgagatcgaaagctggtaaaaatatgagaattacatgtcatgtgatctgtggcattagaatcaatgatccatttgtatgaagaggaaataagacaagtttacttatctcagcgtctaccggagacgttgagataattttaataatatgagttttactattagaaatcatttcaaccaaaaccttatattcctatgccttcaaatgaattaaaggaaaacaacagaacctaatcacgtgaacagtacagtaggctaccactgaaggacactttccaatcaatgtcggatggctggagttctactagagacgtgctggaaatgaccgggcgattgttggggaagaaaccagaagtcaaacggaggccacacatgctcccgcgcgctgtcgcataaaactagagctgaaacttggatcggcgcatgagcaccactcgcggaagctccagtcactgttttctgaggaacagtcggaaataatgttgcgtctctccttgggcctttggtgagatcgaacggacacccaaacttgatgacccaacaactagtttctcaacccgaaaaattcaaaaaaattagacatccaaaattagctcaaaggctatgatacaatataacaagacaaggaataattgggaaatctcagtgtgttttctctattaaagtaagtgtgtatatatatagattacaagcctttattaaggcaattcctaagaaccctctatcaattaattagcctataattacgtaattttctttaattatgtacggattatattcacactcgaacaactaacttagattcatgttggttcaggatttatacaatcttccattggtttgagctctttgattatcttatgttattgtaggtaagtgcaaattctgtatatggatttacaggagctactattggccagttgccatgtatagaaatatcctctagcgttacaagctacggtatgctaatacaagattctccctaattaatatggtgtttttttattcaatttgatctaacatgttgttggttgcaaaggtcgacagatgattgaacacactaaaaagcttgtagaagaaaaattcaccataacaggaggatatgaacacaatgcagaggtatgcaatctggaatttagatgagtattataatgtagtattttgaaaatgtttattggttaaaatgatgtagttattttaaaatgttgctccatttagaagtattttggtttgtttttaagagtttcacatcataaaattcataaatttaacctttctttgcattaaacacaccattttgttttattgtttaatccagcatcacacactaatttattatctttttgtccttgttttatacctctctgtttgatttgctgtcactcacactctctttgttttgtgcattaatcaatttgttcttattatagatgtgactttagtttcgtttccaatgctctcagtcctagtgccattttagtatcattatgcgtctttcaaaatcaaataatcttccaaattgaattttgaaatttcaaatttgttgttttggatttacacaatatcttagttcaattcaaacagaagactttaggtttgagaactgattgattttcactggaaaactaaattgaaccaattcaatttcagtttttgatttggtttaggagagagcattcggtcggtttggttctgaaccgaactgaatttgaaaaactgaaaaccaaagtctttaaaattataaaccgaattaaattgatattatagaaaaatcgaaccaaatcaaactgataaatatcagtttggtttgggtcaacaaaaaccgaaattcccaaattatagcaactagcctaagaatggcctcctcaaattgcaacaaccaacaagagaatgacacttaatattaatttgttccaaaatcaatatataacatttgtaaatagacaaggaataattgtgtaatttcagtgtgttctctattgaagtaagtgtgtatatatacagattacaagaccttgttaagtcaacttctaagaattctctatcaatcaattaacctatgattatgtaatctcctttaattatatataaattatattcacactctaacactccccctcaagttggagcatagatgtttattatacccaacttgttaggaaaatattctattcgagactcatttaaagttttggtgagtagatcacctagttgctctcctttcttcacataactagtgaagattaaattttcttgaatctactcatagataaaatgacaataaactttaatatgcttagtctatttgtgatatactggattagaagccatgtaaagagcagtctgattatcacaccaaagtttcgtgggtgacgcaacatccataccaatttcattcagcagatgatttactcggcattagatttggacactacattttgcttcttatttttttaagacataaggttttcttcaacaaatacactagtcgatcttttatcacttttggttcccactggatcagcattagaaaaacactcgagtgcagtatctcgagtgcagtatgcccataatcaccgtagactatactaagtcatttagttatgataaatactatacatttagttatgatatttatgaattatataaatactataaatttagttaaaattgcatataaaaatatatgcattttgttagttatttcattacattttgatattttattacactattactattattattgagcgaaaatcattattaaaatgcattacattatttttttaattaaatataatattaaaaatgagaattttaattatctaatgaattaaaattttatatggtactttatatttaattttcagattatttaaatgaattttattaatttattatgaaccatatatttaatataaaaataattttaacatttaatttataaaaaaaaattagaaatacatgaaaagtgagaatttattaatagttacagcaggttaaacacacacttctgcatgtttttatattatatgtggcaactatgcatgttaagattagtacaaaagcaaagaaaaatcctcacttgtagacaacccaaaaagcagaggaaaccactgcatcaagaagaaaaaaacggggtgggcagattatcttcaatgagtgggagggcaacaagagaaggaagaaagaagaaagagagaaggagaaaataaaaaaaatccaaatgatgttcttcaagcaaaagcagaaaagcaaggtgagagggaggagaaaagatgaaaaaataaaagaaagaacctggatttgcatctatccattcatgagtttggatttcctcttacttctttttttttttcttcttccgacaactagaaccgccttgtgtaactaggacgtgccttgtctgcgcctgcatgagggcgcctcccgcctgacaccactcaaggcgccagggctggcgcttggtgcgccttgcgcctcaagcgcgcctttcacaacactggtaataatataataaattatcttgttattaatattatgaaaataaaattttagtattggtgttagtaatatataataaataatatttatttatttataaatattgtctatttatagaaaattaatattaattttagttatatttatatattttttatttttttattatatatatatattatattattattctatgttctataatctatattgtaattatattctatatttatattttatgaaatagtgttttgaaataataatattatttattgtataaattgttatattatatagtatatataatttgtataatttagaaatttatatattatttttggttaatattattatttaattataaatatattatttataattatagttatatatatatgtataaatttatttatatatattaagaaattaatatttatcttctatatattatataaacataatttatcaccaactatattttatatattttaatataattgaagaaattaatatctagttggattaaaatatataaattgtagttagtgatgcattttatgcatatatacatgtacatttaaaatatataaaaaaattttttaactttgtccgcttttttaaaaaggcgttgccttgtgccttgcctcttgcctaaggggatagaaggctccatcgccttagtgtcgcctcgcgtgcaaaagccctagttatgaataattcgcctgagaacctgcatatgcgaaggtgatgcttcggcaccaagacatgcacagctttgacttggcgagagcatatggtgattggcaagaatggacggtgatagaaggctctattgccttggtttttcagttttgttcaaaactgaatcagttgattgcacacctagaagaaatacagaatttggttattttggtttttacaatttttttatcaaactgaccaaatgctattccctcatgcaaaagcagtcaattcttggtgccaactttaatcatattctcctgccgagatcattagcaacttccgcaaattggctggcttctgtttctgtggagtttatgattggagagaaggctagatatgttggtggaattggaagttaaataattttatgaatggagagaaagctagaacttttgatggaattgaagtttgctaagtgatgtgggattttaaattgattttgctgttctgctggttgtcttatttattcaccatatagatttattgagtatactttattaatgtattagactaggctatccttttctttcggtgcactttgttcctcgtttgttctaaaatccacaaagcttttggcatttcatatcaataacacattgtggtaagctttatctatagggaaaaagcgtcgtagattaccacgtgaaaaatattatgcatttttgtctatttggaaatttgaatttcaaatcatgaacataagaatgcaaaaatgagtttcatttacggaatatgtggatgtagtaagctaatcctctctattttacatttctatatgcttggtatttattaaaattttcttacatacctgttttgagtacaatataggtcaaaagcagtcctactttttatggtaatagcttgacctgatcgaagtgataatttcaggttatatatggggacacagattcagttatggtacaatttggtgtgcctactgttgaagaagcaatgaaattgggaagagaagctgctgaatgcatcagtgggactttcataaaagtaatttttatcttgaattctctgatttagaatttaactatttgatagtctttgcaaattcatttttagttaaatcagccaggatagctaatgaagagcctttgaaatacagcaactatgataagctttgaatggtaattttgaactccaattaaatgaattggcagtgtactgtgatttttcgcaaaaaaggaaaggcagggttgaattactcaattgagatattatttcctgaatatgaacttctcaaatatcaaaattgttagtttaattgttgttcttttttcttttttggtctcaatatagttgcctctgtctcacacatgcatttttttgtgtgattgttttagtgtagttttcttcatatgacttatggatttcttaacttttagcaaatgagtaaaataaaccaaatctaagtctgttattttccacgagattcctcttatacagctgaaattgaacacagccaaagttactatactggttttatgttcaatatttttatacctttaattgagtcaacttcacaaggttactcgcgtgagttggtgacatttaataattttttctgtgcaaatgttgctaggaagttattgtcggataatgctacagcgcatcatgatacataagttagaatgatgtctgaataaatcagtcatttatattttaaaaatagtcgtttctattttaaaaagataatcactgggggcttgaagactatctagtctggcaacagtgggaagagacatgggaattctcattatgaaatttgttgaacttggtttctgtttgctatagctatatgattttttcagatatggatacacctgtagtgttcatccaacttgaattctaattaaatgctaaggtttggaagctgtcaaattagctttatgtgattcaacttttggcctttttttttgccttttttttctatcaacttttttaatggttatcatttaactaactcatttcatttttgtatttgggtttttgaatgtgaagcctatcaagctagaatttgagaaggtttattatccatatctgttgattagcaagaagagatatgccggtttattctggactaatcccaacaaatttgacaaaatggatactaaaggtgaatacaacctctcaaacatctgagcttctggatattgtttgggaataatctattgacttttgtcatatatcttgggcaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaaggtgtgaaattgtattggttagggtcctacatactgtccttaaactgctgtataagaacatctaaatatcttgtgtcatcaacatggatattttcttcctgtcctaatttattttgtttaacagggtctgacaaagactggagacgattacgaagttaaggcagctcatgttgaacttgctgaacgcatgcgcaaggtgctttttttttagtgttcttttttcctcctttgtagaagggcttgttcttttgctagaattctggagagaggaattaaaagtctgggaccttaataatcttgtttttatttagataagttgagattaggctcaaaattctataggaatttaaaatcagataggatgggatttttagatttttaaagattttatttaaacttcttaattattctggtgggcaaaacataaagtactactaatctacgttgaaacaaggaaaatagcaaagaaaagaaactactgatgccatctgtaagaacctggatagattcttcgatggattttggagattaggtgagagagaaagtagatgtagatgagaagagaagggaggagaaaatagagtagatgagaagagaattaggagagtagagaggaggattctgttattggttattcttcaatgatcaatacagatagttgcttcttatttatagcagacccacaactaatttctgttgcaaccaattttccctcgaaaaccaacagctcacacacacagctgtactaagctcctctgtttttcctcctaaaatacataacaccatcttatcattttgaatgatattcaccaacatgaaacgctccaaggcacaagccataaagaagccaagaaaactatcttactctgcaggatgtgggaaaacaaagtttaataatctataatacagcataagatcttattctgtttgttattgacagagagatgctgccactgctccaaatgttggggatcgagtaccttatgttattataaaagctgcaaaaggtgccaaggtagttttctgggatttggttttgtttaatacctttttctctaaattacttggtacggtactctgattgttcaatttttatatgcaactgataatcaggcttatgagagatcagaagatcctatctatgttctggaaaataatataccaatagatcctcagtattatcttgaaaaccaaatcagcaaggttagtttggtatatgttttgtacaaagccaaatagtgattctttttctaatattcattaattataaatgttgatattgcagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattaggtatattttatttgtattccactttatctctgccttagcactacccatattggctgcaaggcactaattaggtacttccaaggtctctgtggatggtgggaggccctagattacttgctatagcttttcggtttcttgtacttttaattggtaaaaggtactttgtaatttgtaaatcaactgaaattgagatctctatttcagcgcctctatgacccaaagatttccttaaacactcatatggttgcattcatttactttcccgagttatataatatatttgagatatctgaggttttagaggagaaggcaaatttggcaccatttatttatttatgatttatcaagttcaatgattgcacgatttttcactctttttctcattctgcttagtgaacttctgttcatcacagactaagagtgcaaagtaggattctgtattctttctcacttacaataataccaattatttaagatgttttactatctccttaattgatcagatctattgtaatagagtgaaaatcaaatgcacctttatctctagatattttgtgcattcaaaattccatatgcgttttttatgtaataattagtgctccttttttctgcttttgctgtgggtatttcctcattgattatgggattctatccttctttgctgccatacgaatacaggcattatatttatttgaagagcctaatactattaatttggttttggaccagcaattcagatcgaactctctgcacacactgtaaaggaagagaagcagaattgtactgcaaaacggtttctcaaggtagacatctcttctgaattctgaatcatgataatttgtgtaattgtaatagagatggtgtttattaaacagaattcatggattgattgcagtgtccgaactggagttgctttttgggaggctgtggacacagtgccaagagtgccagggctctctccatcaggatgttctttgcactaggttttattttctttccattctgttaccattaaataatattctttgaaaaaaatgtttgcggggtttcctggtagctaaattattagctcaaattgtaactaaatattttatttttctttaataaaaagaaagaaagaagaatcacagagcacatattctacgatactttgtaatcactgatttttgttatgcttcatatgtccacagtcgggattgcccaatattctataggagaaagaaggcacaaaaggacatggctgaagccaaacggcagttggataggtggaacttctaatctgataacaaggttagtatcttttattctcggagtcattgtttagcttttacatatttgtgaattaacaaatctgatgttaactcactagggaaaattcggaagtcagtttgtcacagaggcctgcaatagttgagggtccaactgacttgaaaggttgcatatctgattggctagtattaaggcagaagccgtgtcgagacatgagttctaaaatggcagattttctggttgattgacctttcgcaggctttgatagtcgggatgtcgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttaattttggtctatcttggattactggtacatctctggccattttttgctgtaacttgaatgtcattacgatagccgttctaacagcgcatctttagctgagccaatataatatgacaagtaaagccatgctatttcagtttgaacgttgtggaaaataagtgtaagatggtgtttggcttaatttatgattataagcaccaaatgtttattaactattgaaaatttataagcatttaaaattttaagtgattttg

SEQ ID NO: 3 - DNA, 4063 bp, Manihot esculenta
  source 1..4063: mol_type = other DNA
ctatctctcattctcactctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacaacccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgtccctctctcacgagtacacttcccaatctcgtagcattatatttcagcagctggaaattgattatgtgattggagagagtcataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagctgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttatatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcacaaaaggacatggctgaagccaaacggcagttggataggtggaacttctaatctgataacaagggaaaattcggaagtcagtttgtcacagaggcctgcaatagttgagggtccaactgacttgaaaggttgcatatctgattggctagtattaaggcagaagccgtgtcgagacatgagttctaaaatggcagattttctggttgattgacctttcgcaggctttgatagtcgggatgtcgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttaattttggtctatcttggattactggtacatctctggccattttttgctgtaacttgaatgtcattacgatagccgttctaacagcgcatctttagctgagccaatataatatgacaagtaaagccatgctatttcagtttgaacgttgtggaaaataagtgtaagatggtgtttggcttaatttatgattataagcaccaaatgtttattaactattgaaaatttataagcatttaaaattttaagtgatttt

SEQ ID NO: 4 - DNA, 3276 bp, Manihot esculenta
  source 1..3276: mol_type = other DNA
atgaaccgaaatggcaacaccaggaagcgccccccgccgacatcacaacccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgtccctctctcacgagtacacttcccaatctcgtagcattatatttcagcagctggaaattgattatgtgattggagagagtcataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagctgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttatatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcacaaaaggacatggctgaagccaaacggcagttggataggtggaacttctaa

SEQ ID NO: 5 - protein, 1086 aa, Solanum lycopersicum
MNSGNSKKRSAAATPSPQQQQKHHAAMEEEDLDEDVFLEETLLQYEEDSQVLRELEESNSIKERLQKWKRPSLSQAYIAQTQSISFQQLEIDYIIAESHKELLPNSSGQAAILRIFGVTKEGNSVCCHVHGFEPYFYISCPPGMGPDDISRFHQILEGRMREANRNSNAPKFVRRIEVVQKRSIMYYQQQASQPFLKIVVALPTMVTSCRGILDRGIQIDGLGMKSFLTYESNVLFALRFMIDCNIVGGNWIEVPVGKYKKAARNLSYCQLEFDCLYSDLISHSPEGEFSKMAPFRILSFDIECAGRKGHFPEPTQDPVIQVANLVTLQGQDQPLIRNVMTLKSCSPIVGVDIMSFDTEKEVLRAWRDLIREVDPDIIIGYNICNFDLPYLIRRAEVLGMAEFPVLGRIRNSRVRVKDATFSSRQHGTRESKEITIEGRVQFDLFQAVQRDYKLSSYSLNSVSAHFLNEQKEDVHHSIISDLQNGNAETRRRLAVYCLKDAYLPQRLLDKLMFIYNYVEMARVTGVPISFLLSRGQSIKVLSQLLRKAKQKDLVIPNAKQVGSEQGTFEGATVLEAQAGFYEKPIATLDFASLYPSIMMAYNLCYCTLVTPEEVRKLNLPPECVNKTPSGETFVKSTLQKGILPEILEELLTARKRAKADLKEATDPRVKAVLDGRQLALKISANSVYGFTGATVGQLPCLEISSSVTSYGRQMIEHTKKLVEDKFTVLGGYEHNAEVIYGDTDSVMVQFGVPTVEEAMKLGREAADYISGTFIKPIKLEFEKIYYPYLLISKKRYAGLLWTNPDKHDKMDAKGIETVRRDNCLLVKNLVTECLYKILVDRDVPGAVQYVKNTISDLLLNRMDLSLLVITKGLTKTGDDYEVKAAHVELAERMRKRDAATAPSIGDRVPYVIIKAAKGAKAYEKSEDPIYVLENNIPIDPQYYLENQISKPLLRIFEPILKNASKELLHGDHTRSISVPTPSNSGIMRFAKKQLTCIGCKTPLSGSDRTICKHCKGREAELYCRSVANVAELENLFGKLWTQCQECQGSLHQDVLCTSRDCPIFYRRKKAQKDMAEAKTQLDRWNF

SEQ ID NO: 6 - DNA, 20888 bp, Solanum lycopersicum
  source 1..20888: mol_type = genomic DNA
atgaactccggcaactctaagaagcgatcggcggcagcaacgccgtcgcctcaacagcagcagaaacaccacgcagccatggaagaagaggacctagacgaggatgtatttctcgaagagacattgcttcagtacgaagaagattcacaggtccttcgtgagctcgaagagagcaattccataaaggagaggcttcagaagtggaaacgtccttctctatctcaagcttacatcgctcagacacaaagcatatgtaagaaaattttaattgagaaacattttgctcttaatttccgacttcatgccttcgttttttttgtttctgcttgtttaatgtattgttcgtttctttttctggaagctcttttttcttgtttccctagatttttaaattatttggtgaatttggtggagtctatctcaattatggtgttgaatgtttatggtttctgaaggtaattcatttggtgttaagcttttcttacaaaggatttcaagtcagctggtgcggagtagcacggcccggctgctcttttcaacaatgtgtgcagatgttttatttgatttaatttgatattatagcgtgcatttgatgtaattataatatagtttacgaacttcagtatataagcatatataacaacaacaaaatacccaatataatctcgcaaggtgggtatgggagagggtcgagtctacacagatccttgtaagggtagggagactgtttcctatagaccctcggttcaagaagagatgataagtaaacaataataacaacaagatagtaaaataaacaaaatggaagaaacagacaagaaatactagagatttatgataaaacaaacttaaaaatgatagtaatactcaaggatgaaaatgaaaattgcttggcaacctactatccctctaccatgatactcaacctccacaccctcctatcaagaatcctgtctttggtaagttaaagttgcaatatgtcttgtctaattacctttcctcaaaatttcttagtcctacctcttctcctcctcatgccatcatgaccaaacctctcacctccataacgaggcaactgtgtttttcctctatatatgtctgaactatctcaacctccctttctacatcttatccaccacggggttcactcccatcatgttgttgtcctgaatctttttattcttaatattatctctcatgatgtcctcacacattcatactttcatcctattgacaaactttcttaattagccgacactctgcccaaacaacatagttgacctaaccaccactctgtagaacttaccttttagtcttaacggcatatactaatcacacaaaacaccaaatgtgagcctccatttcattgatcccgctctgatacgatgtgtgacaccatcgttaatcttcccactgccttggattactaaccccaagtacttgtaacttcctctctggggatgacttcagtatcaagccttacgtccccatctgcttcattaatcacatcattgaacttgcagtccaagaattctgtcttagtcctgctcaacttcaaatctttagactccagggtctgtctccgaaactccagcttcgcgttaactctgctttgcgtttcagtcaatcatgaaagagaagtgctttgagtcccctctcactgtccttacccgggtcttggttccaccgtacatgtaatcactctaaggttaactacatggacacctctcacctcccaacatctccataaaacttcgcttaggactttgtcatatgcttttcaaggtcaacgaacactaaatttaagtccctctctctttacattgtatatagttttgacttaaaaaggaatgagtgagcctacacttgtctaaaagctacatctgggcaatgctctttaactcgagtttttttgcagttgagatttagtcccttttatgtacttctgattttttaaaacagttaagctaaactagtctaaatgtgtatttggtaaggaaaatattttttgaaaattgttttccaagaaaatatgcgggtttcttatttgtggaacttgttttcttatgcccagtacgaaattcaatttcttcatattttaggaatgggaaaatattttctataaattttgaccaacatgacatggaaaaatagaaaatataatactccataccaaacacaacctaagtgtgtatatgtggaagccattttcgtttattataggaatttgaagtccattttactttaaaatttaagttcaagattcttattttgtagttctttttatttatatatttaattaatttggaatattcttatattttattctttccttggtttgtcttcatgtaaataaatggttctgtacttaattaaggagtataattcaaatttttttttgttttcattgttttatgactctcatatttattaaatatcagaaaacctaaaataaaattttgaactttgaaggcagaataggaagaaaagtacctacttttggattgctcatattgacattagtctgatgccgtgtataggtattgacaaaaaatagaaacctgttgttgctccaaaaattttgagccttctctttaccaagcaaaaagaaaccaaaaattttgaagtcttttcgtgctcttggtatgagcaacaaattattttaattcctgtgctcaaatggaatcctattaagcaagtttttatattgtcggacttggtggtggacaatctatgactcaagtagagtcacaatgcacgtcttgtacttattatcttttgataactattcaggtgtttattaatgaataattttgacttaagcgtttgtatattgagttgagggcaatgtgtacatgtggatcttataataatttagtgaacaacaagaagttggatttttaggtaaaacaaaaatgtttcaaaatacatcaattacttcatttagctctaactttcacaatcacattctgaaagttccgttgaaatttggtggtcctttctcagcgtttcagcagctggaaatcgactacattattgcagagagccataaagaattgctacctaattcttctggtcaggctgccattttaagaatcttcggggtcacaaaggaaggtttgtcaattttcaatgctgttgtcatatgcatttgcaattcttgttgttgtattgtagctatggtgctattgatctagaaagatgatttattctggcattccaagtaatataatagtctcctagaggtataaatgcacgccatgaaatgggagttgttgttttttaatcggtgaagctgttttttcataaattaaaaatagttgagagcgttctgagggctgcaagcttgtgaggttcgctgataccaaccacagtttcaaacatagattttctcttgactgtgtataatgtattggtacaaatttgagttgaagttgcatcttagtggattctctagttgtaatattttccttttgtgtttctgttctatttgtttaaagaggttcttgtgccatttcctttatagaagaaaattatagctttttttttagtattttctatgctatcatattacactagatattagaactgaacatgaaaagcttaagtggaggttgtacttggtagggtgaggagtgagttcagaaggaacacaataccttggaatccatcactaatatatttattgaggttgacgtagcttgtcttccacttactaagaaatgtctgtttatgccctggtcaggaaatagcgtctgttgtcatgtacatggatttgaaccatacttctatatcagctgccctcctggaatgggccctgacgatatctcccgttttcaccaaattcttgaggtatgttgacctgcttgtttgcataaacaggctatgattgaattcttcaccagtttggtttattaatggcaaatgctgcagttgctatattattcttgttggttggtgtagggtaggatgagagaggcgaataggaacagcaatgcccctaagtttgtccgtcgcattgaagtagttcagaaaagaagcatcatgtattaccaacagcaggcttctcagccttttctcaagattgtagtggcattgccaacaatggttaccagttgccgtggtatattatctgtttcacttattcaattatactgtatttttctgatgaaggtgcttttttgattgagtcttatcttttgtgcttgtggcacacgcctcgggacacagcacacacagtaaaagtgttattgatagaaatatcaagatgtaagacagtgctggaaagtagatgttttcttatgttgttcagttgggttaattttacccctgtatttagcactgaacaactagtgaactctatcattgcgtcctcatatatatatatatatataatattttaagattgcaacagaacctagaaagttgggaagtccattaaaaagatgtctttgattcctactagtattttgtggtgcttacggattggatagagaggaacaaaaggtttattgatggcatccgaactcccatttcttctctgaagctaagtatttgatcagtcttcttagttgggtcaaccagttctctgtaattactgtagagtctttcctagaatttatcagctccgtagatctatgttgatagttgtgaacttgtgacagctttttttgccttcctgggagcagataatctctttgtaatgcttgcatcttcttgatgctttttgttaatgatatccttcttatttcattaaaaagaaggaaaagattacaacagaaattaacaggctaagtcattcatgtttcaaactgcaaacagctgcagtttagatcttgaagttctggcttgtttccttccaaaccacctaacatagctgaaaggtactccaacgccctctgttcatttctcattttacatccagtccagctgtaataagttcttagactgtccttggcattacccattttatcccaaaagtgtttaaaaaggtttctcaattgagcagttgtgtggggttgtgctatatttaatatggcattttgcttgccagtcttgcctgctcttgccatgataatttaatgtattccttgacattgagtcatataaataatatacttgcatcttatgcttctggtaaaatttctccttaattttctcacttgagtttacttgctgaaatctggtgaataatatgttcgacagaatttggattatgtttttcaacatatggatgcaacagaattgcgataaaatgtttgacttctgattgattttatagaattgattctattaccgattccttttcctgcttttcccacctgattgaaatgtaaaatggggcaggtattctcgatagaggtattcaaattgacgggcttggtatgaagagctttcttacctatgaaagtaatgttctttttgctcttcgcttcatgattgactgcaatattgttggtggcaactggattgaagttccagttgggaaatacaagaaagctgcaagaaatctctcctattgtcaattagagtttgattgtctgtatcctaatgcatgatgaatatatgacgtaagagtgcattctttctatccttaagggatcatcatctggaactttcttaacattttcttttagctattctgacttgataagccattctcctgaaggagaattttcgaagatggctccatttcgcatattgagtttcgatattgaatgtgctggtcgtaaaggccactttccggagcctacacaagatcctgttattcaagttagtcccaatttattctatatatctggaaaatatatgttgtttactttcctattgctgataagattttaacttcattctaggtggccaatctggttaccttgcagggacaggatcagccattaattcggaatgtgatgacccttaagtcatgctcaccgatagttggggttgatattatgtcctttgacacagaaaaagaagtattgcgggcttggagggtaatattttttgcatgaaatttgtattttaattagatacagtttccaccttttctcttcgtttgtgtggtggtggaatgggggtttgttggacagacgagcagagattttgtgtgtgacagttgattctttcaatttggcttcatgtgatggcacttacccgtttacacggtttcttttagcgtgaaagtttcacattgtccgtgttgttttatgctccagattgtaatgcacaagccgaagagtgaagcaggctgaacagactaatttggacaagttagaagttaaatattatggaaaaaggagtagaaataatgacggaaataaactttcatgggctatttaaacttcccttacttggcatatgaatcttggttgattggtgctgaggaagtggtgggactttctcatagttaatatgaacatagagtgtttggatatgccatttcagtctcatacattagtttgcacaattgcataagactattccagaatactttcctgaaaacaatattttgtaagaaaaagatgtctctactattagtaataccgatgaccaattcatgttttctcttaactttcttttgacttgcaggaccttatccgggaggtggaccctgatataataattggatataacatctgcaactttgatttgccatatctcatccgggtaatgagcttctgttttcccttttttgattggtaaaaggtattgtattctgactagcaagcttcttatgcagttatatgtttaaataattgctccaaaacaataatatgagagctaaagtgaataagatttgctgggcaacttcataaaaaaaaaaagatttgctgggcaagcttaacaatttgtttgttaatatagtagccaaaagtattgtttcttaattattaactggattaagagttgaggactataaaaagtccgtaggcagaagtgaaagaaagcactacatgataaatcgttgtttagctacaccattgtggtcaatgaagtgggaaagaactatgaggtctcagcagagacaaaaacataaggtgatttctttccatttgttctatccttggtggacagagttacttggttcatgttgctagtgggatgtgacaggtattccatagaattagttgagatgtgtgcaagttggcccggacaccacagttaataaaaaattaaaataaattgtggtttaactgtgatgctgatggtggaactgaagtggaaaaaattgtttcttcccttcccaaaagagattgggaaaggctaggtcatgtttgtttttccatgtgtaatgttgaactactgaaggcattttatcttcttatcagagagctgaagtcttagggatggcagaatttcctgtgcttggacgcatcagaaacagcagggttcgtgtaaaggatgcaaccttttcttcaaggtcattaatatctttttgtttgttatcttctgtttgtttagaagtacttttaggtttctactttatctgtaggtaatacaaaatggcatttttttccgcttggtgtatgttgactatatttgtatctaaccactggcctgttcagttttggatttatgttccatcactgtatgtggtttctgaaatatgggattatatactaacctaaaacatgttccaattagccgctgcttatcagaacctttgtaatgtttctccttgtaataggacagtaaaatggagtaactggtatcgcccatatttttcctcctagagcattgtttcatttccataggtggagttgcagcacttcttaattcaaacatttcccaaaaattcgctcgattgaaactcaaagatcagtcttcaactagttgattgtgatcgtaaaaggaaatttccctgaggctatgctgaaattgatttaatcttcatctaagtgtgattagggttgtcaaatggatgagtcaaaatggcctgggttgataaaataaccaacccaatatttatttgcgctgaaatggactaaacagcggttcataacccaacccacccaattcttgctaagatttaatttccttgtttgtacttctattaaaaaaaattacctattaaactgtttttccctttactagagctatatatatgtcagaaaaaatgttgttatgagaatattttttgacaaaatgtttcatgggtcaatttgggctatcaaccaacttttttgatggtctgagttaataatgagtgggtcaacttgggagggtcgtgcttttcatgtgctaattttgccactcctatttgtgatcatcaaagtctaaaagaaaaaaatatcccaagggtgacaagtacaattgagttttgcgtttagtgaagctgactttatcaaaaaaaaacttgagaagctgtggactgatgtatgcgttttgtgttgcagacagcatgggacaagggaaagtaaagaaataacaatagaagggagagttcaatttgatttatttcaggttttgttcattatggctgtatcaataagctgttctaattgctgtagaaagaactttacttaggaaaacagttgaatgccagtaaatctgttattctcgtgtattgcaggctgtgcagagggattataagctaagttcttactcactaaattctgtctcagcacattttctgaatgagcaggttagttttgctcttctatccatagtttcttgtcacaactcacatactgctacatctgcccacaaccatgtaccttctgccgataaatttttacatgattttgagcgatgaccctacatgcactcttgtttatgttcaaacacttcaatgctctttatttttgctttatgtacccgatatcttaaaactcactggtccatttaatcagagtcatgagttgatccaccataggagaagcgttacctaccaggagtttctgtgacctctgattaaagttggagggatgccatccatatcacgtctctacttatgttgataactaagttgagtgattccattagtgccattgctcatgtatatttaagtaacattagtaggtcttgcggtaatgctttgtagtttttttgtgtacagggtttgttagcttgaaggattacaagtctacttgctagctttctattttgattttggcatgttaattgtgaaattaatggcttgagctgtatttgatctacatgagttcgtaaagaaaaaatctgtgattgcgaccttgagttcagtaaaagaacagaagaaatactcaattttgtgcaagaaacatgtgtaaaaagttgtgattttttttaaatacaagtgcacttgctaaactctgttccttttccagtcaccacaacacacatttatgtggaaaaaaaggtaacttcaacatggtctcgctaccagctatcacataaaaatgtcaagtaccattgaacaaaacatcttaaatttaataattttaagtatcttatttctgctttagtgtctattcggggctgcctagatgcaatggaagcatgaccagttggagagagaagattccagtgagcaaggaggagttgggggaactcgcaactatttgtgtctcgtgttgatatggagaaaaacaaatggaagagctctttttttttttggaaaatgataatgtcaaatggaagaactcattttaggaataaaatagggagagccatgaatcaaagatcaaatgaggagatcctattcaattaaattgttgttcagagtagccatgaacagcaacgagaagcgcacacagcatcgaagaagtgaggattttcattaattgcccattgttcaccgtctttctagctcaagtggaacaacaacaacatgccccacaagtggggtctgtgattggtagtgttacacatactttactcctttcctgggaggtagacaagttaattcccatactctcggctcaagaaaaaaacataacaacgcaatctggggaaaagaaataactgaagtgaagaagcgacggcaaaatactaaagatagcatgacaaaacattttggagaaaaaaaaaagaaataatatctataacaagaaaatactataatcaaagtacaagaaataacatatactgatagaaatcggagaacaagaaactataagaatactgctacaactactagtactcaagcacaagtgagacaacagtcaactacttaatagccttcttcactaatttttgtcttccatagactcctatctaaggtcatgtcctcggtaagttaaagttatttcatgtccagtctaatcatcttccccgtattctttggtctagttctacctctcttgaaaccattttaaacccaacttctcacacctctgcattggggcatccgtacatttcctcttcacatgtttgaatcatcttagtctcgaatctgtcttctcctccacctttacccaaatatcttcatttctaattttatctctcctagtaagcccacacatccatcttagcgtcctcatttccacaacttaagtcttctaatgtgagaattcttgactggaaacactctgccccatacaacatagttggtcatttctagctccagtgcattctttaattaagttctctgcgccggtgcactattatttttattaaattacttgtgaagtttaatttcagatctttatccagtggggcagaaaggatgacatcttaatcatattgttttgacctttattataaacatatcattgtatttgttgttatctgaaaattgagatggagggtcttttatacctttgttttcatttatgttcttttatgtccataatctgttcattcgtcttgtttcccctatccttattcctattgaatatacttgtcttgtttagagcaagtaatttttttctaatactactgctatgcctgcagaaagaggatgtccatcattcaattatatcagatcttcaaaatggcaatgcagaaacgaggcgccggcttgctgtatattgtctaaaggtgtatagttctgttccggttgaaggaaattattgaattggatttgattgtgtgctgagtgttgcttatttcatagaatagttaattcttttagattttttttgttttagacatgcatacaattttctttagataagggatatatgtaaaattctgctccaagaatatttagttttaatttcttaatgcttatagagacagccttttggtgtagaatgttccactcgaaagatgtttgcatgtaaagaaaccacatataacttcattgttttcatcaaagaagatactgttacaacattaagggtgtgatctagtggtcaataaagatagaactattgtttgattttttattatgccaaatgttgatacacctctgtcatcctttcccaaatacagttggacgtaatgattgtttcatgtagataacgagcaatgagtagtttggccaatgaattatagaacgtagtaattggtattctagatgagttcaaatgaaatttaattttctgatgtgaatattcagtctcacttagcaagaaaactgagtgctttagttccgttacaggatgcttatctcccacaacggcttttggacaaattaatgttcatttataactatgtggagatggctcgagtcactggtgtacctatatcttttcttctctcaagagggcaaagcattaaggttagtcctatacttgcttcattttcctctagtgtaaaattaagtaaggtctcatttatttgcaattaatgaaggtcttaatcttaattattctaatctcctctatcttcaggtgctttctcaacttttaaggaaggcgaagcaaaaggatcttgtcataccaaatgcaaaacaggttggatctgaacaaggaacatttgagggggctacggtaagaaaataagtaacacccatcatatagctggatattttatcttgtaaagaaaagttagcacttgtaaagctcctgttgtgccaagctttatatttggtatattactcttgttaaacatatgtttcgagtattagttgaggtgcgctcaagtttggtcctggacaccatagttatcaaaaatgaacatgtatgtttggaggatatcaaatctgactatcacacaccttttatatcctttgatgttgatttcttaggttagaggtactaactgtaagtcttgattcttttgtatttttcctttcaaaatatctggtcacaggttttggaggcacaggctggtttctatgagaagccaattgcaacattggattttgcatctctatatccatcaatcatgatggcatataatttatgttactgcacgctagtaagtggtcttgtttgagtcttgaccatgatatttcttttttacccgagtgttcaattgatgttggtgaaaccttagaattaagtgctgagcattaagatcttcacgttaatgcttccggttctaagttttggttttcttccctcatgtctaggtaactcctgaagaggttcgcaaactgaaccttcctccagagtgtgtcaacaagaccccttctggtgaaacgtttgtcaagtcaaccttacagaaggtaaatatttaactgttggtttaattccggtatttatgttcttgtttcgattatgataatttaattgttttgtcagggaatacttccggaaattcttgaagaactattgactgctcgtaaaagagcaaaagcagatttaaaggtgttcactatatttttgatcttttatatttttctactatctgttgcctcattttgctttgtatcttgttttttgttgtcattttgtttcttagaacccttctctgattaactttctttttgaaccgagggtctatcatatacaacctctctaccccacaaaggtcggggtaaggtctgcttacaccttaccctgcccagaccctacttatgggattacattgggtatgttgttgttgtgttcagtctttggaatttggttccatctaatgtttgaaagaatcattctggatttattgggttttgatccaactagccttgggtttaatcaaggacatcaatcctccacatctatgataatatgcagttggcatatgcatatatctcaaatgttttaattctatgggtaagagacaataactaagcatagagaatccaaaatctgtttttttttggttaaatcaggatacttgattgttgattgtgaagtagggcttttaactttttacttgagatttccttttgtttgtatataggtacactcaatactggcctcctttcactagatttaatggctctaaaagtttcctttttaaggaagtgataaaaggaattcgtagtgttttcatttgtcagtgttacactactgcaaccaagcacttggtgtatatttttacatattcacatcctagcactgagcatcttatttccacaacactagtattagatgctattggacaatgcagatgactagtttttttcttcttattttcttgccgttaaatggacaaatcattttttcttcagaagtttgattgtcatggaaacttttaaagtggctctggaccctcttgtaacctgatatgttatggcaagattgtaaaatccataactacttgcaaagagcttctgagggacacaatgtaatttcatgctatgtagattctttttatccttatgccttgcatttgaatagatgtaaattattaaaccaacatgttcctctcatgttatgtcatatttattaatatttgatgcaatatgatgtgtagcttgattctagcataagaaagttccattttaccaatctctctaaacttcggttctttaaattgtcgtttgatccacgagagttctcaatttgccaccaccatggctccatatccagcttctgtcaactgcatcttttttcttaattttctgatgcatcaaattagtataaagaatccagtttcttgaagtgaactgcctatctttttgagaagatattgttgtattcctcagcattaaggatatgctggagacctccaaaaatttgttcccaacaagagaaaaaacaaaaaggctaattgattacgaagtgccaataatatccaaaatttgtgcagcttcttcaatcccttcctctttacaccaaaaatataaagtagtcaggcagttccatttgactttctctattgaattagcattgttttcaaaacatctgctatttctttctttccaagtagaccaccaaatgcatgatggtattgtactccactttttcttagattttctcccccctctgctgatccagcaactaagtagatctgttgtatgctcaggcataacccattctgtgcgtgttagtccgagaaaaaggttccacaattgtgttgtatttatgcaatggaggaacaagtggttgttggtttcctcagtctcagagcagcaaaaggcaatatagaaactcctccttttcaatttctcatgtgtggagcatgctcttctggcaaccaaccaagagacactttatcttggttggcaccttgttcttccaaatctgcttccacgggccaggcttaccacctggatgctccttcacctccttaataatagcttgttaacagaaggcttccatatctagagtgtttccatatggttgaatctttttgtgccggtaacccattgaacccgttcagaacatgtaatagctcagccactctcccaatcttccagtcatttaaaggccttctaaacacaatattccatccatgagtagaccatacctcagccactgtttcttccggattggtacacaaggaaactgatctggaaataagaccatcagtgtctcatttccattccaattgtccttccagaataggattttagtcccatttcccatcttgtagactatttgtctacttaggtcaggccaaaaatttctgattattctctaagcttgacaccctataagtgctattcacagagctgaagacccaatttccattcaacccgtacttgttgactatcacctccctccacagggcttgttcctcagtgttgaacctccatagccattttgaccaaaggcatctattttgcaaccccaagattctgattccaagtccaccggattgtttatgtaactgcgcagtctgccatttgactaaatgcaaatccttcccttctttattccctatccacaagaaatctcttctcagtttgtcaagtctttcttccactttggcaagcgtaggaaataaagacataacataagttggaatattgtcccagcttgcatttggtggactatatgaataggaaggaaccaaagatgtttagaaaacaagagttgttctctgcagaaactgaagatgaactgtctagtccttttctttttttggtgtaaacacggatatcctcaagaagaagaggatgttcccagtaattttgttgtaagtattactggaatactttctgagtattcctttttgtttataatacaagttaccttcttcaaaaaaaaaaagttggaagagagtccaacacagagtttattgaagtgactgtccctcccaaagagatatactgagctttccaattggccaattttttctgtcttttaaattatgccattccaaatttcttgctccttgtgtttgtgccccaatgacatgcccaagtaagttgtgggaagctggtgaactgcctatcagaagcccgatgtttttacttgagtattagtctactcaccattttttgcatatctaacagatgatgtgtagataaatatgtgttatcctctgaactttgaattatgacgatatattttgggtttcaggaagccacagaccctcgagtcaaagctgtcttagatggccgtcagctagctttgaaggtaattcatgcattgttagtcaatatccaactttgtaactgtgttctttcactttttcttggcagatatatgaatttggttctctttattattggtactacaatgattttagaactataaagaatttagtcaggtcaataactactgatgtcttctggtttcagatcagtgcaaattcagtatatgggtttactggagctactgttggtcaattaccatgtctagaaatatcttcaagtgttactagctatggtaatgcaattattactagcaactttattacatgacactgaactactggaactttgttgtatagaaagccaattcaagtacttgctttttgaacttgtttgttcttcctaggtcgacagatgattgagcacacaaaaaagcttgtggaagacaagtttacagtacttgggggctatgaacacaacgctgaggtacattatctgaagtttttatagatgttatagattcccaattaatatcttatgggtgttgtcagagtgtcactggttggttagcactaaattattaactttatcaaaaaattgtcctctaattgtgattgttttgtgatgccaggttatatatggagatacagattctgtgatggtgcagtttggtgtgcctacagtggaagaggctatgaaattgggcagagaagctgctgattacattagcgggacatttatcaaagtgcgtcctaaacttcggtatatttaattgtatctaaaccaaccttttctaaaaaaaccagtagcatctaaacacttcttcagatgatcacttcagtgcagtttctttgttcttcgttaaccatcattttctcttgagacaagtctgagcctgcttacatgccacaactacagttatccatgaagtatttatattctctaatcagttggtcagttattgctatttgatgatgcccattagttggttttggtatttcatggttatagcatatttgtgattagagtatatatcatgaagaggaagaagtgaaaaagcatcagaaactagtacacttattgtattcgatcagttcaacttcttcttgataagtagtattgatatacttattccacttgatgcaattttgtaaatttatcatggtttatggtattctagtgaggaaacttgttaactcgtgagatccttgctaataaagagaatcctaaaatgtttaaatattgagatgtgttctcagcagtgtacctatgtatttttttaatctcttctactaaaatttatttttcattttttccttctgctgctggtggcatatttgattttcagccaataaaactagaatttgagaagatttattacccatatttgctgattagcaagaagagatatgctggccttctttggacaaatccggacaaacatgacaaaatggatgctaaaggtgagctctgtcgaaggcctatccttttcatctttcagcatattcttggaacttctctcacccttcaatgccaacctttaatttttttcatattgttatccttgaaaaattagcaaatcaaatgtgttacctcaaatttaactattcttactgtattcaaattgttcatttcttaggtgcatattgtgtttgataaattgttacagttctaccctttttcaccagggattgaaactgttcgaagggataattgtctcttagtcaagaaccttgtaaccgagtgtctttataaaatactagtagacagagatgttcctggtgctgttcagtacgtcaagaatactatttcagatcttcttctgaaccgcatggatctgtctcttttagttattactaaggtgagaatactcttaattcaggaaaacctggggcatttatttcttgcaacttgtttgaatattttggagttgttagtttatgtgattagcattatctaacttctgagggttaatagggtctgacaaaaactggagatgattatgaagtaaaggcagcacatgttgaacttgcagagcgaatgcgcaaggttggataaactcaatttatttgcatatctttttaaaaatataagtagattccttcaagaagggttcagttacttttgtcctttgtctgtgaagatcgatgtatggcatcttgcacacaagtatatgctgcttgatatttgttcgattaagctgttgagtttcccatctaaattatgttcccattatgtattgaatttgttccttatttctggtggcaaggctgctgtcatccaaatttttgatggtttaatattcaagtactctaaataaggctaaaccagacgtcactatatttgagttatcttgttgtcagtagctcaacttttctgaagttctgtgctgatttttgtgccaaggattccagtattcctcatatttgctgttaatcaaatatgttggaagattaataactccattatttgcttttgttaccctatgagctatgttagaacatatgcttgtccttcattggatttggattaggtgttcagactcctttacatagtatccaagtcagacttgtaagcacggtttcacaattgtttctcattaatataattgtgaagaaatgaacgttggggttagttcaataccaaaagttagctcatgaggaggattctggaggcctatataaggagaccacaaccttaaagcgcacgaaggggagaattttcaagaccatataaagttagggcaacccttataactcatgataggaggatttcctgactattagaaggttcattgtactcagaaggaaatagaaaagtgcaccctcaagggtgtgccctagaggtcaatgaagtgggttgagaatcacaaggtatcatgtttaaatctcagtggaggcaaaagccctattcttacctcttgtttgacagagttaagcagtaccttgtggaattagtcgaagagtgtgcaaactggcccagacctcactctttatctaagaaaagaaaaagggaagcacgtttattagaatatatagttccactcgtcaaaaaggttgatcagaatataaagttactaaaaggctcatacctatccttttgttttgttggcttactttccttctccctcttgttaattcttacctgtaatggtttcatgtgattttataagatataatttgttcaacccaaagacaatagaaggtaaaaggaacaggttgtctaattgacattcattttaaggtacattttatatagtgtcgtgttttgcgtgctctcttttaacattgtattcaagtggcagagtgagctgcgcatttaactattgcagaaaccacatttctgagattcttatggttttagcgcaccataaaaaatgaaattgatgaaaacctttattttcaatgacagcgagatgctgctacagcacctagcattggtgatagagtaccttatgtaatcattaaagctgcaaaaggggccaaggtaatcatccaactgatttagtatgtgattataatctccacattttgtgtgcttgtgtcaaatcacggttatattatgacaagcaggcatatgaaaagtcagaagaccctatatacgtgttagagaacaacatacctatagatcctcaatactatcttgaaaatcaaattagtaaggtcagtatggctcgattttttcgttttaattaacatgctcattgtaactgttttaatggaagtgttttatatcagccacttctgaggatttttgagccaatactgaagaatgcaagtaaagagcttcttcacggagatcacacaagatccatctcagttccaaccccttcaaacagtggtattatgagatttgccaagaagcaactcacttgcattggatgcaaaacaccactcaggtgctctattgtctttcttgtccctatatgttttacaagataagattgtttttcatttctaaggagaatccctccttcctctcaaaattctccttgacttgtgagttactgacatagggtttgtacttccaacctaggagcactagtgtaccttctctaagctgtatattggttgttagttttaagtttctgttgaaattgtaatttatcctggaagagccattgtaggagctatacttgcatttcatgctttgtcactgacatgaatagtaagtaggaatgttgagataaagactaccttagagtttggcagtagttaatatgtttcaaaatggatatggtgccattttctataaaagcttcttgttgatgggcctttttttaccaatgtttattaaacctccaaatcaatgaacctgatttatgaaataaacatctgtcccaatttttatttttatttaatttcttgggcaaaaaaggttaaaagtgatgtgggcctaggagatcatagcagtgtgaaggctcccacaagcttgctaacaattttgcgcctctgtctctgtttgtgcatctctctttgacaagagatgatgagcggatgtattgctttcttcttttttggaagaagctatagtaaaggttgcagaacagcttattaacttatttgctccttcatccagtggctctgatcgaacaatctgcaagcactgcaaaggaagagaggccgaactttattgcagatccgtggctaatggtatgctaggaattcatctcatgttcttttgtacatctcctttttaatttcgtaaggaggagatgacgtggcatatctatttagatgatgtctctagggaaggactcagagctatgaactgcttgaacgttgcttgctaattgtttataactgtattaaatgcagtggctgaactagagaatctatttgggaagctatggacacaatgccaagaatgccaaggctctctccatcaggatgtcctatgcactaggttcgagttgctttctacttttactgatagatacgtgcctattgctagttattcctctcccctttcattcttctaaattatcttgattttgaccagtcgagactgcccaatcttttatcggaggaagaaggctcagaaagacatggctgaagctaaaactcaattagatcggtggaacttttaa

SEQ ID NO: 7 - DNA, 3261 bp, Solanum lycopersicum
  source 1..3261: mol_type = other DNA
atgaactccggcaactctaagaagcgatcggcggcagcaacgccgtcgcctcaacagcagcagaaacaccacgcagccatggaagaagaggacctagacgaggatgtatttctcgaagagacattgcttcagtacgaagaagattcacaggtccttcgtgagctcgaagagagcaattccataaaggagaggcttcagaagtggaaacgtccttctctatctcaagcttacatcgctcagacacaaagcatatcgtttcagcagctggaaatcgactacattattgcagagagccataaagaattgctacctaattcttctggtcaggctgccattttaagaatcttcggggtcacaaaggaaggaaatagcgtctgttgtcatgtacatggatttgaaccatacttctatatcagctgccctcctggaatgggccctgacgatatctcccgttttcaccaaattcttgagggtaggatgagagaggcgaataggaacagcaatgcccctaagtttgtccgtcgcattgaagtagttcagaaaagaagcatcatgtattaccaacagcaggcttctcagccttttctcaagattgtagtggcattgccaacaatggttaccagttgccgtggtattctcgatagaggtattcaaattgacgggcttggtatgaagagctttcttacctatgaaagtaatgttctttttgctcttcgcttcatgattgactgcaatattgttggtggcaactggattgaagttccagttgggaaatacaagaaagctgcaagaaatctctcctattgtcaattagagtttgattgtctctattctgacttgataagccattctcctgaaggagaattttcgaagatggctccatttcgcatattgagtttcgatattgaatgtgctggtcgtaaaggccactttccggagcctacacaagatcctgttattcaagtggccaatctggttaccttgcagggacaggatcagccattaattcggaatgtgatgacccttaagtcatgctcaccgatagttggggttgatattatgtcctttgacacagaaaaagaagtattgcgggcttggagggaccttatccgggaggtggaccctgatataataattggatataacatctgcaactttgatttgccatatctcatccggagagctgaagtcttagggatggcagaatttcctgtgcttggacgcatcagaaacagcagggttcgtgtaaaggatgcaaccttttcttcaagacagcatgggacaagggaaagtaaagaaataacaatagaagggagagttcaatttgatttatttcaggctgtgcagagggattataagctaagttcttactcactaaattctgtctcagcacattttctgaatgagcagaaagaggatgtccatcattcaattatatcagatcttcaaaatggcaatgcagaaacgaggcgccggcttgctgtatattgtctaaaggatgcttatctcccacaacggcttttggacaaattaatgttcatttataactatgtggagatggctcgagtcactggtgtacctatatcttttcttctctcaagagggcaaagcattaaggtgctttctcaacttttaaggaaggcgaagcaaaaggatcttgtcataccaaatgcaaaacaggttggatctgaacaaggaacatttgagggggctacggttttggaggcacaggctggtttctatgagaagccaattgcaacattggattttgcatctctatatccatcaatcatgatggcatataatttatgttactgcacgctagtaactcctgaagaggttcgcaaactgaaccttcctccagagtgtgtcaacaagaccccttctggtgaaacgtttgtcaagtcaaccttacagaagggaatacttccggaaattcttgaagaactattgactgctcgtaaaagagcaaaagcagatttaaaggaagccacagaccctcgagtcaaagctgtcttagatggccgtcagctagctttgaagatcagtgcaaattcagtatatgggtttactggagctactgttggtcaattaccatgtctagaaatatcttcaagtgttactagctatggtcgacagatgattgagcacacaaaaaagcttgtggaagacaagtttacagtacttgggggctatgaacacaacgctgaggttatatatggagatacagattctgtgatggtgcagtttggtgtgcctacagtggaagaggctatgaaattgggcagagaagctgctgattacattagcgggacatttatcaaaccaataaaactagaatttgagaagatttattacccatatttgctgattagcaagaagagatatgctggccttctttggacaaatccggacaaacatgacaaaatggatgctaaagggattgaaactgttcgaagggataattgtctcttagtcaagaaccttgtaaccgagtgtctttataaaatactagtagacagagatgttcctggtgctgttcagtacgtcaagaatactatttcagatcttcttctgaaccgcatggatctgtctcttttagttattactaagggtctgacaaaaactggagatgattatgaagtaaaggcagcacatgttgaacttgcagagcgaatgcgcaagcgagatgctgctacagcacctagcattggtgatagagtaccttatgtaatcattaaagctgcaaaaggggccaaggcatatgaaaagtcagaagaccctatatacgtgttagagaacaacatacctatagatcctcaatactatcttgaaaatcaaattagtaagccacttctgaggatttttgagccaatactgaagaatgcaagtaaagagcttcttcacggagatcacacaagatccatctcagttccaaccccttcaaacagtggtattatgagatttgccaagaagcaactcacttgcattggatgcaaaacaccactcagtggctctgatcgaacaatctgcaagcactgcaaaggaagagaggccgaactttattgcagatccgtggctaatgtggctgaactagagaatctatttgggaagctatggacacaatgccaagaatgccaaggctctctccatcaggatgtcctatgcactagtcgagactgcccaatcttttatcggaggaagaaggctcagaaagacatggctgaagctaaaactcaattagatcggtggaacttttaa

SEQ ID NO: 8 - DNA, 3261 bp, Solanum lycopersicum; identical to SEQ ID NO: 7
  source 1..3261: mol_type = other DNA

SEQ ID NO: 9 - protein, 1097 aa, Gossypium hirsutum
MNKGGNSRKRPPPPSSHQPSPALKHQATTPTPPPPSTEEDFVDEDVFFDETLMGEEDVESLILRDFEERQALASRLSKWARPPLSDAYISQSRSIVFQQLEIDYVIGESHKNLLPDSSCQAAIIRIFGVTGEGHSVCCLVHGFEPYFYISCPPGMGPDDISHFHQTLEGRMKEMNRNSKVPKFVRRIELVQKRSIMYYQQQKSQPFLKIVVALPTMVATCRGILDRGIQIDGLGMKSFITYESNVLFALRFMIDCNIVGGNWIEIPAGKYKKTAKNMSYCQLEFDCLYSDLISHAPEGEFSKMAPFRILSFDIECAGRKGHFPEPSQDPVIQVANLVTLQGEDQPFIRNVMTLKSCSPIVGVDVMSFDTEKEVLLAWREFIREVDPDIIIGYNICKFDLPYLIERAQTLGIAEFPILGRIRNSRVRVKDTTFSSRQYGTRESKEVTVEGRVQFDLLQAMQRDYKLSSYSLNSVSAHFLSEQKEDVHHSIISDLQNGNAETRRRLAVYCLKDAYLPQRLLDKLMYVYNYVEMARVTGVPISFLLSRGQSIKVLSQLLRKSKQKNLVIPNVKQAGSEQGTFEGATVLEAKAGFYEKPIATLDFASLYPSIMMAYNLCYCTLVTPEDVRKLNLPPECVNRTPSGETFVKPELQKGILPEILEELLAARKRAKADLKEAKDPLEKAVLDGRQLALKVSANSVYGFTGATVGQLPCLEISSSVTSYGRQMIEHTKKLVEDKFTVLGGYEHNAEVIYGDTDSVMVQFGVSDVEAAMNLGREAAEHISGTFTKPIKLEFEKVYYPYLLISKKRYAGLFWTNPQKFDKMDTKGIETVRRDNCLLVKNLVTECLHKILIDRDIPGAVQYVKNTISDLLMNRMDLSLLVITKGLTKTGDDYEVKAAHVELAERMRKRDPATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALICNSDRTLCSHCKGREAELYYKTVSQVSELEELFGRLWTQCQECQGSLHQDVLCTSRDCPIFYRRKKAQKDMVEAKRQLDRWNF

SEQ ID NO: 10 - DNA, 10295 bp, Gossypium hirsutum
  source 1..10295: mol_type = genomic DNA
atgaacaagggcggcaattcacggaagaggccgccaccgccttcttcacaccagccatctccggctttgaagcaccaagcgacgactccgacgcctcccccaccgtcgacggaagaagatttcgtcgatgaagatgtcttctttgacgaaaccctgatgggggaagaagacgtggaatccctaattctacgcgacttcgaggaacgtcaagctctcgcttctcgtctttccaagtgggctcgccctcctctctctgatgcctacatttcccagtctcgtagcattggtaagtttaagaagcaaatattttgctgtaaatatttgatttttaagtttttttgggggaaattttgagctctgccttcttcttctcttgctggtgttttagttttccaacaattggagattgattatgtgattggagagagtcataaaaatttgctacctgattcttcttgtcaagctgccattatccgaattttcggggttactggagaaggtatttattcgagaatttttcaagtgatattagctgctgagtactaaaaagcatataatattactgaccgtgcctgttattaatttgaaagaatcccaaattgtacattttatgtcaatctgatgaattgcttatattggcaacttgtaggacatagcgtttgttgtcttgttcatggatttgagccgtatttctacattagctgccctccaggaatgggtcctgatgatatctcccattttcatcaaacgcttgaggtatttggttttaccacttcctcaggttatgcatttaccttttgcgagtggcttcttaattgtttcagcaacatggtttcagggcaggatgaaggagatgaacagaaacagcaaggtaccgaagtttgttcggcgtattgaattggttcagaaaagaagtattatgtattaccagcaacaaaagtctcaaccatttctcaagattgtagtggcattgccaacaatggttgctacctgtcgtggtaacattcctgattcttttgtatggtatttattctgtttgggtttccttcagtaaatgaaatgccttattgtagttgaaactatttttaacttacttttttctctgtcatgaaatcttaatcatgtacataattgaatacctagaaaactgcctagttctttttatacctggaggctttttaattgccaataatttgcaggcattcttgatcgaggcatacaaattgacggccttggtatgaagagcttcattacatatgaaagcaatgttctttttgcactacgtttcatgattgattgcaatattgttggtggtaattggattgagataccggctggtaaatataagaagacagcgaagaatatgtcctactgccaattggagtttgattgcttgtaaacactttttttgtctattaagagcatttcaactattccattgtttctttatcctcctggtttaattctcttcccttattgttcttaggtattcagatttgattagccatgcccctgagggggaattctcaaagatggctccatttcgcatattgagttttgatatagagtgtgctggtcgcaaaggtcattttccagagccttctcaggatcctgtaatccaggtcttattcctctgttcaagtcacccatttgatttgggaaaaattggaaaaatgttattaagtttgttcatgggatcctagttcacgttgacgaaggattgattgaaagctaacctttcattttctttcaccagctctgaagaaattttttgtttctttctaggtagcaaatctagttactttgcaaggagaagatcagccattcattcgtaatgtcatgacccttaagtcatgctccccaatagttggtgttgatgtaatgtcttttgatacagaaaaagaagtcttgcttgcttggagggtactgattttgtctttaaaaccttctctgttttacttcaatacttaggcataaaaaagaagttatccatttgttcatattactgaacaccatgagtgagtctttttttttaatcttaattgttcatgacatgtttgatatgtttgaagggattcattaaccaacttcctgattcacccctgttctacctttatgtttgtcttcttgtatttcattcgataaatcatttaaattgaatcagaaaactgccagaaatgcaagaaccaggaaaacaaatgctttttcaagccaaatataaagctttcattactgcaattcacttaagcactaatgaaatcatcaaaatattatcagtaatcctaagtatatgtctaggaacctacttctttaagtgtcagtggcttttaagctgatattatgataaatttccattttacaaaaccggttccttgaagaacataggcaatctcacatcaatctcgtatgagaagaaagtgtttcttttgcttacactgcaccttttttaatgagaaggttttgcttgcaggaatttatccgtgaagtggaccctgatattataattggatataacatttgcaagtttgatttgccatatcttattgaggtaaatgaaaaattagcttctgttttattctccatgaatgttgctgcagcaaaggtttctttttggttcattatgtagggtttcctttatttgtataggagttgtgtgattgaaccatctgacatttaatcttctggttaacagagagcacaaacacttggaatagcagagtttccaatacttggccgcatcaggaatagtagggttcgtgtaaaagatacaacattttcttcaaggtaaagtatctgttatgggtgttattcttataattgctgctgttgttagccttattttaaactggcttctcacattgcaattgcaaagaatactaataatctccacatccttataaattaatttgtttctcctcattgccttcccttttatgtttttgaaactattaatttactcctatcaaattagaatttcttgttcctgcataatttccgatggtgaagttagttgcttctgttactaaagaagtgttaaaaagttctgcctttctaaggctctgtttcatacaaggctataacccaaagggtcatatgaggagcttagtgtgaatcaatcttgtattcattttcctttagctgcttccatctataactttactgaattcacttgtcactaattttcagagtgaggttatgaagaaggaaagttatgttgtctgtaacttacttcaactattgattgaaagcgtttaactgagcgtttgttactttgtttcatcatacttcctgccctagcataaataactattgtgagttttcttccagtttctggttgagttaagagttactttcctggtatatattctggttattgatgctggcttgttttacagacagtatggaactagggagagcaaagaagttactgtggaggggagagttcaatttgatttgctccaggttttatgcatgcagtttttctactttatgagtttgtaatttttatctgagttcaaatgattcatatttacaaatttgttagctttgtttctttttgttgtaggctatgcaaagggactacaaactaagttcttattcattgaattcagtttcagcacactttctatctgagcaggttagaatttctcttctaagattatggctacttctgtaatcatgttttaatagttaaaatatgttgttagattctgcaattggttcctagggttaatatattcctaagggttcttttgcagatttgtttacctttcgacatttgttgtttccttttgtttgtcactcttctccaaaatttgaactcaagttttgcccttgcattgcttttgtacagaaagaggatgttcaccattcaataatatcagatcttcagaatggaaatgctgagactaggaggcgtcttgctgtctattgtttgaaggtgcatagttggttttagtctaacttgcaaaacttgagtgcacaaactattatggttgtagattcttttgacttttttttatgtctcatcaggaatttagaggctaaaagaaacttgagtccatgtccttattgttggatagaattgtttatttgactttcattttgattgattattgttagtgattgaatttagtttgatttgctctttatcttcatgtgtattcagctcattaaatgcctgagcctcttgagacatttgtgatagtagctcccttctagttggtttcatagaaatgccctattgtttagtttttctattaagagcttgttaggtgtcttttgggccatgtttttgcatctttcttttctgatacaagggaggcaatgcccagggaataggggaagggatattgctatgggcatcaaaccccagccttgtagaagtctacctcctaagtgaggtgggtgaaacatttggtgctaaagcccaagttccttttgcatttaatttacatacataatatattaccagagttgtatttctacaagtattttgagttgttcaagacatgagccttttgctaatggttattccaatttctttacagctaagatatataagatgttaccagcatcagctgtagcttccattctaaaatcctacgtagtgatctttattttttgatcttatatgtctattacatcagcagacattatactctactcttcagagtgcattggttgagttgaagagcttaagtttctgatcatctatgactgaatcagtattctctttcttgtatggttgcaggatgcttaccttccccaaaggcttttggataaactaatgtatgtttacaattatgttgagatggctcgtgtcacaggtgtgcctatatcttttcttctttctagaggacaaagcatcaaggtttgtacaatttgtaagtttccgtggatgaatcaagtgattaatagttcaaatttaaaatctttgttgattttttcaggtgctttctcaacttcttaggaagtctaagcagaagaaccttgttattccaaatgtcaaacaagcaggatctgaacagggaacgtttgaaggtgcaactgtaagtttgttctcaaatttcttatgctagtaactttttgccacctaaaaactacagctacctcactaaaagattaaacagacatgcatatcatgttgatgaactctgtttcaggtgttggaagcaaaagcaggattctatgaaaagccaattgcgacactggattttgcgtctttgtatccatcaattatgatggcatataatttatgctattgcacattggtgatacttcaaatttatcttgcttttaccatctcgttcagctgttattttatgtttcttttatttatgttcctttttgtctgatcaataattggtgggacttttgggcaaattctgatcttggatggtctttgtgaaggtgactcctgaagatgttcgcaaactcaacctcccaccagaatgtgttaacagaactccatctggtgaaacatttgttaaaccagagctacagaaggttatattattaaaatgtcaattttgtcatagatgattatttcctctataaataagtaaactttagaggttttattttattttcttgttgatgaataccgaaaatctaactgattagaagaatgaaaatcttcaggggatacttccagaaatattggaagagctattagctgctcgtaaacgagcaaaagctgatttgaaggtgattacaataattttattttaattgcacagttctgcttcctattgctattggttctatattgtgccatcaaaatattaagtcttcttctttgcctccatttttttactcatattctttatatgaatgtaggaagcaaaggatccacttgagaaagctgttctagatggccgacagctggccctgaaggtaggatcattacttttttcttatcatccgttttcaaatggtaattatcgcattctggggttgaaataaatgtttccttgtatgcttttcattactttgatctgatttattatttatttcaggtaagtgcaaattctgtatatggatttacaggagctactgttggtcagttaccatgtttagagatttcttcgagtgttacaagctatggtacaataaaatgttcttattaacagcattgctaaaaaaattcccatccctataaatgatttaaaatgcattattctagttttgtttgatctaacatgttgtttaccttgtaaaggtcgccaaatgattgagcacaccaagaagcttgttgaagataaatttacagtgctagggggctatgaacacaatgctgaggtaagtcatcccaaaagctctagttatatatgtcatgaacttgatgcaatatgctaatcaaaatgaactgagattcggttaagcatttgattgaaggtttaaagttggaatctcaaggatcatttaaaaaaaaatagtttctgatgcatccataacatggatcatctttcctttcatatgttatctatttcagctttcccaagttaaggctgttttgtcttacctggttcttggaagtttaattttctcgtctggccaaggagattagagtgatacagtatttgggcctgcatatgtgtacttattgttgcttgatagtcttgcatcacattattcttacggatattgtggcagaaatcatactaaataattttttataatcaggttatatatggggacacagattcggtgatggtacaatttggtgtgtctgatgtagaagcagcaatgaacttggggagagaagctgcagaacacatcagtggaacttttacaaaagtaagacttcttttatagtaaatagtgccacatcttttaaaattgaagaaaccctaattgcccagtgtttaaataggaaggggaagtgttctgcaactgtctgagccttaatgtgttacacttggttataatattctggtcagctctcatcttgaatcataatgtaaacgtggcggctacctctagggccatggatgttaataatctcattcatagcaaattatcacttaaaagttaatttttgtgttttttgtcaacttctggttacttgctggtttatctgttgttaatattgtcttcattttctgttcttttttttttccccctttttctatattggtttggtgtttttagcccatcaagctagagttcgagaaggtttattatccctatcttttgattagcaagaaaagatatgctggtttattttggacaaatccacaaaagtttgacaaaatggacactaaaggtaaacaagaaacctcaaccatgtggacttctgaaatttttattgaacttgaatgctaattcatctttcatcatgaacaggcatcgaaactgttcgaagagacaattgtttattggtcaaaaaccttgtaactgaatgcctgcacaaaatactgattgatagagatattcctggtgcagttcaatatgtcaagaataccatttcagatcttctcatgaatcgaatggatttgtcacttttggttattactaaggtgacaagaatatcctttattcctggtttaatgatgctttagtcactgtgaagattttgtaatgcttggtctaaatttgtgttggtctttattttagctgtttgttcctgctcctatgtacagggtctgacaaaaactggagatgattatgaggtaaaagctgctcatgttgaacttgctgaacgtatgcgcaaggtttgtattatgctttttcttggtatatctcagcatgttacatataatatatgtttgaggtacctaattataatacttatatatatggtggtagcattgtcttcagatgacttaagtttaatttctttggatacttgttaaacagtcaaatattgcatgtgcttactgtaagttttatgctgagtttgcatgttatcatatatttctagataattgttagatgcaataatgattgctactatttttcaatttctaaatgagataatgagctgttgcctttaccagatttcagttgtagttgctttcttctaaggccgcctgtttgatatcctctataaaatgctgattgattttagagcattaggatgaactttttctctatacttgacagcgagatcctgcgactgctccaaatgttggggatcgggtgccttatgtcattattaaagccgcaaaaggcgccaaggtaatcttgttgatgattttttcataaaaaaataaaactattgcaaatgattgtattatttatatatcctggtatccaataccgtctatgaattcaggcttatgagaggtcagaggacccaatctatgtgctagaaaataacatccccatagatcctcaatattaccttgaaaatcaaattagcaaggttagtttagtggtttaattgtttcagtttagtctatgacaaaattttcccttgttttaacaattgtattaatgtaacagccgcttttaagaatttttgagcccattttaaagaatgctagcaaggaacttctccatgggagtcacacaagatccatttccatatcaactccgtcaaacagtggaataatgaaatttgcaaagaaacaacttagttgtattggctgcaaagcgctaatttggtataaatctacttcattctattttttatatttaaaggatcagtaatattttctgtaaagaaattgaggatccttgttttatgatgtttgctttttactattctgaagatgaccagtgtctcaattcttatgattttgtgcttcataagccagattttctatgctattttcaatgtcgaaatgctttcaacagacttcccctcaattgatttttccataaaatgatctcccccccccccaaagatacacacactcttaactttgaaaattgatcgaacctagaacggcctttagagaggtcttgcaaaatacgaaatatgcattgggagatgtataggtgtaaaagttcttagaagtatctgcaagaactttatagagtccacctgcatattgattactgcacaccatgcaaatatatggcatatcttacatgtataaataccatttgggttttccctcagcaattcagatcgaacactctgttcacactgcaagggaagagaggcagaactttattataaaacagtttctcaaggtgtgcatatttttcctttactgtttttctgaattaaaataacattttttagaactggaggagccaaaggtcttttgacattatgattgtatgaaatgattgcagtgtcagaactagaggagctttttgggaggctatggacacagtgccaagagtgtcaaggctctcttcaccaggatgtgctctgcactaggttttaccatctcctttctctttgtgacttgtgatgaataatgttaatgctgttttagtttgacaattcttttttcctggaaaatccttggtaaaaccaggatcttgcaatgcattagtgtagtagtgctcaaccaatatagaaccatgcagattatgtgcttccttttttggaaaagaaagggaatgacaggctgcctattaagtgtgactgcatcttattgcttcgtcctttctttattttaattataaactatgttttatcatgtttacagtcgggattgtccaatattttaccggagaaagaaagcacagaaagatatggtagaagccaagcggcagttggaccggtggaacttttaagtctataacaggttgttttcagctgctcaataaacgcatcttcccgttaatgagcatttcacaggctgtgtatggatcccctggtatctcaacaattgagtcaagaaaacctctagagtgtttctatcccaaattttaaagttaaagattagaaaccaggcggagcttctcttttttggaacaaagaaagttgaaaatctggacataaagctttatcagttaagattcttcagttttcctttaaagcttctgttcctcaatgtctttacaaattgcaggttttggcttgtgcaatgctgagcgatgtcagcatgttctgaggtctattttcaattttgctgtatcacggtttgcagtggtccatgtaatgcgaatgtgctattatctataatcttttccgtatttcagtgcacaaaatctctactttgtatcattacatgcattaagttggtgggtaggcgatgctgctacgtgttgtattccctttacctaatatgatacatatatattcaccttgtttttactagatttgataatttgtatgtatgtctagtcacatcataaattattaaatatgtcaacatcttaaaaat

SEQ ID NO: 11 - DNA, 3620 bp, Gossypium hirsutum
  source 1..3620: mol_type = other DNA
atgaacaagggcggcaattcacggaagaggccgccaccgccttcttcacaccagccatctccggctttgaagcaccaagcgacgactccgacgcctcccccaccgtcgacggaagaagatttcgtcgatgaagatgtcttctttgacgaaaccctgatgggggaagaagacgtggaatccctaattctacgcgacttcgaggaacgtcaagctctcgcttctcgtctttccaagtgggctcgccctcctctctctgatgcctacatttcccagtctcgtagcattgttttccaacaattggagattgattatgtgattggagagagtcataaaaatttgctacctgattcttcttgtcaagctgccattatccgaattttcggggttactggagaaggacatagcgtttgttgtcttgttcatggatttgagccgtatttctacattagctgccctccaggaatgggtcctgatgatatctcccattttcatcaaacgcttgagggcaggatgaaggagatgaacagaaacagcaaggtaccgaagtttgttcggcgtattgaattggttcagaaaagaagtattatgtattaccagcaacaaaagtctcaaccatttctcaagattgtagtggcattgccaacaatggttgctacctgtcgtggcattcttgatcgaggcatacaaattgacggccttggtatgaagagcttcattacatatgaaagcaatgttctttttgcactacgtttcatgattgattgcaatattgttggtggtaattggattgagataccggctggtaaatataagaagacagcgaagaatatgtcctactgccaattggagtttgattgcttgtattcagatttgattagccatgcccctgagggggaattctcaaagatggctccatttcgcatattgagttttgatatagagtgtgctggtcgcaaaggtcattttccagagccttctcaggatcctgtaatccaggtagcaaatctagttactttgcaaggagaagatcagccattcattcgtaatgtcatgacccttaagtcatgctccccaatagttggtgttgatgtaatgtcttttgatacagaaaaagaagtcttgcttgcttggagggaatttatccgtgaagtggaccctgatattataattggatataacatttgcaagtttgatttgccatatcttattgagagagcacaaacacttggaatagcagagtttccaatacttggccgcatcaggaatagtagggttcgtgtaaaagatacaacattttcttcaagacagtatggaactagggagagcaaagaagttactgtggaggggagagttcaatttgatttgctccaggctatgcaaagggactacaaactaagttcttattcattgaattcagtttcagcacactttctatctgagcagaaagaggatgttcaccattcaataatatcagatcttcagaatggaaatgctgagactaggaggcgtcttgctgtctattgtttgaaggatgcttaccttccccaaaggcttttggataaactaatgtatgtttacaattatgttgagatggctcgtgtcacaggtgtgcctatatcttttcttctttctagaggacaaagcatcaaggtgctttctcaacttcttaggaagtctaagcagaagaaccttgttattccaaatgtcaaacaagcaggatctgaacagggaacgtttgaaggtgcaactgtgttggaagcaaaagcaggattctatgaaaagccaattgcgacactggattttgcgtctttgtatccatcaattatgatggcatataatttatgctattgcacattggtgactcctgaagatgttcgcaaactcaacctcccaccagaatgtgttaacagaactccatctggtgaaacatttgttaaaccagagctacagaaggggatacttccagaaatattggaagagctattagctgctcgtaaacgagcaaaagctgatttgaaggaagcaaaggatccacttgagaaagctgttctagatggccgacagctggccctgaaggtaagtgcaaattctgtatatggatttacaggagctactgttggtcagttaccatgtttagagatttcttcgagtgttacaagctatggtcgccaaatgattgagcacaccaagaagcttgttgaagataaatttacagtgctagggggctatgaacacaatgctgaggttatatatggggacacagattcggtgatggtacaatttggtgtgtctgatgtagaagcagcaatgaacttggggagagaagctgcagaacacatcagtggaacttttacaaaacccatcaagctagagttcgagaaggtttattatccctatcttttgattagcaagaaaagatatgctggtttattttggacaaatccacaaaagtttgacaaaatggacactaaaggcatcgaaactgttcgaagagacaattgtttattggtcaaaaaccttgtaactgaatgcctgcacaaaatactgattgatagagatattcctggtgcagttcaatatgtcaagaataccatttcagatcttctcatgaatcgaatggatttgtcacttttggttattactaagggtctgacaaaaactggagatgattatgaggtaaaagctgctcatgttgaacttgctgaacgtatgcgcaagcgagatcctgcgactgctccaaatgttggggatcgggtgccttatgtcattattaaagccgcaaaaggcgccaaggcttatgagaggtcagaggacccaatctatgtgctagaaaataacatccccatagatcctcaatattaccttgaaaatcaaattagcaagccgcttttaagaatttttgagcccattttaaagaatgctagcaaggaacttctccatgggagtcacacaagatccatttccatatcaactccgtcaaacagtggaataatgaaatttgcaaagaaacaacttagttgtattggctgcaaagcgctaatttgcaattcagatcgaacactctgttcacactgcaagggaagagaggcagaactttattataaaacagtttctcaagtgtcagaactagaggagctttttgggaggctatggacacagtgccaagagtgtcaaggctctcttcaccaggatgtgctctgcactagtcgggattgtccaatattttaccggagaaagaaagcacagaaagatatggtagaagccaagcggcagttggaccggtggaacttttaagtctataacaggttttggcttgtgcaatgctgagcgatgtcagcatgttctgaggtctattttcaattttgctgtatcacggtttgcagtggtccatgtaatgcgaatgtgctattatctataatcttttccgtatttcagtgcacaaaatctctactttgtatcattacatgcattaagttggtgggtaggcgatgctgctacgtgttgtattccctttacctaatatgatacatatatattcaccttgtttttactagatttgataatttgtatgtatgtctagtcacatcataaattattaaatatgtcaacatcttaaaaat

SEQ ID NO: 12 - DNA, 3294 bp, Gossypium hirsutum
  source 1..3294: mol_type = other DNA
atgaacaagggcggcaattcacggaagaggccgccaccgccttcttcacaccagccatctccggctttgaagcaccaagcgacgactccgacgcctcccccaccgtcgacggaagaagatttcgtcgatgaagatgtcttctttgacgaaaccctgatgggggaagaagacgtggaatccctaattctacgcgacttcgaggaacgtcaagctctcgcttctcgtctttccaagtgggctcgccctcctctctctgatgcctacatttcccagtctcgtagcattgttttccaacaattggagattgattatgtgattggagagagtcataaaaatttgctacctgattcttcttgtcaagctgccattatccgaattttcggggttactggagaaggacatagcgtttgttgtcttgttcatggatttgagccgtatttctacattagctgccctccaggaatgggtcctgatgatatctcccattttcatcaaacgcttgagggcaggatgaaggagatgaacagaaacagcaaggtaccgaagtttgttcggcgtattgaattggttcagaaaagaagtattatgtattaccagcaacaaaagtctcaaccatttctcaagattgtagtggcattgccaacaatggttgctacctgtcgtggcattcttgatcgaggcatacaaattgacggccttggtatgaagagcttcattacatatgaaagcaatgttctttttgcactacgtttcatgattgattgcaatattgttggtggtaattggattgagataccggctggtaaatataagaagacagcgaagaatatgtcctactgccaattggagtttgattgcttgtattcagatttgattagccatgcccctgagggggaattctcaaagatggctccatttcgcatattgagttttgatatagagtgtgctggtcgcaaaggtcattttccagagccttctcaggatcctgtaatccaggtagcaaatctagttactttgcaaggagaagatcagccattcattcgtaatgtcatgacccttaagtcatgctccccaatagttggtgttgatgtaatgtcttttgatacagaaaaagaagtcttgcttgcttggagggaatttatccgtgaagtggaccctgatattataattggatataacatttgcaagtttgatttgccatatcttattgagagagcacaaacacttggaatagcagagtttccaatacttggccgcatcaggaatagtagggttcgtgtaaaagatacaacattttcttcaagacagtatggaactagggagagcaaagaagttactgtggaggggagagttcaatttgatttgctccaggctatgcaaagggactacaaactaagttcttattcattgaattcagtttcagcacactttctatctgagcagaaagaggatgttcaccattcaataatatcagatcttcagaatggaaatgctgagactaggaggcgtcttgctgtctattgtttgaaggatgcttaccttccccaaaggcttttggataaactaatgtatgtttacaattatgttgagatggctcgtgtcacaggtgtgcctatatcttttcttctttctagaggacaaagcatcaaggtgctttctcaacttcttaggaagtctaagcagaagaaccttgttattccaaatgtcaaacaagcaggatctgaacagggaacgtttgaaggtgcaactgtgttggaagcaaaagcaggattctatgaaaagccaattgcgacactggattttgcgtctttgtatccatcaattatgatggcatataatttatgctattgcacattggtgactcctgaagatgttcgcaaactcaacctcccaccagaatgtgttaacagaactccatctggtgaaacatttgttaaaccagagctacagaaggggatacttccagaaatattggaagagctattagctgctcgtaaacgagcaaaagctgatttgaaggaagcaaaggatccacttgagaaagctgttctagatggccgacagctggccctgaaggtaagtgcaaattctgtatatggatttacaggagctactgttggtcagttaccatgtttagagatttcttcgagtgttacaagctatggtcgccaaatgattgagcacaccaagaagcttgttgaagataaatttacagtgctagggggctatgaacacaatgctgaggttatatatggggacacagattcggtgatggtacaatttggtgtgtctgatgtagaagcagcaatgaacttggggagagaagctgcagaacacatcagtggaacttttacaaaacccatcaagctagagttcgagaaggtttattatccctatcttttgattagcaagaaaagatatgctggtttattttggacaaatccacaaaagtttgacaaaatggacactaaaggcatcgaaactgttcgaagagacaattgtttattggtcaaaaaccttgtaactgaatgcctgcacaaaatactgattgatagagatattcctggtgcagttcaatatgtcaagaataccatttcagatcttctcatgaatcgaatggatttgtcacttttggttattactaagggtctgacaaaaactggagatgattatgaggtaaaagctgctcatgttgaacttgctgaacgtatgcgcaagcgagatcctgcgactgctccaaatgttggggatcgggtgccttatgtcattattaaagccgcaaaaggcgccaaggcttatgagaggtcagaggacccaatctatgtgctagaaaataacatccccatagatcctcaatattaccttgaaaatcaaattagcaagccgcttttaagaatttttgagcccattttaaagaatgctagcaaggaacttctccatgggagtcacacaagatccatttccatatcaactccgtcaaacagtggaataatgaaatttgcaaagaaacaacttagttgtattggctgcaaagcgctaatttgcaattcagatcgaacactctgttcacactgcaagggaagagaggcagaactttattataaaacagtttctcaagtgtcagaactagaggagctttttgggaggctatggacacagtgccaagagtgtcaaggctctcttcaccaggatgtgctctgcactagtcgggattgtccaatattttaccggagaaagaaagcacagaaagatatggtagaagccaagcggcagttggaccggtggaacttttaa

SEQ ID NO: 13 - protein, 1079 aa, Phaseolus vulgaris
MSASRKRAAAPPKPPPSKQALLTQEEDMDEDVFMEQTLISEDEESMILRDIEQRQALAARLSKWARPPLSADYVKQVVFQQLEIDYVIGESHRELLPNLSGPAAIIRIFGVTKEGHSVCCNVHGFEPYFYICCPPGMGPDDISHFHQTLEGRMREANRNSNVGKFVRRIEMVQRRSIKYYQQSDSQSFLKIVVALPTMVASCRGILDRGIQLDGLGMKSFWTYESNVLFALRFMIDCNIVGGNWIEIPAGKYKKIAKSLSYCQLEFDCLYSDLISHAPEGEYSKMAPFRILSFDIECAGRKGHFPEPSHDPVIQIANLVTLQGEDQPLIRNVMTLKSCSPIVGVDVMSFDTEREVLLAWRDLILEVDPDIIIGYNICKFDLPYLIERAANLKIAEFPILGRIRNSRVRVKDTTFSSRQYGTRESKEVAVEGRVQFDLLQVMQRDYKLSSYSLNSVSAHFLSEQKEDVHHSIISELQNGTPETRRRLAVYCLKDAYLPQRLLDKLMLIYNYVEMARVTGVPISFLLSRGQSIKVLSQLLRKARQKNLVIPSVKQVGSEQGTFEGATVLEARAGFYEKPIATLDFASLYPSIMMAYNLCYCTLVTSEDARKLNIPPESVNRTPSGETFVKANLQKGILPEILEELLTARKRAKADLKEARDPLERAVLDGRQLALKISANSVYGFTGATIGQLPCLEISSSVTSYGRQMIEHTKKLVGDKFTTLNGYEHNVEVIYGDTDSVMVQFGVSSVEQAMNLGREAAEYISGTFTKPIKLEFEKVYYPYLLISKKRYAGLFWTKPDNFDKMDTKGIETVRRDNCLLVKNLVNDCLHKILIDRDIPGAVQYVKNAISDLLMNRMDLSLLVITKGLTKTGDDYEVKAAHVELAERMRKRDAATAPNVGDRVPYVIIKAAKGAKAYEKSEDPIYVLENNIPIDPQYYLENQISKPILRIFEPILKNASRELLHGDHTRSISISTPSNIGLLKYAKRNLTCIGCKAVLGKDLHTVCSHCKGREAELYCKTVSQVSELEKLFGRLWTQCQECQGSLHQDVLCTSRDCPIFYRRKKAQKDMSEAKSQLDRWSF

SEQ ID NO: 14 - DNA, 14118 bp, Phaseolus vulgaris
  source 1..14118: mol_type = genomic DNA
agaatatctcaactcaactaaaggaattgtattatcccagccacgtgccgcgcgcgggaaaatctcactgtacaaacacccaacaaacaggcgggaactcgttaacctttcactccacactctcccacgcttccaaccttctctcttccgattccaatgagcgcctcccgaaagcgcgcggcggcgccgccgaaacctccgccgtcgaagcaagccttattgactcaggaagaggacatggacgaagacgtgtttatggagcaaaccctcatttctgaagacgaagagtccatgattctccgcgacatcgagcagcgccaggccctcgctgctcgcctatccaagtgggcgcgtcctccactatcggccgattacgtcaaacaagtcggtgagtattttcagttgattttttttcgtttcttatttggtgttagggttttgttttctcccgtcattagtggaattttattgatttgtgctctgagtgtctgttttttgcttttagtttttcagcagctggagattgattacgtgatcggggagagtcacagggagttgctgccgaacttgtctggacctgctgccattatcagaatctttggagttacgaaggaaggtctgctcgtgtttctgctatctttatgctacacaatttggtttcttagacaattggagataaatgttgctggtttgtttgtttatagtgttgtgcgatctcgtagggaagaaagaaaacctgaatgacccactcaacgaaccgttgctagttttagaagtttgaaacaataaagagaacgatagtagtcgatgatgtgtttgagcgtttctgttggttttctattattaagtcaagtgtggtttacattaggagtgtttggagtttcgtttggttcagtttttgggggaaaacttgtaggcacagaaccagtaagttcttagaggagaaattgattaaaccaaatcaatctcaattgttttggttcagttctgccatgtcagtttttgatcaaccactttttctttaataaacactaagttgacttcacttgtttttacacgatccactatcaactaattgcccctaaggcttattattaaaagcataatcaatcgatataaataaaaaattaatgtaagaactttcatgacaagtagaaatctaatctatgaatatgttaattttgggcaatatatgcatagatagcaaactacacaattttattctggttctgaagcggaaaactgaaacaaactgtatgtatgcttaagaaaccgaacttagttgggtttgttaggtcttttggttgccaaattaagatttatcaacctagtgcacagatagtttgtttgattccagatatgaagtgatgaatgtttcagaaaagggcaaattgatgcttttgacaaacttgtgctgaatatatttttgtatagcccaacatcatgtttggattttactttcttgcatactcaggtaccttgcataattcatgtctggattttccaatggttacaaatgtcaaacaatgaattttgtgcctagaaataatctgttttgagagaactagtcaagtgtttgttgaaaaagttagtaaaaataaaaattatatatgacctatcattgtacttatgattatggctctcaagtcagccatgcagcaccttgtaggaattatataattaacttatattttgcttgtttgtcaggacacagtgtttgctgcaatgttcatgggtttgaaccatatttctacatctgttgccctcctggaatgggtccagatgatatctctcattttcaccaaacacttgaggtatctggtttagtacctgaaaatacatttttttttctggtttttaatgtagggttcatttatttaacttgcaaactgcttttattacttgcttttgtcatgcttcagggaaggatgagagaagccaatagaaatagtaatgtaggaaaattcgttcgccgcattgaaatggtgcagaggagaagtattaagtactatcagcaatctgattcccagtcctttctcaaaattgtagttgcactcccaacaatggttgccagctgccgtggtaaacttatatattttaaatagacattctattttctaatatattaaccatcttatcaaaagtcagaagtccagaattactttatttatgtttaagctgcgttcttggttcttattggctgtattcttagtttgttgtgttatggtctcacacactttgggctaaatgttaaatcttagcttcatgcatcttgcactagtatgtctgcatttttttgacagttgtctttgttgaatttacttcatttatgaatcaatgtttcttaccaatatttcaaaacatgtcaggtattcttgataggggtattcagcttgatggtctgggaatgaagagcttctggacttatgaaagcaatgtactttttgccctacggttcatgattgattgcaacatagtaggtggaaattggattgaaattccagcaggaaaatataagaagatagcgaaaagcttgtcttattgccagttggagtttgattgcctgtatcctctttcatgtgtctatctgtgttaaagggttatgggagtattaaaatctttagatatttctgttctccccttaacaatattataggtattctgatttgattagtcatgctccagaaggggaatattcaaagatggctccatttcgcatattgagttttgacattgagtgtgctggtcgtaaaggtcattttcccgagcctagccatgatcctgttatccaggtctctttgccttctctttttgccactacaaattagtgcactgtgttattgtttttcacctgaaaattctcttttctttcagattgctaatttggttactttacaaggagaggaccagccacttattcgtaatgtgatgacccttaaatcatgctctcctattgttggtgttgatgtgatgtcatttgatacggaaagagaagtcctgctagcttggagggtaatttcattgtgtgcttatccatttttatacttatgaagcataagaatttcagcaaaccccccaatttttattcacaagtctccctaaataaactgttttttggatatgcaattaaataactgacatcttgcatgttttgtttcataggatgatgttttgtagttcttataactttggcaatggtcattgccattaaatatctttggtttcttaaacttgtatatcatgagtcaatttgatattatttaactcatgaatggattataatcttttctctattacacccttatattttgatggaggtgttctttttcttgcaggatttaattctggaagtggaccctgatattattattggatataacatctgcaaatttgacctgccatatcttattgaggtgctgacatgacttttaaacagtacacttttagagttgataactgtaaggttctgttatttaaaaccaagttcttgtggttattgttttaatttaatttgttattttcttgttttttcagagagctgcaaacctgaagatagcagaatttcctattcttggccgtattaggaacagtagagttcgggtaaaggatacaactttctcgtcaaggtaaggagatatagtcttctgtcatgtacatgtttcctcattattttataatagtcgtagtctggtggatttttaggttttgttagtttcatcaactttaatgaatatagaaacttgatttctaaaatgaatcctgcattttacattcttaacattttcaatgacttttggaattcgtttctttttccttttattttctgagtccatgtcatttgactatctaccttattgatacttgatggttttgtaggcagtacgggaccagggaaagcaaagaagttgcagtagaagggagagttcagtttgacttactccaggttatgccaatattgttttgctttaattgccctgtgatggactctctaagtttattgcttaacaggtttatttgttatgtttccatttgcaggttatgcagagagactacaaattaagttcgtattcactaaattctgtctcagcacacttcctttctgagcaggtaaagtcattcttttgacatcatttcaacaattatttcctccctttattgtgtattggcacaatggtaaggctattgcattgtgacttggagatgataagttcaaatctggaatgcctctcttcttagggtataagcatggttatcaaactcgcaagtcaactcgttcgaatttacgttcccactcaccctgggcgagttgactcggaaacaaactcgttttttgatcaaactcggtaaactcacactgaactcgtgagtttgcaaccgagttgacgagttaagagggtttttctttaaacccaaaacgacccgtttctgaacccaaaattagacaaactcacattgccaacgttagggtttgctcgcgttctttgtggtggtgtgtctgtttcaattgtggtcgtggtgcgctgttacgattgtgttggcgttggtggtggcttttgtggtggcgtttgtggcgacgttcgtgatcgtgttccgtaattgtgatgtagcgtcactctgtttgttgtggtggtgcgtcgctccgtttgtcctggtcgtggtcatggtgcatttgtttgagtttagggttttcttggccaagagaatattgagggttttttaaatctgctgggaagtttttttaaaatttgggccgtaagagagaatatcaattggagaattttttgtcaattgaattttattttaatttcagaatattctatttttaaattttttgttaatttaactaggcataacccaagaacatcccccatttattaaaataagtaaaaatgctccaggatataatgtttatataggacccaattacataatgttttataatatttatattatatgaatttatttatttgagatattttgaacttatataattctcattcactactaaatgtagttattttattaatataatggacttttgttttaaatattttgattaattgtatgatttgaaatatttttttcatacgaaataaactcttacgagtctagagagcctccatgagtctacgtagtctctcgagtttgataaccttggatataaggttgtgtacatgtacccttcttcgatcccattaagtgggatgggagccttgttcactgggtcacccttttattcctttattttctgtttcaaatacatcttggaagtgggagctgttaatgggatttggatcatggttggaaagtactattaacacatctttatattaatatataaatactatacctgtctagttatagcagttgatgaagttgctgatttacatgcatagactcatgatttgatatgaggattggccatttcaattggattgcaatgcagttgttattttacagatacgcatttcaattggtctttttatttgctatttgtatgtttgcatgtgtgtctgtgctctttttctgcattttgacttatttatatgcttaccattatctagacagcttcttttgctggaatctcgtctttgttcttggatgttatagtggttgtcaatttattagcttagtaactgcttgttcatatagtgcagaaagaggatgtccatcattctatcatatctgaacttcagaatggaaccccagaaactaggaggcgtcttgctgtgtattgtttgaaggtttgctttctggttcttttgtcctaggatgctttattgtcatctttttaaactattattgtctattttttcttttgtttcctatctagttctcactattataaatgttgaagttgacaataattactttgtaactcagttgatgatgaaaattgtgatctgactgattgtgaaacagttttggtttgcgttgcttgttagttttatggaatacttctgtgtgggacggatttaccatgttatttagtgacccttggcttctgttgggaagttgtaaagaaggctctgtttcatttctccttttgatgtgtaacagataaggaattttactaaattttgttggccaagctgtcacttaagttggcagtaactaattctaaccaactgattccagatattatttgacagtaatatactaagaaaagtttcaatattatcctattatacttaataatttagctttaaaaagttttattgtttgtgattattttctgggtgaattgcttgtggttctttaacctattaaaatgttgtgtatcagatatggtgaatccattatcggtattttacgtgttagagaaattgaccattcaaaattcatactctgcccgtcaatgtgcaggatgcatacctccctcagcgacttttggataaattgatgctcatttacaactatgtggagatggctcgagtaacaggtgttccaatttcttttctactttccagaggccaaagcattaaggtaagcgatcaaagttcctttttaattaacaatgctgagaatcacttaaatgattgactcattaaatttgtatcagtaattccttgttttcttttcttcctttccaggtactttcacaacttcttaggaaggcaagacagaagaatctggtcattcctagtgtcaaacaggtcgggtctgaacaaggaacattcgaaggtgccactgtaagttcattcctttctttttcatattcattattttaacggttaataggcgtttattaactgattctttccactttcaatttctatacattcatgtttgtgtgtatgcatcttctaatatgaaatgaaatagattgtattagctacccattcatacaatacaactattcgatgccttgtattttctgagaattttatttatattttgtctcaacaaattagattatattcggaattgagcatgaatgaattggatattcaaaggctcatagcatcatgaactctttacagcaagattgcattgcattaaagttgtcaaatttgagtctatggtaactcattaaggttctacaaacttaatactttacctaaaatagtattaaaaatacctaatcatgtatcgaaacaaaatatccaagtgacacaatggacaaatcactgaaatacaattatccaagtggatcaaatgaacaagggcttcacatctattacctgcatttgactgagcctaccagagttaaatcgagtgtgggtatccctgagctttcgagtgagttgccagtttgtcaactatgctttgtataattttctcctctatattcatgaaacaatcacctttgtcaatgtatattatttttggttagatagaaattaggatattgtattttcactttacagttggaacaaagaaagacaattctgctatatagcattgcctttttagtttagggaaagtaggtaagaaaataactttatcctaataatgacaaatgtacagtgagattttattctgatttagtttcttgatttaaaaatgttttgaaccaggagaaagatctcctagggtatcccctttacaagggtttcccatatttggcacattaaagctaaagcttagttctcaattgatacaaactgaatgccaccttgattaaactaaacgccctattcttccatcctctccaactaacagaattttctaactgttgatactttactgtaactagtaactattgatgttttggaatccctaacatgttgggcttgtttggtttgagattttttttttctatttttatatcccgtttttaaggatttgtggagacaatgaaaacagtaaacaaagtgatttttttaaattatttgtgaaaatggaaatagtaaaaaatattttcttaaatcaatttggtccttagctatgaattggaaattttattgttaaaatgttcatatgttatgtactgcttaagcaagcctataaacaaagatctatacaatctggatttgcagttgtttgtagatacttgttatttttccctcttgttcttctgttaatgcaagaaaatattgaaaacatgctcaaatttcaagtttctttattgacagagatctacataagttttggagttgattcactaaccttctccctttattttccttttggacatttcagtcgtcaagttgttgccttttgatgcatgatttttgaattacaacttgcaggtattggaggcaagggctggattttatgaaaaaccaattgctacgttagattttgcatccctgtatccatctattatgatggcctataatttatgttattgcacactggtaaatatttattgttttgttagttttgcttaagcatctaagttttgtttctttagaaagaaatatggatatatattactttattcaccggaatagttcagttttttttttctatgttaactattctttaaggtgacttctgaagatgctcgcaagctgaacatccctccggagtccgtgaacagaactccatctggtgaaacatttgttaaagcaaatctgcagaaggtatgttgtttgaaaaattactatttttcctttttctttgcagttctatgctgatttcattgtagaggttaatgcttatatgaagttgaggcactgatgtaatgaaaataaattcagggaatacttcctgaaatacttgaagagctattaacagcccgaaaaagggcaaaagcagacttaaaggtatcctttctatacttgtgaaaattgacgttgggtttagattgttttgctgtcaactgttttgattaattcaaatccactaattgaattctgtatattatatgtgcctgtaaatgatttctatcaaacatctactacaggaggcgagggatcccctggagagggcagtactagatggtaggcagctggccctgaaagtaagccgagcctcttaattttgtgtcttttttccttgtccttttgtcttttggtatttgttcatttctcactactgctttgtgcgttttaataactgtgttaattcaatatttcttggtattgatacattttaaactccatttctacttacttttgatcagattagtgcaaattctgtgtatgggtttactggggctaccattggtcagttaccatgtttggagatatcatcgagtgtaacaagctatggtattgctttcatctcttagtttgtttggtgtgtgtttctaaattatctttttgttgaatgtaaatagcatctccattgatttatgatgtttgattggatgcctctaaaggtcgacaaatgattgagcacacaaaaaaacttgtgggagataaatttacgacacttaatggctatgagcacaatgttgaggtataatatttataaattttattattttgatacctatcatactctatggggtttcatgggctagaagtcgaagctatcttaggtttttattggtccttattactgcattctagtctggggaatttaacattgaatcaacaatataaactttctatccttgccaataaattttacatttccagttatgcatttgtttcttaaatttaatgtagtcgtatcttttccttatatgctttaccaccataacaatgacaaatgacttgcaaagtaatgtcattcttgctactaggtaatatatggagacacagattcagtcatggtacaatttggtgtttcttctgtagaacaggctatgaacttggggagagaagctgccgaatatataagtggaactttcacaaaagtaaccgtttatttagcgttttattttattcttttatgatagcctgtgaaagtgatggagttctaaacatgaaaactgtattgtgtttttcttgaattatttttatggatcacttatttcaacaatatgaaaaatgttcctttttatatcttctccaaacaatgaagtaacaattgattctgaaatcactgtgttttgtttgctcaagattcaaataaaaatgagcccctcatcctacaaagcaacatttgattattccaatcattcccctgattcttcaaatatggaacaaaaagtttcatattttttcaaataatttagattcaacaattgtaatgttatttctgaatttatattcatgttaattacaacttcacaaaaattgcagctagtctaaggttttagtctgcagattgtttaagcttcagcttcagtaaacctaatcttaattctataaaacttattattttatttgctaccttgatttgtccttatttgttgtcgttttgattgtgaagcccatcaaactagaatttgagaaggtttattatccatatctattgattagcaagaagagatatgctggtttgttttggacaaaaccagacaactttgacaaaatggacactaaaggtaaacattactctttaccttcctctggatttgctgaagtgcctcttaaactaaattttcattgaacaggtattgaaacagttcgaagagacaattgtttgttggtaaaaaacctggtgaatgattgtcttcacaaaatactgattgacagggacattccgggggctgtccagtatgtcaagaacgcaatttcagatcttctcatgaatcggatggatttatctctgctggttattacaaaggtggaagtatattgaatctacagaccatgcaatggtctctctttgctatttgcttttagtggaacattcagatatttgatgctgagtgcattaccaaaataattcctctgaacttggcgccaacatgcttgctttaacagggtttaacaaagacaggagatgattatgaagtaaaggcagctcatgttgaacttgctgaaaggatgcgcaaggttggctagataatcaaatatatatgtggttttgtctttacccatttttatttttttggaaaattagattcttgtggcaaaggaaaaagaaggcaaggttatagtacatgatcatttgctctatatgtgccaaaacatcaaatggtatattagaattacacttgaactatgtagggaaataagaaataatttatttataaaaatcacaccaagtacattctcagttctatttgtaacaaacaatctctttaaaatacaaaagtatgctaaaataaataagaagtttctaaagatattatctcttaatcaagtttggatatcctgaagatattctaataaatcactatttacagcaattaccatgaattatttttaattttgatgtgggattctatatagtcacctctcaggtcctgtaaattaatgtttcaaatgtttgataagttatttgtggaactatacagtattgttactggattgaaaactgttttaggcttattgagtttgccatgtcccagcttggtaggccgcaaatgtatttgatttgattttgtaattgacagcgagatgctgccactgctccaaatgttggagatagagtaccttatgtaattatcaaagctgcaaaaggtgcaaaggtaataccacatgttcccctctcttttttattatagttttttaaacatttgtttagattttctcttgactaacatgtgtaagaaaatcaggcgtatgagaaatcagaggaccctatttatgttctagagaacaacattcctatagatcctcaatactatcttgagaatcaaattagcaaggttggtttatccctttcttagatgttctggatttccaaattgctgttctttattagcttcattgtattgcagccgattctgagaatttttgagccaattctgaagaatgctagcagagaacttctccatggagatcatacaagatctatttcaatttctactccgtcaaacattggcttattgaaatatgctaagagaaaccttacctgcattggctgtaaagctgtactagggtatctatctccccctcttttcatagtgttgtcttaaagttccgggttgattttaattttatatattccctttcttgtgtttgcttcaagatttttgtgaaggaaagacacaacaggaactatgaagaattaacatttatgcacatgctaacattttcaataactaggctgtatatttgagagatttcctatagattttaaatattttagaactataggccagcaatatattatagaattgttatgaaccctttcatgttaccaatttactttaagaagacatacgttctttatgaagatgaacacaagaccccaaaaaaaaatagcacatgttcggggaccattctttcagaatagttgatttaatgaatttcctgtcgtctgtaaattttatccctacttttcaaaatgtttctgtagatactacctgtgagataacattttgtggaggaggtggcataggtttgctctagaaattgaaattttgatttggtttcccggagtcttgtcaggaaagccagattcttcaggtgttatatatgaatcataagactaagagactaagttttgtgatggagattaaaacctttgcagtttacacctataggcatcaagttgtatttccactagtgaaatacctgataatatttatgtcttgtgttgaagattatttttatgaattataatgtacagtatatgtcagtattctgaaggtctcaagtttttgttttatttggttcagcaaggatcttcacacagtctgttcacattgcaaaggaagggaagctgagctatactgtaaaacagtatctcaaggtagggcatccgttgcactctgttcaatttgaaatggtttttggttttgttgtactggatggttacagttatttttaattggacaaaactcacaaaacaagtggtggtgtacttgtagtgtctgagttggagaagctttttgggaggttgtggacacagtgtcaggagtgccaaggttcacttcatcaagatgttctctgcaccaggtatgttcatttagtgaaattgcttcaggttctcaaacttttggttttaagaagcaccatattacagtattaattaattattcgctgatttctcgtgtactattttgttgtagtcgggattgtccaattttttatcgacgaaaaaaggcacaaaaagatatgtccgaagcaaaatcgcaattggaccggtggagtttttaagttttgccaagtcagttttttctctattttcttccatattcggtcctttgtcttcatactgatctcgaccatgaattgcatccatcccattaatccaaaatccaatatttgcaatagaaatcactggaacatttatctttatatgtaggaactaatctaaggttttatattctaaaatagaaaatttgaggtttaataaattttggtcatcagattgtatttttgagtaatatttgaactttctcgcaagattctgcaacaatgatatagaaataatgtattattttgaagaaaaaaaatcttttgacattatgactattagtagattatgaaattttttatacccttgtaatatttttattttaaaaaaataataagtaatataaggatctaaagttttttagtatttagcattaaaaacagtgaacgaaatttcatattttaaaaatttcaaattaaattttaataggaaatttatttttaatagaaaaaattgcatttacaagattcttataattaacatttataccaaaataagtttttaatagaaaagattgcatttactttatgttacattcatcttatacttatctttattttgtaaaaataaaggaacacttatttcccttattttatttaaaaataacattaattataaaaattaaagttttaaaatttatttttaaaatttaaacacaaaatgagtgatttagttatatagaatttatcttttttaagttcttgaaataaaattcatgtagtaaacaattaggctctgtttgttttcatggatgtactgttcacaaggaaagaagtgaggggatattaattgtttggattcattgttttgcagggaaaaggagaagaggatatacctgtgaacattaaatatcctcttccctcaaaatgaagagatgtagagggagaatcacatcagctgtaatatttacgaatttaccttcattcatctacaagggtttttaatattaaaaaacaatatttt

SEQ ID NO: 15 - DNA, 3556 bp, Phaseolus vulgaris
  source 1..3556: mol_type = other DNA
agaatatctcaactcaactaaaggaattgtattatcccagccacgtgccgcgcgcgggaaaatctcactgtacaaacacccaacaaacaggcgggaactcgttaacctttcactccacactctcccacgcttccaaccttctctcttccgattccaatgagcgcctcccgaaagcgcgcggcggcgccgccgaaacctccgccgtcgaagcaagccttattgactcaggaagaggacatggacgaagacgtgtttatggagcaaaccctcatttctgaagacgaagagtccatgattctccgcgacatcgagcagcgccaggccctcgctgctcgcctatccaagtgggcgcgtcctccactatcggccgattacgtcaaacaagtcgtttttcagcagctggagattgattacgtgatcggggagagtcacagggagttgctgccgaacttgtctggacctgctgccattatcagaatctttggagttacgaaggaaggacacagtgtttgctgcaatgttcatgggtttgaaccatatttctacatctgttgccctcctggaatgggtccagatgatatctctcattttcaccaaacacttgagggaaggatgagagaagccaatagaaatagtaatgtaggaaaattcgttcgccgcattgaaatggtgcagaggagaagtattaagtactatcagcaatctgattcccagtcctttctcaaaattgtagttgcactcccaacaatggttgccagctgccgtggtattcttgataggggtattcagcttgatggtctgggaatgaagagcttctggacttatgaaagcaatgtactttttgccctacggttcatgattgattgcaacatagtaggtggaaattggattgaaattccagcaggaaaatataagaagatagcgaaaagcttgtcttattgccagttggagtttgattgcctgtattctgatttgattagtcatgctccagaaggggaatattcaaagatggctccatttcgcatattgagttttgacattgagtgtgctggtcgtaaaggtcattttcccgagcctagccatgatcctgttatccagattgctaatttggttactttacaaggagaggaccagccacttattcgtaatgtgatgacccttaaatcatgctctcctattgttggtgttgatgtgatgtcatttgatacggaaagagaagtcctgctagcttggagggatttaattctggaagtggaccctgatattattattggatataacatctgcaaatttgacctgccatatcttattgagagagctgcaaacctgaagatagcagaatttcctattcttggccgtattaggaacagtagagttcgggtaaaggatacaactttctcgtcaaggcagtacgggaccagggaaagcaaagaagttgcagtagaagggagagttcagtttgacttactccaggttatgcagagagactacaaattaagttcgtattcactaaattctgtctcagcacacttcctttctgagcagaaagaggatgtccatcattctatcatatctgaacttcagaatggaaccccagaaactaggaggcgtcttgctgtgtattgtttgaaggatgcatacctccctcagcgacttttggataaattgatgctcatttacaactatgtggagatggctcgagtaacaggtgttccaatttcttttctactttccagaggccaaagcattaaggtactttcacaacttcttaggaaggcaagacagaagaatctggtcattcctagtgtcaaacaggtcgggtctgaacaaggaacattcgaaggtgccactgtattggaggcaagggctggattttatgaaaaaccaattgctacgttagattttgcatccctgtatccatctattatgatggcctataatttatgttattgcacactggtgacttctgaagatgctcgcaagctgaacatccctccggagtccgtgaacagaactccatctggtgaaacatttgttaaagcaaatctgcagaagggaatacttcctgaaatacttgaagagctattaacagcccgaaaaagggcaaaagcagacttaaaggaggcgagggatcccctggagagggcagtactagatggtaggcagctggccctgaaaattagtgcaaattctgtgtatgggtttactggggctaccattggtcagttaccatgtttggagatatcatcgagtgtaacaagctatggtcgacaaatgattgagcacacaaaaaaacttgtgggagataaatttacgacacttaatggctatgagcacaatgttgaggtaatatatggagacacagattcagtcatggtacaatttggtgtttcttctgtagaacaggctatgaacttggggagagaagctgccgaatatataagtggaactttcacaaaacccatcaaactagaatttgagaaggtttattatccatatctattgattagcaagaagagatatgctggtttgttttggacaaaaccagacaactttgacaaaatggacactaaaggtattgaaacagttcgaagagacaattgtttgttggtaaaaaacctggtgaatgattgtcttcacaaaatactgattgacagggacattccgggggctgtccagtatgtcaagaacgcaatttcagatcttctcatgaatcggatggatttatctctgctggttattacaaagggtttaacaaagacaggagatgattatgaagtaaaggcagctcatgttgaacttgctgaaaggatgcgcaagcgagatgctgccactgctccaaatgttggagatagagtaccttatgtaattatcaaagctgcaaaaggtgcaaaggcgtatgagaaatcagaggaccctatttatgttctagagaacaacattcctatagatcctcaatactatcttgagaatcaaattagcaagccgattctgagaatttttgagccaattctgaagaatgctagcagagaacttctccatggagatcatacaagatctatttcaatttctactccgtcaaacattggcttattgaaatatgctaagagaaaccttacctgcattggctgtaaagctgtactaggcaaggatcttcacacagtctgttcacattgcaaaggaagggaagctgagctatactgtaaaacagtatctcaagtgtctgagttggagaagctttttgggaggttgtggacacagtgtcaggagtgccaaggttcacttcatcaagatgttctctgcaccagtcgggattgtccaattttttatcgacgaaaaaaggcacaaaaagatatgtccgaagcaaaatcgcaattggaccggtggagtttttaagttttgccaaggaaaaggagaagaggatatacctgtgaacattaaatatcctcttccctcaaaatgaagagatgtagagggagaatcacatcagctgtaatatttacgaatttaccttcattcatctacaagggtttttaatattaaaaaacaatatttt

SEQ ID NO: 16 - DNA, 3240 bp, Phaseolus vulgaris
  source 1..3240: mol_type = other DNA
atgagcgcctcccgaaagcgcgcggcggcgccgccgaaacctccgccgtcgaagcaagccttattgactcaggaagaggacatggacgaagacgtgtttatggagcaaaccctcatttctgaagacgaagagtccatgattctccgcgacatcgagcagcgccaggccctcgctgctcgcctatccaagtgggcgcgtcctccactatcggccgattacgtcaaacaagtcgtttttcagcagctggagattgattacgtgatcggggagagtcacagggagttgctgccgaacttgtctggacctgctgccattatcagaatctttggagttacgaaggaaggacacagtgtttgctgcaatgttcatgggtttgaaccatatttctacatctgttgccctcctggaatgggtccagatgatatctctcattttcaccaaacacttgagggaaggatgagagaagccaatagaaatagtaatgtaggaaaattcgttcgccgcattgaaatggtgcagaggagaagtattaagtactatcagcaatctgattcccagtcctttctcaaaattgtagttgcactcccaacaatggttgccagctgccgtggtattcttgataggggtattcagcttgatggtctgggaatgaagagcttctggacttatgaaagcaatgtactttttgccctacggttcatgattgattgcaacatagtaggtggaaattggattgaaattccagcaggaaaatataagaagatagcgaaaagcttgtcttattgccagttggagtttgattgcctgtattctgatttgattagtcatgctccagaaggggaatattcaaagatggctccatttcgcatattgagttttgacattgagtgtgctggtcgtaaaggtcattttcccgagcctagccatgatcctgttatccagattgctaatttggttactttacaaggagaggaccagccacttattcgtaatgtgatgacccttaaatcatgctctcctattgttggtgttgatgtgatgtcatttgatacggaaagagaagtcctgctagcttggagggatttaattctggaagtggaccctgatattattattggatataacatctgcaaatttgacctgccatatcttattgagagagctgcaaacctgaagatagcagaatttcctattcttggccgtattaggaacagtagagttcgggtaaaggatacaactttctcgtcaaggcagtacgggaccagggaaagcaaagaagttgcagtagaagggagagttcagtttgacttactccaggttatgcagagagactacaaattaagttcgtattcactaaattctgtctcagcacacttcctttctgagcagaaagaggatgtccatcattctatcatatctgaacttcagaatggaaccccagaaactaggaggcgtcttgctgtgtattgtttgaaggatgcatacctccctcagcgacttttggataaattgatgctcatttacaactatgtggagatggctcgagtaacaggtgttccaatttcttttctactttccagaggccaaagcattaaggtactttcacaacttcttaggaaggcaagacagaagaatctggtcattcctagtgtcaaacaggtcgggtctgaacaaggaacattcgaaggtgccactgtattggaggcaagggctggattttatgaaaaaccaattgctacgttagattttgcatccctgtatccatctattatgatggcctataatttatgttattgcacactggtgacttctgaagatgctcgcaagctgaacatccctccggagtccgtgaacagaactccatctggtgaaacatttgttaaagcaaatctgcagaagggaatacttcctgaaatacttgaagagctattaacagcccgaaaaagggcaaaagcagacttaaaggaggcgagggatcccctggagagggcagtactagatggtaggcagctggccctgaaaattagtgcaaattctgtgtatgggtttactggggctaccattggtcagttaccatgtttggagatatcatcgagtgtaacaagctatggtcgacaaatgattgagcacacaaaaaaacttgtgggagataaatttacgacacttaatggctatgagcacaatgttgaggtaatatatggagacacagattcagtcatggtacaatttggtgtttcttctgtagaacaggctatgaacttggggagagaagctgccgaatatataagtggaactttcacaaaacccatcaaactagaatttgagaaggtttattatccatatctattgattagcaagaagagatatgctggtttgttttggacaaaaccagacaactttgacaaaatggacactaaaggtattgaaacagttcgaagagacaattgtttgttggtaaaaaacctggtgaatgattgtcttcacaaaatactgattgacagggacattccgggggctgtccagtatgtcaagaacgcaatttcagatcttctcatgaatcggatggatttatctctgctggttattacaaagggtttaacaaagacaggagatgattatgaagtaaaggcagctcatgttgaacttgctgaaaggatgcgcaagcgagatgctgccactgctccaaatgttggagatagagtaccttatgtaattatcaaagctgcaaaaggtgcaaaggcgtatgagaaatcagaggaccctatttatgttctagagaacaacattcctatagatcctcaatactatcttgagaatcaaattagcaagccgattctgagaatttttgagccaattctgaagaatgctagcagagaacttctccatggagatcatacaagatctatttcaatttctactccgtcaaacattggcttattgaaatatgctaagagaaaccttacctgcattggctgtaaagctgtactaggcaaggatcttcacacagtctgttcacattgcaaaggaagggaagctgagctatactgtaaaacagtatctcaagtgtctgagttggagaagctttttgggaggttgtggacacagtgtcaggagtgccaaggttcacttcatcaagatgttctctgcaccagtcgggattgtccaattttttatcgacgaaaaaaggcacaaaaagatatgtccgaagcaaaatcgcaattggaccggtggagtttttaa

SEQ ID NO: 17 - protein, 1085 aa, Glycine max
MSNNASRKRAAPPSQPPPAKQAVMTQEEEFMDEDVFLNETLVSEDEESLILRDIEQRQALANRLSKWTRPPLSADYVAGTRSVLYQQLEIDYVIGESHRELLPNSSGPAAIIRIFGVTKEGHSVCCNVHGFEPYFYICCPPGMGPDDISHFHQTLEGRMREANRNSNVGKFIRRIEMVQRRSIMYYQQSNSQPFLKVVVALPTMVASCRGILDRGIQLDGLGMKSFLTYESNVLFALRFMIDCNIVGGNWIEIPAGKYKKTVKSLSYCQLEFDCLYSELVSHAPEGEYSKMAPFRILSFDIECAGRKGHFPEPTQDPVIQIANLVTLQGEDQPFIRNVMTLKSCSPIVGVDVMSFETEREVLLAWRDLIREVDPDIIIGYNICKFDLPYLIERAANLKIAEFPILGRIRNSRVRVKDTTFSSRQYGTRESKEVTVEGRVQFDLLQVMQRDYKLSSYSLNSVSSHFLSEQKEDVHHSIISDLQNGNAETRRRLAVYCLKDAYLPQRLLDKLMFIYNYVEMARVTGVPISFLLSRGQSIKVLSQLLRKARQRNLVIPNAKQAGSEQGTFEGATVLEARAGFYEKPIATLDFASLYPSIMMAYNLCYCTLVTPEDARKLNIPPESVNRTPSGETFVKSNLQKGILPEILEELLTARKRAKADLKEAKDPLEKAVLDGRQLALKISANSVYGFTGATIGQLPCLEISSSVTSYGRQMIEHTKKLVEDKFTTLNGYEHNAEVIYGDTDSVMVQFGVSAVEEAMNLGREAAEHISGTFTKPIKLEFEKVYYPYLLISKKRYAGLFWTKPDNFDKMDTKGIETVRRDNCLLVKNLVNDCLHKILIDRDIPGAVQYVKNAISDLLMNRMDLSLLVITKGLTKTGDDYEVKAAHVELAERMRKRDAATAPNVGDRVPYVIIKAAKGAKAYERSEDPIYVLENNIPIDPHYYLENQISKPILRIFEPILKNASKELLHGSHTRSISISTPSNSGILRFAKKQLTCIGCKALLGKGYHTLCSHCKGREAELYCKTVSQVSELEMLFGRLWTQCQECQGSLHQDVLCTSRDCPIFYRRKKAQKDMGEAKLQLDRWNF

SEQ ID NO: 18 - DNA, 14693 bp, Glycine max
  source 1..14693: mol_type = genomic DNA
gggaactcattacgctgccactccacacactttccactctcactccgcctttctctccctcccaattccaatgagcaacaacgcctcccggaagcgcgcggcgccgccgtcgcaacctccaccggcgaagcaagccgtaatgactcaggaagaagagttcatggacgaagacgtgttcttaaacgaaaccctcgtttccgaggacgaagaatccctcattctccgcgacatcgagcagcgccaggccctcgccaaccgcctctccaagtggacccgtcctcctctctccgccgactacgtcgccggaactcgtagcgtccgtgagtgttttccgttaattatttccctttttctttggttctagggttttgtttttccgtcttattagtggagtttgattgatttgtgttctgtttttggcttttagtttatcagcagctggagattgattacgtgattggagagagtcacagggagttgctgccgaactcatctggacctgccgccattatcagaatatttggagttactaaggaaggtttgatcgtgtttctgttttcttccatactgcaaaatttggctgcatagacaattgcagataagcattgctggtttgtctgcttataatcttgcatggcgttgtagggaaggaagtaaaaaagaatgcttgcttgaggatgtaacgattgaccgatttagaaattagaaacaaaacactagtagttgatgatacatttgagcgtttctgttgttgggtttctatcagtaaaccagtcagtttgcttttaattggaggaatggattaaaccttttgttttttttacttgatccacttatcaacaaatttgccccaaaagtcttattaaaagcataatcaatagatataaataaaaaataatgtatgaagtttcatatattagtagaaaactaataatctataaatatgtgaattgtaggcaatatatgatgcacgactaactacacaatttcagtctgggtttcataattgaaaactgaaccaaactatatttacgtaaaggaaaccaaacttatttgtgtttgtttggccttttgattgccaaattaagatttatcaacctacacatatagtttgtttgagtccagatatgaagtgaccaatgcttcagaaaggggcaaattgatgctctgacaaacttgtgctgaatatatctggtatagctgaaatcaaacccaacatcatgcttggattttactttcttgaatactcagttgccttcttacataattcatgttatgattttccaattacattacatgtatgaatttaagtgctcagaaataatctgttttaatagaaccaatcaagtgtttatttgaaaaaggtggtgtaaaaattatatatgattatcattgtacttatgattatggttctcaagtcaaccatgcagtgccttacaggaattatataataaacatattttgtttgtttgtcaggacacagtgtttgttgcaatgttcatgggtttgaaccatatttctacatttgttgccctcctggaatgggtccagatgatatctcccattttcatcaaactcttgaggtatctagtttagtatctgaaaatactttcctttttccagtttttagtttagaaaagggaacaggcttcatttttttataacttatgaactgctttaatttagtatcttttgtgtcctgtgatgcttcagggaaggatgagagaagccaatagaaacagtaatgtgggaaaattcattcgccgtattgaaatggtgcagagaaggagtattatgtactatcagcaatccaattcccaaccctttctcaaagttgtagttgcactcccaacaatggttgccagctgccgtggtaaacttagatattttcagtagacattctattttccagtatattaatcttcttatcaaaactcagagaagtccagaattacttgagtgatgtttaagctacgttcttggttctcattggctgtacttttgatattgtcatagtatggccatggcatacactttaggttggttaagtgttaaattttagtgtcatgcatcttgacactagtatgtttgcattttttgtttttacagttgtctttctctacagcaattacctcatgcatcaacatttcttgctaatatttcaaaacttggcaggtattctggataggggtattcaacttgatggtttaggaatgaagagcttcttgacttatgaaagcaatgtactttttgcccttcgcttcatgattgattgcaacatagttggtggaaattggattgaaattccagctggaaaatataagaaaacagtgaaaagcttgtcttactgccagttagagtttgattgcttgtatcctctttcatgtgataatatctgtataaatgggctgtgggaataccaaaatctttagatttttctgttctccccttaacagtgttataggtattccgaattggttagtcatgctccagaaggggaatattcaaagatggctccatttcgcatattgagttttgacattgagtgtgctggtcgtaaaggtcattttcctgagcctacccaagatcctgtaatccaggtccgttgttctctcttttcacacctgcaaattcaagcacaagtttggttttttttaactgaaaattctcatttcttacagattgctaatttggttactttacaaggagaagaccagccatttattcgtaatgtgatgacccttaaatcatgttctcctatcgttggtgttgatgtgatgtcatttgaaacagaaagagaagtcctgctagcttggagggtatttcaattgtgtgtttaaccatgtttatacttaataagcataataatttcaagaaaacccccaaatttgattcgccagtccccctaaataaactatttttttatatgcttgttaagtatctgacatcttgtatgctttgtcacatagtatgatgttttgtagttcttacaacttggttaatgttattatatcatttgtttcttacacctgaatatcatgagtcaatttgataatgtaactgttgaatgaattataatcctttctctattatgcttatagtttgatggacgtgatttttttattgcaggatttaattcgtgaagtggacccggatattattattggatacaacatctgcaaatttgacttgccatatcttattgaggtgttgacttgactttgagattaaaacacttagagttgatagctgtaaggttttgttattaaaataaggttgtgtgcaagttgttgtggttactgttctaatatagtttaatttcttgttgtcttgttttcagagagctgcgaacctgaagatagcagaatttccaattctgggtcgtatcaggaacagtagagttcgagtaaaggatacaactttctcatcaaggtaaagagatacagtcttctgttgtgtacatgttttctctcattatttttttagtagttgtattctggtcggtaggtaggttgtgtatagtttcatcaattttaatgaaaatagaaacttggtttctaaagtaaatcctgcactctatatccttagcattttctgagtttcggaattactttgtgtatgcttttattttctgacttttcaatcatttgactatctaccttatcgatacttgatggtttggtaggcagtacgggaccagggaaagtaaagaagttacagtagaagggagagttcagtttgatttactccaggttctgtcaatattgttttgctttaactaccttgtgcttgggcacaccgagtttattgcttaataggtttatttgttttgttctcatttacaggttatgcaaagagactacaaattaagttcttattcactgaattctgtgtcatcacacttcctttctgagcaggtaaagtcattcttttggcatcatttcaagcaattttttccccctttattttgtgtgccttggcacaatgttaaggatattgccttgtgacttggaggtgatgtgttcaaatcctgtaaagcttctctgcttggggtataagattgtgtacatctaccctccttagatcccattatgtgggagccttgtacattgggtcacccttttatctttttttttattcctttattttctctttcaaatacatctgggaagtgggaactgttaatgggacttaaatcatggttggaaagtacaagtaacatctttatatttatatacaaatataagatatttatctatttataatcatagctgtagttgcagatttacatgaataaactcatgatttgatgtaagcattggtcatgttagttggattgtacaagagtctactatattgggaaaaactcaagttccacattggctagagatagtgccaagacaaagtatataaatggaggacaaccttgagccttatgagctaacttttggggttgagtttgacccaaacccacattctaagatgatatcagagtttatcctagatccattaacgaaccacccaccatattatccacgcaccaaacctaaaagtgttaggcgtgagggggtgtattggaaaaaatccaagtcccacataggctaaagataatgccaagatagagtatataagtgagggacaacctttcccctataagataacttttggggttgagttaggcgcaaatccaaattctaatagattgcaatggagttgtaatttgatagatatacatttcaattggtcattttatttgctgtctgtatgttcctatgtgtgtctatgcacttctccatttgtgacctatttatatgcctaccattttctaaacagcttcttttgctggaatctcgtctttgttcttgaaagttttagtgcatgtcaatttattggcttagtcactgcatgtttgtatgttacagaaagaggatgttcatcattcaattatatccgatcttcagaatggaaacgcagaaactaggaggcgccttgctgtgtattgtttgaaggtttgtattctggttcttttatctagggatgctctttgttgctacctttcatctaaatgattattgtccagtctttttatgaagttctcattggccggtaaactttatggagatgaactagtggttataactagtttggaagtctcgattgatgatgaaaatcatgatgtgattgataagcacaacatttttggtatgcattgtatttttatgaaatggctctgtttcatctatttttatgaaatggaagcaagataatttttaacttcattttattgaccatttctgctacctttttatgaggccttagtttttcaagatcacctaacaatttgacaagatttgctcaaatatatttttctcaatgctaatacccaataggttcaataattttttaacctaaaatacttaataatgcatttagattgaaaaagttgtaatgcttgttcttttacctattaaagtgctgtgtgtcagatattgtgaatccattatgggtatattacatgtgaaaaaaaactaagtgaaataaattgaccattcaaaattcatactttgcttgtgttaatgcaggatgcatatctccctcagcggcttttggataaattgatgttcatttacaattatgtggagatggctcgagtaacaggtgtcccaatttcttttctactttccagaggccaaagcattaaagtaagcattgaagttcctttttcttatcaatcattagaaaatagaaatgcataaaatgattgatcattgaatttgtagcagtaattgcttgttttcttcctttccaggtactttctcaacttcttaggaaggcaagacagaggaatctggtcattcctaatgccaaacaggctgggtctgaacaaggaacatttgaaggtgccactgtaagttcattcttttctttttcatattcattattttaaatgattaattggcattgattcatagattctttccactttcaatgtatgtacatgcatgtttgcatgtgtatggatcttccaatatgaaatgaaatatattgcattagctacccagtcatacaactattctatgccttgtattttttgagagttttatttatatttcatcctcaaattagattacatattgaattgactcatgatgaagggaattagaaattgaaaggttctttactccaagcttgctttgcattaatgttcttaaattcaagagcctatgtaaacttgtggagggttgtaaactcataagactacctaacattgaaataatattaagaatacctaatcacatatccaaacaaaatagattaccataatatcaaatctatgaaactagaaatagacaaatcactacaatccaattatccaagaggatcaaatgaacaagggcttgaatcttgtcacagaacaaatgggaatgcaaaggtcgaatgggcttatcactcttagaagaagtgacagagttggataaaggtggaaaaagatcactgtaaggatatttaagctcattgaagataacatctttggaaacatataatttgccattaggagagagacacttgtaacctttgtgagaagtggaataaccaaggaacaagcactcatgtgacctgaaatcaacaaggtgaatcatgcgtgtctcatgcgtgagcagtagtgcctctacttctccaatcagaagtggttcaaacttactttcaatcactgagatcactggactgtactcctctggaaggccttcaagaatcgcgtcaaggtgttcatgcggaagaataggatctccacacgatccaagctcgactatacatttggtgcgcaaaagaaactcacattgttttctgctcaagcattgtagttctgagtttgttcgaagtattctagctttcgcacgtgtttgcttttgaaaatactcatggatctgctcccaaagctgatatgaatgaacgcattcaataatttgagataggattgaaactgaaagagtcaattgaagccacgaaaagagaatttgatcttgttgttcccaatcagtgtatgctggattttcatttccagcttcacaatctgtctctatgagaaagcacatcgggatctgaggacttgccacgaaatgctgaagtttatgagccttcatgacaggttctacctgttgccttcatagcaggaagttagaatcgtctaacttctgagaaatctgatgaaaacaaggaagtagcggatgcgaaattcagatttgtcgccatggattgacctcaagctctgataccatcatagagtttgagagagagaaaaagaagaagatgaaatatattattattgattcaaaatgaaaaggtcagttacaaaactgattctgtttccttatttatagagaaaacaaatattgttacacaaaacctgactaaggcagttaactaaccaaagttagttattgctctaacagaaataatagcttctaacagaaaaatagaaataacaaaatcacagctgtcctaactgaatcataactgttctaactgctgccctaattgaatcacaaagtaaaacttacttaatacactaatagagttgagttggcgtgatttctcaacctgaagttgcaaacttgtcagactttacgagtcaacttcaagtttatcgaccatgctttgtataattttctcttctatattcatgaaacaatatctttcgtattttttggttagatagaaatgtggattccgtatttacactttacagttggaacaaagaacgacaattgtgccacctagtattgcctttttgatttagggaaagtaggaaaaaaataactctatcctactaatgacaaatatacagtgagatttattctattcacaagaaaatgattttatttcttgattgaaaaataaaatgaacaaggagaaagatctcctccaagggtatccccttaacaagggtttcccattactactacatcagagataaagctcagttctcaattgatacaaaacagaatgtcaccatgattgagctaaatcccctgtccttccagtctctctagctaactattgaattttctaaccattgatacgttttttactctgactagtaactattgatgtttgggaatctctaacagcatgttaggcctgaaatcaagctcaatcttaaatgccctcttgttcttcagttaataaatgaaaaattgaaagaattctcaaatttcaagtatctttatgtatggagatctacataatattattggagttaatgttctacaccttttcctgtatttgccttttggacgtttcacttgtcaagctgtgttgccttttgatgcatgcattttgaactttacaacttgcaggtattggaggcaagggctggattttatgaaaaaccaattgctactttagattttgcatccttgtatccatctattatgatggcctataatttatgttattgcactctggtaaatatttctttttttttttagtttttccttaagcttctgagttttgttgtttctttaaaaaaaattatgagtactactttattcatcagtttagttcaattcgtatgggggtaacaattggtacttttttttttttttgttttactgttctttaaggtgacccctgaagatgctcgcaagctcaacatccctccagagtctgtgaacagaactccatcgggtgaaacatttgttaaatcaaatttgcagaaggtatattgcttgcgcttactatttcactttttcctttagggctctatgctaatttcattttagagtttaatgcttatatgaagttgaggcactgatgtaacgagaataaatttagggaatacttcctgaaatacttgaagagctattaacagcccgtaaaagggcaaaagcagacttaaaggtatcctttctgtacttgttgtgaaaattgactttggatttggattattgaactgtcagctgtagtgtttgattaattaaaaattcactaactgagttctatatgttatctcttcctgtacatgatttctctccatatttctacttcaggaggcaaaggatcccctggagaaggcagtgctagatggtagacagctggccctgaaggtaagctgtgcatgctattgttttatgtcttttttccttgtcctttctcttttgatagttgttcagttctctccactgcaaagtgcgtactgataactgctaattcttggtaacattttcgattcctattttcttttggtcagattagtgcaaattctgtttatgggtttacaggggccaccattggtcagttaccatgtttagagatatcatcgagtgtaacaagctatggtattgctttcatctctaagtttgtgttgccgtgtatttctaaaattatcccttgttgaattgaaataacatctccattgagttcttattattatgtggattggatgccttgttaaggtcgacaaatgattgagcacacgaaaaaacttgtggaagataaatttacgacacttaatggctatgaacacaatgccgaggtataatatttagaatttttcttgttttgatgcttgtcatgctctgaggtttcattggttagacagtgaagctatcttaggtgtttttattggtccttattacagcattttggcctggggaatttaacattgaaccaacaatataaaactttctatccttgccaataaattttacacttactgttatgcatctgttttcctaaatctaatgttgtcatatcttattttcatgctttactaccataatcaattagccagatgacttacaaagtaatctcatactaggtaatatatggagacacagattcagtcatggtacaatttggtgtttctgctgtagaagaggctatgaacttggggagagaagctgctgaacatattagtggaactttcacaaaagtaacaattcattttgtgttttattttttttctttagtctttgtaagtttgagacttttgtaaatagcctgtgtaagtgatgaaatcctgaacttgaaactgtttttattttctccaaacaatgaagtaatgattgattcttaaatcactttattttttatttaacacaagaattcagtaaagatgccccctcccccgggcttgcaaaacaacatattctcaatttgattattacaatcattcccctatttcttcaaatatgggacaacaatttacatagtttttcaaatagttcagattcagcaattatgctattatttatgacattcattattcatatacacattaattacaactttacaaaatttgctagctagtttagtggtagtgtacaaagagatgaggagtaacactgtagtggtctttgtacttcaggaacttgcatcaatgtgtaacgttatatgttgatgcatttatggtcttggtctgcatatttttcaagtttctgcttcagcatctctaaacttaattctatcaaccaattaacaaaatatcagaaaagaaatcctcaacttaaattttctagagacttcattcttgtttaagtgtgtttgaagtcaactaattattttaattttttactttgatttgtccttacgtatttgttgtcattttgattgtgaagcccatcaaactagaatttgagaaggtttactatccatatctcctgattagcaagaagagatatgctggtttgttttggacaaaaccagacaactttgacaaaatggacactaaaggtaaacattactctttaccttcttgttgctttgttgaagtgcctcttaaactaaatatgcattgagcaggtattgaaacagttcgaagagacaattgtttattggtcaaaaacctggtgaacgattgccttcacaaaatattgattgacagggacattcctggggcagtccagtatgtcaagaatgcaatttcagatcttctcatgaatcgtatggacttatcacttctggttattacaaaggtgggaagtctattgaatctacagaccacctaatggcctttatagctatttgctttttgtggagaattcagatatttgatgcaagtgcactaccaaaataattcctttgaacttggcactaacatgcttgctttagtagggtttaacgaagacaggagatgattacgaagtaaaggcagctcatgttgaacttgctgaaaggatgcgcaaggttggttagataatcaaatgcattggtggttttgttttcacccattttagtttttggaaaaatgagatttttttggcaaaggaaaacaagtcagttatagtatatgatctatatgtgccaaagcatgcaaccaaatattagaaaaaaccatgaattatttttaatctagacgctggcattctatattgtctttgctcaggtcctgtaagttgatgtttctaatgtctgggaagttatttatggaactatacagtattgttactgctcttaagatttatatttattgttttaggcttattgagcttgtcatgtctcagttctttgggccataaatgtattttatttgattttgtaattgacagcgagatgctgccactgctccaaatgttggagacagagtaccatatgttattattaaagctgcaaaaggtgcaaaggtaatacatgtccccctctttttttgaagttttttaaacatttgtttagattttctctagactaacgtgtaagaaatcaggcatatgagagatcagaggatcctatctatgtgctagagaacaacatacccatagatcctcattactatcttgagaatcaaattagcaaggttggtttattagtttattagatgttttgaatttctccaatgttttctgctatttattagcttcattcatgtgcagccaattctgagaatttttgagccaattctgaagaatgctagcaaagagcttctccatggaagtcatacaagatctatttctatttctacaccgtcaaacagtggcatattgagatttgctaagaaacagcttacctgcattggttgtaaagctttacttgggtatcattttttttttctcttttcatagtgttgtcttgaggctctaagattttcctggttgattttacttttatgtattcccttttttggtgtttgcttcattatatttttgtaaataaaagatacacaggaattacaaagcgctaacaatttatgcatttgctaactttttcaatagctgggtttgcgtatttgagagattttctattgacttgaaatgtataagaaacatagtgcagcactatattatggaattattatgaacctttcaaattacctactttaagaataaatgtgctttatggaagtgaacgtaagaccccaacaaaaaatagcatgaatgtaataggggtgaaactccttcagagattatgttaatgaatatatgagaacttttgggcatctcagtgacatagttggtttaattgaatttcatatcctctgtaaatttctctccctacttttcaataatgtttctgtagatactacctgtgagatagcattttgcactggaggtgccattgatctgtgtgctattatgagaccttgaaatcttggtgtgagtttcctggagtattttattatggttttatcttgagccttaacaggaaaacaagattcaacttatatctccactagtaatgtacctgataatactcaggccttttttctaagacattatttgtatgtattatgatacacaagtatatctcagttatacaaaggcctaataatactatttttgttgcttttggttcagcaagggttatcacactctctgttcacattgcaaaggaagggaggctgagctgtactgtaaaacagtatctcaaggtaggcatctcttgagctctctgttcaatttccaatcgttttgttgtactggatggttccagatgtttttttattggataaaacaagtggtggtgtgactgcagtgtctgagctggagatgctttttgggaggttgtggacacagtgtcaggagtgccaaggttcacttcatcaggatgttctctgcaccaggtatgttcattcagttaaatattatatcattgagggattgcttggggggctagagttctcagatttttggttttaagaggcacaatatttcagtattaaattaattaatcacatcctggagttacggtggttttacccccttctccctgggtttgtttactgtcctggcataacatgcatcaacttggtttcttcttactcatgtcttcccatctgcttttgtagatttcgtacaagaacttgtaatttagttgtggtttctcattgttagttcattatcactgatttatcatgtactatggtgttgcagtcgggattgtccaattttctatcgacgaaaaaaggcacagaaagatatgggtgaagcaaagttgcaattggacagatggaacttctaagttttgccaaggtcttttttttccctcaattttcttccccctttagtcttttgccttcataatttttttttcagtcttttttgtcttcataatgatttagtcaatgaatgacatgtaggaatgtcacccattaatatatccaataattgcaatacaaacagctggaacatttatcttttcgtctcaggaagttataaaatattttataaaataaataaaaaattgaggatttatgaatttttagtaaatcataactggatatcttggtatcattttctgttaagattctacacttttgggtatttacatccacatgcacatgcatgtgctctaccttgcataatgttcagttttcagaacttttatttcaaatttgccccaaataaatcatatttgagtattatgccctatcaattttcaaccattaccatcaatcaccccgatagtttcaagtaatgcttttatagagagtaagaatgcttcagtaaaaattgttattgttgttttgtgtgaaaaaacgttagacatcaatcattttggaaaggagggaatattattttttttaaagaaaaggaggaagtattagtgaaccctgatatatatataccattcctagtttgtttattggggttgatataaataaataaaagttacaattgtaggtatcattaaatttgccaattttgggaactgaaggaaaacctctggtgatgatatcctcctacgacaagctcttgtgactatttctatttctggaaggaaatgcatgtagcgaagagcctaatttcttgcacggtttagttcaacatatgattttgcgaaatttggttaatgtgcagaatttgaccttgcggatctcttcgaaaccaatggacacaaatacaatctggtgtttgccacaatcctgacatttgtaatgtgagtaaaagcccacaatttgtttactgaaagatttgtgtggaatcctcaaatgatagtttttaagttatcggatgacatcaacctggttccaagcagtaactgctgaattattttgatatttaaaaaattatcattcgaggatttagtcagttgatgcaaagcagtcttttgatatggaaatgagggtatactgtaacgtaaggatatgtcaacgggatgagttatagatgtgaggcggtgtctgttacaaaaggatatcctagtaattgacgcgtgttctgatattttggtgcatgattaaatcaacagtttcactc

SEQ ID NO: 19 - DNA, 3915 bp, Glycine max
  source 1..3915: mol_type = other DNA
gggaactcattacgctgccactccacacactttccactctcactccgcctttctctccctcccaattccaatgagcaacaacgcctcccggaagcgcgcggcgccgccgtcgcaacctccaccggcgaagcaagccgtaatgactcaggaagaagagttcatggacgaagacgtgttcttaaacgaaaccctcgtttccgaggacgaagaatccctcattctccgcgacatcgagcagcgccaggccctcgccaaccgcctctccaagtggacccgtcctcctctctccgccgactacgtcgccggaactcgtagcgtcctttatcagcagctggagattgattacgtgattggagagagtcacagggagttgctgccgaactcatctggacctgccgccattatcagaatatttggagttactaaggaaggacacagtgtttgttgcaatgttcatgggtttgaaccatatttctacatttgttgccctcctggaatgggtccagatgatatctcccattttcatcaaactcttgagggaaggatgagagaagccaatagaaacagtaatgtgggaaaattcattcgccgtattgaaatggtgcagagaaggagtattatgtactatcagcaatccaattcccaaccctttctcaaagttgtagttgcactcccaacaatggttgccagctgccgtggtattctggataggggtattcaacttgatggtttaggaatgaagagcttcttgacttatgaaagcaatgtactttttgcccttcgcttcatgattgattgcaacatagttggtggaaattggattgaaattccagctggaaaatataagaaaacagtgaaaagcttgtcttactgccagttagagtttgattgcttgtattccgaattggttagtcatgctccagaaggggaatattcaaagatggctccatttcgcatattgagttttgacattgagtgtgctggtcgtaaaggtcattttcctgagcctacccaagatcctgtaatccagattgctaatttggttactttacaaggagaagaccagccatttattcgtaatgtgatgacccttaaatcatgttctcctatcgttggtgttgatgtgatgtcatttgaaacagaaagagaagtcctgctagcttggagggatttaattcgtgaagtggacccggatattattattggatacaacatctgcaaatttgacttgccatatcttattgagagagctgcgaacctgaagatagcagaatttccaattctgggtcgtatcaggaacagtagagttcgagtaaaggatacaactttctcatcaaggcagtacgggaccagggaaagtaaagaagttacagtagaagggagagttcagtttgatttactccaggttatgcaaagagactacaaattaagttcttattcactgaattctgtgtcatcacacttcctttctgagcagaaagaggatgttcatcattcaattatatccgatcttcagaatggaaacgcagaaactaggaggcgccttgctgtgtattgtttgaaggatgcatatctccctcagcggcttttggataaattgatgttcatttacaattatgtggagatggctcgagtaacaggtgtcccaatttcttttctactttccagaggccaaagcattaaagtactttctcaacttcttaggaaggcaagacagaggaatctggtcattcctaatgccaaacaggctgggtctgaacaaggaacatttgaaggtgccactgtattggaggcaagggctggattttatgaaaaaccaattgctactttagattttgcatccttgtatccatctattatgatggcctataatttatgttattgcactctggtgacccctgaagatgctcgcaagctcaacatccctccagagtctgtgaacagaactccatcgggtgaaacatttgttaaatcaaatttgcagaagggaatacttcctgaaatacttgaagagctattaacagcccgtaaaagggcaaaagcagacttaaaggaggcaaaggatcccctggagaaggcagtgctagatggtagacagctggccctgaagattagtgcaaattctgtttatgggtttacaggggccaccattggtcagttaccatgtttagagatatcatcgagtgtaacaagctatggtcgacaaatgattgagcacacgaaaaaacttgtggaagataaatttacgacacttaatggctatgaacacaatgccgaggtaatatatggagacacagattcagtcatggtacaatttggtgtttctgctgtagaagaggctatgaacttggggagagaagctgctgaacatattagtggaactttcacaaaacccatcaaactagaatttgagaaggtttactatccatatctcctgattagcaagaagagatatgctggtttgttttggacaaaaccagacaactttgacaaaatggacactaaaggtattgaaacagttcgaagagacaattgtttattggtcaaaaacctggtgaacgattgccttcacaaaatattgattgacagggacattcctggggcagtccagtatgtcaagaatgcaatttcagatcttctcatgaatcgtatggacttatcacttctggttattacaaagggtttaacgaagacaggagatgattacgaagtaaaggcagctcatgttgaacttgctgaaaggatgcgcaagcgagatgctgccactgctccaaatgttggagacagagtaccatatgttattattaaagctgcaaaaggtgcaaaggcatatgagagatcagaggatcctatctatgtgctagagaacaacatacccatagatcctcattactatcttgagaatcaaattagcaagccaattctgagaatttttgagccaattctgaagaatgctagcaaagagcttctccatggaagtcatacaagatctatttctatttctacaccgtcaaacagtggcatattgagatttgctaagaaacagcttacctgcattggttgtaaagctttacttggcaagggttatcacactctctgttcacattgcaaaggaagggaggctgagctgtactgtaaaacagtatctcaagtgtctgagctggagatgctttttgggaggttgtggacacagtgtcaggagtgccaaggttcacttcatcaggatgttctctgcaccagtcgggattgtccaattttctatcgacgaaaaaaggcacagaaagatatgggtgaagcaaagttgcaattggacagatggaacttctaagttttgccaaggtatcattaaatttgccaattttgggaactgaaggaaaacctctggtgatgatatcctcctacgacaagctcttgtgactatttctatttctggaaggaaatgcatgtagcgaagagcctaatttcttgcacggtttagttcaacatatgattttgcgaaatttggttaatgtgcagaatttgaccttgcggatctcttcgaaaccaatggacacaaatacaatctggtgtttgccacaatcctgacatttgtaatgtgagtaaaagcccacaatttgtttactgaaagatttgtgtggaatcctcaaatgatagtttttaagttatcggatgacatcaacctggttccaagcagtaactgctgaattattttgatatttaaaaaattatcattcgaggatttagtcagttgatgcaaagcagtcttttgatatggaaatgagggtatactgtaacgtaaggatatgtcaacgggatgagttatagatgtgaggcggtgtctgttacaaaaggatatcctagtaattgacgcgtgttctgatattttggtgcatgattaaatcaacagtttcactc

SEQ ID NO: 20 - DNA, 3258 bp, Glycine max
  source 1..3258: mol_type = other DNA
atgagcaacaacgcctcccggaagcgcgcggcgccgccgtcgcaacctccaccggcgaagcaagccgtaatgactcaggaagaagagttcatggacgaagacgtgttcttaaacgaaaccctcgtttccgaggacgaagaatccctcattctccgcgacatcgagcagcgccaggccctcgccaaccgcctctccaagtggacccgtcctcctctctccgccgactacgtcgccggaactcgtagcgtcctttatcagcagctggagattgattacgtgattggagagagtcacagggagttgctgccgaactcatctggacctgccgccattatcagaatatttggagttactaaggaaggacacagtgtttgttgcaatgttcatgggtttgaaccatatttctacatttgttgccctcctggaatgggtccagatgatatctcccattttcatcaaactcttgagggaaggatgagagaagccaatagaaacagtaatgtgggaaaattcattcgccgtattgaaatggtgcagagaaggagtattatgtactatcagcaatccaattcccaaccctttctcaaagttgtagttgcactcccaacaatggttgccagctgccgtggtattctggataggggtattcaacttgatggtttaggaatgaagagcttcttgacttatgaaagcaatgtactttttgcccttcgcttcatgattgattgcaacatagttggtggaaattggattgaaattccagctggaaaatataagaaaacagtgaaaagcttgtcttactgccagttagagtttgattgcttgtattccgaattggttagtcatgctccagaaggggaatattcaaagatggctccatttcgcatattgagttttgacattgagtgtgctggtcgtaaaggtcattttcctgagcctacccaagatcctgtaatccagattgctaatttggttactttacaaggagaagaccagccatttattcgtaatgtgatgacccttaaatcatgttctcctatcgttggtgttgatgtgatgtcatttgaaacagaaagagaagtcctgctagcttggagggatttaattcgtgaagtggacccggatattattattggatacaacatctgcaaatttgacttgccatatcttattgagagagctgcgaacctgaagatagcagaatttccaattctgggtcgtatcaggaacagtagagttcgagtaaaggatacaactttctcatcaaggcagtacgggaccagggaaagtaaagaagttacagtagaagggagagttcagtttgatttactccaggttatgcaaagagactacaaattaagttcttattcactgaattctgtgtcatcacacttcctttctgagcagaaagaggatgttcatcattcaattatatccgatcttcagaatggaaacgcagaaactaggaggcgccttgctgtgtattgtttgaaggatgcatatctccctcagcggcttttggataaattgatgttcatttacaattatgtggagatggctcgagtaacaggtgtcccaatttcttttctactttccagaggccaaagcattaaagtactttctcaacttcttaggaaggcaagacagaggaatctggtcattcctaatgccaaacaggctgggtctgaacaaggaacatttgaaggtgccactgtattggaggcaagggctggattttatgaaaaaccaattgctactttagattttgcatccttgtatccatctattatgatggcctataatttatgttattgcactctggtgacccctgaagatgctcgcaagctcaacatccctccagagtctgtgaacagaactccatcgggtgaaacatttgttaaatcaaatttgcagaagggaatacttcctgaaatacttgaagagctattaacagcccgtaaaagggcaaaagcagacttaaaggaggcaaaggatcccctggagaaggcagtgctagatggtagacagctggccctgaagattagtgcaaattctgtttatgggtttacaggggccaccattggtcagttaccatgtttagagatatcatcgagtgtaacaagctatggtcgacaaatgattgagcacacgaaaaaacttgtggaagataaatttacgacacttaatggctatgaacacaatgccgaggtaatatatggagacacagattcagtcatggtacaatttggtgtttctgctgtagaagaggctatgaacttggggagagaagctgctgaacatattagtggaactttcacaaaacccatcaaactagaatttgagaaggtttactatccatatctcctgattagcaagaagagatatgctggtttgttttggacaaaaccagacaactttgacaaaatggacactaaaggtattgaaacagttcgaagagacaattgtttattggtcaaaaacctggtgaacgattgccttcacaaaatattgattgacagggacattcctggggcagtccagtatgtcaagaatgcaatttcagatcttctcatgaatcgtatggacttatcacttctggttattacaaagggtttaacgaagacaggagatgattacgaagtaaaggcagctcatgttgaacttgctgaaaggatgcgcaagcgagatgctgccactgctccaaatgttggagacagagtaccatatgttattattaaagctgcaaaaggtgcaaaggcatatgagagatcagaggatcctatctatgtgctagagaacaacatacccatagatcctcattactatcttgagaatcaaattagcaagccaattctgagaatttttgagccaattctgaagaatgctagcaaagagcttctccatggaagtcatacaagatctatttctatttctacaccgtcaaacagtggcatattgagatttgctaagaaacagcttacctgcattggttgtaaagctttacttggcaagggttatcacactctctgttcacattgcaaaggaagggaggctgagctgtactgtaaaacagtatctcaagtgtctgagctggagatgctttttgggaggttgtggacacagtgtcaggagtgccaaggttcacttcatcaggatgttctctgcaccagtcgggattgtccaattttctatcgacgaaaaaaggcacagaaagatatgggtgaagcaaagttgcaattggacagatggaacttctaa

SEQ ID NO: 21 - protein, 1103 aa, Zea mays
MNSGGRGGKRRGPPPLAPSGAAAKRVNPSPGTPHVPPPASAAAAVEEDMMDEDVFLDESILAEDEAALLMLQRDEALASRLARWKRPAIPTDLAAGCSRTVAFQQLEIDYVIGESHKELLPSSSGSAAILRIFGVTREGHSLCCQVHGFEPYFYIGCPSGMGPDDISRFHQTLEGRMKESNRSSNVPRFVKRVELVQKQTIMHYQTQQSQPFLKIVVALPTMVASCRGILERGITIEGLGSKSFLTYESNILFALRFMIDCNIVGGNWIELPAGKYRKAACIMSYCQLELDCLYSDLVSHAAEGEYSKMAPFRILSFDIECAGRKGHFPEPTHDPVIQIANLVTHQGEDQPFVRNVMTLKSCSPIVGVEVMSFDAERDILLAWRDFIREVDPDIIIGYNICKFDMPYLIERAEVLKIAEFPILGRIRNSRVRVRDTTFSSRQYGVRESKDVTIEGRVQFDLLQAMQRDYKLSSYSLNSVSAHFLGEQKEDVHHSIISDLQNGNPETRRRLAVYCLKDAYLPQRLLDKLMYIYNYVEMARVTGVPISFLLSRGQSIKVLSQLLRKAKQKNLVIPNIKGQGSGQDTFEGATVLEARAGFYEKPIATLDFASLYPSIMMAYNLCYCTLVPPEEARKLNLPPEFVNKTPSGEIFVKPELQKGILPEILEELLAARKRAKADLKEAKDPLERAVLDGRQLALKISANSVYGFTGATVGQLPCLEISSSVTSYGRQMIEHTKKLVEDKFTTVGGYEHTAEVIYGDTDSVMVQFGVSTVEDAMKLGREAADYISGTFTKPIKLEFEKVYFPYLLISKKRYAGLYWTNPEKFDKMDAKGIETVRRDNCLLVKNLVTECLHKILVDRDVPGAVQYVKNTISDLLMNRVDLSLLVITKGLTKTGEDYAVKAAHVELAERMRKRDPATAPTVGDRVPYVIIKAAKGAKAYEKSEDPIYVLDNNIPIDPQYYLENQISKPLLRIFEPILKNASKELLHGSHTRSVSISTPSNSGIMKFAKKQLTCLGCKAVISGASQTLCSHCKGREAELYCKTVANVSDLEMLFGRLWTQCQECQGSLHQDVLCTSRDCPIFYRRRKAQKDMTEARLQLDRWDF

SEQ ID NO: 22 - DNA, 29421 bp, Zea mays
  source 1..29421: mol_type = genomic DNA
ggacctcctctcgtttcgtattaaaccctacctcgccagtaatggcgggaactcccagtttcagtccccgcacttcccgcctccctctccccctctcggtcccaccttcgtccctccggcgatgaactcaggcggacgcggcggcaagcggcggggtcccccgcccctcgctccctccggtgcggcggcgaagagggtcaatcccagccccggcacgccgcatgtgcctcctcccgcgtcggcggcggccgccgtggaggaggacatgatggacgaggacgtgttcctggatgagtccatcctggcggaggacgaggcggcgctgctgatgctccagcgcgacgaggcactcgcgtcccgcctcgcgcggtggaagcgccccgcaatacccaccgacctcgccgccggatgctcccgcactgtcggtatgacccggtttcccttttctctcccggcgctcaccctccttatccgggtcttcatggattcagtgttccgagatgtgtttgttggggtctgacaggctgcgtgtggagtgcgttggggtttggatttagtactgctttgtcggattgataatgatttgctgtcaaccgagtgtcatttggatagggttataataattttggcaatgctgttttagtcaagttgccttgatgcggtctggagctaggcaaaattttatgcagatatatgttctcttagggttgtagtgttcaggaacttgggacaatggtactttgcaagtttgggcaaaaggcgagcaattattgtttcggactaatatgtgcgttagtatatcaggttgattagtgaagtttatcgttaccatctttgtatcccagggtagttttggacagaaagaaagacactttttcagtgtcttatgcacttttcaaaactgtgagtctgtgactgtttttctaatcatgagtgatgcaactagctgctatctatgaatcatagatataaatatcaaatcaagggttaacggttttaaaggtagtacaacttctgccatcctacttcctagaagtatctcataaacctctatttttttgtcttgtacaatttcacagtcctagctcaaagttctgcttgtatcacctgccaatctgagatggatattaccatcaatcatgatagaggatcataaaatcacctagcagttttccaaatttggtgcctctgaattaatttatgaagcgaacctattttctgtcaaacattatacttatatatgtttatacacttttgattcactttgagttacgagccttaaaactttaaactctggacttgcttaacagtcaacttgtacatatgggtggtaatgggctctaaattttacactataaaattaaaggatcggatcgattaggatcaggctctattcctattcatttttgaactaaaattaattaagggctcaaacaaattgtgaagaagcatctggatcatgacccattaccacccctacttgtacatgctcaatgctaattgagattcaatgttttttgctacttaaatagatagtcacctagttttgttttaatttgaactttgtatgaacatatattcaggatagttggcttcatcttacactttctactcttctgtttgtattggatattctagcttttcagcagttggaaatagattatgttattggtgagagccacaaagaattactccccagctcatctggttcagcagccatactcaggatttttggtgtcaccagagaaggtaatatcttgagtattctattatctttcaggaaagaaatcgttgtcatatagttttggctgaactctgatgctaataaccatgtaacatgtgtaggccacagcttatgctgtcaggtgcatggatttgagccatatttctatattggctgtccttcagggatgggtcctgatgatatatcacgctttcatcaaacactagaggtttgtacttgcaatcattaccttgaactcagcttctaaggaaatagagtactttggcagtacatggtttccttatggtttcttcaaactgtactacaggggaggatgaaggagtcaaatagaagcagcaatgttccaaggttcgtgaagagagttgagcttgtgcagaagcagacaattatgcattaccaaacacaacaatctcaacctttcctcaagatagtagtcgctctgcctaccatggttgctagctgtcgtggttagtggcattcttaagatacaatacatctattattgctaagtcttgtagattgacctcactatcttttatttatttaattgatggataacctatggagcaattttatttttggttccatgtcaatgtattatcatgtgattttatgatatctacttcattcctctgatcatccaagtttttttccctttgctgtcttagggctagtttgggaaccctattttcccaagggaaattagttcattttcccttggtaaaatagaatcccttaaaaaatggagttcccaaactagcctttaaatggaaattgtttgcacattataggtatcctggaaaggggcataacaattgaaggccttggttcaaagagttttctgacatatgaaagcaacattctttttgcccttcgtttcatgattgattgcaatattgttggtggcaactggattgagcttcctgctgggaaatacaggaaagcagcctgtatcatgtcctattgtcagctagagctagattgcttgtatccttctatacttaattgaatgctttcatctatttactaaactgtgatgttgttcagttatgtctattcttaacacaactaacagatactcagatctggtaagccatgcggctgaaggagaatattctaagatggccccgtttcgcatattaagttttgatattgaatgtgctggtcgcaaaggtcatttcccagaaccaactcatgatcctgttattcaggttgagttcattattcatgtatctgatactcgttaccatttagtttcaatacaggtctttatgtttacaatttattgatttactcctttggatttaaatagatagctaatttggttacacatcaaggagaagaccaaccttttgtacggaatgtcatgactcttaaatcctgctctcccattgtgggagtcgaggttatgtcatttgatgcagagagagatattctacttgcatggagggtatgtgttgcttgctgtctttggagttaccaaattcaattgaacgtgctgtatggttgtatattgcctttatcttctttggaaaatgaatttatagcgcctttatccatggtattttcttcatttgccagtaatggtttcattctctttgccttttgtatccacattttgaatttgtttacccttttacttgctatatgcggttaacatttgttttttttgctaaaactaaatgtcattattcctcaagaaaaaaatttaaaacaaacacacttattcctgaaaagaagttaaatacattttcttattgtagacaaaggacttggccgaaaattatctgattcttatttggaaataatttgcaggatttcatacgtgaagtggatcctgatatcataattggatacaacatttgcaaatttgacatgccctatcttattgaggtacaataacacctgtagagtgttcagattttttaggccaattgacaatcagccatatttggtacaatttgattacacacatttgcagagggctgaggttctcaagatagcggagtttccaatacttggccgaatcagaaatagtcgtgttcgtgtccgtgatacgaccttttcctcgaggtcacttcttgtacctctatcctctatgcatttaatatttattacttcgccttaatcatacaattgtcttagtcaataaaatatctgttacatattctgttgtccttttatagtaacatatttagacaatttcctttttttgtggtgtgtgaggggtaggtgctagcatatcattgaagcactatgttgcttagttttattgtatgcttgcatgtgtgtctcatgtttttttttatttttgtgctttatttgattctaccgctgcatctatttatctgattctggattagctggcatacaaatcttgctcatttgtcttaaggagaaaagtcacatatttttttagatgtcatgttaccgtataatgagctgtaccatgttcatatttgatatttcacacatttcattcacagtttctgtgggtagtcatattttatgctgttatagtgggcgcactaactagtttaacccaaaagcttaagcttttaggagaaggtagacaatccacttatacacttcaacatcctcactcacgtgcggccagatagaaagacataaacgtggaaataattcatgtgtagctagagagaaaggcacaaacttggaaataaaagggtggaggcacaaataaagcctctgccaggattcgaactcgagacctctgactttgataccatgttagagtggacgcactaaccagttcaacccaaaagattaagctgttaggagaaggtagacaattttctactatatttaagtgtgagtttcttcgtgtgtcacctcctacagcgagtgcaacaagcgaataaatttcccttcccgcgcggaataaatatccaaaaatggggtatggaggaggatttgaaccctgcttgcttggtataaagcgcttggcttctaccactgtaactcatgtctatttgtgttatataaaggagatatatgtcatggaccttcggtccatgggggtagctgtcttctccggcgaggttatacccctctgccgtaggcttggttctagggtagcagccctaggcgcttgagggtcattgcaagagagagagattggtttgataatgtattccttcttcctttgtctcctgcgtacaggcctatatataggccaccggttgaccaactaggcaaccactaattaggcaagtgactaattgaaattatctcccctaaatttaggtctatcaatttagccaccaaatcagtcactttcctttgccatgccttccttgcgcgcgctttcctttgccatgccttccttgtgcgcgctttcctttgccatgccttccttgcgcgcgctgctgccagcgccccctgtggcctctcgcttttggcgcacataggtgcgaccccacatgagtgaccgctccctatgatgcccatgacatctccccctcccttgaggaccagctcgtcctcgagctgccatagacttacctaacacgacttaaggttaagccttttacatctcggcttacctttattatttaagacgaaaatacaaaataattgtggaactaaaatataaatttgaactgcagctatgtcctcctgtcctcctggatcccaaggaaagagctgaactgcggccttcacgttggatgaaaggtggaggaggaaccaactcgttcttatgttgggtctgtccaacaccaaggcaggtgcccgaatgaaggagacgaccctctttggccgtgcaggggggctgcatacccagatctcgacttctgcagggggttcaaaaagtatagatgagacctggtggttgggcacgcaggctgcgagttggtgcagcgatgagctgatcagcaagtgtagcttccgcaccgcccgcctaacaaggaagccgcgcactgcggcttgcaggcgcaccacgacctgctcatgtgatgacggccatggggtggccctggaggccatagcaaggtgtttctccccacgaagggactgtacctggcggcatgccaggaaccctcgcgctgccgtttgaagccgcaccgctgcttggagttccttctctgtcttctccttgtagcggtggaacatcacaacaaaccgatcccactgctctgcgaagcggccaaaactctctttgagccgagtgaatgcatctggaattggagagggcgggtgggagggcgttgcggcggctgatggcgcggcggctggtggtggtgagggatgggcggctggtggtggtgaggataacctggagctgataccaggtgtcatggaccttcggtccatgggggtagctgtcttctccggcgaggttatacccctctgccgtaggcttggttctagggtagcagccctaggcgcttgagggtcattgcaagagagagagattggtttgataatgtattccttcttcctttgtctcctgcgtacaggcctatatataggccaccggttgaccaactaggcaaccactaattaggcaagtgactaattgaaattatctcccctaaatttaggtctatcaatttagccaccaaatcagtcactttcctttgccatgccttccttgcgcgcgctttcctttgccatgccttccttgtgcgcgctttcctttgccatgccttccttgcgcgcgctgctgccagcgccccctgtggcctctcgcttttggcgcacataggtgcgaccccacatgagtgaccgctccctatgatgcccatgacaatatactacaaatatatgcatgtactaataattcgaattttttttataatcttgtttgaataaatatctcatttaagcaaggtttacctatatgatatatagaaaccctagcaatgtatgggcaactaattagtcacttatagacttcaacatctgcattatgatatccactgtatttctatcttttagcacaaacatggttgaaatttgtgatccatagagagctttaggcgcctagatctacatcatgcccgtggttttcacaatccccagttcccaacttgttacaattttcattttttttcactaccaatattaatgctttggactttcttgatttgcaagttgcaactggaaatgaaattcatcatgaaaaaatagaaatggaaacgaaagatttatgatttttttcatgtaaaacgaaaataatgacgaggttgttgtacatatctaggcaatatggtgtgcgtgaaagtaaggatgttactattgaaggaagagtgcaatttgatctcctgcaggtagcgatccactttttcttgacaattaaatcactgtttgttgttttcattagattgtttcatgtttatcttcactacctggatatgatgcaggctatgcaacgggattacaagttgagttcttattcattaaactctgtatcagcacactttcttggggagcaagtaagttcaaacatttcaagtgctctatgttgctatcagtcattgtactcgtaaccattttgctgcccttatttctttttctcgaaaaacgcaggagaactgcgttttagatatatattaagaagagagagacaagggtctaaagagacccaaaaaacaacgcactccttatggaagccagtaacactcgtacatgaaaggatccataaaaaacacacactccctacaacaacctatgcagggggcagccaaggagagccctttggctccaacaatttcctagcttcttcttttctcatctgcatgctccataatcaggaaggctgggggaccctccattaaaaatggctctattgcgatgcttccaaatcatccaagatcctaaaatgatgagggagtcaagaccttccctgaccagaccatttgatgtctctgataccatctcccaccaatccataaaggaaactgtatctggctgtggggctaaactatgcagcccaaacgttcttagcaggttgtaccagaaaactcttgagaagacacaacccacgagcaagtggtttagggtttcaccttgcttgtcacataaaagacatctgatagcgtgattcaatccccagtccaacgggcagtccgcgacagtggctgcagtggcttccgtgggcgaaatatgtcttcaacatggcgtaccagacctcgctgcgggacacgccattccgggtcgtctacgagcgcgacccgctgtctatccggtcctacgaaccgggtgacacacgggtagcagccatcgccaagactatggaggagcgccaggagttcttcgcggatgtctgctattgcctcgaacaggcgcaggcggtccagaagtggcactacgacaaaacgcaccacaatgtctcccaccaggtgggcgattgggtgcttctctgcctccgtcatcgcacgacagcttcactgcctcaagaggtcttcggcaagctcaaaccacgcttcttcggccctacaacaacgtcgctgtccgcctcgccctgctgccatggacccgtctccacgacgtcttccacgtgggtctcctcaaaaagttctagggactaccatcggaggttcctccgcctctgccgctcatccaccatggggcggtgatcccggagccggaatgagctgtcaagacacacatcgctcgctgggtgcgccaggtcttggtacagtggaagggccaaactgcggcgtcagcaacttgggaggacatcgagccgttcgtcaccaagtatcctgctttccagcttgcggacgagttgcctctcgaggaggggagagatgtcatgtggggactcaagtatgctaggtgccgtagggctagggatgtgcggcgggcggctgagcgcagcaacacgctggaagtagctgtcctggttggataggtcaggtaaccgccaggagagaagttaggaaaggtaaggattagtgttagcttagatcctaaagtatagggctgattagttggggccatgggccatatatatatccatgtaagatgcactttggaggtcaacaaagatatatcctatctccttgtcttcactaagcctgtggccgaggaattacctcgtcggagaagatggctggcggctacgaactacgtagccgcggtccagctactcgagggtacacaacccttgacaatatagaccgtcatgctagtgaggacatgcttcactagaaagcactagaattcttctccctgctctactcatcaagtcagccttctagtttggtaggcgctctgtaattttgtccacaaactgctgagactgattcctggataatttatgtaaagacaaggggagacccaaatatctgcaaggaaaggttgcaatctcataaggcagccagctcataatttcctcaatagtctcctcggggcatttgatagggtacacatttgatttctgttgattatagtagaggccagatgcatcaccaaaaagctggaggatatccagagaaattgaaatatctgtagctgtagggtgcaagaacatcaccagatcatctgcatacttcgagactctgtgaagccatttccttcttgacagttgctggagtaagcctacttcctcagctttggaataaagaaggccaagaacatccatagcaaggataaatagcatcggggagagaggatctccctatctaagccctcttctatgcttaatcagcttacctgggacaccattgagcaacacctgagtattggaagaggctaataggctaataatccacagacaatgtctcaccagatatgtccaaaacccatatgctgcattacttcaattaggaagggccaagagacagagtcgaaagcctttgctatgtccaacttgaaaagcagtctaggcatgttttgttgatggaacaaccttgaggtttgttgcactaacatgaaattatcctggatgaaccttccttttatgaaagcgctttgacttggggaaaccatctcagtcaatctttttgctagccgattagccaggatcttagtaaccagctttgcaaaactgtgcactaggcttattgggcgaaagtccttaacttgatcatccatctgtttttggaaccaatgtaatatatgccgtgttgagtttactgaaatgatcaaacttcctgctccaaatagcagccataatatcatcttttatagtgctccaacagaccttatagaaacgtccaatgaacccatttggaccaggggccttatccgaaggtagctcattgatggttctccatacctcatgctctgagaaaggagagtcaagatctgatagctcatgtcttggtagaccaagggcctctagatcaattgtcctttctctgagtagaagtcatccatcaccgatgcttttttgatatggtttattaggatacggtctccatccaccaatttagtgagtttagtgaccaggttcttccttttgcgatgccttgcatgcatgtggaaaaactttgagttagcatccccttctttaagtcatgtgatccttgatctcagtcttgccatagtgcgtttgaaagaagcaagcaatagggtctgttttttttttccagtttattcttgagccagacctcagccagggtgagggttctaatatcctgcgccatctcgagcctatgtaacaactcctttgccaaggccaactgagatctaacatgacccacccgcttctcactccaaccctgcaatcttttagctgtcaccttaagtttcttgtcaagggccaggaaaggagaatggtcagtttcaaccgagtcccatgctgcctggacagtttcatgaaagccttcgattttggtccaaaaggacttgaagtgaaatctttgttttccaaacatgttatatctaagacccaagatcagggagcagtggtcagagtccagagaagttgcactctggaggaggacatttgggaacattattttccaatcaactgagctgaatttgctgtccttgtgtttgaaccttttttgtgcttgaccagtgttagttctggcttcgaataaaaggctttgatgtggtttttattgttgctgttggtggtgttgactatatttctaataagtgacaacaacaacaacaacaacatagccctttgtcccaagcaagttggggtaggctagagataaaacccacaagaaacaaggatagaaaaaaaagaaaaagaaaaaaaagacaccgaaagggtaaaagaaagacgatagtgttaagggtaaacagaaaaaaggacagatcatggttcgggcacgttgattgctagtctcaaagcgcacatatccttagctaattctttggagatattccacttcttaagatctctcttagctgccccatcccaagtcattttaggtcgccccctacctctttttgtattatcaacccgctctaaaaccctctaagcaccaacgctttcggaggtctcctttggacatgtccaaactccaaaccatctcaaccgatgttgaatcaatttctccttaaatggtgccaccttgaccctatccccaatatcttcatttcagactcgatcccttgtgtacccacaaaaccaacacacatttctgctacactcaattgttggacatttctctaatgcacatgttagctgcacaccattatactaagaagaaaaacaaggtcccataaataaccgatacagacaaaagaaaaagccccctcatggtgtccaaacctgaaaatacacaaaaggatccatccctagataaaatctaaaatttaacgaccacagagcagaccaaaagaaccctaagaagtttgggggggcaagagataaaagttgtgtaagacctttagctcctgccagcttccaggcttccctttcttgcgctacttgagacaggacaacccccgtgctggtttgcagcaaatgtcaccttagctggatgaccaaagttttcttgtattgtggaaaggtgctagtgagctggttgaggagaacaaacgaaggggtcttaacacttcgatcatctaaggcgcatgaattctttgaaaacattgaaatagattgcatgacatgtcttcagtcttctagataattgatgttcttttgcaaaatgacctatgtagatggatttatttgtatcgatatcatttttgctactatttactcacaatgattttttatctcaaaagcgcaggagaaccgcgcctctatatcaagaagaaagaaatacaggtcaaagagacctgatacatggtacaaggtctccttaggaaggccaaaaacacaaaactgaaaagatccatcaaactcacccagcaacaccctagtctttaggaaggcatgaaagacctttagccccaaccacttcccaagagcgcctctcctccctagctaacctaagggacaaagccaaactatttctaagagtacaaccataaaaacacaactgtttcgaagcttccaaattgtccatgcccccaaggcagcaagcgagttgaaactctttctaggtagaccagtgaccattcctgagatttgctcccaccaattcatgaaagatgaaagccctgtttgggagccagactttgaaaaccaaaaggtttaaggtggttaaaccagaacactctcaaatggtttttccaagccaccaagattccaccactagccccttctgaaggcaaaaaaacataatcagcaaattcagaccccaacatggagactatggtacttctaggaacagattgcatcttagtttcctgcagacacacaatctcagcctttacagaaagaactaattctctaactgtatcttggcgtgcacgagaattaaggcctctcacattccaaatcaggattttggatggatccattaagaaagataatacgacacccattagacatacaaattgcgcccaaaggactgcaacacccttactgaagagggacggactcaggggctctccacccaaacaaagctgaaagagcctccacctcactatgagtcagggggcgcttaaaaagtctagcatattcatccaaggcctcctgactaatcccctcattctctgcaataatatgcagggatctcatgactttcctggtagttctctctcctaggtcttgtatatcaaattccactccaacactcgtaacacgacgacttcttggcatgagagcagcaggtggggaaggtgaaagatctgcttgcataccagctcttggaacatgaaggatcctagatgaatgcttggtgattcctttgaaaaactcattttttctagcttcaaaactggctgcctcatcaggttggacttcagcagcatctctaggatcaggggcatcaacagtagtcccaggcacagagttacctaaggactctgaaggaggtagacaaggcttctctgcagaattggcctcctcgacatgtatcacctgttccacctgaatcagggcttgtttgcttttgaatctgcttctcgaatatactctcataggagcttcaacagtagatggcaagaaaaccactaatggatcgccaggttggggcgaattcgcgccagcccgttccagcggcccaacaacagaaacgtgttcaggtcccacagtagccagcccagttaagccaagatggcccaacgaggataaaactggaggattgttcatcttctccacagatggcacagcctcgggaccccgcacgccatccacgctttctgtcgacagagcctcgccaaccgcgtcaatctcttcagaagcttgatacgaatcttgagagacagcaaccaactccatgtgtgctgcagaaggtgggcccagaacaccagtagggccccctaggtcttcttcaccaagctcgagggcatgtcagcagttggaacgacgcccccagccccgcagagacgactgcctcaatgaatgctcgtgtcttcccaaagcaccgggagacgaggaggcctgcgaagaacgaacaccacccgcccttccaactcccgcggagtgcaacagaccgagccggagcagagccgaccgctgccggtcctggacaccacccgaccgtcgaggagggtcctggaatcccgggtcatcatcagcgccatgcccatcctcctgaggggactgagagaccgcagacgaatccacaggatccatcggaaatgttgttaaaacgacgttgtaagtcaggcaacgcttagcttgtgcctccggcccgtgctcgacgatatgcaactccatagaacgatgaagcttcaccggaccgaaacaccaagctctcaacagaaacgaagagtaattagtcttgttcacggtggcgggaaacagctccaaaacgtaacacgagtcatgaagcagttgcgaaaccgtggagaggtcccacgcgtgtgccggaatgtcgtggacctcgatgtccaccagcaccgacatggtagacgacaatgcatgagcgaatctagaccatcttttgaacattaggttgaagcgaggtccacggagcgctcttccctcatccaaaacccgagtcgcgatttcttcatctggcaggaccaagagaaaatcctcaggcatggactgaggaattgtcatatccgcaatgtcgatgccaaaattgcatgccacttcctccaacacctcgtcgaagattgacctcctccctagccatggcaattgagaattcaaccacacaggagggtgtatttgtcacgggagcctgggagggcccccccccccaggagcagcagcaggagagcaaggagggacgtgcagagcctcgaaaacttgggcgcccaagattttatgctccaaagctccacgacctttcttcgaatgtcttctctttctcttattgtggacgatcacctgttcttcatcacttgccaaagccaccgcatccagtggtggagcaattcttctccaaactggtgtcttccactggaacacctccctcgatggagtggcccaatccatgctggacagcctctgccacaccgaaggccgtggaccacgatgactcaaacgctgccacaccgtcgtcctcttcttgctttcaggttggagggtgcaccaagccgcttggtgaccaagcttgaggcacttgaaacaccgtgtaggatggcggcaagcagccacgagatgtgaggcggataggcagttgtaacaccttcctcggagatccacctgagaacctcgacgaggaggcagcacagcagccggatgcctcttcctgcgctccaccagaatccagccgtcttcgtccttcccctgccgtccaagagtccggccctgcaacatgggagagctggcccttgacgcaggagcaagaggagcccgggcagaaggaacatgaggagaggagcgagtgcctctcttggaaccggagagcacaacctctttgtaggaacctggagtggagcatagcccgtttgacaaggaggtgggagtggaagaggaatcgctccacctctgggattttgaacgacctacctgcgaagccagacgggaaagtccaacaggaccagccggcgacggagatgagagtacctagagggggggggtgaataggtgatcctgtaaaacttgaacttaatgccacaaaaacttggttaaacgttagcacagtattgccaagtggctagagaggagtctcaacaaaacacaataaccacaagagatcaatcacagagatggcacagtggtttatcccgtggttcggccaagaccaacgcttgcctactccacgttgtggcgtcccaacggacgagggttgcactcaacccctctcaagtggtccaaagacccacttgaataccacggtgtttgcttttcttttctatatcccgttcgcgaggaatctccacaacttggagtctctcgcccttacaaagatgttcacaaagaagcacggagtaagggagggattagcaactcacacaagacacaaagatcacagcaagtacgcacacacaagacccagacttaagctcaaaagactagcacactagaacggagctcaaatcactagaatgtcgaacaagtgcgcaagaatggagtgtgagtgatcaagagtgttcaaggaatgcttggtctattccttcatgcgcctaggggtcccttttatagccccaaggcagctaggagccattgagagcaatccgggaaggcaattcttgccttctgtcgtctggcgcaccggacactgtccggtgcaccaccggacaccgtccggtgcggatttccttccttatttggcgaagccgaccgttggcagccttggagccgttggcgcaccggacactgtccggtgcacaccggacagtccggtgccccctcccgaccgttggcttggccacgtgtctcgcgcggatcgcgcagccgaccgttggcccggccgaccgttggctcaccggacagtccggtgcacaccgaacagtccggtgaattatagccgtacgccgttaatttattcccgagagcgccaagttcgcctgagccagcctggcacaccggacactgtccggtgcaccaccggacactgtccggtgctccaccggacagtccggtgcacccagacaacgctggctttggctgaaacagagctatctttctccaatttgttttctcctgtttccaacacttagacacaatacattagtctttaaaacaatgtactaagtctgagaaacataccttttgacttgatttgcactttgtccaccaccttgcatatattaacacttaagcacttgtgttggacactaaatcaccaaaacacttaaaaatggcccaagggcacatttccctttcaggagaggggctgctggttggtcgccgggtccagagtggagtccggcgaggggctggtggtgggatgggctccgacatgggaaggggtgggagggaggggagaggacgagacgggagccatactatgccctgatagtcttactcacaatgattgattctaatattatttccttcttttgagaaccctttctttttctttcctaattattgaatttttgagcagaaagaagatgttcatcattcaattatatctgatcttcaaaatggaaacccagagacacgaaggcgacttgcagtttattgcttgaaggtcagttaaatataccttttttgttttaattgttactgaactggtgatgacgtgatgttttacagagttgttggtaagttgcggccactttgttattgttgctgatggagcttttaagtacgccacagaagcatttagaccaatttattcaaattaataaatgcttacatattgctctacacgtgtcaacgtttgagcaccaattttgcattttcgaattttctcagcaacactagggctttggaaaattgttacattatttagtctgttttttttcaatttcccactggccagtaggtctgccagtagctagtcacaagctagcatgaaacagtacatacatacacatacatacatcaacatagccttttagtcccaagcaagttgtagtaggctagagttgaaacccaacaagatgtcaccaaaaggagaaagagagaaaaagggaggggaaaagtttggagagcactaaaggaaaaaggcctaatcacggttcagccacgtggatagcttctttccaagcacttctatccaaacacaactccattgagatattacattccttcaagtcccttttgattgtctcctcccatgtcaagttttgtcgacctctacctttcttcacattattgtcccgtcttatgatgcctctatgcaccggtgcctctggggtcttcggtggacatgcccaaaccatctcaaccggtgttgaataagctttttttcaattggcgctactcctagccgatcgcgtatgtcatagtttttcactcggtccaatcttgtgtgcccacatatccaacgcaacatacacatatcagctagcatgaaacagtgctattattaattccactgtatcatctgatttccctcacttgttccagaaaaatgtgtaagctgatttgtagcatttttcttggagcctacatcaaaccagttttgacttttgcctttgtaaaaacaggatgcttatcttcctcaaagactgttagataaattgatgtatatctataattatgtggaaatggcgagggtcactggtgttcccatttcgtttctactttcgaggggacaaagtattaaggtgagaacaagccaatcatgctattggaaatatcacactctagtaccctaacaatagttacttttgtgccacaggtcctctcgcaactactcaggaaagcaaaacagaaaaaccttgttataccaaatataaagggtcaagggtctgggcaagatacctttgaaggtgcaactgtaagttgagggacattatctgccatatgtaacccatttgtcatttcctgaccacagctattacagttattaggatatttatatttgtctattacaatctttttagtttgctctgtacactgaaactatctttgcattgcttgacttgaactgaggatatagatatttgtcatcacaggcttaagaaaatctagtgactagttaagctatccccacttattacaaaatttgtacttgatgcgtgtggaaatgaaatacatgtgcagagttttctactctagaaacatggttttgtgtatgctagctatctttccatctgaagcaataaactttcatcagttttcccaccttctgaattgatcgtgcaatgaacaatttagttttgtttcttttgtactataaggaattgctgtaatctatgtatccttcaatttttaggttttggaggcaagggctggattttatgagaagcccatcgcaactttggactttgcttctttgtatccatccatcatgatggcatataacctttgctactgtactttggtaggatttctgatgcttatttagttataactattgctgcatgctctagaaaggcctttggtctctataatggatgccaatttactagtttttatttaatttttgaggtgccccctgaggaggcccgtaaactcaacttacctccagaattcgtcaataaaaccccatctggtgaaatatttgtaaaaccagaactacagaaggtaattgcatcctgccttgataatagctgttcgaacttcgaagatgtgttctctaaccaagcttatgaacacaatatcagggtatacttcctgagatccttgaagaactattagcagctcggaaaagggcaaaagcagatttaaaggtattttttcaatgcatctgtaagaaaattgtactatctctgttctaaattataagccgttttggcctttctagataaatcacagttttttggcttttctagatacatagtcttttacttactatatgtctaaacctagcgtatatctaggtgcatagcttgctatgtaccttgaaaattcaaaacgacctacaatttagaacggaggcagtagatctttcacactcgcaaactatgcatcatggatatgtataaatggatatgatttttttcccagaaaaccctttacacagatacattttactattttttaaataaataaataaacaataaatgtatcagaattctggaaacagtagcagccaaaagaagcatcaaaattacctacaatattggtgttttattttttcccagttttacgatcagtgagaaggaaactgagcatctccattcactaatttttaactaataaagatcataaatcacacaataaaacatagaagtaatgtaggagccctggtgtaaatgaagtactggcagtctggcacagtggcactagtacttgaatttgttgggtcatgccatctacttgtgtctgcgaaaaatcatggtcaacacatagcaaacaacactagatccggaaagagaacaaggaaaatcaaccaatagctcaagtactgaagaacaaaactagatagtgatttggaaaggaaggtcactggaagtggctttgacttgctactgaatcgtcatcagtatcacaagcgtggtggaggtgcatcatcagcaactcagcatggtggaggctcatctgtgttgcgcttgtgtgtgctttgacgcaggcatgttgaatcttcatgggcgttgtgaagttgcatgcttgccaacatggtgaagcatgtggctgcagccagtgggcggaggtgcttgactgtgctgcatcgatgcttgtgtgggtgggtggcagagtgatacacctagtatgcgttgggtggatgaggcaaaggagatggggtcatcgggggttaactggagtctgccctgatacttattgtggtttatcgaatttatttttattccaatacgttgctgatatttattgaagccatgtcctgtgcgtatctgtatcagatacttggatacgctgcccacccagtgtatccatgcatcatagcttgcaaaaagctagatattcatgcagtgctaagtctttgtcccatgttaaacttgccaattatttgttcaggaagcgaaggacccactcgaaagggctgttcttgatggtcgtcaacttgcactgaaagtacttccatattgtttcccctgattccatcaaaacattgattcaaatttgaactacttgaataatgacattgttaaattgcttatgtttcagataagtgcaaactcagtttatggtttcactggagcaactgttggtcagttaccttgcttagagatttcttcaagtgtgaccagctatggtaagccgtattttaacataattaagaaattcattgtatgatttgttagctattttgatgtcaagattcttgcactgaagcattttggccacacaggacgacagatgattgaacatacaaaaaagcttgttgaagataaattcacaacagtaggaggttatgagcatactgcagaggtaaagtagggttgcattgttcttatacaaatctgttggattccctgttaattttttctgcttgatctcacaaacttcaagcctccaacttttgctcttgtatcttcatgattgatgattgtgtttattaagttgcatgcaccaactagatcaacccaaaagcgcttaagctgatagaaagaggcggacaattcacctatattatattccaacaccccctcacgacgagccacttttgtgtcatagacgtgaaataggagcgagcaacaactatttttattttaattgtgctaaccaggattcgaactcgtgacctctggctttgataccatattaagttgcatgcaccaatcagatcaacccaaaaacttaagctgataggaagaggcagacaattcacttatattatattccaacaatgttagagctaatgtgtaatgcctaatgtaatgggcttaggaccaactactcctaatataaatatatctcccaaccttggttacggttatggttttagttccaccaacatggtatctagctaagttttatttttcctctccctctcccacctctgccagtcgccgctggctccacctcccgccggttgtcggctgctccagccgcagactcccattccccgtaggctgccggccttctctccccccggcagctccatccgccaaattgtcttcccgcttgcctcgctcccgccttgcctcacccatggtggccctgtctatcgactcctccagcgctgccctgtcgggtgcctttcccctaggcgtggatgcgctgctttcgcctagcgtcaccgtcgagcttggcgcggtcctctccctcgcgcgcttggggatcggcccacaactcctacacagtgcttcgcctccgccctcggcaacctcggctccggcagtccttcctctttccgacgcgaccccctccccttggcgcggacgcgctcgccctagatagaggtgctgtggcatctcgcgtagccaccacaggtgacttctccccaccttcgctgcctgtgtattccctactcgacgctgccgccctccgtggcgccctccttgcccctcacttcccctacttgggcccgatgagcggcaccggggggttgtacacacttgcataggcgcttggcgcgttctcggatgcgacatgtcctgtccacttttcgctgatctacatccatccactgacgctcactacttctcagcgtcgcgttcccaacgatgttctcgccgctgctcgtcagcgcacacaggctgctgctcgtgcctaggagtaggatcacgcggccgtcggcacccttgagtgccaacttatcgacaccactcacctgtttggaggtgtctcgggtgatgactctcactccatgcacgacattgaagccaacaacacccttcttacaccggatgcggtcatcgccagtcttcatgcttaggcgacagggatcccaagcatacgggccctcgtcgtcctcgtcccggactgggcccagtaccctcgctagtgggaccaagtcctgccgactcgcacctctatgccctcgacgaccacatcctcatcgatgcggtcgtggcgacgtaggcctagtatcgaatgaacaacatgatcctcagcactctcacggtcgatgtaggacgccgtccgcgggggggggggggggtgggggtggggggggcaccgcttgccagacatgagttgctatcgaggttcagttcctccgcattgtcagatccggatccttcatgtggacaccagcttgtccatgtggatctctccgtcacttagtactgcttggagatgaataccatggcatacatcctccgtaaccttggcttatcagtgacggacgagacactcgttttcaacctcctgcgcgggctcagtcctcgatacgcgtacctacgagccatcctcacgtggttcaccttgttcccgtccttctctcgggtctgcgatgatctcttgctcgaggaactcacgtctgcgactatcaccaccaccgacgcctcgacgccctctacagtggtagccccaacgggcatctcctactgtctccaggggtgtgccccctcgttcgagtgcaaccccctcgtcctctagcgcccctcatacagttgccaactccgtttgtggtcgtcgtcgtcgccagggcggtcgtggtcgcgacgacggccccggtggtcctccggcggcccatggcggtggctcgtggccatccttctacaacccgtggattggctccatctccatgtggtctggaccgtttgcgggtactccttggttgtgtccaaccaaccttctacattgcttcgctcccattggcgctaccggcacacccgactccacttcagcaccagccgggtctcctgcctccccttccctcccctcatgcgccctcgtggggcccttgcggtggcgggtgggacacaatctcttgctgggtcgttcaacacgatgattctggcactgtccatcttggtcactgactgggtgactgactccggtgcctcgtaccacatcacccttgacggcagtactttatcttccaccgttccccctcacccttctcttcctccattatcatcagcgatgggtcggttctccccgtcacctcagtaggtgatgttctccccagccccttccgcctcaagaccatctttattgctcctaatattatttggagttttctttccgttcgtcaatttaacactgacaactcttgctctatggagtttgacccctttggcttgtttgtgaaggaccttgctaccaggacccttcttgcgtggtgtgatagctccgggccgctatacacacttcgcctgcctgcctcctcttccatgacctccgcctcgcatgctctgaccgctgctacctcatctatcacttggcaccgccgctttgcccacctcgggcgtgatgtgatgtccaagttgtctagcagttctgttatctcatgtagtcgaagttcttttgagcatttgtgtcatacctgtcagttaggtcgatatgtttggcttccttttccctcttctagaacagcaagcgattttgatttggttcactgtgatgtttggacatcccctgttatcaacatttttggcaataaatatacttgctcattctggatgattactctcatttcttatggacttttcctttgcgcttgaagtctgataaatgctccatgctctctcacttcttcgcttgggtttacactcagtttggaccatccgggctgtccagtgtgacaacgggcaggagtttgataactcagcatcttgtgacttcttccttccacacagcgttcagatgaggatatcgtgcccctacacttctcattatggcagagttgagcgcatgattcgcaccacgaacaacgtcatgcgccccctgctgttccaggcctacctacccgctcgttactgggccgaggggctagccacttctacctatctgctgaattcctccccaccaaggcggttcgtcacctcaccccctactttgcactctttgtcacctttccctcctacgatcaccatcgcgtgttcgggtgcgcttgctatcctaatctctctgccactgtcgctcacaagcttgctcctcgttccactcgtgtcttccttggatactctcctgatcacaaggggtaccggtgccttcactcttcacccacagagtcctcgtcttccgtctttgatgagtctgactttcccttctctgtttcccctttcgctgcctcgccctcttagtacgacaccttccttgaggcagactatgtggttcccgctcttgcacccttgcttcttgcacccccaccgtgccatgcgcagccccgctaccccccgtgctgctccacgtggcaccgtcgtctcttgctgccacgcgcggccctagagtctcccgcgtagccccggtgtctcccgcgccgccacatgcagcctcagagtcttactgccacgtgtggctctgccatctctcgcgcaaccttgtgtgtctccggcgtctcccgcgccatctcgcgcagaccgagcgtccctcgtgctgccacgcacggccccaccgacgttcgccgagttggccttttcatcacgcttcgcggaccccgttcacgtctaccagtgatgtggttgggtcgatgccctggctccctctcgcgaggagccgttggtgtatcaccccattgccctccaccgggacctcgggcatgtccacccaatggttacctgacgagtcgcctgtgtcctccggcctgttgaccgcctgattctcacgacgtccacctcttcggcgctctcccccgagccatcctatgtccgtagtgcgctcaccaaccccaactctatggagtacgaggccttgcaggcgaaccacacctgggacatggtgccccgcttctctggcaccaatgtggtcaccgagaaatggatcttcaagctgaaggtgaaggcagatggctccttggatcagtacaaggctcgctgtgtcttgcgtggcttcatgcagtgtcccagggtggactatgacgagaccttcaacccagtggtgaagcctggtatcattcacactgttctgactctggctctctcacaggcttggccaatgcatcaactcaatgtcaagaatgccttctttcaaggcactctgacggagatagtgtattgcacttagccgattagttttgtcgacctagctcacctcgacttggtctgcaagctcaaccggtccctctacggcctcaagcaggtgcctcgagcttggtacagtcgctttgccatcttactcgcataaggcttcgtcgaggccaaggcggacacctccttgttcgtcttatgtcgtggcctggacacgacgtacctcctctacgtcgacgacatcatcttcatgacctcccccggcctcctacgacgcatcatcgtgatgaaggacctgagtgagctgtagcacttcccagggatcatcgtggagcgccggtcctagggcttgtttctccaccatcaccagtacactgtcgacatcctcgagcgtgttggcatggctgactgtaatccctgtacgaggcaaggcctgctcggacaacgtcccctggtagctgatcccaccagctaccggagtcttgctaggacgctgcagtacctcgtcttcacccaacccgacatcacctatgtcgtctagaaggcgtgccttcatatgcacgccccgtgggagccacatctcatcgctatgaagtggatcctgtggtacctccggggcactatcgacttcgtcgtgttccttggcgtcagtcttgtctcctggtccttgaagcggcaaccaatcatctcctgctccagcgccgaggccgagtaccgcgctgtggctaacagtgtagccgaggcagcgtggcttcatctcctacttcaggagctacacagtccgctgacacggagcactcttgtctactgtacaacgtcaacgccatttacctctccaccaaccccgtccagcacttgcgcacgacacatgtcgagctctacctccacttcgtcgcgagcgtgtcatcgttggggatgtgcgagttcttcatgtcccgactacttctcacagggggttagagctactgtgtaatgcctaatgtaatgggctgtgtttgtgtaatgggtttagtctcaactactactattataaatatatctcccaaccctgcttagggttagggttttagttccaccaacagattgtcctatgagtattgtagcctgatctagggatatcaactgaaaaacaactcaacatatataaaaggtcaacccatgcaaaccactatctaaaactgacactacattgtgcgcatgaaaatttgattccattagtttttagctagacatcagcttagtgacaccgaattagagcttcaccgtgcgcccaagaaccatgagcattgctcaagaccttgttcttgtgatattttgccctcatgttcaatttcttggaagtcactgtagccagtttttttatttcagcaagctttttgcctgcttatttcttgggtctgaagatgcctttcactcattttctgaaataactgatacagaaaatttggtgacttcacaggtaatttatggggacactgattctgtgatggtacagtttggtgtttctacagttgaagatgcaatgaagttaggaagagaagctgcagattatattagtggaacctttaccaaggtgaacagacaaacatgaggctttggaatgagtcatacagcatttgagtagaacattggcttgtttgtacgcattttagaattgtcgtacttgataccatccatgaccttttttgtgatcaagctccaatcgttatctcaccgaaacctatttggaacagtgaaaccagttaagagaatgctccttttcaagaacaaaaaaaaaatctgaaccctttaaacttctgagttctaactgcaaggttactgcctcatagtagtttctagctttattagctccaattcttatctagtcagctatctgttctaatctatcgctgtctgtattcttcccatctggccaaatataggaaatttgacactggtcaaaatagaatgaaaaaattgtccaagctttcatttgcttttcatttaagttccaattcacacgttgcaaaagatttgtaacgtatccagctaagctttcatttgcttttcatttaagtttcaattcacacgttgcttatgacatctttctcacagcccatcaaactggagtttgagaaggtctattttccttaccttctaatcagcaagaagagatatgctggtttgtactggacgaatcctgagaaatttgacaaaatggatgcaaaaggtattccttttttgtttagctgtgtaaagcataaactattttcattggatgtggacacagatgttagttatccttcctttcaaatgtcttctatcttgcgtgcaggtattgaaactgtaagaagggacaactgtttactggtaaagaacctggtaaccgagtgccttcataaaatactggtagaccgtgatgttcctggtgcagtccaatatgtcaagaacaccatatctgatctattaatgaatcgtgtggacttgtctcttcttgttataacaaaggtgggcaaattcaactgttttaagatgaattttatattgtgttgcaactcttttgactgtttgagatgcacagggtttgacaaaaacgggagaagattatgcggtaaaagctgctcatgtggagcttgctgagaggatgcgaaaggtatgaacctaggtgttattaccatatcttgatgatattcaacccattcttaagatatggtgtcgtagtgctgctgaaaaatcatggttttcttgagcagagagatccagctactgcacctactgttggtgaccgtgttccttatgtaataattaaagcagcaaaaggggcaaaggtaaaaaagtctgcatcatgtgctgcggtattatctgtcataatgtggtagtgtgtttgatacatttgtctttctgcatgcaggcatatgagaaatcagaagatccgatttatgttttggacaataatatacccattgatcctcaatactaccttgagaaccaaattagcaaggtttgtgccctatgcttcacctagtattcatagcctctccccttctgctttccatttttttttgtaactgaccacttgtcaatcggcagccacttttgaggatctttgagccaattctgaagaatgccagcaaagagttgcttcatggaagccacaccagatctgtttcaatctcaactccttccaatagcgggataatgaaatttgcaaagaaacaattaacttgccttgggtgcaaagctgttatcaggtgacacatgcactacattgcacagatgcaataactatgcaaccagtgcctataaactcttaaccgtttgcgctgcattttcaccctgcagtggtgccagtcagacactttgctcacattgcaagggaagagaagcagagttgtactgcaaaacagtagcaaatggtacgtgctgtacactctgaaatgcatctacattatgcctttggaagcactatgctcctgagctaaccactgacagatcaaaattttgctttccgcagtttctgacctggagatgctctttgggaggctatggacacaatgccaggagtgccaaggatctctgcaccaggacgttctctgcacaaggtttgctgctttcccctcaatccaacacctgctcattacttggttccagtcacaagattaaaactcgtatgcaatcgaaccactgcagtcgggattgccccattttctatcggcgaaggaaggcacagaaggatatgaccgaagctaggctgcagcttgatcggtgggacttctgagcttggaaattctcacatggctgcctgcgtctgcttatattgctgtggaagacaggaagctaccgtcgaccgcatcataatgcatgtgaagatgagactggacttctgcgagcatccagtcacacttgcgtccgtgtcacgtagactgacttgccagtacgattcattagcatgttttctgcttaggttttgcgtaatagtttaggtgtgagttggtacttggtagatggtaggtgtcaccttcctgtggctagtgccgagaaaaatgcttggtcctgagatgaggtgctgattatgtacagggcagaagcaatctggagcagttattatgtaacagagtgagacgagacaatttgaatggcacatgagttctcta

SEQ ID NO: 23 - DNA, 3809 bp, Zea mays
  source 1..3809: mol_type = other DNA
ggacctcctctcgtttcgtattaaaccctacctcgccagtaatggcgggaactcccagtttcagtccccgcacttcccgcctccctctccccctctcggtcccaccttcgtccctccggcgatgaactcaggcggacgcggcggcaagcggcggggtcccccgcccctcgctccctccggtgcggcggcgaagagggtcaatcccagccccggcacgccgcatgtgcctcctcccgcgtcggcggcggccgccgtggaggaggacatgatggacgaggacgtgttcctggatgagtccatcctggcggaggacgaggcggcgctgctgatgctccagcgcgacgaggcactcgcgtcccgcctcgcgcggtggaagcgccccgcaatacccaccgacctcgccgccggatgctcccgcactgtcgcttttcagcagttggaaatagattatgttattggtgagagccacaaagaattactccccagctcatctggttcagcagccatactcaggatttttggtgtcaccagagaaggccacagcttatgctgtcaggtgcatggatttgagccatatttctatattggctgtccttcagggatgggtcctgatgatatatcacgctttcatcaaacactagaggggaggatgaaggagtcaaatagaagcagcaatgttccaaggttcgtgaagagagttgagcttgtgcagaagcagacaattatgcattaccaaacacaacaatctcaacctttcctcaagatagtagtcgctctgcctaccatggttgctagctgtcgtggtatcctggaaaggggcataacaattgaaggccttggttcaaagagttttctgacatatgaaagcaacattctttttgcccttcgtttcatgattgattgcaatattgttggtggcaactggattgagcttcctgctgggaaatacaggaaagcagcctgtatcatgtcctattgtcagctagagctagattgcttatactcagatctggtaagccatgcggctgaaggagaatattctaagatggccccgtttcgcatattaagttttgatattgaatgtgctggtcgcaaaggtcatttcccagaaccaactcatgatcctgttattcagatagctaatttggttacacatcaaggagaagaccaaccttttgtacggaatgtcatgactcttaaatcctgctctcccattgtgggagtcgaggttatgtcatttgatgcagagagagatattctacttgcatggagggatttcatacgtgaagtggatcctgatatcataattggatacaacatttgcaaatttgacatgccctatcttattgagagggctgaggttctcaagatagcggagtttccaatacttggccgaatcagaaatagtcgtgttcgtgtccgtgatacgaccttttcctcgaggcaatatggtgtgcgtgaaagtaaggatgttactattgaaggaagagtgcaatttgatctcctgcaggctatgcaacgggattacaagttgagttcttattcattaaactctgtatcagcacactttcttggggagcaaaaagaagatgttcatcattcaattatatctgatcttcaaaatggaaacccagagacacgaaggcgacttgcagtttattgcttgaaggatgcttatcttcctcaaagactgttagataaattgatgtatatctataattatgtggaaatggcgagggtcactggtgttcccatttcgtttctactttcgaggggacaaagtattaaggtcctctcgcaactactcaggaaagcaaaacagaaaaaccttgttataccaaatataaagggtcaagggtctgggcaagatacctttgaaggtgcaactgttttggaggcaagggctggattttatgagaagcccatcgcaactttggactttgcttctttgtatccatccatcatgatggcatataacctttgctactgtactttggtgccccctgaggaggcccgtaaactcaacttacctccagaattcgtcaataaaaccccatctggtgaaatatttgtaaaaccagaactacagaagggtatacttcctgagatccttgaagaactattagcagctcggaaaagggcaaaagcagatttaaaggaagcgaaggacccactcgaaagggctgttcttgatggtcgtcaacttgcactgaaaataagtgcaaactcagtttatggtttcactggagcaactgttggtcagttaccttgcttagagatttcttcaagtgtgaccagctatggacgacagatgattgaacatacaaaaaagcttgttgaagataaattcacaacagtaggaggttatgagcatactgcagaggtaatttatggggacactgattctgtgatggtacagtttggtgtttctacagttgaagatgcaatgaagttaggaagagaagctgcagattatattagtggaacctttaccaagcccatcaaactggagtttgagaaggtctattttccttaccttctaatcagcaagaagagatatgctggtttgtactggacgaatcctgagaaatttgacaaaatggatgcaaaaggtattgaaactgtaagaagggacaactgtttactggtaaagaacctggtaaccgagtgccttcataaaatactggtagaccgtgatgttcctggtgcagtccaatatgtcaagaacaccatatctgatctattaatgaatcgtgtggacttgtctcttcttgttataacaaagggtttgacaaaaacgggagaagattatgcggtaaaagctgctcatgtggagcttgctgagaggatgcgaaagagagatccagctactgcacctactgttggtgaccgtgttccttatgtaataattaaagcagcaaaaggggcaaaggcatatgagaaatcagaagatccgatttatgttttggacaataatatacccattgatcctcaatactaccttgagaaccaaattagcaagccacttttgaggatctttgagccaattctgaagaatgccagcaaagagttgcttcatggaagccacaccagatctgtttcaatctcaactccttccaatagcgggataatgaaatttgcaaagaaacaattaacttgccttgggtgcaaagctgttatcagtggtgccagtcagacactttgctcacattgcaagggaagagaagcagagttgtactgcaaaacagtagcaaatgtttctgacctggagatgctctttgggaggctatggacacaatgccaggagtgccaaggatctctgcaccaggacgttctctgcacaagtcgggattgccccattttctatcggcgaaggaaggcacagaaggatatgaccgaagctaggctgcagcttgatcggtgggacttctgagcttggaaattctcacatggctgcctgcgtctgcttatattgctgtggaagacaggaagctaccgtcgaccgcatcataatgcatgtgaagatgagactggacttctgcgagcatccagtcacacttgcgtccgtgtcacgtagactgacttgccagtacgattcattagcatgttttctgcttaggttttgcgtaatagtttaggtgtgagttggtacttggtagatggtaggtgtcaccttcctgtggctagtgccgagaaaaatgcttggtcctgagatgaggtgctgattatgtacagggcagaagcaatctggagcagttattatgtaacagagtgagacgagacaatttgaatggcacatgagttctcta

SEQ ID NO: 24 - DNA, 3312 bp, Zea mays
  source 1..3312: mol_type = other DNA
atgaactcaggcggacgcggcggcaagcggcggggtcccccgcccctcgctccctccggtgcggcggcgaagagggtcaatcccagccccggcacgccgcatgtgcctcctcccgcgtcggcggcggccgccgtggaggaggacatgatggacgaggacgtgttcctggatgagtccatcctggcggaggacgaggcggcgctgctgatgctccagcgcgacgaggcactcgcgtcccgcctcgcgcggtggaagcgccccgcaatacccaccgacctcgccgccggatgctcccgcactgtcgcttttcagcagttggaaatagattatgttattggtgagagccacaaagaattactccccagctcatctggttcagcagccatactcaggatttttggtgtcaccagagaaggccacagcttatgctgtcaggtgcatggatttgagccatatttctatattggctgtccttcagggatgggtcctgatgatatatcacgctttcatcaaacactagaggggaggatgaaggagtcaaatagaagcagcaatgttccaaggttcgtgaagagagttgagcttgtgcagaagcagacaattatgcattaccaaacacaacaatctcaacctttcctcaagatagtagtcgctctgcctaccatggttgctagctgtcgtggtatcctggaaaggggcataacaattgaaggccttggttcaaagagttttctgacatatgaaagcaacattctttttgcccttcgtttcatgattgattgcaatattgttggtggcaactggattgagcttcctgctgggaaatacaggaaagcagcctgtatcatgtcctattgtcagctagagctagattgcttatactcagatctggtaagccatgcggctgaaggagaatattctaagatggccccgtttcgcatattaagttttgatattgaatgtgctggtcgcaaaggtcatttcccagaaccaactcatgatcctgttattcagatagctaatttggttacacatcaaggagaagaccaaccttttgtacggaatgtcatgactcttaaatcctgctctcccattgtgggagtcgaggttatgtcatttgatgcagagagagatattctacttgcatggagggatttcatacgtgaagtggatcctgatatcataattggatacaacatttgcaaatttgacatgccctatcttattgagagggctgaggttctcaagatagcggagtttccaatacttggccgaatcagaaatagtcgtgttcgtgtccgtgatacgaccttttcctcgaggcaatatggtgtgcgtgaaagtaaggatgttactattgaaggaagagtgcaatttgatctcctgcaggctatgcaacgggattacaagttgagttcttattcattaaactctgtatcagcacactttcttggggagcaaaaagaagatgttcatcattcaattatatctgatcttcaaaatggaaacccagagacacgaaggcgacttgcagtttattgcttgaaggatgcttatcttcctcaaagactgttagataaattgatgtatatctataattatgtggaaatggcgagggtcactggtgttcccatttcgtttctactttcgaggggacaaagtattaaggtcctctcgcaactactcaggaaagcaaaacagaaaaaccttgttataccaaatataaagggtcaagggtctgggcaagatacctttgaaggtgcaactgttttggaggcaagggctggattttatgagaagcccatcgcaactttggactttgcttctttgtatccatccatcatgatggcatataacctttgctactgtactttggtgccccctgaggaggcccgtaaactcaacttacctccagaattcgtcaataaaaccccatctggtgaaatatttgtaaaaccagaactacagaagggtatacttcctgagatccttgaagaactattagcagctcggaaaagggcaaaagcagatttaaaggaagcgaaggacccactcgaaagggctgttcttgatggtcgtcaacttgcactgaaaataagtgcaaactcagtttatggtttcactggagcaactgttggtcagttaccttgcttagagatttcttcaagtgtgaccagctatggacgacagatgattgaacatacaaaaaagcttgttgaagataaattcacaacagtaggaggttatgagcatactgcagaggtaatttatggggacactgattctgtgatggtacagtttggtgtttctacagttgaagatgcaatgaagttaggaagagaagctgcagattatattagtggaacctttaccaagcccatcaaactggagtttgagaaggtctattttccttaccttctaatcagcaagaagagatatgctggtttgtactggacgaatcctgagaaatttgacaaaatggatgcaaaaggtattgaaactgtaagaagggacaactgtttactggtaaagaacctggtaaccgagtgccttcataaaatactggtagaccgtgatgttcctggtgcagtccaatatgtcaagaacaccatatctgatctattaatgaatcgtgtggacttgtctcttcttgttataacaaagggtttgacaaaaacgggagaagattatgcggtaaaagctgctcatgtggagcttgctgagaggatgcgaaagagagatccagctactgcacctactgttggtgaccgtgttccttatgtaataattaaagcagcaaaaggggcaaaggcatatgagaaatcagaagatccgatttatgttttggacaataatatacccattgatcctcaatactaccttgagaaccaaattagcaagccacttttgaggatctttgagccaattctgaagaatgccagcaaagagttgcttcatggaagccacaccagatctgtttcaatctcaactccttccaatagcgggataatgaaatttgcaaagaaacaattaacttgccttgggtgcaaagctgttatcagtggtgccagtcagacactttgctcacattgcaagggaagagaagcagagttgtactgcaaaacagtagcaaatgtttctgacctggagatgctctttgggaggctatggacacaatgccaggagtgccaaggatctctgcaccaggacgttctctgcacaagtcgggattgccccattttctatcggcgaaggaaggcacagaaggatatgaccgaagctaggctgcagcttgatcggtgggacttctga

SEQ ID NO: 25 - protein, 1085 aa, Beta vulgaris
MNGGNSKKRTAPQQQPSPAQQKHHAAMEEEEFDEDVYYSQDGLIDEDTQILRDIEERSAKKLRLSKWARPQLSESYKSQSQSIVFQQLEIDYVIGDCNKEILLNRSGSAAIIRIFGVTREGHSVCCHVHGFEPYFYINCPLGMGPDDISRFHQVLEGRMREANRNAKVPQFVRRIELVQKRSIMYYQQQSSQPFLKIVVALPSMVATCRGILDRGIQIDGLGTKSFMTYESNVLYALRFMIDCNIVGGNWIEVPVGKYKRTGKSLSSCQLEFDCLYADLISHDPEGDFSKMAPFRILSFDIECAGRKGLFPEPTHDPVIQVANLVTLQGEDKPFVRNVMTLKSCAPIVGVDVMSFDSEKELLLAWRDLIREVDPDIIIGYNICNFDLPYLIQRATALGIAEFPVLGRIRSSRVRVKDATFSSRQYGIRESKEVTIEGRVQFDLLQVMQRDYKLSSYSLNSVSAHFLGEQKEDVHHSIISDLQNGNAETRRRLAVYCLKDAYLPQRLMEKLMFIYNYVEMARVTGVPISFLLSRGQSIKVLSQLLRKAKQKNLVIPNSKQAGSEQGTYEGATVLEARAGFYEKPIATLDFASLYPSIMMAYNLCYCTLVTPEDIRKLNIPPECVNKTPSGESFVKPELQKGILPEILEELIAARKRAKADLKEAKDPLEKAVLDGRQLALKISANSVYGFTGASIGQLPCLEISSSVTSYGRQMIEHTKKLVEDKFTVVGGYEHNAEIIYGDTDSVMVQFGVSTVQEAMKLGREAAEYISATFTKPIKLEFEKVYYPYLLISKKRYAGLFWTNPDQYDKMDTKGIETVRRDNCLLVKNLVTECLHKILIERDVPGAVQYVKSTISDLLMNRLDLSLLVITKGLTKTGENYEVKTAHVELAERMRKRDAATAPSVGDRVPYVIIKAAKGAKAYERSEDPIYVLENNIPIDPQYYLENQITKPILRIFEPILKNASRELLHGNHTRSISISTPSNSGIMKFAKKHLTCIGCKALISGSNKTLCSHCKGREAELYCKTVANVSELETLFGRLWTQCQECQGSLHQDVLCTSRDCPIFYRRKKAQKDMGEAKVQLDRWSF

SEQ ID NO: 26 - DNA, 20806 bp, Beta vulgaris
  source 1..20806: mol_type = genomic DNA
cttacaaacatacctcgcgcctaaaaacataactggcgggaaatctctcatctctctctctctgtttgtgtgtgtgtcaattgttgcagccatgaacggcggaaactcaaagaagcgcacagcaccgcagcaacaaccttcacctgcgcagcagaaacaccatgctgcaatggaggaagaggaattcgacgaagatgtatactactcacaagacggcctcatcgacgaagacactcaaattcttcgcgacatcgaagaacgttccgccaagaagcttcgtctctctaagtgggctcgtcctcaactttccgaatcctacaaatcacaatctcagagtatcggtaattattttcctcttttatcagattctagggtttaggcgagggtttattattcaatgtgcattttgggggtttttcttgttgcagtttttcagcaattggagattgattatgtgattggagattgtaataaggagattttgttaaatcggagtggttctgctgcgattattcggattttcggggttactcgtgaaggtaaagaatgattttttatgaatttgcttgcttaatgatgcaaactatacattttggcaaacttttgttttgattatatgaaacatggtttataggtgtgtagttagggtacagtctatctgttggactcaccttttttttaaatgtttgcattcttattgcaccttaccatattgagcttctggtatgatgggaagttttggaatgaaatgaagaattatgtaaaacgagtccaaggagaagatgacataagcttggaaattcaaagttcaaccatctagtagaaatgattatttcttgtttttgattagacgggttgtgccaatgtttggtcatattattgacattttgggtgagaagaagagaatggcaattttttttccctcttttctatgaagtggcatttagtggcaacattcaatggaaactcattctgggttcaaaattataaagaaaagagaagactgcatgccaataacatcgtcccttctaatacgtagaagctagtgggtttttcatttcacataaattttttgcttcatattgttctctcttgaattagtcaaagaaatctacctagacacttacattttttatggacatcaatgatggagggaaagggaggggaggcaaggagagggaagggggaggaaaggaaggggagggaagagtgagcttgcagtttctttacaattttttttgacaaaagaattggtatggttgatgttatggatagaggaagttgttttaaactcactttagagatgtgaaaaacaaatagggacatccacatgtaggaagagtagaattcattgtgagagaagagtataatgtataatgctatggtggacagagtttaagaactcttttgatcttgaattactttgttctgacctatagacatttgattgacatactcatcgtagtagatttttacattattgttaaatttgttgttcagcagtgcctcgtgcgtaaatttttaccattttgtgattttttttccgtttgctacacggtcttgtatgtgtgtggtgtgggttcatcccaaatataatatgcttttgtatggaaatggagttgaaacatatatttcatttggtcttgagtccatctattacaacttgtgacttgttgtaatctgattaatgcttctcgtttcactgacattgctgttttaatgggtaccttctgaactttatgaaattccatttttgcttagtaatttatttattaaccaacttatggctcttagaaaccgaaaatggatgttttactacgtattgtcaacttgagatatagcacacaaacattttgcatttctatatggaaagctttaagttccattatttatgcagtgcatatttctatttgagtgttactcttagattttaacatatacatagactaatgattttattacttcccttgtccccaaacaattttcctcattactctatcattatgtcctagaaaaatttcttttctctcttcaaagttcaaagtaaaagtcaatgaatttaatattacttttctctctttatcacgccaaccctctctctctctctcaaaatattgttttgttatctcccgactaaatcttgctgcttacattttaataactattcttcagaactttgtcaaagtcaaccaggaagttaatcttaggacaaggggacttagggagtaatagttattaagtgtggtaatttcacatttttctgggatgtataaagcctaattatgttatctatattgcttaaagagtcagggggatattttattttttaattttgcagtcttggtgtctggatttctaatgagcatctaaatttatcatggtttactctaccaaacagggcacagtgtttgctgccatgtacatggatttgaaccctacttttacataaattgtccgttagggatgggtccagatgatatatcccgttttcatcaagtcttggaggtaatgcgtttttgctttgctattccatatattctgtcactgacattatatgcccctaagagttgcggggtagaatatgaaatcagtgactccatatgtggcttctaatatttattgtgatttaggggcgaatgagggaggcaaataggaatgccaaggttccccaatttgttcggcgcattgagttagtgcagaaaaggagcataatgtattatcaacagcaaagttctcaaccttttctgaaaatagtggttgcattgccttctatggttgctacttgtcgtggtaaatacaaattttctcaactgtcctatctgatcctgtcatcctttcccaatgaaaaagaggaacaagaaaatgaaagagatgtagaaaggagattgggaaaaagaaatattataaatttttgtgacaaagctaaatatgaatgattttttttggcgtcgtcagtaatctatcttattagggtttgacttcctatcgttgccatagattagtacccttgtgtgtagccattgtttaaatttcttgtacgtgtatgcttctaggtttagggttatgcattgcttaaattgtgccgtgttctaaactgccagggattcttgatcgtggcattcagattgatggccttgggaccaagagttttatgacatacgaaagcaatgtcctctatgctctacgtttcatgattgattgcaacattgttggtggaaactggattgaggttcctgttggaaagtataagagaacaggaaagagcttgtcttcttgccagctggaatttgattgcttgtatcctttttcttttaattttatattttagggtcttgtttggcagtagcgtttgaggtagcggttagcggtttgacaagattaaaacgctacttaagaaaattatgagtgtttgttgaggtagtggttgtagtagcgggtagtagttgggagtagattacgaatagtgatcgtggaacgttattacaagtaatgtttgagatttagaggtagtggtctagcaaataaacactatataatagcatagggtagatttattaaccaatgttttttgtttctttttccttttactgtttattaattgttttattttatggcaatttaatgttttttttacaatagcagagatgtaatttaaatatttatttaaagataaaatattttacaagtatataaattttttggggtttgaatattttcttagattcatacatttttttcaggagctaaaaagtcgtacactcctttttgtgtaacttctttgaaaaaataaatattgaatctaactattgaacgaatatatgaaattgttgtagttcatttcatattagaataaatgttatttattgtctcttacaatttcaacatctacttttgccaaacacttctaattaaacaattaattttgctaatcgctgacagctaagccaaacacttctaattaaacaattaatttaatcgctgacagctactagtagataactagcagctacccccaacacctacaggtacagctactgcaaccactaaacaactactagaatcgctagttttgacaaacatgccctaggtatcttatattcctgtttgtgaactattttcatggataacttcccttcccagtgtatatggaggaacataatttcggaacacaaggcatcttttttcccgtctcttttttgtggattcccttgattgtgctaatagatatgcagacttaattagccatgatccagaaggagacttttcgaagatggccccattccgcattttaagttttgacattgagtgcgctggtcgcaaaggtctttttcctgagcccactcatgaccctgtcatccaggtaaaattttgttttccacttgccaacctaaattatggaatcacttctaaaagcttgtggcgttagtcacttggccctgttaattttgtaagctaactcaagtgatttacttccattgttaggtggcgaatttggtaacattacaaggtgaagataagccatttgtccggaatgtcatgactcttaagtcatgtgctcccattgttggtgttgatgtcatgtcctttgacagtgagaaagaactgttgcttgcttggagggtaaaacttctcatctgtgaagatagtttcctactttctctgatcatattttaaaataattcttgataagtcagcctacatcaataggcctgaccttaaaccccacaatgactgatgcctacaagaccctctttatagtttactgcatctcttggatatggatactttacgtatgatagtttaaatttgttgtgaaaacatataaaagctgatgcatctgtgtgaagttataattttcaagttcccaaaattgacactctggaagtgtcacagaaacatgggctgtaaatgatgggggtggctagatgagtgattttttgcttgtttgtgttgttttgtgaggtgaatccgatttgaactactatggtactctttcttggtttgttttaggcttttagctaagtggtggtcgtcgaactgaaagattggagtgattcagttcaagcagtgcttcatatgagggctacattgttctaaatgctgtttggtggtagctttgtgactttcttgttgaatgcaagattgcaagtcatcatcttttccctatctaagataccagcaacttttcgtcctcctttgatgctatactcgacataacatatgagttcttttggcacatgatttaatttttatgttctatctacaggatttaatacgagaggtagatcccgacataattattggatacaacatctgcaattttgacttaccatatcttattcaggtatatttctttttcatctatctaatttttcgtgtcacactgacaaacatatggatgtttggaggggtacacacaaaaacttatgaagagttagatatacaggtagcagttggacgcacatacacacacaaaagtgatcagtgtgcatttgatgttataggaggcaccatatcggctttaggaattcctttcattccccagcttcctgtaattgtataaaggccttatatgaccctattttaatgacagagagcaactgccctaggaattgctgagtttcctgtccttggtcgcataaggagcagtagggttcgtgtcaaagatgcaacattctcttcaaggtgatctttctatattgaggattcatgtatttttcttgaaattccctccgcctcatattatttgcatgttattatattttgggatgtcccatcacttctgaagtctaaaatattagatactctctagcacttcttcattagagcagcaacatttttacttgagtcacgcactccccatttttttgaattattatttttaacttttaaaggtcggtgtcacaacacttttccattagagttagaccatttctttgttagagtcaaaccaattttcactcaaagactcaaagtcacacctctcaattttatttatttattgatttattattattgttttattattgttgttgttgttattattattattattattattattattattattattattattatgattatgattattattattatattttttaaggttggagtcacattacttgctcattactcacaagtcacaacacttcatcattagagtcataacgctattcacttgaaatcatcccccagataattatttattttctttttaatggtaagagtcacagcacttctccagtagagtcacaacttttctcatttctcacataaagatgcattctcatgtgaacttatatgtctctgtctctctttctatataaagttcacatgagaatttttcttatatgagaacgtgaggatagtggtatagtcatccatttagtaaatgtgtaatcctttattaagatatcacatggtaatcctatttcggaagattggtccactaagtcttagtgagtcacctttgtgatatttaccactgggatgatgcttgatcacgaattataagaacattaaaaagcgtgtttccagaaattacttcagtatccactttggggaaataccaggatattatctgttctaaaaccaccagaaaaaaatagctcgtgaaactataactatgttttatactctttgctgccttttacttgggaaccttttttttagggttatgctatatgcggccctaagggttgcatataagacatacatacactaccttaaatggaaataagttgtgaatttatggaaaaattatgcaacaatcaatgtaataaatgatcaattaatacatattcttttttcatttttagctacttatatgcaagggctgcatatagcttttccctttttttatttgactccttattttgtagcgtaagggggagggttattggtgtataattatgttccttcattgcattatatgctgaagagctcatttttttccttattggtgtataattatgttgcccaaaacttagaaccaaagaaaataagctattattttgatctccatttgcctttctgtcactttgacattcctccccctcttccctcccttgggttctcactgaccttaaatttggaccagaagagtccattgcactgctaccctaggcccactttcaagcccacatctattatgaaaaaattgaagtgctaacatgcctgaaaaggccttgcaactatacaaggagttgttaatcttcagagaagtatgctttggtgtctagctggtacatttctattttggcttccgcatttggtcataaggaaggactgataaaatcaacgggagtattacaaaaaattggcaagatttatcgaatatttgggcctgtcgtggggtgtgttaaaaattgatctgaaaaagatgactctgttttgtttgtttaatatctgatggtgatggaggatttgttgataatattacctggtgtgatttagtatattaacttcttgccattgaaaacattgttgaggcaccatgttagcttgagactttgagggtcgtggagctcaaataggaacatgcacccacatgcatgtagatgatggtgcacccccctttaaactaaccattggaccatatttagagtccatggtttataaaaagatgttatttgtttccttaaatcgtgcaatttcttttaacttttgaaaaaaacggttattgatgttataatccttaaataggtttattacttttatctagaatatatgttctttaaattataattgtatcttcatctattacacgttctctttgtatgacccacttttttatgactcatgctcttttaaaaatgtagaatatatatatttatttatttatttgttttgaagtgtgttctttttagctggaatgtatgttctttaaacttaaattgtatgttcttttcatatgacacgtgtttttcaatgaatagagattagacatgttttttaatttaaaaccatggtttcttttggtatgttcttccatctgatttagttctttgttttcatctatgatttcttaattattgtgtgaaatttaaattgtaagttgtgtgaccattaactctatataaactcttgaacaagaaaataattcaaactctttgacgagattccttttggaattaaatctaaccgatagttcttgtaatataaatcgtatgttcatttgctatatgttcatttgattatgacctatgttcttttattagtggaaattatacatgttctttaattatgaattgcgttctttttacctagaaagattattgcaaaaagtttgtgttcttttatatgaaatatgaatggtatgttcttttcggatgcacaatgagcactgtgcaccctgctgcataatccacgctttggtcatcgagtggcctgaagtcagatagatctttttttctcctcaccccccccccccccccccccccaaaccccttctgatttcactttctcttcaaaattttaggatatcattgaaacttatgttgttttacaggcagtatggaataagagaaagcaaggaagttacaattgaggggagagtacaatttgatttgcttcaggttttgctttgtcctttgtttgtgtaggttaacgcttgcctcagatgtataatcatgttaatatcttctgctgcaggttatgcaaagagattacaaactaagttcctattctctaaattctgtatcggcacatttccttggtgagcaggtaattctaaagcaactactagccaggtcgtatgtgtaaagtgttaatttcacgtgtttatcgtgctgaattctaagggcagattttttttttgaagtctggactaaatgatgcattctaaatcaatattttaaggattgccccaaggactttgaataattatcatacttcttgtatgtttgaattggtggtatcaaattgggaactaatgacaaaattgtgcatctatcgggttattgaatgtccaatatatctaatttaagcttcttattcatatcccctaagactatgttttcagcttattttatacttcaggaagatcatatgggaactattaaattgttttatttaaatattctaaagcatacatcaaactttctttctttaatcacaaaaagttcccatttccatttataaaatgcacatgcaagcaatggcttaggtcttttgttcggaagagatacagtgcacagaacttgtataggcaatgcctgcttttacctagcatctaacatgtaaaagatctatgtgtatttaagtaatttaatcgtgttagttttcttccctttcatctattaccatagagctatcatgttgttttttggtatttgataatttctccttgtttaaggtcagtgttctagaatctctctttgctattactttctctagctgcatagattgatcaaaattgatgcaaaaaagattaagaatctatctttttgttctccgtgtaatacttctgtatatgaaaaagctgttatgttcctccgtaggcaatttgttatagtgcttgtcttccccattggactccctgtctgaatggtggatagctattgttcctacatttggctgtacagttagcatggtgtatataaaaacgttgtacgtaagaactttgtttgaattgtagctcctctgaaatctttatattctgtacgtgttaagatgctaatttgaggtggaataatggctttccttatgacgcatgtaggtagagggaaggtgtgactttttggggttagctagtagcctagtatttagtatgctgggatatttgatgtgcaagccttttcaaattcatttaaagcttaatgctttccttcttcaactttgcctattggttgggaatatgtagactggggtgttctttataatctacatcgtcttctatttttgtgatttagttaatgcttattaaatattaatttgcttgttgcagcattagagaagaatcaactttggaaaaatttccttagcgagctaccatatacttacgaataatgcatctgacatgattttctttgcagaaagaggatgttcatcattccataatatctgatctacaaaatgggaatgcagaaactaggagacgtcttgctgtttactgtttaaaggtaaactattttaatttggttaatttatttcatacggaaatgtttgataatgtatttgggtgcttttttccccccttgcttgtacaactctgtgtgcgtgcttatgtgcagatacacattataagagaaggtttatgttttagaattatggctgaaccttaaatttcttagtgaaggaacaagcagaaagcagatcattcttaagatgctattttgtagaatgttttctgtaaaggattgcttttagttctgcaagataatccaggttgtacaggtaagtggatgatgtatttggtttcctcatggtatgtaattaggcaagcaaactaacttcagcaatcagcattgtaaaggttgagcaaattcttgaaatatattcctaatatctataaatattgattttacatcatccatgggtatatgtttttggcttcatattgggatctcattgtttaattatgagcagactttgaacccaaaggtaccctcggtgaacataacagtcatatgagcggtacttgtttaactaaacaaggaaaaaagttgctggtctcggttcatatagacgcatgctatgcctagtctcttgatttgaccgacacgtagctggtaacagctggcgagaagcaaaaagtaagaagtgtgcagaagtaattgaacaagtggcaagtagaatactaattattggtattattgtctaatagatgtcagttgagatgttgtagttttatacttttacatattgaaaattagcttcgacagttctcaggcatgtgcttatgtatatggttgggaaaatggtagcttgtgaaccgatgtagttagcatggcttggtagagattgcttggcagtgaatgctaaacatggtgaacatgctgtctttcatgaattataggatgcttaccttcctcagcggctgatggagaagctgatgttcatttacaattatgttgaaatggctagagtgacaggagtccctatatcatttcttctctccagagggcaaagtattaaggtaacatgcttaattttttctgccaaatggttcgcctatcttatgctagagtcaactttatttattttcgttctgcaggttctttctcaacttctgaggaaggcaaagcagaagaatcttgttataccaaactctaaacaggcaggatctgagcaaggaacgtatgaaggagcaactgtaagtttgacctttagacagtcttgtttgattcttattggattgcatcttgatctaattagatagaatatactgttatttatgaaattttcttatatctgtagaaacaataaaaatcgtcatctctcctgcattattggattatgtaaaaatagcagaataatcaatattgtcgggaatatgtctagttttttgaatttttttccactttatttatgtatattgtgattgtatcactcttgcctgactgtatgtgtgaacaatactactggaagcatttatctgttagtctttatcttattttttctttcgttgtatttgctataggtgttagaggcccgagcaggtttttatgagaagccaattgcaacgctggattttgcttcactatatccatctattatgatggcgtataatttatgctattgtacactggtaataactttttgttggacaacaggcatcactgacttctattacttttgtcctgagatattctttggtttatctaagacatcctgttcaggatataatttgcttcttatacttgagttccttacatgtgcttgtaaaggtaacaccggaagacattcgtaaactcaatatacctccagaatgtgttaacaagactccatctggtgaatcctttgttaagccggagcttcagaaggtacagatttaatttccctacgcatttaccatcctcttcctgtctacactcttaagtcttgacctgcaaaggcatcacgttttatggacacctcaaaatggaagttctgataaatattgcatgatttcaccattttattagggaatacttccagaaattcttgaagagctcatagctgctcgtaaaagggctaaagcagatttaaaggtgtcctttttcaaacttggttaacgttcctaatctgaatcttgtctctgagatatgactgttcctatctttcaacccattaatgattatgttgtttgatcataggaagctaaagatcctctggagaaggctgtcctagatggtcgtcagctggccttgaaggtatgcccaaggccttttctttcaacaatatatacgttattgttattcttgtcacttaggattagctgctgagaatggttggtgtttgtcagatccttgttttttgttttgttatgatcttttaaaagagcttctttccagtttgcatccttcaggtctcatttcttgattacaaatattctacctcaacctctaatcttattttgccttcctttcagataagtgcaaattctgtttatggatttacaggagcctcaattggccaattaccatgtttagagatttcttcgagtgtcacaagctatggtaatcttaattttttttgcatgatcttgtgtttatcatcttttgttgcaactctgtttaggtatctaattcagcaatgtgaatgcaaaattcacaaaaacttttggaagtacaaatttggaggggaaggagtgaagatacttttgcatatactatagcatagcactccacccatcaaataaattaaaaaaataaccatagcgttccctacgttgaccaaagtgctgcttttagagggtcacctatagcttgctatggagtatggagtggttagaattacacttagtcttgtttcccttacctcctcaattgcatagaggttgttttactggactctatcggagttgggtgatgcatactctcattttctactctattggtaggtgattggtcaatatctgatttagtcatacgagcaattgagcaagatgctgaatagcaaactacccgagtcaataaatgttagtgttcgagcatggctcgggcatagaatgacatgctcgaacgagctaataggtcttttgtttgggcttgggtcttcaagcatttagctagctcggggttggttcaagctcgctcgcttggtatttcgaggcaacaattgagtctctcgagctgtcgagcattagaaaacttaattaaatcagtgagcctagtgatactcgttgaaaatggattgtcacgatgattttatgagtaatttaactagattcaattttgtcgccaatgttttcccgtaacaagtacctatggccatcttttatacatacacttgcaagtccatgattccaaagtcttatctttataagcttttattttgctttcaatgatctgcagtaattttaatatagtatttcattgtcgtggttattttttttgagcttgatcgagttattaatgagtttgagacgagtttttaaatgaacttaaatttttgcaaacgttaactagttgaatacaatttgtgtttacgtttggctcttttacggaaggattttaaattttttaattggtcttggcgtcttgctcatttattcaacaaatatgatttgaccaagcttgttagcgagccattcacagacgtacggcttgttggagccctatttgggtcttgttctttttttgcttctttttcctgttaatattttccctttgtcttctgcgacttactcttcgagatgttgaaggcttgaagctaaaaatattagagccagagctcaagggttacttggactcaatggcataggagataggactatggtgcaatgatgcttttttagagagctatatttatgccatatttttccttctatattgttactttgcaaagtatacattcttctgtgttctgtcaattgccttcttttcatagtgtgccatataggagatcgttatattggtttatctatgcttatgaggtgtctttcattgtacaggtcgtcagatgattgaacatacaaagaagcttgttgaagataaattcactgttgttggaggctatgagcataatgctgaggtaaatcataccctcttagttctgtgaagtccatcaggatgaattattttcttatttttgtcataattggatgttccttactatgacttctcaatttctttggttgcaaactcataatgcttaagatccccatttttgtcaattccaaagtgatactgtctatactctatagtttcctctttgtgcagtggaagtgaaaaggaagatttttttcttctttttttttctagttgtgcagattttacattttattccacaagatttccttttccatgtttgttaattgttgcctcaattattgcacttattatgtcataacatttctagtttcagggtcttatgttcaataatatggtattgcttgtggtgtttttgttttaagagaatttcggtgctgtatattgtttattagacttattacttttttccatttgtgatttttgaattaagttgcactttttcaacccagattatctatggagacacagattcagttatggttcaatttggtgtttccactgttcaagaagctatgaaattaggaagggaagctgctgaatatataagtgccactttcacaaaggtaagtcgtcgtccatacttactagtattcctccatgtaatactgctgattgttgagtatagtctccttatatcgcttggatatttggaaattaattttgataatataattacagtgctccattttgtacaagacgatcttcctgctatccttttttggggctgctctccaaagatattctacttaccttaaatgtaagggtatgttcagcaaaaattagctgttgtagtagcttagctttaagattagccgttagctgtggcctgtgggagtagctgttagctataaataggagctgacacttagagcttgctgtcaaattagctgtttgaatatgtattcggcaccaactagctgttgaaatacaaaactaaataggatatggaactttataacttaatttggtagttttgccaaccactacttcaaaagctagttttgttaggtttcgcaaaacactatttaaaaagctagaagataccaataatgccattcaccagtttttgattaacactagtatcggaattttacagtgtaaaagctactccaaaacccgatgttggagatgctataaagttacccttcccatggacttgaacctccaccttctgtctctactttttctcttttattgctttatcattttttcgtttacaattgcaactttttattttcatttactcaatctctatccaattatccatttgaaaattttattctcattttaaccattttcttaatctttgtgccaaaatctaacatgaaaatcttatgagattagaggtagtaatttggttttcttattagataaataagtgttaataaattatatctgtttctttgggagaaacttaagtagaacttatgtgtttgaatgtccacatttttgatgagaaattggcaactcccttacttttccaatcagtaagcccttcattcacacaatccttgttctggctgagtggactacatcctggagtcaagagtcatattcatgaagcctacggtcaaatggttaatgaagaaaaaacatcaactggctaccctgcaaaaagagaccgacactaatactatgatactgtcaaatctacgtaataaatgtattagcactcagccataatgctagataagtgttttcgtcagcaacttgaaattagttatgagttttgagttccttgagatgttagaaatctgacctatatgtagtccatgcttctaatataagcaactggctctcctcctgactgtttgttccaatcataagacttgcttgactcaagtgtgtgtcaattggactaggcggaaaggaaaaaatggagcaagctaggtataaagaagctgatcccttttctccgatatactgcctctagtcataaagtgctccagatggtttcgcatatggagaaatgagggggcagggaaaggaagaattatatattcttggaagaagagtaggctgagttcgaaggttgagggggcatgatgcgaggattcaggaacagtagataagtatgtatgtgcatggaaagaaagatttgcttcaacttattatcttaaccttagtggaacttcttttcaatttattcatgaaggtgtttctctctttttgtcttctgttctgatggcctctttctccttttttcttattatcccttttaagtgatctgaaaggcaaagataaatttgactaatcgaacttagtctaagttgatgggattgaaacatttcatggagtgttttcactgatggaccagaatattcacccataatctcattttaacaatttggttaaactttcaaaagaattgagtcttaaagtgagaacttcacattttagtggttggctgaaagttgagaatgttacagacttttggtttaaaaagacgtgatccctgtgcatttttgactgttttctcccttgcattgctctttgtatttgaatatacatttgtatgtcaattgcagcccatcaagctggagtttgagaaggtttattatccatatctgttgattagcaagaagagatatgctggccttttctggacaaaccctgaccaatacgacaaaatggataccaaaggtaattactgggataatttgtgttattttttttgatttcctagttatggttgtcaattgagtattaattttctgttcttgttgcgtcatttgtcaaatttcattaacaggaattgaaacagttaggagagacaattgcctactcgtcaaaaacttggttactgaatgccttcataagatacttattgaaagagatgtcccaggagctgtccagtatgtcaagagtaccatctcagatcttttgatgaatcgtctagacctgtctttgttggtcattacaaaggtgggaaagtatgaatcatgattataacaatgttattctatactccctctgatcaagtataaactggtttagcatatttcacaaatatcaagaatttattgtcttgatattgaacccataatttttttaaaatattataaaatcacacttaatattgtgttggtattgtaggacatatattttggaattaaaaatatttgttgaatctgcatatattttggattggggagagtgatattttccatgaagcttgcctactctaattattattgtgggttctctgaaagttttcctttcttccccttctggtttagctgatatctgtttctttcagggtctaacaaaaacaggggaaaactatgaagtaaaaacagctcatgttgagcttgctgaacgcatgcgtaaggtgtgttttctgatatattgcatattggcctcgtaactcttgatgtcgctgttgcctaaaaactaaataaagatagaattctaaaagctgtgatctatgtcctcagggatgtcattaaggttttgttagcagaatccctgcaaattagctcaaatgtatgtttatatattaaattaatggttgctcatgataatgattcatgatgacgagttggggggggggggggttctctggccgttgagataattaacatctaaaggaatgaagagctgttcatgtgaacggtactgaggggtggagtactggagggataaaaagttagctgagatggttcatctatagaatgagattccaaactagaatataattctattaagtatgaagtagctaatcttttttctcccttaatatgtcagcgagatgctgctactgcacccagtgttggtgatagagtaccttatgtcattattaaagctgccaaaggtgccaaggtacgtgtgtgcccctaaatcgtttatattaaaatgtaggctctgaaatgcaggatattaaataatgaattattatcaacaggcgtatgaaagatcggaggatccaatatatgtgctggaaaataatataccaatagaccctcaatattaccttgaaaatcaaattaccaaagtaagtaatcaatagctcgatacgatatccctgtcaaatttgatttcttgtatgaagttgtttctgatggttgttgcaaatcttgtagccaatattgaggatctttgaaccaattttgaaaaatgcaagcagagagttgcttcatggaaatcacacaagatccatttccatctccactccttcaaatagtggcatcatgaaatttgccaagaaacacctcacatgcattggctgcaaagcattgattaggtagtttttgtaccactccctgtttacctatttctacattagactagcttataacatctatgctttgtgacctccctcacttccatgggtgaagaattgctttcactatttaatagtgtgggttacatgctggtcatgacaatatgctggatctaaaatgctaaatacatcaagtgcctgtttcgcgtcttttttgatggatatgtgactgttggctaatggagttattaggagtttagggtacctggtctgtttttttattggatatgtgactgttggctaatggagttattaggagttcaggacctggtcttttttttattggatatgtgactgttggccagtgaagagtgtaatgtttagaaatcttcataatctgaatatgagcttggctccatttgatcgggtgtaaaacattttcagtgtaaaatactttttgtggaaaacaatttacaatggaaaacacaattcccaaattagttttccgttgtttggttccatgtaagaaaaatgatgaaaagaaaggaaaataaaaaagaaagtaaggaggtagggagaggagtgaaggagagataagaaaaatgaaggaaagcaggggaaaatggttttccatcattggaagagcaattttactcctagggaaatggttttacaaactaaataatgtaaaattgaaaatagaggaaaacagtttttcaggaaaacattttacgccttaccaaacggaacctaattgtctgagagtggctctgttgtgcagactgacagttgttagtgcagatatgctacctctgtcattagctatgtgcgttttgccaaatgagtttcctatctaaccagacatgccaaactgcatggtgttttatatatttacccacaatctcttcttattttcttgcattttttttgtcatggcgtatcttcaagctgtttagttcgtcatgctctattgcaagatattttcagtttcttgacttttgtgtctgattatatattgttcccttcttttgtgggacacttcagtcttgagatttctttttgacatatttacctggtattggattagtggctccaacaaaaccctttgctcgcattgcaagggaagagaagctgagctttattgtaagacagtggccaatggtatgtaagataacactcatatttccatctaccatttagatttaaattcacggtgcttacggttggaaaacattatctccttacagtttcagagcttgaaacacttttcgggaggctgtggacacaatgccaggagtgccagggttcccttcatcaggatgtgctttgtacaaggtctgagctattgcacctttattgcttccaactcctccccccccccccacccacccccccgggcaaattgttttgtatgctcgtcaccttgtaaacacaagcctgttccaatgttgtcaattgttgactgctctgatgcttctcttgactcagatcctctctataactcaaaatgcattactcatgttgcatttttatttttccttcagtcgtgattgcccaatattttatcggagaaagaaagcacaaaaggacatgggtgaagccaaggttcagctagatagatggagcttctgatcaaggaggttgcaagtttgcaactctgtacagttagttttcctcaatgtaccatttcctttgtaaggctctttgttgagtaacttttatatggtcatgtaacggaatgcgagaaactctggttgaagtggtcgaaatgaagaattccttgccactgggtcagctcaaggatgttatcagcgatgaagtgaaactcaatttattgtcgtgccataaatatcatccgcttcatttacgacaagattaatttatggtccctaagagatacaggcaagacttttggcgccctgctcaaccttcggatgcacgagagtgaggaagggggataacagagtccacgtttttctacattttgtcaatcatgtgaaagagatgaagttccctagtggcatccgaaagaagtcggataattactgagtcggacaggattcaaagttcaatattcaatatctggttcctacctgtaaaaccattccagtcatccatagtagaacttaatatccctgcgtcaagcaatctgctattatcttttgtacatagagtgtagatttctggagtcattagagttgtgtaggattgagctattcgctttttgttgacgagattaaggatgtcggtttgcaactagctttgtaaaacagaggtgtcttcagtgatcaaaaatataattgctccatgtgttagctagcttgtgttattatatggctgactgaaatgtaggcgcgacatgattttcagtacatctgta

SEQ ID NO: 27 - DNA, 4097 bp, Beta vulgaris
  source 1..4097: mol_type = other DNA
cttacaaacatacctcgcgcctaaaaacataactggcgggaaatctctcatctctctctctctgtttgtgtgtgtgtcaattgttgcagccatgaacggcggaaactcaaagaagcgcacagcaccgcagcaacaaccttcacctgcgcagcagaaacaccatgctgcaatggaggaagaggaattcgacgaagatgtatactactcacaagacggcctcatcgacgaagacactcaaattcttcgcgacatcgaagaacgttccgccaagaagcttcgtctctctaagtgggctcgtcctcaactttccgaatcctacaaatcacaatctcagagtatcgtttttcagcaattggagattgattatgtgattggagattgtaataaggagattttgttaaatcggagtggttctgctgcgattattcggattttcggggttactcgtgaagggcacagtgtttgctgccatgtacatggatttgaaccctacttttacataaattgtccgttagggatgggtccagatgatatatcccgttttcatcaagtcttggaggggcgaatgagggaggcaaataggaatgccaaggttccccaatttgttcggcgcattgagttagtgcagaaaaggagcataatgtattatcaacagcaaagttctcaaccttttctgaaaatagtggttgcattgccttctatggttgctacttgtcgtgggattcttgatcgtggcattcagattgatggccttgggaccaagagttttatgacatacgaaagcaatgtcctctatgctctacgtttcatgattgattgcaacattgttggtggaaactggattgaggttcctgttggaaagtataagagaacaggaaagagcttgtcttcttgccagctggaatttgattgcttatatgcagacttaattagccatgatccagaaggagacttttcgaagatggccccattccgcattttaagttttgacattgagtgcgctggtcgcaaaggtctttttcctgagcccactcatgaccctgtcatccaggtggcgaatttggtaacattacaaggtgaagataagccatttgtccggaatgtcatgactcttaagtcatgtgctcccattgttggtgttgatgtcatgtcctttgacagtgagaaagaactgttgcttgcttggagggatttaatacgagaggtagatcccgacataattattggatacaacatctgcaattttgacttaccatatcttattcagagagcaactgccctaggaattgctgagtttcctgtccttggtcgcataaggagcagtagggttcgtgtcaaagatgcaacattctcttcaaggcagtatggaataagagaaagcaaggaagttacaattgaggggagagtacaatttgatttgcttcaggttatgcaaagagattacaaactaagttcctattctctaaattctgtatcggcacatttccttggtgagcagaaagaggatgttcatcattccataatatctgatctacaaaatgggaatgcagaaactaggagacgtcttgctgtttactgtttaaaggatgcttaccttcctcagcggctgatggagaagctgatgttcatttacaattatgttgaaatggctagagtgacaggagtccctatatcatttcttctctccagagggcaaagtattaaggttctttctcaacttctgaggaaggcaaagcagaagaatcttgttataccaaactctaaacaggcaggatctgagcaaggaacgtatgaaggagcaactgtgttagaggcccgagcaggtttttatgagaagccaattgcaacgctggattttgcttcactatatccatctattatgatggcgtataatttatgctattgtacactggtaacaccggaagacattcgtaaactcaatatacctccagaatgtgttaacaagactccatctggtgaatcctttgttaagccggagcttcagaagggaatacttccagaaattcttgaagagctcatagctgctcgtaaaagggctaaagcagatttaaaggaagctaaagatcctctggagaaggctgtcctagatggtcgtcagctggccttgaagataagtgcaaattctgtttatggatttacaggagcctcaattggccaattaccatgtttagagatttcttcgagtgtcacaagctatggtcgtcagatgattgaacatacaaagaagcttgttgaagataaattcactgttgttggaggctatgagcataatgctgagattatctatggagacacagattcagttatggttcaatttggtgtttccactgttcaagaagctatgaaattaggaagggaagctgctgaatatataagtgccactttcacaaagcccatcaagctggagtttgagaaggtttattatccatatctgttgattagcaagaagagatatgctggccttttctggacaaaccctgaccaatacgacaaaatggataccaaaggaattgaaacagttaggagagacaattgcctactcgtcaaaaacttggttactgaatgccttcataagatacttattgaaagagatgtcccaggagctgtccagtatgtcaagagtaccatctcagatcttttgatgaatcgtctagacctgtctttgttggtcattacaaagggtctaacaaaaacaggggaaaactatgaagtaaaaacagctcatgttgagcttgctgaacgcatgcgtaagcgagatgctgctactgcacccagtgttggtgatagagtaccttatgtcattattaaagctgccaaaggtgccaaggcgtatgaaagatcggaggatccaatatatgtgctggaaaataatataccaatagaccctcaatattaccttgaaaatcaaattaccaaaccaatattgaggatctttgaaccaattttgaaaaatgcaagcagagagttgcttcatggaaatcacacaagatccatttccatctccactccttcaaatagtggcatcatgaaatttgccaagaaacacctcacatgcattggctgcaaagcattgattagtggctccaacaaaaccctttgctcgcattgcaagggaagagaagctgagctttattgtaagacagtggccaatgtttcagagcttgaaacacttttcgggaggctgtggacacaatgccaggagtgccagggttcccttcatcaggatgtgctttgtacaagtcgtgattgcccaatattttatcggagaaagaaagcacaaaaggacatgggtgaagccaaggttcagctagatagatggagcttctgatcaaggaggttgcaagtttgcaactctgtacagttagttttcctcaatgtaccatttcctttgtaaggctctttgttgagtaacttttatatggtcatgtaacggaatgcgagaaactctggttgaagtggtcgaaatgaagaattccttgccactgggtcagctcaaggatgttatcagcgatgaagtgaaactcaatttattgtcgtgccataaatatcatccgcttcatttacgacaagattaatttatggtccctaagagatacaggcaagacttttggcgccctgctcaaccttcggatgcacgagagtgaggaagggggataacagagtccacgtttttctacattttgtcaatcatgtgaaagagatgaagttccctagtggcatccgaaagaagtcggataattactgagtcggacaggattcaaagttcaatattcaatatctggttcctacctgtaaaaccattccagtcatccatagtagaacttaatatccctgcgtcaagcaatctgctattatcttttgtacatagagtgtagatttctggagtcattagagttgtgtaggattgagctattcgctttttgttgacgagattaaggatgtcggtttgcaactagctttgtaaaacagaggtgtcttcagtgatcaaaaatataattgctccatgtgttagctagcttgtgttattatatggctgactgaaatgtaggcgcgacatgattttcagtacatctgta

SEQ ID NO: 28 - DNA, 3258 bp, Beta vulgaris
  source 1..3258: mol_type = other DNA
atgaacggcggaaactcaaagaagcgcacagcaccgcagcaacaaccttcacctgcgcagcagaaacaccatgctgcaatggaggaagaggaattcgacgaagatgtatactactcacaagacggcctcatcgacgaagacactcaaattcttcgcgacatcgaagaacgttccgccaagaagcttcgtctctctaagtgggctcgtcctcaactttccgaatcctacaaatcacaatctcagagtatcgtttttcagcaattggagattgattatgtgattggagattgtaataaggagattttgttaaatcggagtggttctgctgcgattattcggattttcggggttactcgtgaagggcacagtgtttgctgccatgtacatggatttgaaccctacttttacataaattgtccgttagggatgggtccagatgatatatcccgttttcatcaagtcttggaggggcgaatgagggaggcaaataggaatgccaaggttccccaatttgttcggcgcattgagttagtgcagaaaaggagcataatgtattatcaacagcaaagttctcaaccttttctgaaaatagtggttgcattgccttctatggttgctacttgtcgtgggattcttgatcgtggcattcagattgatggccttgggaccaagagttttatgacatacgaaagcaatgtcctctatgctctacgtttcatgattgattgcaacattgttggtggaaactggattgaggttcctgttggaaagtataagagaacaggaaagagcttgtcttcttgccagctggaatttgattgcttatatgcagacttaattagccatgatccagaaggagacttttcgaagatggccccattccgcattttaagttttgacattgagtgcgctggtcgcaaaggtctttttcctgagcccactcatgaccctgtcatccaggtggcgaatttggtaacattacaaggtgaagataagccatttgtccggaatgtcatgactcttaagtcatgtgctcccattgttggtgttgatgtcatgtcctttgacagtgagaaagaactgttgcttgcttggagggatttaatacgagaggtagatcccgacataattattggatacaacatctgcaattttgacttaccatatcttattcagagagcaactgccctaggaattgctgagtttcctgtccttggtcgcataaggagcagtagggttcgtgtcaaagatgcaacattctcttcaaggcagtatggaataagagaaagcaaggaagttacaattgaggggagagtacaatttgatttgcttcaggttatgcaaagagattacaaactaagttcctattctctaaattctgtatcggcacatttccttggtgagcagaaagaggatgttcatcattccataatatctgatctacaaaatgggaatgcagaaactaggagacgtcttgctgtttactgtttaaaggatgcttaccttcctcagcggctgatggagaagctgatgttcatttacaattatgttgaaatggctagagtgacaggagtccctatatcatttcttctctccagagggcaaagtattaaggttctttctcaacttctgaggaaggcaaagcagaagaatcttgttataccaaactctaaacaggcaggatctgagcaaggaacgtatgaaggagcaactgtgttagaggcccgagcaggtttttatgagaagccaattgcaacgctggattttgcttcactatatccatctattatgatggcgtataatttatgctattgtacactggtaacaccggaagacattcgtaaactcaatatacctccagaatgtgttaacaagactccatctggtgaatcctttgttaagccggagcttcagaagggaatacttccagaaattcttgaagagctcatagctgctcgtaaaagggctaaagcagatttaaaggaagctaaagatcctctggagaaggctgtcctagatggtcgtcagctggccttgaagataagtgcaaattctgtttatggatttacaggagcctcaattggccaattaccatgtttagagatttcttcgagtgtcacaagctatggtcgtcagatgattgaacatacaaagaagcttgttgaagataaattcactgttgttggaggctatgagcataatgctgagattatctatggagacacagattcagttatggttcaatttggtgtttccactgttcaagaagctatgaaattaggaagggaagctgctgaatatataagtgccactttcacaaagcccatcaagctggagtttgagaaggtttattatccatatctgttgattagcaagaagagatatgctggccttttctggacaaaccctgaccaatacgacaaaatggataccaaaggaattgaaacagttaggagagacaattgcctactcgtcaaaaacttggttactgaatgccttcataagatacttattgaaagagatgtcccaggagctgtccagtatgtcaagagtaccatctcagatcttttgatgaatcgtctagacctgtctttgttggtcattacaaagggtctaacaaaaacaggggaaaactatgaagtaaaaacagctcatgttgagcttgctgaacgcatgcgtaagcgagatgctgctactgcacccagtgttggtgatagagtaccttatgtcattattaaagctgccaaaggtgccaaggcgtatgaaagatcggaggatccaatatatgtgctggaaaataatataccaatagaccctcaatattaccttgaaaatcaaattaccaaaccaatattgaggatctttgaaccaattttgaaaaatgcaagcagagagttgcttcatggaaatcacacaagatccatttccatctccactccttcaaatagtggcatcatgaaatttgccaagaaacacctcacatgcattggctgcaaagcattgattagtggctccaacaaaaccctttgctcgcattgcaagggaagagaagctgagctttattgtaagacagtggccaatgtttcagagcttgaaacacttttcgggaggctgtggacacaatgccaggagtgccagggttcccttcatcaggatgtgctttgtacaagtcgtgattgcccaatattttatcggagaaagaaagcacaaaaggacatgggtgaagccaaggttcagctagatagatggagcttctga

SEQ ID NO: 29 - protein, 1089 aa, Capsicum annuum
MNNGKSRKRSAQAAEETPSPQPPQKHHAAMEEEDLDEDVFLEETLLQYEEDSAVLRELEESHALNERLQKWKRPPLSQAYVSHTQSISFQQLEIDYIIGDSHKELLPTSSGQAAILRIFGVTKEGNSVCCHVHGFEPYFYISCPPGMGPDDISRFHQVLEGRMRESNRNSNVPKFIRRIEVVQKRSIMYYQQHVSQPFLKIVVALPAMVTSCRGILDRGIQIEGLGMKSFLTYESNILFALRFMIDCNIVGGNWIEVPVGKYKKAAKNLSYCQLEFDCLYSDLISHSPEGEFSKMAPFRILSFDIECAGRKGHFPEPTQDPIIQVANLVTLQGQGQPLIRNVMTLKSCSPIVGVDVMSFDTEKEVLLAWRNLIREVDPDIIIGYNICNFDLPYLIRRAEVLGMAEFPVLGRIRNSRVRVKDATFSSRQHGTRESKEITIEGRVKFDLFQAVQRDYKLSSYSLNSVSAHFLNEQKEDVHHSIISDLQNGNSETRRRLAVYCLKDAYLPQRLLDKLMFIYNYVEMARVTGVPISFLLSRGQSIKVLSQLLRKAKQKNLVIPNAKQAGSEQGTFEGATVLEARTGFYEKPIATLDFASLYPSIMMAYNLCYCTLVTPEEVRKLNLPPECVNKTPSGETFVKSNLQKGILPEILEELLAARKRAKADLKEATDPRVKAVLDGRQLALKISANSVYGFTGATVGQLPCLEISSSVTSYGRQMIEHTKKLVEDKFTVLGGYEHNAEVIYGDTDSVMVQFSVPTVEEAMKLGREAADYISGTFIKPIRLEFEKIYYPYLLISKKRYAGLLWTNPDKHDKMDAKGIETVRRDNCLLVKNLVTECLYKILVDRDIPGAVQYVKNTIADLLLNRMDLSLLVITKGLTKTGDDYEVKAAHVELAERMRKRDAATAPGIGDRVPYVIIKAAKGAKAYEKSEDPIYVLENNIPIDAQYYLDNQISKPLLRIFEPILKNASKELLKGDHTRAISVPTPSNSGIMKFAKKQLTCIGCKIPLSGSDRTICNHCKGREAELYCRSVANVAELENLFGRLWTQCQECQGSLHQDVLCTSRDCPIFYRRKKAQKDMAEAKTQLDRWNF

SEQ ID NO: 30 - DNA, 31203 bp, Capsicum annuum
  source 1..31203: mol_type = genomic DNA
cctaaaccctcacttccccaaactccggcgaaccaccaccatgaacaacggcaaatcacggaagcgatcggcgcaggctgcggaggagacaccgtcacctcagccgccgcaaaaacaccacgccgcaatggaagaagaagacctagacgaagatgttttcctcgaagagacattgcttcagtacgaagaagattcagctgtccttcgcgaactcgaagagagtcatgcgcttaacgagcgtcttcagaagtggaaacgtccgccgctttctcaagcttacgtctctcatacacaaagcatatgtaagaaaattgtaatttttgaatttatgtcctttttttttggttaatttaatgcaattttgttcctggagctcgcttttcgtgttttcacagtttaatcggcttatttggtgaatttggattaggctgtgtcaactgtggtgctgaatgtgtagtgttcttttagttgtttcgtttgataaccgtggtgtctgggccattttgagctcctcgactaattccacgggatacttgccacctcccactcccacaaagaagtgaaaacgttctctctctcaagcttacatctctcatacacaaaattcaatttttttatgcttaatttttgaattcatgtttttttgggttaatttatagacaattttgttcggggtgctcattttttgtgtttatccaatttttttttgatttatttggtgaatttggattacgatgtcttaatcatagcgctggatgtttagggtttctgtagttttttctttggtcctttctttttttgatagccatagtgttcggaccagcttgtgctcaccttactaattcaactggatacctagcacccagtggcggagccaaaatcttcattgagggggttcaaaatatggaaaagtaaatgcatgaaaatggtccaacatctattttatatacatacaaaaataattttaaccttatataaatagtgcaattctccgttgaagggggcccgactactacctggcttcacctagaggtggcaaaatcaaactcaacccacccaacccattcaagtttgagttggttattaactcatccatttattagctcaacccattaataaattgggttgatatataacccgaattgatttttgagaaatcatgtaaaaatattttaaagaggttttcttttttagtttgatatgttatatatagccataataaagaaaagaataattttattcggtactaaaaatgtataaaagaacatataaagcaattaaaacttagtaaaaattgggagggttgggttctaatctgttttgacccaaacaactttgggtcgggttgggtcttgacccacctaaatttgactcaacctgccaattgccacccctagcttcgcctttgcttcacgtccactagcatcagttaccggtccaccaaggctagagcagatggaaagaaatcacccagtgttttttgcttcgtctaggaattgaacctgagatctcatggagctcaaatccacttcatagtttagggtttctgaagttgtttctttggtcttaagcttttcttacaaaggatgtcaagtcagcgggtgcggagtagccctggatcgactgctcttttcaacaatttgtgtggatttttgtttgatttaagtgatattagtgtgttcatttaatgtattataaaagttttgaagtccttttgtgctcttggagcaacaattattttagttcctgagctcatgtagtcatctggaatcttattaacgttgttacacttgatgaagggaggacaatgtatgacttaagcagagtcacatgcatgtcttgtacttatctttcgacacctacctggtgtttattaatgaagctttttgatttatccgaaaaaaaagtttgtatattgagttgagggaaagatgtacatgtggatatatataataattcactgaaataagaaggtatattgttaggtaacataaaggtgccaaaatagttcaattatttcaataagttctaactttaacatttgcattctgaaagttctgttgaaatttggtggtcctttctcagcgtttcagcagctggaaattgactacattattggagacagccataaagaattgctgcctacttcttctggtcaggctgccattttaagaatctttggggtcaccaaggaaggttcgtcaatgtttccttgtgtatttgcaattctcattgttgtgttgtagatatggtgctattgatctggaaaataagatatgattctggcatttcaagtaatataatagtctcctataggtataaatgcacgttgtgaaatgtgaatagtttcttttatcgtcgaagtagtcttttaaaaattaaaatcaattagagcgttctgggtgctgcaggcttgtgaggcatgctgataccacaatctggaacttagattttctcttgaccatataaaatattggtacaaatatgagttaaagttgcatcttagtggatttctctagttgtagtatttttttttttctgtttctatttattttgtgccgtttcctttacagaaggaaattagtactttctatgctatcatattacactagatgctagaaatgaacatgaaaagcttaagtggaggtggtacttggtaggttgaggagttgagttccgaaggaactcagcaacttgggatttatcattaatgtatttattgaggttgacgtagcttgtcttccacttactaagaagtgcctgtttatatcctggttaggaaatagtgtctgttgtcatgtacatggatttgaaccatacttctatatcagctgccctcctggaatgggccctgatgatatctcccgttttcaccaagttcttgaggtatgttgacctgcttgctgccttgtttgcattagcatgcgatgattgcattcttcaccagtttggtttattaatggccaatgctgcatttgctatactattcttgttggttgctgtagggtaggatgagagagtcgaataggaacagcaatgtccctaagtttatccgccgcatcgaagtggttcagaaaaggagcatcatgtattaccaacagcatgtgtcacagccttttctcaagattgtagtggcattgccagcaatggttaccagttgccgtggtatattttctgtttcacttatttacttatactgtatctttaataaagttgcttttctgatttagtcttttcttttgtgcttggtcacatgccgcatacaggcacacgtagtaaaagttttattgatagaaatatcaagagctaagacagtgctggaaagtacaataggattttctctccagattcaaaatctgatcaattttgtgtcaaactactgaactctatcattgcgtgcttctcatatgtatattttaagattacaacagaaattagctgtctaagtcattcatgtttcaaactgcaaacagtttagatcttgaagtattgctggatcgtttcctgctaaaccacccaaatcagctgaaaggttctccaatgccctctgttcatttctcattttacatcctgcccagctctgaaaagatcttggaccgtccttaggattacccatttcatcccaaaagtgtttagaaaggtctctcaattgagcagttgcgtgggcttgtggtacatttaatatggcattttgtttgccagtcttgccatgataacttttagtgtattacttcacgttgagtcataatttaaaattatatacttgcatcttatgcttcctgtgaaattgctccttaatcttctcacttaagttcactaactgaaatatggagaaggatatgttcgatagaatttgggttatattcttcaacatagttggatgtaccagaattgcaataaaatgtttaacttctgattgattttatagaattgattctattaccaatccctttttcctgctttttccacttgattaaaatgtaaaatgtggcaggtattctcgatagaggtattcaaattgaagggcttggtatgaagagctttctgacttatgaaagtaatattctttttgctcttcgcttcatgattgactgcaatattgttggtggaaactggattgaagttccggttgggaaatacaagaaagctgcaaagaatctctcctattgtcaattagagtttgattgtctgtatcctaatgcatgattgaatctatgacctaagggtgcattctttctatccatatgggatcatcactctgaatttccttaacattttcttttagatattctgacttgatcagccattctcctgaaggagaattttcgaagatggctccctttcgcatattgagttttgatattgaatgtgctggtcgtaaagggcactttcccgagcctacacaagaccctattattcaagttagtcacaatttattctacatatctggaaaatttatatcgtatactgtcctgttgctgacaagattttaacttcattttaggtggccaatctggttaccttacagggacagggtcagccattaattcggaatgtgatgacccttaagtcatgctcacctatagttggggttgatgttatgtcctttgacacagaaaaagaagtattgctggcttggagggtaatatttctgcctgctatttgtattttaattggatacagtttccaccctttctcttcctttttgtgtggtggtgggaatggagggttgtgggataggcaagcagactgtttgtatgcagcaagtgattctttcaatttgatttcgtctgatggcagttacccttataaacggtttattttagtgtgagtttcacattgtccgtgtcgttttctgctccagattgtaatgcacatgcagaagaatgaagaaggcttaacagacgtaattaggacaagtgagaagttaaaatcatggaaaaagaagaagaagaagaagaacaacaaaccctgtgtattcccacatagtgaggtctggggagggtaagatgtacgcagtccatacctctacctctaaagaagtagaaaggctgtttccgatagacccccggctcgagacacggaatactaaacaaattcataggaaagcatggaacaagatggcataacataaatacgacacccattagtaatagtaaacagagaaaaataaacagattcgtaataaagcatgaaacaaagtattataacaagaataatacccccaccaagtaattccctgcactagcgacccaaactgaccctagccttctaccctaattcgcgtcttccaggtcttcctatcttgggtcatgtccttagtgagctgtaactgctccatgtcctgcctaatcacctctccccagtattttttcggcctacccctacgcctcctaaaaccatccacaggtagcctctcacacctacgaatcggggcatccatgcccctcctcatcacatgtccgaaccatctcaatcggacttcccgcatcttattctccactgcagtcacaccaaccttctccctgatagtctcattccgaactctatccccctagttatcccatacatccaacgcaacatctgcatttcctccaccttcattttttgaatgtgggagttcttaactggccaacactccgctccataaaacatggccgaacgaactgccaccctgtagaatttgcctttaagcttgggcgacacctttttatcacacaacacccccgtaacgagcttccacttcatccatcctgccccaatacggtgcgagacatcctcgtcaatctcactgttaccctgaatcacggacccgagatacttgaaactatccctcttacataccacctgtgattccaacttcactaccacctcattctccagcctcacatcattaaacttgcattccaaatactctgtcttgctgctactcaacctgaaccctttagactcaagagtttgtctccacacctctaatttgtcattcacaccccccgcgtctcatcaatcaaaactacatcatctgcaaaaagcatacaccaaggcacctctccttgaatacgccgcgtcaatacatccatcaccaaagcaaacaaaaagggactaagagtagattcctgatgcaatcctgtcaagacagtgaaatgctctgagtctcctcccgccatcctcacctgagttttagctccatcatacatatccttaattgctctgatatatgccaccgggactccactcacctccaagcatctccaaagcacctccctggggactttgtcgtatgctttctccaagtcgataaacaccatgtgtaggtctttcttcctttccctatactgttccaccagccttcgtaccaggtgaattgcctccgtcgtcgagcggccaggcataaatccaaactagttctccgaaatagacactatccgtctcagcctcacctcgatcactctctcccaaatcttcatagagtgactcaataacttaatacccctatagttattgtaactctgaatgtcacccttgttcttataaaaagggatcataatactccacgtccaaacctcgggcattttcgccgtcttgaaaattccgttaaacaatccagtcaaccaccttaaaccaacctcgccagaaaacttccaaaactccacaggtatctcatcaggccccgtcgccctaccccttcacatcctgccaacagtctctctaacctcctctaccttaaaacgtctacaataactaaaatctcgacactcctctgagtgctccagctcccctaacccaatagctctatccccctctccccctcgtcattcaagagcctatgaaaatacgaccgccatctattcttaacgtggccgtcctccaccaacactgtaccgtcctcccccttaatgcacttcacctgatcgaggtcacgatccttcctctccctagccttagcgagtctaaacaactttttttccctcctttctcctgtaaccccgtatacaagatctcaaatgctgccgtcttagccgccgtaaccgctaacttagcctccttcctagctaacttgtactctttcctttttacccgcttctcttcttcgtccttattctcaaccaacttagcatacacccctttcttggtctccattttcttcttaacctcttcattccaccaccaatccccccgatgatgcccggcccggcccctagaaacacccaacacctcactagcattctccctgatgcacttagccaccctatcccacatactatccacgccccccctatactcccacacccctattcccaccaacttctcccctatctcccacgcattcactggcgtcaagacgcctcacttaattctaagtctacactcctcaccccttctcttcctatccttctttgtacccaaatccataaccaagagcctatactgggtcgaaagattctcactcgggatgaccttacagtccttacacaacgccctatcccctttcctaagcaacaaaaagtcaatctgggtcttggctatcgcgcttcggaaggtgattaggtgatcgtcctttttcgggaagcccgagttcaccaccaccagcccaaaggacctcgcaaaatccaatagagtagccccctcttcatttctttttccaaaaccaaaatcaccatgtacatcaccaaagcttcccggtaacgccccgatgtggccattgaaatctcctgctacaacaatcttctccgagctaggcacgcctctcaccatctcatccaaagcctcccaaaaccgcatcttctcctccccatccaagcccacttgcggcgcataagcactacacacgttcagggtaaactccccaatgaccaacttaatagtcatcaacctattgctgatcctctttacctctactacctgccctctaagctcttcgtctactaagatgccaactccagctcccagagtaccaaagcttgtaaccatccacatccctagccttagaccctacccacttggtctcttgaacacacgcaatattaatcctcctctttctaagaatcttcacaagctctgtggacttaccctgaagtccctatattccaagacccaaccctcagcctaccaactctatcaacacgtcttcctctaccgcccctgaggccaaaccttagcctccctcccacccccgcccctacaacagcccccgccccaaccatcaacgcccatagccactactaccagaaaatataagaaaatataaacaatggtagtaacaaagcagcagcaggaaatacaaggctcacacaaattagaagaaatactcaagaaacaaaactaaactcaaataggtagaaaaaaaacaaaaccaaggtaaggacaaaactaacagtaagaaaaatagagcacactaatggcctggcaccaaggacagccaaacaattaataaggagcggtgcaagtcacgaatatactatagcagacacgcaagtcaagatacaggagataatatgataaataatatgaagcaacataagaggttcaaaaacagcaagacaataaagacaccctcaaagcacacactatatccaaattttaagaacaacaactactagaaatccaaataacagatagaaaatcccatgcaaattacaggcaacgaaaatcccacgcaaatcccatggaaaaaagaagaagttagaaataaaaatgcaaattaaatttttatgggctatttacacttgccttacttgccatatgaactttgttgattggtgctgttaatatgaataaagggtgtttggatatgccatttcattctctcatacattagtttgtacaattgtgtaaaactatcccagagtaccaacttacttttttcttttattttcttttgacttgcagaaccttatccgtgaagtggaccctgatataataattggatataatatctgcaactttgatttgccgtatctcatcagggtaatgagcttcttcattcttattttattggtaaaaggtattatattccgggtagcgagcttcttatgtagttccctgtttaaacaatggctccaaaacaatgaaatgagagctaaagatttgctaggcaagcctaatggtaggttagttaatgtagtaaccaaaaatgttatttcttcattattaattggagtaagagatgaggagttcaaaaaatccattggcagaagtaaaggaaagcactacctgataacttgcggtttagctgtgaagctgatggtggtactgtagtggaaaaaatggtttcttcccatcccaatagagattgggtaaggctaggtcaagtttgttgttccatgtgtattgttgagctacttaaggcattttatcttcatatcagagagctgaagtcttagggatggcagaatttcctgtgcttggacgcatcagaaacagcagggttcgtgtaaaagatgcaaccttttcttcaaggtcatattatatctttttatttgttatcttctgttcgtgtagaagtacttttaggcttctacttcatctgtaggtgaaaataccatcagtactgaacgacattctttttcatttggtgtctgtctgttgactcttgtatgaaccattggtctgttcagtttgcatttatattccatcactatctgtgatttctgaaatatggcattttaatactaacctaaaacatgttccaattagctgccgcttatcagaacctttctgatatttctccttgtaatagacataaagtggagttattgagatctccatatgttgcctcctagagcattgtttatctccaaaggtggaatcatagcgcttcttaattcaaactttcccaaaatttcacactactgaaactcttatatcagtctccaactagttgcttgtgatcataaagcaaatttccccaaggctatattgatcccgatcattcttcatttaattgtttgtgattagggtgtcaaatgggtgggtttgttgaatttggacaggtcaaaatggggtgagttaataaaataacccgcccgatagttattttggctgaattgggctaaaagatgggtcacaacccaacccacccaattcttacttagttttaatttctttatttgttctcttataattttttattacctaataaaactatttccctcctttattagagctatatatacacatcaaataaaaatgtccttatgaaaatattttgacaaaaattctcatgggtcaatttgggccaaatatcaaccagtttttttgatggactgagttaagggcgtgacaaatacatgcaactgagttttgcgtttagtgaacctgaatttatcccaaaaaacttgagaagctgtggactgatatatgtgttttgtgttgcagacagcatggaacaagggaaagtaaagaaataacaatcgaagggagagtcaaatttgatttatttcaggttttgttcattatggctgtagctaacacctgttttaattgctgtacaaagaaatttgccaggaaaacaacaaatgtcactaaatcagttattctcttgtttcgcaggctgtgcagagggattataagctaagttcttactcactaaattctgtctcagcacattttcttaacgagcaggttagatttttctcttctatctatagtttttcgtctcatactcctacatctgtctacaacattgtaccttctgcgagtacatttatacatgattttgagctgtgaccctttatgggctcttgtttgtattcaaaacttcaatgctctttattttttactttatgcatccggtatcttaaactcactggtccaattgattggactctgtgtgtaggatccactataggtgaagcactccctaccaggagcttcttcatttctagtgctcgaacttgtgacttttgattaaagttggaaggattccatccataccacatctctacttatgttgatagccaagtttgagttattccattagggccattgcgcatgtatatttaagtataacataagtaggccttgtggttatgctatgtaatttccttgtgtacagggttggttagcttgaaggattacatgtatacttgttagctttctgtttttattctgtcatgtcgattgtgaaattaatggcggcagctgtggttgatctgcatgagttagaaaagaaaaaaatctgtgattctgaccttgaaatcattaatgaacagaggaaacactcaattatgtgcaagaaacatgtgtaaagagttatgatttttttcctgcaagtgcatgtactaaaatctgatccttttccattcaccacagcacatatttatggggagaaatgggtaactacaatatggtctggctaccagctatcacataacaacctcaagtaccattgaataaagcatcttaaatttaattgagtctaagtatttcatattttctttggtgtctattttctgctgccaagatgcaatggaagcattgccagatggagcgaaagaagattggacatgagcaaggaggagttggggaacttgcccctctttgtgtcttgtgttgatatggagagaaacaaatggaagagctcttttaggaattaaaaaagggagcaattaatcaaagatccaatgaggagatcccctattcaatagaattgttgtttagagtgaccataaaaagtaacatggaagtgccaaatcatctaagaagtctacactttcatttatcgcccatcatttgccctctctttctagctccagtgcattcttttaataagttccatattatttttattctgtaatttattgtgaagtttgatttgagatctttatctggtggggcagaaaggaagacatcttaatctatgttgctcgggctcttcgaaaatgtcaacgtgtgcgtgttggattcggcaaaactagtgtatttggagaatccgacaagggtgcgacatcaaaagtgaagagtctgcgcaacttagatcttaatcttattgtttgacctttattactaaaatatcattggattcggtgcaatctgagaattgtgatgtaggggtcttttatacctttgttttccttcatgttcttttatgcccatagtctgttcatttgtcttgtttctctatcttcctattgaatatgcttgtgttgtttagagtgagtactttttttctctaatactactgctactcctgcagaaagaagatgtccatcattcaattatatcagatcttcaaaatgggaactcagaaactaggaggcggcttgctgtatattgtctaaaggtgtattattccgttccgcttgaaggatattactgaattggattttattgtgtgctgagtattacttatttcatagaacatttagttcttttaagatttttttatggctttggatttgcatacaattttctttagatgggtgatatacacataaattctgctccaaaaatatttaatttaaaatttcttaatgcttatagcaacacccctttggtgtagaatgctccacttgaaagatatttgtatgtaaaaaaacaaatataatatcatcgttatcatcaagaagatactgattacaacattcaaagatgtgacctagtggtcaataaagataaaactatggtttgatttggattatgccaaacattgatacacctctgtcatctttttcaaaatagttggacgtattgattgtttcatgtagacaatgatcaatgggtagttttggcgaatggctaataaaatgtagtgattaaaattgtagatgagtttggatggaaatgttatttgctgatgtgcatattcattctcatttaacaagaaaactgtatgccttacttccattacaggatgcttatctcccacaacggcttttggataaattaatgttcatttataactatgtggagatggctcgagtcactggtgtacctatatcttttcttctatcaagagggcaaagcattaaggttggtcctgaacttgcttcattttccactggtgaaaattttgttcacttaatgaagttcttaatcttaaattctttgattctcccctatcttcaggtgctttctcaacttttaaggaaggcgaagcaaaagaatcttgtcataccaaatgcaaaacaggctggatcggaacaaggaacatttgagggcgcaacggtttgtgaataagtgtttgtgtatcatatagctggatattttaccttgtaaagataagttagcgcatgcaaagttcctgttttgctaagccttatttatggtatattacttttgttaaacgtatgcttggaggattaatcttgacatcacacaccttttatttcctttaatgttgatttcttaggttaaaggtactacctgtatgtcttgactcttgtgtatttttttcctgtcaaaaatatctgtacacaggttttggaggcccgaactggtttttatgagaagccaattgcaacattggattttgcatctctatatccatcaattatgatggcatataatttatgttactgcacgctggtaagtgttcttgttcgagtattgaccatgctgaatgttctattgatgttggtgaagccttataattaagtgctgagcatcaagatcttcacgttaatgcttccagtggttttcttccgtcctgtctaggtaactcctgaagaggttcgcaaactgaacctccctccagagtgcgtcaacaagaccccttctggtgaaacatttgtcaagtcaaatttacagaaggtaaacataactggtatattttcttttttacattttggttcacccctccctaggagctcccaccttttcacttgcaaactttgggcctaactcacaccccaaaagctaactcaaagggagaggattgcccaagcctttttaaagagaccatcatctcattaaccaccaatgtgagactttttcttattctttaacaccccacctcactcccaggactagacatctggtgtgtggtcccttttaagtttggggtcccaacattaaatgggatgggtcctgctctaataccatgtaaaattaggtcttgggcctaactcacaccccaagagcggggttgagttaggttcaagatccattctttacatggtatcagagccagacccattccaattctttgttcaccgatgttgggtcccatattatattgtccttgctctggttaataaggtctaggcatgcgggaaagtgttaagagtcccacatcggaaaagggatggatagttggtctccttatatggacttggacaatcatccttttttgagctagcttttggggttgagttcgactcaagatccattctttggtgactcgaaccacaacttcaaggttgaaagtggagggtgcttaccatccgagcaacctcctcttatccaagtggcttgattctggcacttatgttcttgtttcgattatgataatttaattctttttttgtcagggaatacttccggaaattcttgaagagctattagctgctcgtaaaagagcaaaagcagatttaaaggtgtgcactaaatttttatgatctatgttgtttctgcgttccctattagcatatttgctttatattggtgttcaatcttggtgtttgaaagaagttccattctggatttattgggggtgctttttgaaccaactagctttggggataatcaaggacatcagtcctccacatctaaagataatattcagtcagaatatgaatatatatctcagatgattcacttttatgggaaagggacaagaaataagagagttaaataagcatagagaatccaatatctgttttgttttggttaaatcaggatacttgattgttgattgtgaaataggacttttaacttttttcttacatttccttttgtttgtatatagaaagaaggtacactcaataatgccctcctttcactagatttaatggctctaaaattttccttttctagcaaggggtaaaaggaattcttactcttttcatttttcagtgtttcactaatgcaaccaagcacttggtgtatatttttacgtattcacatccttgcactgagcatcttattcccttaacaccagtattagatgctgttgaacaatgcagaattcatgtctagcttttgttcgtatttgcttgtagttaaatgaacacaacaacaacaaacccagtgtattcccacctagtggggtctgggggggtaagatgtacgcagtccatacctctacttctaaagaagtagaaaggctgtttccgatagacccccggctcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggaggggtggggggtatgaaccggaggttggtgacggagggcagcaaggagcggcgacgggggtcggcgacggggaccggaggtcagggccggaggtcgggtcgggtcggcgccgaaggtcggggaccgggacgagagagagcaaatgtgaaattttttctacagatgtttgattgtcttgaaaacttttgaaatggctctgggccctcttgaaacctgatatgttagggcaagattgtaagatccttaactacctgcaaagaggttttgatggacacaacgtaatttcatgctatgtagattctttttatccttatgccttgcattttaatagatctacgtatgtactaaactattaaaccaacatgttcccctcatttatttcatatttgtcaatatttttttgtaaaaggttcatatttgtccatattcgatgcaatatgatgtgtagcttgattttagcatacagaagttccattttaccaatctctctaaacttttgtcctttaaattgttttttgatccaagtgaattctcaatttgccacctccatggctccatatccggcttctgtcaactgcattttgtttcttttatgatgcatcaaattacgataaagacgacagtatcttgaaataaaccgcatatcaaaagcctgctatttttactcgagtattagtctactcaccttctttgcatatctaacagatggtgaatagataaatatttgagttatcctctgaaatttgaattatgacagtatatttgtttcaggaagcaacagatcctcgagtcaaagctgtcttagatggccgtcagctggctttgaaggtaattcttgtattgttagtgaatatccaatttttaactgtgttatttcactttctcttggcagatatatgaatttggttctctgtgttataggtattacaatgggattaggactgtgaagaattttgtcagatcaattaactactgatgtcttctggttttcagatcagtgcaaattctgtgtatgggtttactggagctactgttggtcaattaccatgtttagaaatatcttcaagtgttactagctatggtaatgcaattattactagcaatttcgttacatgacactgaacaattgaaaatttgctgtatagaaggtaattctaccacttgttttctgaatgtgcttgtttttcctaggtcgacagatgattgagcacacaaaaaagcttgtggaagacaagtttacagtacttgggggctatgaacacaatgctgaggtacataatctgaagttaaatcttatattctaaatcatatatgttagagaattccaatcaagatcttatggatgtagtcagtgtcactgttggttgaggtatcaactgttgtctacttatgtggtctcgggaaattctttcatcatgagcctagcttgggagctgagttagttagcaacatcacacatggagtatgctactacagttttaaacgttgtcgagttcaaagataaagctcaagtgcaaaattactccaatgagtatggtggataacagcagacagagttgatagagattctcatttctaaggtcaggattttaacaacttaactgggaccaattctgggagtttctggactgcattgggtgatgccaaaagatatgaagagtttatcccaatgcccccatggatagattactggttgcaggcggaagcagatatggcagacagttccagtgtgtggaatatcaactattcggctagagaggaataaaatgtgttttgatgagagaaaagagcatttgtctttcgcaagatatagattcatgaataatcggtatgtttctgagtgggatatgctggaagattttgaacaatttggaaaacaatatgttagaagattaagcacagcctctaaactttatggattcctggatgaaaagactctaattgtaggactttcttctgttcttttgtagtgccttcttcgtgctagctttgataatattaccttactttattatcaaggagggaaaactgttttaactgtgaccaccttcgggtgttcctcatcaggactatgcactacaataacccgtataagggtcaaaaagagctaaaaagtgtgataagagatactcttactagggggaagacacagaatcaagctgagttgtattgtcttttcaattctggtgtaaagagaactacgttgttgatgcagaatctctcgtagatttttttgactccttgtgagagttgtggttagactggctttgctgtatggggcggagtgttggctatcaagaaggcccacatccagaagatgaaggtgatggagatgagaaggcttcggtggatgtgtgggtatactaggaaagataggattagaaatgaggtaatttgagataaggtgggaatggcttcggtggaagccaagatgagggaagcgaggttgagatgatttggtcatgtgatcagtaggggaacagatgccccagtgtagaggtgtgagtggttgactagggatggtttcaatcgaggtagaggtagaccgaagaaatattggagggaggtgattagacatgacatggagcagctccaacttaccgagggcatgaccttagataggaagttgtggaggaggcggatttgggtagaaggttagtattagtttaggatgttgtatcatttgttgtatcacttggtgtatcttgtatatggtattattggatgtgttgatttctgttgtatcttgtccttttactattccttgtctagaatgttttattttgagccggagatctatcagaaatagcctctctatctcacctttgaggtagtggtatggactgcgtacacttaccctccccagaccccacaaggtgggaatatactgggtatgttgttgttgttgtaagatacagggatccctttaatttgtaattgctctcaacttccagtaccaccttggtgttgccgaatacaaagttcccttactcaaaaatcaaaaatgcttgtttgagtgttaatgagagaaaggttgtggaaatcacaaagaaactttatatgaaagcaagtttggggatggtggggggtggggtggtggtgaaaaagtttccatctgattaaggtatgagagagacttgagggtgtctaatagagctttactcgcgaagcaatttggggaactgaaattatggttggggaaatctattagagcattgtgggacaaactggaaaaacacacttgtcttaaagtgggcaatggcacaaagataatgttctggaggggtgcttggctaaacacgaactcattgaaggatgaattccctgatctgttttccttatgttcaaaccaagatgctaccgtgggagattgctgcactcttcaaggatggaatcttctctgtaggagacagctaaatgactgggaggtggatagagtggcgaaatttttgatggatattgagggtttcacaggtataaccaatggcgctgatactattagatggagatataacaatgatggtgttttttctgtttgtaggatgtacaaaagagacaacaggtctgcttgtatagggaacccgattccatggaagaacatctgggagagtataataccttccaaggtgaagtgctttacttggttggttgccagaatagcatgtctaacccaggaggtcctctagaaaaaaggcttccctgttgtgcccagatgttttctatgtatggatgctacagaaacaatagtcacctctttcttcattgtagattcacagctcaaatctggagaatgttcttcagcctcacagaagtcaagtggacattgcctgaagacactgctgattattgagttgctggataataagagggggtagtaagtgtcggaagaaatggtggaagccaatccctgcagttatctagtggtcagtgtggatggaaagaaatggaagatgctttgaagtatctaattccattcataaagtgaatattgtattgtatcttcacgttttggtgtaaaggacattgtacagaggatgtagatgatatagtagtctttttaggatctttgcaactatttctctatttttgtctttttggaggttacatgctgaggaatacaacactaccagatctcaaataaataaagctacaaaagaggaggaaggaagggtgggaggacctatcagattcttctttacggactggccctcttggaatggcataatgaaagagaagtatttttagaggaacgtcatatttaaagaaaacaactaaagtagtatcactttttggaaacagatggagatgcctccttatgtgatacttttccaattgatctctattctctagctaacagatagcggtatttgtaaggagcatggaggaatactatacacaacaacatacttagtgtggaggaggaatactatgagacctgacatttgggagaaacatgcaaaatctgggagatatctctttttggaagcaaaaatggtgcaaatagggaattttacaagcccctttatgtggcactttttcagccaattatctctaggctctagctatcagatagccatatttgtaaggtcatggaggagggatacgagacctatgattttggagaaacagtggaacctggcagatattagaatttaatagctagcttggattactatataagcaattaatgttgcttatggtctgagataattagaaatggagatatgagaagggtggcatgcgctcactgagtcattttacaacaaattgctaactaggtagaaaaaaagatttcttcatgcattaatatgtttttggaagcacctattaaatgtttttttcacctggccagcgcccagcagcatggaggtcatggagaatcttatgaggacaatttgcatcacatggtgcttcatgtacaggtgggaaaaaaatgtcaaaatttcatactgacctgattgcacatagattactgagtatgatgtggcacgtgtttggagttgaatggtcaatatcaagaactataacatatctgatggaaatttagaaggaagaggcaaatgagagtgaggaaattagctcctttgatgcttgtgtggacaatatggcgcgaagatagaagagttcaaatgagtagcaaataccaacatactaactcgttggaatatttcgttttttggcatagtttcagaacacagtatcctagtcattgttgactgcgagatgttcttgtgtggcagaattgcccctccacttcactagattattaacgtttataaaaaaattgttcctctaagtgtgattgtttcatgatgtcaggttatatatggagatacagattccgtgatggtgcaatttagtgtgcctactgtggaagaggctatgaaattgggcagagaagctgctgattacattagcgggacatttatcaaagtgcgtcctaaccttctcttttgttttcgtatttttggtatctttccttgtcctttattttcgtacttttggttttcttctatttgaaacccgctgataatcctctcctgtaaattgatggaacatgattcctttttctgctgagtacatgtcatccgtaaattaaatgatatttcatcacatgtatagtttgtgttgaagtgtgtgaccatctcatctaaaaacttaagctgttaaagagaacacactttaattatctaattatattcttaacacaccccctcacgtgctggcctgattctttttcatggcccaaacacatggaaattcttttttaataatgggtagcggtgagattcaatcccaggacctctgcctgctccgataccatgttgaagtgtgtgaccatctcatctaaaagcttaagcggttagagagagcacactttaattatttaattgtattctcaacagtttggaataggttaccagtggtgctttcctggatcattagattatttgtatctaaacctctcttctttttttttttaaaatcagtagcatctaaacactttgtcagatagataattcagtggagtttcttgttcctttttaatccttttttcttgaaacaagtcttaacctgcttacatgccacaactacagttagccatgatgtatttatattctctaatcagttggtcagttattgctctttgaggattcccattaggtggttttggtctttcatggttccatcatatttgtgattagggtacatatcatgaaggaataagtgaaaaagcatcagaaactataaaactctctgtccttgatcagttcagtttcattttgataagtagttttggtatacctattcaacttgatgcaattttgtaaatttatcataagtttatggtattctagtgaggaaacttgttaacttgtgagactccttgctaatagtagaatcctaaaacgtttaaatattgagatgtgttttcatcagtgtactgatattatttttgatcttctcttctactaaatttcttcttccgtatcccccttctgctggtggtacattgattttcagccaataagactagaatttgagaagatttattacccatatctgttgatcagcaagaagagatatgctggccttttatggacaaatccggacaaacatgacaaaatggatgctaaaggtgagctctgtcgatgcctagccttttcaccttcagcatattcttggaacttcactcgcccttcattgcgaacctttaattttttttcatattattatccttgaaaatttagcaaatcaaatttgttttcttctctgtttactgagcctactcagttgtttgtagctctagctctgatgtaaatttttgggatcgcagccttagtgctgaagatttatatacaaacttgttacctttgtcaaaaaaaaaatttgttgcgtcaaatttaagtattcttacattagtcaaattatctattgcttaggtgcattttgtgttgataaatcgttacaaatttaccatttttcaccagggattgaaactgttcggagggataattgtctcttagtcaagaaccttgtaactgagtgtctttataaaatactagtagacagagatattcctggtgctgttcaatatgtcaagaatactatcgcagatcttctgctgaaccgcatggatctgtctcttttagttattactaaggtgagaatactcttaaatcaggaaaaactgaggtctttgcttcttgcaacttgtttgaatattttagaattgttagtttatgagagtagcatttctaacttctgaaggtcaatagggtctgacgaaaactggagatgattatgaagtaaaggcagcacatgttgaacttgcagagcgaatgcgcaaggttggtaaaacttaatttatttgttgttacttttaaaaggatagtggatatcttcaagatggttcaattatttgtgtcctttgtctgtgaatatcaatttgtggatcttgcgcacaagtatttactgcttgaaatttgtttgacataagtttttaagtttcccatctaaattatggtcccattgtgagtgaatttgctccttatttctggtggcgaggctgctgtcattcaaaattttgatgggtttagtattcaattactctagataaggctggaccagagatcactatgcttaagtaatcttgtcatcaatcgctcaacttttctgtagttcagctctggttttcatgccaaggattccagtattcctcaatttgctgttaatcatacatactagtagccaactgatctaagatgtaaaagtttcacactgtgcacgagtaatattccagtcctatttatatgacttacactggtcacctagaaggatcagattctgatgttcatctcgaagagtttcagaagttatttcaattcttctctttagtctaccttatgatcttgtgcaattacttgctccccttgtcagcatcaacctccaatagaatcgggtaaattcagccttagcagttctatatcctttagacttctatattcaatataaagttagtttttcaactttttgtgcctttcttcaattcgtgcaaacctgacctgcttgactgtgtgtgactgaaaacctaacatacttctaccttttctgtgaactctgcctataaggttatcctaactttgctgtaacaggacttggggggagttggggatctggtagaattaattggcagtgtataaggttatatactttagtttgtaaagttcacagcttaatagctttttggttctgagctttactgtttatacaataatgttattctaatttaggagacaaatcaaataaaaatatcgtaacgttatcttgtagcgtctgatacagtggcacataaacgtgtcgaacaataatactattggttttcaaaacttcaggattggtccccagagcccattatccactgaatttcgggcagtgccgctagccatcagatttctcgattatcagaaaaactttggctattctccttgatcttctacactttttaaaatatgtgttgggagattaatagctccattatatgcgtttgttactctgtgagctatgttaaagcatatgcttgtccttcattggttttgggttgggtgttcagactcttttacatagtacctagagttaagacttgtaagcccagtttcacaattgtttctcgttaatataattgtgaagaaatgaacattgtgcttagctcaatcccaaaagttagctcatgagggagaattttgaagatcatataaggagaacatagccttatagctcacaaggggagaattctcaagaccatacgatgagacccttattactcatgaggggaggatttcctcactgttagaaggttaattgtactcagaaggaacgagaaaagtgcaccctcaagggtgtggtctggtggtcgatgaagtgggttgagaatcatgaggtatctggttcaaatccctgtggaagcaaaaaccctattcatacctctatccaagcctttttggacagagttacccagtacgtgtgcttgtgggggtagtaggtacctcgtggaattagtcaaagagtgtgcaaactggcctggaccacacacttatcgaaaagaaaaagagaagcaggtttattagaatatgtagtatcagaatataaagttacttcaaggctcgtatctatccgtttgttctgttggcttgctttccttttccctctcgttcattctttcttacaatggtttcatgtggttttgtaaaatataaattgttcaacccgaaaacaaaagaaggaaaagaaacaggttgactaaaagacattcattttaaggtacattttatatagtgtcaatgttttgcttactcccttataacagcgtattcaagtggcaggatgagctgcccttttaactcttgctgaacttgctgaaaccacatttctgagattcttatgtttttagtgcacaataaaacatgaaattgatggaacctttgttttgatcgacagcgagatgctgctactgcacctggtattggtgatagagtaccttacgtcataattaaagctgccaaaggtgccaaggtaatcgtccaactgatttagtatgtgattataatttccagattttgtgtgcttgtgtcaaatcatgatgatattatgacaagcaggcatatgaaaagtcagaggaccctatatacgtgttagagaacaacatacctatagatgctcaatactatcttgataatcaaattagtaaggtcagtatgtctcaacttttccttttaattaacatacttatctaactgcttttaacggaagcgttttatatcagccacttttgaggatttttgagccaatactgaagaatgcaagtaaagagcttcttaagggagatcacacaagagccatctcagttccgaccccttcgaacagtggtattatgaaatttgccaagaagcaactcacttgcattggatgcaaaataccactcaggtgctctgttgtctttcttgtccctttagggtttacaagataagcttctgttttatttcttaggagaatcagtccttcctttcaactaagttgcgtggactctttgctttcgatgcagcgcccgtgttggattctccaaaaatactctacttttggataatctgacacgcacccattgacattgtcgaggagtccgagaaacatagcctttcaacatgcttctcaacttgcgatttactgacataggatttgtacttccaacctttgctaggagcactgcctgatagtgtaacttctccaagttgtatattcagttgctaatgttaagttcctgttgaaatcgtaagttatcttagaagagccagtgtagcaagtgtatggggtatacttgaatttcatgcttttgttaccggcgtgaataaaattagtgggaatggtgaggcaaaagactaccttagagtttggcagcagttaatatgtttcaaagtggatattgtgccattttctatagaagcttgttgttgatggacttttaactatgttttcttcacctccaaatctatgaacctgctttatgaaacggtcatttgtcccaatttaccctcttttgtatttctcgggcagaaaaggttaacgtgaagtgttagattcattatcgatgtgttagattcattatagacgtagggtcatcggatttggttttctcttgtaaatatggctgcagtacagcctttgttctgttctgttacttaccaatatatataaagttagcagtttcaaaaaaaaggttaaggtgaacataggagatcatagcagtgtgaaagcttccacaagcttgttaacaatttgcctcttcctctctctgtgtgtgagtgtgtgtgtctctctctctctattgacgagagctgagtggatgtattgctttcttttttttcttggaagaggctataataaaggttgcagaacagcttattaacttccttgctctttcatccagtggctctgatcgaacgatctgcaatcactgcaaaggaagagaggccgaactttattgcagatccgtggctaatggtatctaggaattaatctcatgccctttcctatagctcttttttaatttcctgagaagggggaggatgtagcatttctattcagatgatgtctggggaaggtcagagagctatgaactccttgaacattgcttaatgactgtgtataactgtattaaatgcagtggctgaactagagaatctttttgggaggctatggacacaatgccaagaatgccaaggctctctccatcaggatgttctatgtactaggttcgagttgctttctgcttttactgatagatgtgtcgcttttgatatcatggacattcttaatatttcagatgaagctttcaatcccggccctaaagctctctatcgtttgatttatgataataatagaccctgttattgctggttattcctcttattgcttctctccccattcgttcttctaaatgatccttattttgaccagtcgagactgcccaatcttttatcggaggaagaaggctcagaaagacatggctgaagctaaaactcaattagatcggtggaacttctgaatttccttgccggaaagtcttcatttctgcaaggccgacctgtcattaagccaggtatcgtatttgtgacatctctggtatgaagaaagagtttggaaatgaactgaacaaccaagacactactactatgtctgatgaaaaggtaccaattgtcttcctcaaatccaagacccactaatatacgcacggaaatcgtgttcctgcatgagtttaaacttcaaatcattagcaagattgggttcttaacaaacttttgtagcttccagctattaatgtgactggttagtataaaagagattagatagatgactatggtctataaatattgacgtattgtgatctgtataggctctgttagttactagtaccagtggattgcaactaccacttaaaattctcttctttgatctcattgcatattctctccttgtgccaagaa

SEQ ID NO: 31 - DNA, 3749 bp, Capsicum annuum
  source 1..3749: mol_type = other DNA
cctaaaccctcacttccccaaactccggcgaaccaccaccatgaacaacggcaaatcacggaagcgatcggcgcaggctgcggaggagacaccgtcacctcagccgccgcaaaaacaccacgccgcaatggaagaagaagacctagacgaagatgttttcctcgaagagacattgcttcagtacgaagaagattcagctgtccttcgcgaactcgaagagagtcatgcgcttaacgagcgtcttcagaagtggaaacgtccgccgctttctcaagcttacgtctctcatacacaaagcatatcgtttcagcagctggaaattgactacattattggagacagccataaagaattgctgcctacttcttctggtcaggctgccattttaagaatctttggggtcaccaaggaaggaaatagtgtctgttgtcatgtacatggatttgaaccatacttctatatcagctgccctcctggaatgggccctgatgatatctcccgttttcaccaagttcttgagggtaggatgagagagtcgaataggaacagcaatgtccctaagtttatccgccgcatcgaagtggttcagaaaaggagcatcatgtattaccaacagcatgtgtcacagccttttctcaagattgtagtggcattgccagcaatggttaccagttgccgtggtattctcgatagaggtattcaaattgaagggcttggtatgaagagctttctgacttatgaaagtaatattctttttgctcttcgcttcatgattgactgcaatattgttggtggaaactggattgaagttccggttgggaaatacaagaaagctgcaaagaatctctcctattgtcaattagagtttgattgtctatattctgacttgatcagccattctcctgaaggagaattttcgaagatggctccctttcgcatattgagttttgatattgaatgtgctggtcgtaaagggcactttcccgagcctacacaagaccctattattcaagtggccaatctggttaccttacagggacagggtcagccattaattcggaatgtgatgacccttaagtcatgctcacctatagttggggttgatgttatgtcctttgacacagaaaaagaagtattgctggcttggaggaaccttatccgtgaagtggaccctgatataataattggatataatatctgcaactttgatttgccgtatctcatcaggagagctgaagtcttagggatggcagaatttcctgtgcttggacgcatcagaaacagcagggttcgtgtaaaagatgcaaccttttcttcaagacagcatggaacaagggaaagtaaagaaataacaatcgaagggagagtcaaatttgatttatttcaggctgtgcagagggattataagctaagttcttactcactaaattctgtctcagcacattttcttaacgagcagaaagaagatgtccatcattcaattatatcagatcttcaaaatgggaactcagaaactaggaggcggcttgctgtatattgtctaaaggatgcttatctcccacaacggcttttggataaattaatgttcatttataactatgtggagatggctcgagtcactggtgtacctatatcttttcttctatcaagagggcaaagcattaaggtgctttctcaacttttaaggaaggcgaagcaaaagaatcttgtcataccaaatgcaaaacaggctggatcggaacaaggaacatttgagggcgcaacggttttggaggcccgaactggtttttatgagaagccaattgcaacattggattttgcatctctatatccatcaattatgatggcatataatttatgttactgcacgctggtaactcctgaagaggttcgcaaactgaacctccctccagagtgcgtcaacaagaccccttctggtgaaacatttgtcaagtcaaatttacagaagggaatacttccggaaattcttgaagagctattagctgctcgtaaaagagcaaaagcagatttaaaggaagcaacagatcctcgagtcaaagctgtcttagatggccgtcagctggctttgaagatcagtgcaaattctgtgtatgggtttactggagctactgttggtcaattaccatgtttagaaatatcttcaagtgttactagctatggtcgacagatgattgagcacacaaaaaagcttgtggaagacaagtttacagtacttgggggctatgaacacaatgctgaggttatatatggagatacagattccgtgatggtgcaatttagtgtgcctactgtggaagaggctatgaaattgggcagagaagctgctgattacattagcgggacatttatcaaaccaataagactagaatttgagaagatttattacccatatctgttgatcagcaagaagagatatgctggccttttatggacaaatccggacaaacatgacaaaatggatgctaaagggattgaaactgttcggagggataattgtctcttagtcaagaaccttgtaactgagtgtctttataaaatactagtagacagagatattcctggtgctgttcaatatgtcaagaatactatcgcagatcttctgctgaaccgcatggatctgtctcttttagttattactaagggtctgacgaaaactggagatgattatgaagtaaaggcagcacatgttgaacttgcagagcgaatgcgcaagcgagatgctgctactgcacctggtattggtgatagagtaccttacgtcataattaaagctgccaaaggtgccaaggcatatgaaaagtcagaggaccctatatacgtgttagagaacaacatacctatagatgctcaatactatcttgataatcaaattagtaagccacttttgaggatttttgagccaatactgaagaatgcaagtaaagagcttcttaagggagatcacacaagagccatctcagttccgaccccttcgaacagtggtattatgaaatttgccaagaagcaactcacttgcattggatgcaaaataccactcagtggctctgatcgaacgatctgcaatcactgcaaaggaagagaggccgaactttattgcagatccgtggctaatgtggctgaactagagaatctttttgggaggctatggacacaatgccaagaatgccaaggctctctccatcaggatgttctatgtactagtcgagactgcccaatcttttatcggaggaagaaggctcagaaagacatggctgaagctaaaactcaattagatcggtggaacttctgaatttccttgccggaaagtcttcatttctgcaaggccgacctgtcattaagccaggtatcgtatttgtgacatctctggtatgaagaaagagtttggaaatgaactgaacaaccaagacactactactatgtctgatgaaaaggtaccaattgtcttcctcaaatccaagacccactaatatacgcacggaaatcgtgttcctgcatgagtttaaacttcaaatcattagcaagattgggttcttaacaaacttttgtagcttccagctattaatgtgactggttagtataaaagagattagatagatgactatggtctataaatattgacgtattgtgatctgtataggctctgttagttactagtaccagtggattgcaactaccacttaaaattctcttctttgatctcattgcatattctctccttgtgccaagaa

SEQ ID NO: 32 - DNA, 3270 bp, Capsicum annuum
  source 1..3270: mol_type = other DNA
atgaacaacggcaaatcacggaagcgatcggcgcaggctgcggaggagacaccgtcacctcagccgccgcaaaaacaccacgccgcaatggaagaagaagacctagacgaagatgttttcctcgaagagacattgcttcagtacgaagaagattcagctgtccttcgcgaactcgaagagagtcatgcgcttaacgagcgtcttcagaagtggaaacgtccgccgctttctcaagcttacgtctctcatacacaaagcatatcgtttcagcagctggaaattgactacattattggagacagccataaagaattgctgcctacttcttctggtcaggctgccattttaagaatctttggggtcaccaaggaaggaaatagtgtctgttgtcatgtacatggatttgaaccatacttctatatcagctgccctcctggaatgggccctgatgatatctcccgttttcaccaagttcttgagggtaggatgagagagtcgaataggaacagcaatgtccctaagtttatccgccgcatcgaagtggttcagaaaaggagcatcatgtattaccaacagcatgtgtcacagccttttctcaagattgtagtggcattgccagcaatggttaccagttgccgtggtattctcgatagaggtattcaaattgaagggcttggtatgaagagctttctgacttatgaaagtaatattctttttgctcttcgcttcatgattgactgcaatattgttggtggaaactggattgaagttccggttgggaaatacaagaaagctgcaaagaatctctcctattgtcaattagagtttgattgtctatattctgacttgatcagccattctcctgaaggagaattttcgaagatggctccctttcgcatattgagttttgatattgaatgtgctggtcgtaaagggcactttcccgagcctacacaagaccctattattcaagtggccaatctggttaccttacagggacagggtcagccattaattcggaatgtgatgacccttaagtcatgctcacctatagttggggttgatgttatgtcctttgacacagaaaaagaagtattgctggcttggaggaaccttatccgtgaagtggaccctgatataataattggatataatatctgcaactttgatttgccgtatctcatcaggagagctgaagtcttagggatggcagaatttcctgtgcttggacgcatcagaaacagcagggttcgtgtaaaagatgcaaccttttcttcaagacagcatggaacaagggaaagtaaagaaataacaatcgaagggagagtcaaatttgatttatttcaggctgtgcagagggattataagctaagttcttactcactaaattctgtctcagcacattttcttaacgagcagaaagaagatgtccatcattcaattatatcagatcttcaaaatgggaactcagaaactaggaggcggcttgctgtatattgtctaaaggatgcttatctcccacaacggcttttggataaattaatgttcatttataactatgtggagatggctcgagtcactggtgtacctatatcttttcttctatcaagagggcaaagcattaaggtgctttctcaacttttaaggaaggcgaagcaaaagaatcttgtcataccaaatgcaaaacaggctggatcggaacaaggaacatttgagggcgcaacggttttggaggcccgaactggtttttatgagaagccaattgcaacattggattttgcatctctatatccatcaattatgatggcatataatttatgttactgcacgctggtaactcctgaagaggttcgcaaactgaacctccctccagagtgcgtcaacaagaccccttctggtgaaacatttgtcaagtcaaatttacagaagggaatacttccggaaattcttgaagagctattagctgctcgtaaaagagcaaaagcagatttaaaggaagcaacagatcctcgagtcaaagctgtcttagatggccgtcagctggctttgaagatcagtgcaaattctgtgtatgggtttactggagctactgttggtcaattaccatgtttagaaatatcttcaagtgttactagctatggtcgacagatgattgagcacacaaaaaagcttgtggaagacaagtttacagtacttgggggctatgaacacaatgctgaggttatatatggagatacagattccgtgatggtgcaatttagtgtgcctactgtggaagaggctatgaaattgggcagagaagctgctgattacattagcgggacatttatcaaaccaataagactagaatttgagaagatttattacccatatctgttgatcagcaagaagagatatgctggccttttatggacaaatccggacaaacatgacaaaatggatgctaaagggattgaaactgttcggagggataattgtctcttagtcaagaaccttgtaactgagtgtctttataaaatactagtagacagagatattcctggtgctgttcaatatgtcaagaatactatcgcagatcttctgctgaaccgcatggatctgtctcttttagttattactaagggtctgacgaaaactggagatgattatgaagtaaaggcagcacatgttgaacttgcagagcgaatgcgcaagcgagatgctgctactgcacctggtattggtgatagagtaccttacgtcataattaaagctgccaaaggtgccaaggcatatgaaaagtcagaggaccctatatacgtgttagagaacaacatacctatagatgctcaatactatcttgataatcaaattagtaagccacttttgaggatttttgagccaatactgaagaatgcaagtaaagagcttcttaagggagatcacacaagagccatctcagttccgaccccttcgaacagtggtattatgaaatttgccaagaagcaactcacttgcattggatgcaaaataccactcagtggctctgatcgaacgatctgcaatcactgcaaaggaagagaggccgaactttattgcagatccgtggctaatgtggctgaactagagaatctttttgggaggctatggacacaatgccaagaatgccaaggctctctccatcaggatgttctatgtactagtcgagactgcccaatcttttatcggaggaagaaggctcagaaagacatggctgaagctaaaactcaattagatcggtggaacttctga

SEQ ID NO: 33 - protein, 1086 aa, Cucurbita pepo
MNKGSGRKIRPPPQNPSPAPPAKHQALSQEEDFVDEDVFLDENLLDEDALILRDIEERQALASRLSKWTRPPLSQEYVSQSKSIVFQQLEIDYVIGESHRELLPNLSGPAAILRIFGVTREGHSVCCHVHGFEPYFYISCPPGMGPDDISLFHQTLETRMKEMNRNSKAPSFVRRIELVQKRSIMYYQQQTSQSFLKIVVALPTMVASCRGILDRGIQIDGFGMKSFMTYESNVLFALRFMIDCNVVGGNWIEVPVGKYKKTVRNLSYCQLEFDCLYSDLISHAPEGEFSKMAPFRILSFDIECAGRKGHFPEPTKDPVIQVANLVTLQGEDQPFIRNVMTLNSCSPIAGVDVLSFDTEREVLLAWRDLIREVDPDIIIGYNICKFDLPYLIERAEALKIAEFPILGRIRNSKVRVKDTTFSSRQLGTRESKEITVEGRVQFDLLQAMQRDYKLSSYSLNSVSSHFLSEQKEDVHHSIISDLQNGNAETRRRLAVYCLKDAYLPQRLLDKLMFIYNYVEMARVTGVPISFLLSRGQSIKVLSQLLRKAKQKNLVIPNAKQAGSDQGTYEGATVLEARAGFYEKPIATLDFASLYPSIMMAYNLCYCTLVTPEDVRKLNLPPEHFNKTPSGEIFVKPNLQKGILPEILEELLTARKRAKADLKEAKDPFEKAVLDGRQLALKISANSVYGFTGATVGQLPCLEISSSVTSYGRQMIEHTKKLVEDKFTTLGGYEHNAEVIYGDTDSVMVQFGVPSVEAAMILGREAADYISGTFTKPIKLEFEKVYYPYLLISKKRYAGLFWTNPNKFDKMDTKGIETVRRDNCLLVKNLVNECLHRILIDRDVPGAVQYVKNTISDLLMNRVDLSLLVITKGLTKTGDDYEVKAAHVELAERMRKRDAATAPNVGDRVPYVIIKAAKGAKAYEKSEDPIYVLENSIPIDPQYYLENQMSKPLLRIFEPILKNASKELLHGSHTRSIFISTPSNSGIMKFAKKQLTCIGCKALLSKADHTLCAHCKGREAELYCKAVENVSDLEMVFGRLWTQCQECQGSLHQDVLCTSRDCPIFYRRKKAQKDMAEAKQLLERWNF

SEQ ID NO: 34 - DNA, 14700 bp, Cucurbita pepo
  source 1..14700: mol_type = genomic DNA
atatcgcaaagctgtcttcaaaagtggcgggaaccctactcctcttccgcgtctgccattccacttccattttctgagttcactctcttccacaaaaaccagagccatgaacaagggcagcggtcggaaaattcgaccaccgcctcagaacccttcgccggctccaccggccaagcaccaggcactgtctcaagaagaagattttgtcgatgaagatgttttcctcgacgagaatcttctcgacgaggacgctctcatcctccgcgacattgaggagcgtcaggccctcgcttctcgtctctctaaatggactcgtccacctctttcccaggagtacgtctcccagtcgaagagcattggtgagttgaaagttactgctatgcttctgctcgggttactacatttttttttaatcatttagaaatttgatgcgtgtttgtgtgttttatttacttttttcacggttggtgtttcagtgtttcagcaattggaaattgattatgtgattggggaaagtcacagagaattgttgccgaatttgtctggccctgccgctattctaagaattttcggggttacgagggaaggtttgcatccgtttacgaattataaaattatccgttattctaatgagcgatatgtcccgaagataatggtaaccatttgttagatgtcctaagtatagcttgtgtgactactgacattatcacccctcgcgttaagccatttgcatacatttttcttgctctgtgcttgtgcgaaaactggccctgtggcattgcgagagtaatttccttgaggttaggccacactccattttgtatgtgtatgacgaaggttaaattgtccaattgaggatcagcaagggctggatcatcaatagcgaagagctacaaagaaagaataaaacactcaattttccatggagaaaaattctggtataaattataataacagaataaatattaagatttataaggaaatactatatattctaataacttaggctatagccactgtttgactgagtggaggagagattctctgattctctatgttagaagtcttgtgtgagatttattgttgaaagtttttcttgtttctgtaaagtacgcttctactcatttggtacaccattctgttggatatatactaggacatagtgtttgctgccatgttcatggatttgaaccatacttctatatcagttgccctccaggaatgggtcctgatgatatctctctttttcaccaaacacttgaggtaatgcattatatcaattcacctgttgatactagtgcaatttgaagttctaaaaatctgccgttgttgaattggtgatggtgtttcagactagaatgaaggagatgaataggaacagtaaggcacctagtttcgtgcgccgtattgaattggtgcaaaagcgaagtattatgtactaccagcagcaaacttctcaatcctttcttaaaattgttgttgcactgccaacaatggtcgccagctgccgtggtaattatttatctttttgaagtaatttctcaatggggaggtacaagtcttatatggtctttcattatttatctttcactctttttgtatgcatcgtttaataagttatattttccacgccatttgtatgtctctttgttcttttagaaataattatccgtagagtaacacaaatgctttttatttttttaacttctttgtaatgtttcattttatcaatgaaaacttatgtttctttttctttttaacagtgtccctatgagaagtctgatgtcatgctttatctcatttgtattgttgttaacattctttttccaggtattcttgacaggggcatacaaatcgatggctttggtatgaagagcttcatgacatatgaaagcaatgtgctatttgccctccgttttatgattgattgtaatgttgtgggtggaaactggattgaggttcctgtgggaaaatataagaaaactgttagaaatttatcatattgccaattggagtttgattgcctgtatccttttgatctacaggtgtctgtctttatgtgtattttttgaaatttttattcaagttctacttttagacgagctttttattctggatactttccttaacactcttcatagatattcagacttgatcagccatgcccccgaaggggaattttctaaaatggctccatttcggatcttgagttttgatattgagtgtgcagggcgtaaaggccattttcctgagcctaccaaggatcctgtaattcaggtttaactctttatttcatctaattgtgtgtgctaaaatcaatttcttggcttttcagttatgaactttgtatactttttcttggtttaggttttgaccttttctgaatgactttctttctaagaaaaagaagtttgtttaccctttctgaacgagtctacctatatctaggtggcaaacctagttactttgcagggagaggaccaaccatttattcgcaatgtcatgacccttaactcttgctctcctattgcgggcgttgatgtcttatcatttgatacagaacgtgaagttttgctggcttggagggtattatcatatgacatcctttcataattagttgttcaaatgattgttatcctttctgtaaaatcttaggtgaatcctgcattcatgaaataactaccaatgatatttcctagagtctaaagaactcttttaaagaattcatggtaacttctagacttatgcacaatatttgatggctttattcttgctactgttttcagtatatatgtaatttcatttgttaaattaaagtagtttcttataagaggaagattggctctttaatcaatatatcaatgagtagtcaagactccgtaataacagctaatgtgatgtaggacatctaggctactattctaggcatttattatactttccttagtttcatgaaaattttgtttaagtgatcttttatcttatggtaaacatttttactgtttagttatttttatgttgttcagccagttaccttggcttttaaattctgttccatccctcttacttgtataaataaggagtcgcagtctttgttcattactttttgactacaatgaataaaattccccgcactagtttacatcataatgtcaaaagataagttattagctcagtatcatattaaccatttgcttggttatggattgaataaaacaataattattagacaaagaatgtgtgatcatgtgctcaagtaacaattaacagacgtgagaatgcacatttatcttcacgtctctttataatttataaattgcagtttgacagcctttcttattagaaatgatcgtggatcaagggaaggggacatgaaagtgaattttatgtttattatttcattctttgatgtattataccctagtgacagatggacttttgatgtgtgtagattgtgaatgacgagaacaattttaactctgacgacaatatttggttgcaggatctaattcgtgaagtagaccctgatataataattggatataacatctgcaaatttgatttgccatatctaatagaggtatttataccaatgtatttggtttatgaaatttggagttctttgcaatattctttttcttgtgaagagttcttcatgactgagaagtatttatttgttgtagttgacatggttactagttcacagatgtcttctatgttgtttttctgctcaatctattgataatttgaaaatgctaagacgtctcatagaatttttcccgaatccattgtcaatattttgtgaggtctgaaaatgttgatgccatcacttcaaaactaaatgttgattctatcgattatttctgaactcgtactaatgcactatcctgcttctctgaatgtagagagctgaggcactgaaaatagcagaatttccaatattgggtcggatcaggaatagcaaagttcgtgttaaagatacaactttttcttcaaggtatactagaatttaggttgtctacgactttgatcgtgttatacttttacgcttcataaataaaattttctactgaattattctttcctaaattcaatttttttctgaaaatatattactatgaatatgttgtccattaaaaataatcatttgtctgctgtcaagtagacgggtttgtgtaagcattctggttttgatggacatatggctaatatgaacagacacccaaagtttctttgggaatgaaagccttacttcttgcccgaaggaatctttatgtttttattttctaagaagagtgaagcagttgatgtgattcatccctaaaactatcaaagaactcctatcgtccaattagaaccccttgttagaaggagtgaagatggggccagggttccttgtggagaattttgttgtgaatgaatttcccattgtttgtttatttatgtttgacacgatgtgattcaagaaagaatggtgagccaaaaaatttaataggggaaattagtggaatacattttgaactgatttcttttggtcctacccaccattattttacgtagatgacctattccttgtgccatccttcatttactagtccaatgagcactctacatcgatattgcagaaagttaagttgttaacgactattacttatcaatatcaaaattgagtatttagggacataattgaaaattttaatgttttgggattaaatagatagaactctttgaattcaatgccttaatttttaatttaacgactttcggttgcaacttcatttatttacaagtaagacattgtttttatttttattttattttacttgggtgaacatttaatgctattcaagttatatatgtggtgttttatgttgataaattatactcatgtaggcgaaatacataattccatgattccatccaaaatgtctataccaattttcaatttcatttccagtatttcaaaactttcactattttagctagtaatatgtatgaaaaaaaactaataatggtgaacgttcattaaagtgaatggcaagttcttgtaacattcttcttcttactttgacatgtttcaagtattatactgtttggttacaattaatgattcaattaaaaaccattatgggttggcctaatagtcaagatggatcatgtaattaatgaagggcttatagagaatgggttcaagccatggtggcactaaggtctaatatcttacgagttacctatgcatccaaatgtaatagggtagacagttggcccttgagaatagttgagatgagcacaagctgggcggacactcacaaatataaaaaaaattaatgattgagttacattattttgtaaaatatgaagctttacagttatgctaataaaggcagtatatagagaagatatatgaatggctctaatggtttcactcttacatttatgtatttgttatgttttatgtcattttttttcttggttacaaatgaatttaattgttgattattgcctcagacagcttggaacaagggagagtaaagaaataacggtggaaggaagagtccaatttgatttacttcaggttttatctttatacacattaaataattagcacggatgatgcatgtaagagctttaaatttgatttcattttgtaacataataggtttttaaatgaaccattattaaatactaatatgttgtctgtataaaaccacatatatttgaaatgtatgactagtattattttgatgtattagtttagtgtagttatttttttctaacaattaatatgttctgaacgaattgaaactatcaaatatattattccaagttttgatgcctttttttatcatgttaatgtatgtgtatgttaatctcttgcctttatgtaaaaataaagcatataagataacttacaactattgactataacgttgacacttttttaatctagcaaccaaaagcaaatgttaaatgtaatcaaattacttttagtcttgccagttctaagaataaactggcttaattggttgtagctgcttgcactcttaatccctcaaggggagatcaacagttcccgtacaagaatgaaaaatctttgaggattaaatattgagttacgctgtattttcaatttttaatagaatttttgttttgaatatatcttttacttgaccactaaaagctttttttaacctttagtaaaagaaaacaattgaattccatgaatgtgttacatgtattgaaagtaatatgtgattttatgaagggatgtatttatagttatcttattgaagagtgggaattggtcacaagatgattccaactttctaccttttaatagtttaaagataaagatgttttttaattcatcaaagcttaatttagctcatgtttttgttctcttattttgcaggcaatgcaacgagattataaattaagttcatactcgttgaattctgtttcttcacatttcctttctgaacaggtgatattcaagagtcctcccttagaccttctgatgtgaatctttattataatttgtttctctaggcaattaatatttatgtattcagtcactggtgtgagtgttagttggatttggaacattgtattttgttgtttacgctaataaagagtactacttcatttgaagagggcttcatattatttctttagtttaccaagagcctaggagttataaattatattttacaatttaatttgttagaaacagtgaattatcaagcttccatctattcatggttgttatgatgattcatttttacttctgcagaaagaggatgtccaccattcaataatatcagatcttcaaaatggtaatgcagaaacacgtagacggctggctgtttattgtttaaaggtgccttcctcgactcatttgcatttcagatattaccatctcattcaatattacatttacttatcattgttttttcgaggcaatccattttataattgaaattagtcctgcaaaatagtggtgacgtttgtaagcacgaagaaggatttagtctaccttatttagtgtacatggaggctgtttacttacatcctggttagaatgataaggtaagagttttatcagaaaaagaaggtatgttaggagggaaaattttgaagaatgattaagttaggaggaaattttttgaagaatgattctttcagtgagcaaccccaagaattagtctatggttctgtcattgaaaacttctaccctcttgcatgcatttatgttttagacatcctggtcggttgtgatctggtggaaaccttgatttggtaggcctaagcctttcatgaaaatatataattgttttgtagaagataaatgcagtcttccgtcagccttttctttttctgtccttttgcttatttctgctttctttcctctctctttttagtaaaaaagttgtgtccgtagattttcatatgcttggttttggaatcccgtggagaaatttggaaatgatctaatattattacagccaccaagtgcaattatttttgtttaaactatgcattttcttagctttggaactccattgaggttgttgtttccaatcttttctttatattcttgagtaatatcaaactttcttaaaattcttttggtttcatatttttggggtgcgggaattcaggatgcatatcttccacaacggcttttagataaactgatgttcatttacaattatgtggagatggctcgagtaactggtgttcctatttcgtttcttctttcaagagggcaaagcatcaaggtaatccttctgtcttttaatttttaggcactccttttgacctagcagaattattatcaactttaagaatttgtttccaggtgctctctcaactccttaggaaggcaaaacaaaagaacctcgttattccgaatgctaaacaggcaggatctgatcaaggaacctatgagggtgctactgtatgttcttcgacatcctgcatctttttccttgactcaacaatttattttaatcttattgatgtttttaagtagtaaatgatagaggttccataggtgctcatgccacgaatgtttagtgtagttactttaatgtaggatgatgtctaagctcagaatctttgtattctttgtcggtcctaatctgagtctcaaaaacatgcctcagaattgaaatggttaacatatttgctccattctgactgacttttcttgcatgtgaaaatcctcagacttgagcaaacttaatttggtcgattcgattacttcaattctatgttgctatgaatgtatcagctgaaataggtaatatatctgaataatctcggtttcgaacatgcaggttttggaagcaagggctgggttctatgagaagcccattgccacgctggattttgcatctctgtatccatccatcatgatggcatataatttatgttattgcactctggtgacgcccctaacatattttttggcttatagaatgtgctgttcatttcacgctgggaaattattcttaataaatttgggggaagccttcaactttaaacattcttttgaaggtgactccagaagacgttcgcaagctaaatctgccccctgaacatttcaataaaaccccctctggtgagatatttgtcaagccaaatctacagaaggtcagcgggttaataatttccattcttatgaaagatatctggtttagtttatgcctcctcaacaaagttaaccacctgtttaaaacttacaacttctgcagtaaatattgtttttcaaactaatgtaccaaataactttgatataattcaggggatacttcctgaaatattagaggaattattgaccgctcggaaaagagcaaaagcagatttgaaggtaattgcattgctctttcagtagttggcttgttgcgttacacaatcagtgtactgttccaatttcatgttttgtcagtcaaatttacttcgttttgtcaataatatttacatccatgttgcctttgtgtaggaggcaaaggatccttttgagaaggctgtgttggatggtcgacagctagctctgaaggtacattacattttttttttatgtaccccattctgttagttttttggacttaacatgtttccttctatctttgcagataagtgcaaattctgtgtatgggtttacaggagctactgttggtcagttaccgtgtctagagatatcttcaagtgttacaagctatggtatactaaaaaatacgattaattcttattatccttaattaaattctttacgctcaagctattcatattaatgaattggctactttaaaggccgacaaatgattgagcacacaaagaaacttgtggaagataaatttacaactcttgggggctacgagcacaatgctgaggtaagttaaaattttgttgatggtatttttggaaataaaatataattgacttagctccattttccaagtttatttatgctcagttttacagaggaactcggtattgccttttattaattcaacgatcatttgcatttgtgtctttaaatgggctgtatttgagggaatctatctaattatagttctagttgcacatcaatgaacgaagaagcacatataataatacaaactgaaagaagagaaggggtaaaatttccattttgatttagaagattacaggattatcagtacaaactatttgacgatgaatttgaatactataatagaccagacagttcaagtcaatcccaaagaatcaggactacgtttagctcctctttgtcctgtttaagctctgtacttatggaaattgtattctttccgtagacaacttcttttaagtatttgatagtttctgtagcgttggaatacgatgcctgtagaattgtctatatattttcaattaacaacgatttcccttactagttgaatcactggtctaaaaaacgttttgatatgtctaaatggttgagacagacttgaattgttaaatcattgattttatgttccaacttctaaaagaataataattagattcttgtgttgcatcaatctatatctcattgatgaccttacagaagtcagcgttttatggttttaggtaatctatggtgatacagattcagtaatggtacaatttggtgtgcccagtgtagaagcagcgatgatcttgggaagggaagcagctgattatatcagtggtactttcacaaaagtaagagttgcttgcgctgttgttatgcctttttatgttttttttctcgtttttattggtatactgctaagtgctttgcaagaagttttccatctgcttctgtatttttattatgagtatagtatatgagtaaattaactgtatagtttttctttgaaatccaaagtaaacagtttattatcagatttgtaactttttcttataaatttccctgtgcagtcttagctgatgttttcatcttcattctatttttttttaattgtgtgttcgttcatggtttgtagcccatcaagttagagtttgagaaagtttattacccatatctgttgattagtaagaagagatacgctggtttattttggactaatccaaacaagtttgacaagatggataccaaaggtaatgatggagaagctaaaagaatttgtctttgcccacgtttaaattatttccctccatttcaaatattctatttgacaaccgcaggcattgagacagttcgaagagataattgtttattggtgaaaaaccttgtaaatgagtgcttgcacagaattcttattgatagagatgtccctggggcagttcaatatgtgaagaacacgatttcagatctacttatgaaccgtgtggatttatctctcttggttatcactaaggttggagatatatgtcaaatatttttatttcactttcattaagtgtcaaggaacccttgtcataagtttgcttttaggacattaacttgaaggcttcatagtgcattaaattatttgatccacagggactgacaaaaactggagatgattatgaagtgaaggcagctcatgttgaacttgctgagcgcatgcgcaaggttgtggtgtttcattatagttttttccttgttgtttgactctttatttggtgtcaaattcctacacgtctattttgttgacataatgaacatcggttccactattatctttgcatgggtgtattgaggaaaactattcatgatttgttcgaccatgtagtgttcattagatgcaatcctaggttttcttactgtggcacgtctggcttaagactaatagtagtacagcgaacctaagaaccctcatagatatgcaaacccctagatcataaatgccgaaactccccaatcaaaatggtttcatcccaaataattgtaactcttgtaaattcgatctaacaactttagtaagacctttagatctaaatatccaagtcctctagaatgataaggaattatcccaaggaaaagtaaaacctggattaccttattgatcaaatatctcaagataagaaaacttgtttgagattcaaatcactccacaagcaagatagacatgtcaagcttgaatgattctacacatgctacctattctacatagaattgaaaagaaacttagcatttagttaaaggaaggcacaaatgcttttttactgcatttttcaagtctagcttacaaagacaacatacatgactttgtatagcctcaaaagaaaatccttgacctttcacgaggcgtttcaagagttatataacttttgtactttataaccataattagccactttgtaaatgtaactttaaagtaaatacaaaatcttaaaactcaaattcattaatgaaacaccataactctagattaccatccacccaaaatttgtaaccatccaagaaacttgattcttttttaatgtgacattgcaacttgagtttatcttgtggtaatttgaaaacatttttctcacatctttcttaaaatttgtatattgtatgattgaaatgtttttgttcataccactgcagccctctcatttatgtcatgcgcatacatatgcataaacttatctttagtgacatgtcacttgaccctagaattattaagaatttatacctcttgccactgccaatcctaaagtgtaggaattggaaaattttataccatgaatatggtatcaagtaattgaataacttggatgcatccatcttgactgagctcaagcttcaatgactagatgaccactaattttcccaactttctcatcactgcaatccctatttttagttaacctgtggttgcttagaaattctaacaattccacactctacaagtaccccactaatgattatacttcccccctgcacatgggttttatgtttgcttgcaaggacttggtctcgaagagaaattcatctattagtagatttgctaaattttaattatgggaatgcccaagtaataattttaattatgggactagagtaagctattttttgattctatatattacaagaaacatactaatggaacataaaagttcagatgaagcaaccacacaaaaaagtcccgaaagaaaaacgtgaccaaatagtcactaaagctaatgccctatcccaccaaagccattatctgagggacaaacaattggagagagccatgaaggttccaacaagagtgactgcacgagtcaccttcaaagctgctaaagaccacaaataaatattgaaaatgaaatccttaataataactaataattcctttgaagcttttgaagatttgagctcatcgccaatgattgaagaaattccctccggagaaggaaaagaaaaatagagcttggattcttatagaaaccgttggagatgcttttcataatctgaaatgaggctgctaacactatccctattttattttggtatcctaattgataaatagcatgcttattctaatagattcaaaatcagtgctcctcagcgcagtattttcacacatccatgtgttttctgacccctatcctaaaagaattctggaaatatactattaagtcgcacaagaagtatttgtaaaacgaaagaaaattttcttctttctacatatcatatcttgcattttgtgatgcatattcttatttcatgactccattgaatgaatgcttgcagtaagaaacatggtttttgattctcgtagcctaatccagttcgttgctgtgtctggttttaaaacaagattatctaacttatttctatccttaatagcgagatgctgccactgctccaaatgttggtgatcgagtaccttacgtgatcattaaagctgcaaaaggtgccaaggtaatccatttgatatctcaacccttccaatgctcaatgtgtatgaagattaatattggatgacaatcaggcatatgagaaatcagaggatcccatttacgtactagagaatagcataccaatagaccctcagtactatctcgagaatcagatgagcaaggtttgatctctttattcttttttgttctgttcttttacttcatttacgtagacgttgaaggattatgtctaagttgcagcctcttctacgaatttttgagcccatcctgaaaaatgctagcaaggaacttctacatggaagtcacacgagatcaatttttatttctactccttcaaacagtggcataatgaaatttgccaagaaacagcttacctgcattggctgtaaggctctgctaaggtaattttgaaagttttattatccattttggttttatctagtttgagggcaatattttcttctaatgttcgatgtaggaagttcaataaccttgctatagtgaaattgagaggaactgatttttgtttccattctgcgttacagttttggaaaagcaaaaattgttttaagactagcgctagtttgttgtgtcattttaacaaggaacacaattattatcttttgaatatttttgaaaagattcacttgaaagtccgcagagcaatatgtattgaataaattctatactatacttgactactccagcaaggcagaccatactctctgcgcacactgcaaaggaagagaagctgaactttactgcaaagcggtagaaaatggtaatcttctttgtaagaatttgttccaatatctagctaattgaattctcacctatataaaatttttggtcgttaaccgttgcagtgtctgatttggagatggtttttggaaggctttggacgcagtgccaagaatgccaagggtcccttcaccaagatgttctctgtacgaggtattgttttattaccttcttgacgtaattcttatgatcattactttttcctagtattattagcatgcatgcagatcttctattctagattcaaatgttgttcttgtgcctctgattcatagtttcacaaatgctatattttggtttacagtcgagactgtccaatattttatcgaagaaagaaggcacaaaaagacatggctgaagctaaacagcttttggaacgttggaatttctaataccgttgaaaggttttgttttgctgtttttaccaccatcaagttcatgattttgatattgactggaaatttaatcttgttgctagaagcctccttcctcctgtaaattataattattataattactatgcatttgaaggcaatatatggttttacatcatttgtacattaa

SEQ ID NO: 35 - DNA, 3539 bp, Cucurbita pepo
  source 1..3539: mol_type = other DNA
atatcgcaaagctgtcttcaaaagtggcgggaaccctactcctcttccgcgtctgccattccacttccattttctgagttcactctcttccacaaaaaccagagccatgaacaagggcagcggtcggaaaattcgaccaccgcctcagaacccttcgccggctccaccggccaagcaccaggcactgtctcaagaagaagattttgtcgatgaagatgttttcctcgacgagaatcttctcgacgaggacgctctcatcctccgcgacattgaggagcgtcaggccctcgcttctcgtctctctaaatggactcgtccacctctttcccaggagtacgtctcccagtcgaagagcattgtgtttcagcaattggaaattgattatgtgattggggaaagtcacagagaattgttgccgaatttgtctggccctgccgctattctaagaattttcggggttacgagggaaggacatagtgtttgctgccatgttcatggatttgaaccatacttctatatcagttgccctccaggaatgggtcctgatgatatctctctttttcaccaaacacttgagactagaatgaaggagatgaataggaacagtaaggcacctagtttcgtgcgccgtattgaattggtgcaaaagcgaagtattatgtactaccagcagcaaacttctcaatcctttcttaaaattgttgttgcactgccaacaatggtcgccagctgccgtggtattcttgacaggggcatacaaatcgatggctttggtatgaagagcttcatgacatatgaaagcaatgtgctatttgccctccgttttatgattgattgtaatgttgtgggtggaaactggattgaggttcctgtgggaaaatataagaaaactgttagaaatttatcatattgccaattggagtttgattgcctatattcagacttgatcagccatgcccccgaaggggaattttctaaaatggctccatttcggatcttgagttttgatattgagtgtgcagggcgtaaaggccattttcctgagcctaccaaggatcctgtaattcaggtggcaaacctagttactttgcagggagaggaccaaccatttattcgcaatgtcatgacccttaactcttgctctcctattgcgggcgttgatgtcttatcatttgatacagaacgtgaagttttgctggcttggagggatctaattcgtgaagtagaccctgatataataattggatataacatctgcaaatttgatttgccatatctaatagagagagctgaggcactgaaaatagcagaatttccaatattgggtcggatcaggaatagcaaagttcgtgttaaagatacaactttttcttcaagacagcttggaacaagggagagtaaagaaataacggtggaaggaagagtccaatttgatttacttcaggcaatgcaacgagattataaattaagttcatactcgttgaattctgtttcttcacatttcctttctgaacagaaagaggatgtccaccattcaataatatcagatcttcaaaatggtaatgcagaaacacgtagacggctggctgtttattgtttaaaggatgcatatcttccacaacggcttttagataaactgatgttcatttacaattatgtggagatggctcgagtaactggtgttcctatttcgtttcttctttcaagagggcaaagcatcaaggtgctctctcaactccttaggaaggcaaaacaaaagaacctcgttattccgaatgctaaacaggcaggatctgatcaaggaacctatgagggtgctactgttttggaagcaagggctgggttctatgagaagcccattgccacgctggattttgcatctctgtatccatccatcatgatggcatataatttatgttattgcactctggtgactccagaagacgttcgcaagctaaatctgccccctgaacatttcaataaaaccccctctggtgagatatttgtcaagccaaatctacagaaggggatacttcctgaaatattagaggaattattgaccgctcggaaaagagcaaaagcagatttgaaggaggcaaaggatccttttgagaaggctgtgttggatggtcgacagctagctctgaagataagtgcaaattctgtgtatgggtttacaggagctactgttggtcagttaccgtgtctagagatatcttcaagtgttacaagctatggccgacaaatgattgagcacacaaagaaacttgtggaagataaatttacaactcttgggggctacgagcacaatgctgaggtaatctatggtgatacagattcagtaatggtacaatttggtgtgcccagtgtagaagcagcgatgatcttgggaagggaagcagctgattatatcagtggtactttcacaaaacccatcaagttagagtttgagaaagtttattacccatatctgttgattagtaagaagagatacgctggtttattttggactaatccaaacaagtttgacaagatggataccaaaggcattgagacagttcgaagagataattgtttattggtgaaaaaccttgtaaatgagtgcttgcacagaattcttattgatagagatgtccctggggcagttcaatatgtgaagaacacgatttcagatctacttatgaaccgtgtggatttatctctcttggttatcactaagggactgacaaaaactggagatgattatgaagtgaaggcagctcatgttgaacttgctgagcgcatgcgcaagcgagatgctgccactgctccaaatgttggtgatcgagtaccttacgtgatcattaaagctgcaaaaggtgccaaggcatatgagaaatcagaggatcccatttacgtactagagaatagcataccaatagaccctcagtactatctcgagaatcagatgagcaagcctcttctacgaatttttgagcccatcctgaaaaatgctagcaaggaacttctacatggaagtcacacgagatcaatttttatttctactccttcaaacagtggcataatgaaatttgccaagaaacagcttacctgcattggctgtaaggctctgctaagcaaggcagaccatactctctgcgcacactgcaaaggaagagaagctgaactttactgcaaagcggtagaaaatgtgtctgatttggagatggtttttggaaggctttggacgcagtgccaagaatgccaagggtcccttcaccaagatgttctctgtacgagtcgagactgtccaatattttatcgaagaaagaaggcacaaaaagacatggctgaagctaaacagcttttggaacgttggaatttctaataccgttgaaaggttttgttttgctgtttttaccaccatcaagttcatgattttgatattgactggaaatttaatcttgttgctagaagcctccttcctcctgtaaattataattattataattactatgcatttgaaggcaatatatggttttacatcatttgtacattaa

SEQ ID NO: 36 - DNA, 3261 bp, Cucurbita pepo
  source 1..3261: mol_type = other DNA
atgaacaagggcagcggtcggaaaattcgaccaccgcctcagaacccttcgccggctccaccggccaagcaccaggcactgtctcaagaagaagattttgtcgatgaagatgttttcctcgacgagaatcttctcgacgaggacgctctcatcctccgcgacattgaggagcgtcaggccctcgcttctcgtctctctaaatggactcgtccacctctttcccaggagtacgtctcccagtcgaagagcattgtgtttcagcaattggaaattgattatgtgattggggaaagtcacagagaattgttgccgaatttgtctggccctgccgctattctaagaattttcggggttacgagggaaggacatagtgtttgctgccatgttcatggatttgaaccatacttctatatcagttgccctccaggaatgggtcctgatgatatctctctttttcaccaaacacttgagactagaatgaaggagatgaataggaacagtaaggcacctagtttcgtgcgccgtattgaattggtgcaaaagcgaagtattatgtactaccagcagcaaacttctcaatcctttcttaaaattgttgttgcactgccaacaatggtcgccagctgccgtggtattcttgacaggggcatacaaatcgatggctttggtatgaagagcttcatgacatatgaaagcaatgtgctatttgccctccgttttatgattgattgtaatgttgtgggtggaaactggattgaggttcctgtgggaaaatataagaaaactgttagaaatttatcatattgccaattggagtttgattgcctatattcagacttgatcagccatgcccccgaaggggaattttctaaaatggctccatttcggatcttgagttttgatattgagtgtgcagggcgtaaaggccattttcctgagcctaccaaggatcctgtaattcaggtggcaaacctagttactttgcagggagaggaccaaccatttattcgcaatgtcatgacccttaactcttgctctcctattgcgggcgttgatgtcttatcatttgatacagaacgtgaagttttgctggcttggagggatctaattcgtgaagtagaccctgatataataattggatataacatctgcaaatttgatttgccatatctaatagagagagctgaggcactgaaaatagcagaatttccaatattgggtcggatcaggaatagcaaagttcgtgttaaagatacaactttttcttcaagacagcttggaacaagggagagtaaagaaataacggtggaaggaagagtccaatttgatttacttcaggcaatgcaacgagattataaattaagttcatactcgttgaattctgtttcttcacatttcctttctgaacagaaagaggatgtccaccattcaataatatcagatcttcaaaatggtaatgcagaaacacgtagacggctggctgtttattgtttaaaggatgcatatcttccacaacggcttttagataaactgatgttcatttacaattatgtggagatggctcgagtaactggtgttcctatttcgtttcttctttcaagagggcaaagcatcaaggtgctctctcaactccttaggaaggcaaaacaaaagaacctcgttattccgaatgctaaacaggcaggatctgatcaaggaacctatgagggtgctactgttttggaagcaagggctgggttctatgagaagcccattgccacgctggattttgcatctctgtatccatccatcatgatggcatataatttatgttattgcactctggtgactccagaagacgttcgcaagctaaatctgccccctgaacatttcaataaaaccccctctggtgagatatttgtcaagccaaatctacagaaggggatacttcctgaaatattagaggaattattgaccgctcggaaaagagcaaaagcagatttgaaggaggcaaaggatccttttgagaaggctgtgttggatggtcgacagctagctctgaagataagtgcaaattctgtgtatgggtttacaggagctactgttggtcagttaccgtgtctagagatatcttcaagtgttacaagctatggccgacaaatgattgagcacacaaagaaacttgtggaagataaatttacaactcttgggggctacgagcacaatgctgaggtaatctatggtgatacagattcagtaatggtacaatttggtgtgcccagtgtagaagcagcgatgatcttgggaagggaagcagctgattatatcagtggtactttcacaaaacccatcaagttagagtttgagaaagtttattacccatatctgttgattagtaagaagagatacgctggtttattttggactaatccaaacaagtttgacaagatggataccaaaggcattgagacagttcgaagagataattgtttattggtgaaaaaccttgtaaatgagtgcttgcacagaattcttattgatagagatgtccctggggcagttcaatatgtgaagaacacgatttcagatctacttatgaaccgtgtggatttatctctcttggttatcactaagggactgacaaaaactggagatgattatgaagtgaaggcagctcatgttgaacttgctgagcgcatgcgcaagcgagatgctgccactgctccaaatgttggtgatcgagtaccttacgtgatcattaaagctgcaaaaggtgccaaggcatatgagaaatcagaggatcccatttacgtactagagaatagcataccaatagaccctcagtactatctcgagaatcagatgagcaagcctcttctacgaatttttgagcccatcctgaaaaatgctagcaaggaacttctacatggaagtcacacgagatcaatttttatttctactccttcaaacagtggcataatgaaatttgccaagaaacagcttacctgcattggctgtaaggctctgctaagcaaggcagaccatactctctgcgcacactgcaaaggaagagaagctgaactttactgcaaagcggtagaaaatgtgtctgatttggagatggtttttggaaggctttggacgcagtgccaagaatgccaagggtcccttcaccaagatgttctctgtacgagtcgagactgtccaatattttatcgaagaaagaaggcacaaaaagacatggctgaagctaaacagcttttggaacgttggaatttctaa

SEQ ID NO: 37 - protein, 1089 aa, Vitis vinifera
MPATTATKKRPQPPQPSPSQPQPKHQASSSPLAEEDLDEDVFLEETLILEDEHILRDIEDRRALASRLSKWARPPLSSAYLSSSQSIVFQQLEIDYVIGESHKELMPNSSGSAAIIRIFGVTREGHSVCCNVHGFEPYFYISCPPGMGPDDISRFHQVLEGRMREVNRNSRVPKFVRRIEMVQKRSIMYYQLQSSHPFLKIVVALPTMVTSCRGILDRGIQIEGFGMKNFVTYESNVLFALRFMVDCNIVGGNWIEVPAGKYKKTAKNLSYCQLEFDCLYSELTSHIPEGEFSKMAPFRILSFDIECAGRKGHFPEPTHDPVIQVANLVTLQGEDQPFVRNVMTLKSCSPIVGVDVMSFETEREVLLAWRDFIREVDPDIIIGYNICKFDLPYLIERASVLGIAEFPILGRIRNSRVRVKDTTFSSRQYGTRESKEVTVEGRVQFDLLQVMQRDYKLSSYSLNSVSAHFLSEQKEDVHHSIISDLQNGNAETRRRLAVYCLKDAYLPQRLLDKLMFIYNYVEMARVTGVPISFLLSRGQSIKVLSQLLRKAKQKNLVLPNVKQAGSEQGTYEGATVLEANAGFYEKPIATLDFASLYPSIMMAYNLCYCTLVTPEDVRKLNLPPECVNKTPSGEIFVKSSLQKGILPEILEELLAARKRAKADLKEAKDPLEKAVLDGRQLALKISANSVYGFTGATVGQLPCIEISSSVTSYGRQMIEHTKKLVEEKFTTLGGYEHNAEVIYGDTDSVMVLFGVSTVEAAMNLGREAAEYISGTFMKPIKLEFEKVYYPYLLISKKRYAGLLWTNPDKFDKMDTKGIETVRRDNCLLVKNLVKECLHKILIDRDIPGAVQYVKNTIADLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNTDRTLCSHCKGREAELYCKTVANVSELEMLFGRLWTQCQECQGSLHQDVLCTSRDCPIFYRRKKAQKDMAEAKLQLERWNF

SEQ ID NO: 38 - DNA, 32744 bp, Vitis vinifera
  source 1..32744: mol_type = genomic DNA
cacgtgacgcaccagatcacaaaatcactccaaaaactggcgggaatccagtgttcccctccctctctccctccactctcactccgcactcttccatccacccagccatgcctgccaccacagccaccaagaaacgccctcagccgccgcagccgtctccatcgcagcctcagcccaagcaccaagcatcatcctcacccttggcggaagaagacctcgacgaggacgttttcctcgaagaaaccctcatcctcgaggatgagcacatcctccgcgacatcgaggaccgccgggcccttgcctcgcgcctctcaaagtgggctcgtccccctctctcctcagcctacctatcttcgtcccagagcatcggtatcctctcaacctcatgcttttcatttaatttctagggtttcttcttacatgttagtttatgtattccttcttcttcttttttcttggttttgttttgtagtttttcagcagctggagatcgattatgtgatcggggagagtcacaaggagcttatgcccaattcgtctggctccgcggcaattatccggatctttggggtcaccagggaaggttagtcggaatgagtaaaggaaaattgaaattttcacagcgaacaaacagaaagtagttttcgtctctcatttaattcttattacgattggtcatattcttttctccatttcccggtagtctatcagtgttgctgggggtcttaacttatttaaattactgaatttggaactagaatttgcagttctagtgtctaatttcttcatagttcacgtaatttgtcatgattttatctgattttatcagcatacgatataccttttccatggttttttggagcctgaatttcagtatctatatagatatacagttcatccactttttacaaagcatggtcaaattttgaataaccttctctcaggatgctgattttttctcaactctctagtcgtttgtggcatggaaagtaatcaaatagttgtatcagactcagcctatggaaagttagttgttccatagggtcctcaaaagtgaaaaaactaatattcaaactagttgttccatagactcagcccatggcatttggaaactgtttgttatgatctgtacctccataaaagtacttagatgcattatggtagcattacgataaaactatcataagcttttctgataattataaatccacaacaagatatcttttgtgtatcactcttttggaggatatttaaataatgggcattgtggtactactttgtttggtatctaggagtttgaaaacaattatgccacagttattttaggaatataatgacgctcttgggactatgctccatgggaattaaagaatttctgtattgtttattggttaggacatagtgtttgctgcaatgttcatggatttgagccatatttctacatcagctgtcctccaggaatgggacccgacgatatctctcgttttcatcaggttctcgaggtatgtttggtttactatttgatcaatatgtgtttttgtggattcgagcctgcctgaggtgaaatgttattttttaatttagttgctttgttcactataatataattgggttttatggtgcatcaggggaggatgagggaggtcaataggaacagtagggttcccaaatttgtccgacgtattgaaatggtgcagaaaaggagcattatgtattatcagctgcagagttcccaccctttccttaaaattgttgttgcattgccaacaatggttaccagctgccgtggtaaaaacctaagatttattaaccgctgttcacccaccctaccatccaataagatgtgttctgtacatttaagtaactatattcaaggaaacatatttatttttatcataatatttatggtggttttccacttaatccatttttctgttccacattatgtaatattatgcttttgtaattttcttttgatatacatgatcacatttgcctattttctaattcttgtatcaggaggtctgatcttaataagtttattttacttttcttctctaacaaacttcattcttaattataacaaaacttatattttcttacttcactttcaacattttcttataaaaatgacaggtattcttgacagaggcatacaaattgaaggttttggcatgaagaattttgtgacatatgaaagcaatgttctttttgctctccgtttcatggttgattgcaacattgttggaggaaattggatagaagttcctgctggaaaatataagaaaacagcaaagaacctgtcttattgccagttagagtttgattgcttgtatcctcatgcatgagagggcatgatatctcccctttaatagatgcttcctgaaagccttattcaacttgattctattctcctctggagatatttgtttccttaacaactattcttaggtactcagagttgaccagccatatcccagaaggtgaattctcaaaaatggctccgttccgcatattgagttttgacattgagtgtgctggtcgtaaaggccattttcctgagccaacccatgatcctgttatccaggtttaatacttaattccttaccatcgttatttaaataaaaatttgttgccatgtggtagtgattttgtttctcttttttttttttttttgtgtaaataataatagttatttctttctaggtggccaatcttgttactttgcaaggagaggatcagccatttgtccgtaatgttatgacccttaagtcatgttcgccaatagttggtgtcgatgtgatgtcatttgaaacagaaagagaagtcttgctagcttggagggtatcttttctgtcatgatagtatttctattttaccttacaaaaatgtgcccccttactatttgaggcatgtgcactattagaagtaaaatttttacttcatttgcataagtacaatttatgttcaagaagaattatacgttgtttaacaatctatttacccattggttttttatgttgttaaagatttctacttttcaagaaaatagcaacaacctgaaattttccattcctctcaactttgcatcttattttatttttagtttggatttgttttcttgtttttgataagattttgaatattttgaaaagatatttcatcctttgttgcactctataattaattccaatggatttattatttctgatagaattttcttaaatggttggataaataataaattgttactaaaggaaaatgtatgagggaaagcaagaatgggaaaatctaaaagtcaactgaactcggcaaagccttatatccaaaccactaaaggcctgccacataaagcattttcatcattccactatgcctaagatgttgtttcttagcctcaattcatgttaatctactattattcttgttttggagccttccacctttgaataaatggttccttcttacagatgctgttattggtcattattgtatttcaaaaccttagcatgttttgatccttttcaaagccttattattgttattgttgtatttcaaaaccttattattattattatgggtacatggaattgaacttggaacctttccttttcccccccaaccacttgcctcttgagctagactcactctctcatatttgataagttgtttctattttttttgtttatctttcctgtgttgttgctcatttattaaatttttcattgcaaacataaagctaaatgtttccaaatggtggtatctctaaatgctcacaaattcattattttttattttattttttctcgtttcatatgaaaaattactttactgttggatattagttgggttaacctttcaattgatatataaaatctcaagctgtcaatgcagcctttggtttttatttaagtatatgggaccaaataatttcaaaatattcattatttaagtacatgatatcttgctcttttctattctattgtcttgctcatgatgcacttatcaacagaagtgcacttcctcatgttgtgaatttgtgataaagagataatggattcattacatagtcttgtggcagttgtggttgctttcagtactttccctttcgattcatccaaggagtttgaacgatagaaacttctatgttctccttggatgcatacaaaggagaatatagtaatgcataataggacatcatagattcatggtcctgttctattggagcaaaactgctaaaaaacaaaactaacttcatgtggtgacataatctaatccttatttttgcacatgcttctaatcttgattggtagagtttgtttgtaggactttatccgtgaagtggaccctgatattataattggatataacatctgcaaatttgatttgccatatctcattgaggtaattgaacttcctcttatttgcaaagtgtcgtcatattcaaatctttcaattttatttttaataacattctaatattactgtaattattattagaatatatatgaatatgtgtgtgtgtgtgtatttgttaatacttacactaagatttcctttcttaagtttatgagttagaactgcccaatacttgtgtttgtgataatagctagatataagctaatttactgcacgtgcttctggttcacagagagcttcggtcctcggaatagcagaatttccaatacttggccgcatcaggaatagtcgggtccgtgtcaaagataccactttttcttcaaggttgtgcattactattgttgttgttattattatcttttaggggactgctattattatttgtatcattattattcctataaaaaattgctctgtttacctattaaagaaaaaaaaattattattgttgttgttgctttttaagggactgctattattgttagtatcactaatattgttatcatgttcattattgttatcattttcaccattagtataattatcagaggactgccatatcctataaatgtttttcttcatgtcctagttaacttgttttcatgccatgcttagaattgctagcacaaatagggtttacactattatcattgttattatttttggtgttagtgtttagttttttttttttttttgataggtggtgttagtgtttagtattcttaagtcatgtgttctccctttcttcttcactgaggatagttgtattgaactaaataaaatgaaagatttgaaagtctgattatataaaaaagggtagtagagaggaggaagtttgttctctatcttgaggatgagaggaggaaaaagttggtttgctgttgagtccaaattcttcaaaatctcgttagaggaggttaggggtaagctatcggggagaattgtggagagaagtagtttcttttcttcttggatcaggttcgggaagtctagtttgcataatctattggatggagtgaaagcttattgcagggatgaagttggaaagtggtgtagcaaagtttgggtggaaaatggtggagagttcagattggagcgccattaagacaaggctggcagattcattcatggcatggtaaaattagtggaggctaagagattttctctgtgttttcttgaaggaaggggtctacctggggggtggttggttttggcggaaaagcttcgccaccttggggttgattccctctctgtggttggggcggctcctctctctttcaactctaggactatggaagatgggggtagctcgaatgatgggtcgaaaggctcgtttgcgaatacagttaggaaggctcatggtagattgggtgaagctatttggatttaggtggggtgtgtggaggttcaaaagagaaaggcacaactgaggagatgtttggcggggaggtggagtgagggctttacccttgtcccaaatttgagctctttatatagttgggtggactttaattggcccctgaagaggggagtaaggatttctcaatttggggaagcttttttgctgtttgactttgaggttatttatgatgttgagagagtgtttttaaggggtgtttggaagtttaaggatagggtgttaagcttggattggtggagccctgaggtagggtgctaccaaaaagaggttcatgcagaggaggtgtgggttagagtggtggggcttcctttgcacctctggtatagagggacattaaaaaagataggtgattcttgcgggggctttgtaacaatggatgaggttactgctagtggttagcgaatgcagtgggctagagttctggtgtgttcaaatgggtgcgcaacgtcgaggactttgtagattgttgaaggcttggtaggttatgcaatacaactatagtggtaagtcccaccatggctttctcatgtggaactcaaaggcgaaaccaatggatggaaggaaagggatgagggtgaaggccagtcacatgctgatgagggcgtgaggaggcaaagtttgaatttgcaaaaggaggctgtggatgtgtcatcacctggtggtaggggtagattggtaggtatggtgttggctataatgctagggatggtaggactagatgttgacatgggaggggtttgggtagttggtgagaactgtgtggtggcgtagggggtggattacccctgtgtggaaagtcctttctttggtgtggagtcaggccttaacaagttgaaagatgttaatgagcctgagttcgtggaggccaaatccagtcaaagcccaattcttttagtgaatgggccaagcaatctaggcccgccttgtgtaggcctttcctcttgggattgggatggctctaaaaaggggaaaagggctttcttgtgttttagagatgcagagggcggctttggtgcaaacgtcgatggtgacggggtggtttctcccccaagacaagtcttttcgttgccaatttctctccgattgacgattgatgaagcccttttggaggaaggggtgagttatccatgtcagactcctctttctactcctttaggttttagggctttctcttcttctccttctttgttgggtggatgcgacgcggggtgcttggaggtggcctcgtcgtccagtcgtcagtcggtggaggctagggggaagggtccgcttgtggagttgggcctgttgaagatgactttggatgatgggagtctattggaggtttcgatactcaaaggaaaggaaggcatagctagagaggtcaagacgagctttgataagggaagggactgtgccctaggtaaaaatgatggcacaatagaagagacagggttgttcaacaagttggctaccttttgtaaattcttgggtatgccaacaaagtgctttgaggaggagatttctccttgatagaatcaagggggtaaaaaacacagcaagagaaaaaataaatctgctagttcttgttttgaaagagagcttaagaaattagagtggttggtgaactacaaggggctagatatagaatgtggtaggggtagagaatgttgggcgttatctacagttgtctcatgaagttaagaattctatcttggaacgtaagaggggtcaatgatagtaataaaaggatgattttaaagtctttcattaaaacacaaaaggtaggtttggtctgccttaaggaaaccaaaatccaagaaatgtccatgggggtggttcgcagtctaggaacagaaagattcttagagtggggggtagtgaatgataggggggtagtaggagggattctagtcttttgggataacagagtgctagagccagtgggcatggaattggggttgtattcaatctcttgtcgttttaagagttgtgatgatggttttgtcgagatttttacgagggtgtatgaccctacccttgggagtgttatagaggacttttgggatgaattgggggcaattaggggcttgtggagtgatttgtggtgtattggaggaaactttaatgttgtgagacttctaggtgaacgttttggtgccttcaaactatcttcgacaatgagaaaatttacaaaggtcagtgatgatctccaatcgagggatcttcctttgcttggggggtcttttacttggagtgggggtttgaataatcaagcccattcgaggttagattgcttcttggtatttgagggatgggaaggccattttagttgtgtgttgcaatgtatcttgcctagacccgtgttagaccattgcacgattctgttggatgtgggtgagatgtggaggggttcttctccctttaggtttgagaatatgtgattgaaggaggagggtttcaaggacctagtgaagtcttggtggttggggctcagttttaggggatcctctagtttcattctagtagaaaagttgaaaaaactaaaaggctttttgaagagttggaacattgaggtgttcgacaatgtggttgttagaaagaatttggcccttgcacaagtgggcttttgggattctaaggagttactagggactttatctgttgagaggcatcaagggtagaattttagaaatgggctcttatggaggaaataagttgaagatagaagtcaagtgaagtttgattgaaggagggtgatagaaatactcaatttttttttttcacaaaatggcaaatgcacataggggaatttttttggctaaggtcaagattaatggggtttggtttacaaaggaggctgatatcaaagtgggaattgtccaggatttccaagcccttttgacaaaatctaaggaatggcgccctagtataagtggcatggacttcgaagccttaaataggcaagatgcagtgaagttggaggaaccttttactgagaaagaggtgttcaatacattgttggctctcttaatggggacaaagctccagggccggacggtttttcaatggcattttggtagtgctgttgggatgttgttaaggaaaagtgggattctttagggattttcatgatcagggtagatttgtaaaaaaccttaatgctacttttctagtgttggtgcccaagaaaggagaggctgaagatcttagggatttcaggcctataagtttagtgggtagcctgaataagttattggctaaagtgttggctaataggcttaaaaagatgatcaataaaatggtctccaattttcagaacgcctttgtggaaggtaggcaaatccttgatgctatactaatagcaaatgaagtaattgactcgatgctaaaaagtaataggtctggggtactgtgcaaactggatattgagaagatgtatgatcatgtcaattgagattttttacttgtggtcttggaaaaaatgggttttggctagaggtggattagtcagtgcatttcttctccaagtgtcttgattatagtgaatggtacccctttaggtttcttccaaaactttaggggatttaaggtagggggattcactttctttgtatttgtttgtgcttgctatggagacgcttagttgtctccttagaagggcaagagaagctggctttttgactggtttcaaagtgaatgagaggtggagagggtttggaggttagtcatctactgtttgcaaacgatactttggtgttttgtgaggtctctcatacccaattgatgtacttaagctggttgctatgtggtttgaagtcattttgggtctaaaaattaatttaaccaagagtgagctcattctggtgggtagaacggagaacctagaagagttggctcttgtaataggttgtaaagtgggaatgctctcaaccacttatttggggctcccactaggcgccccttacaattcgttggtggcttgggatgagatggaagaaagattccttaaaagattgacattgtggaaaagacaatatatttccaaaggagggagactaatgttgattagaagtacgttatctagtttgccaatctactttttgtctcttttccggatgccaaggatggtgcggttaaggttggaaaaatttcaaagggacttcctttggggaggcgggaccttaaatagtaagcctcatctgacgaagtgggatatggtttgctttgatagaaggaagaggggcctagatgttaagcgccttcactcgcttaacaaaactctcctttgtaagtggatttggcgctttgctttagaaagggaagccttttggagacaaatcatttatggtaaatacggggaaatggaaaggggttggtgctctaaggaagcgagaggtggctttggtgttgggttatggaagaccataaggaagttgtgggatttagttagttgtaaattgtccttttcggtgggcaatgggaaaaagataaagttttgaaaagataaatggtgcggggatgagctgttaaatgtttcttttcattttttatttgccctttctaattctaaagaagcttgggtggcggagttgtggcagcattctaatgaggggggtggctggaatcctaacttctcgaggcctctaaacgattgggagattgctattgtgaaatgctttttggcaaaactttaagataaagtggtagaaaagggtaaagaggataaagtgtgttgggttgaaacgaagagtgggactttctctgttaaatcactttatgctagcttagagataggaagggtagtgcaattcccttctagtgttgtgtggaatgtgtgggttcctcctaaggtgagtttttttgcataggatgcggtttggggaaaagctttgactcttaaccagcttcaaaggagggggtggtctttggccaatcgttgtttcctatgtttgtctcatgatgagtcaattgaccacatccttttgtattgtggcaagacaagggtgttgtgggagctattattgtctctcttcagagtttattaggtgatgcagtccacagttagagagactctgttaggctaacatggttcctttgttggaagaaaatagaaaaaagtttggagggcgactcctttatacctcttatggacaatttggaaagagagaaatagaagatcttttgagaatgtggaactctcaatccaaaggttgaaatacttgtttttgtgtaacttgttaacttggaccaaactgtttataggtgaaagattgatgtccatagtagattttatttattggttggggtcggtttgagggaggaagtagccttttgttttctcttcctttttttgatgctttttggctgtcgttgtatatatcgtgtgtactttggtgcgtcctttatgacactttttaatacatttctaatttattgatcaaaaataaaaaaggggaatatctaagacactgaaacagattccaattgacaaagtgaatgtttccagtcatgtcacatgtgtagaaacagagcctggatttcggaactagagttctaggaataaaaggaattaagtttctggagggtgataagaagaagatggagaatttttaatgtcaaaacaagatttgtattggcaatgagagaagaatcgaattttggaaacatttttggtgtgatgattcttccttaaggaactctttaaataaatttctgaaaatattatgttcctcaaaatttattatttttacttttaaaaattattttaaaatttaatgatatataaaatatatattttcccgtgaactataaaccggtaacttttcccgttcaatgaccggtctggttctaaaaacattggttcaaattgagacaattagatatgaggtttcatggtatctcaaacttctgtcttcattattactgttgaaacttggaagccttgttgatgttctgtcacacatgctgagggactgaagcacttcataattaaaatgaatgaattttcaattgttctatattccacgtgcgtacctttagttgaacttttgttgattatgttctttctttgaaattatatataaaaaaaattagtattttctaattttctggacttcgagtgtgatgcatcagtagcccatgtggcacttttttgaaattgatttttttcttgaaattgctttgtgtaccctttttttcctctcaaagtaacttatgagaatagtaggcttattgatacttatggttattgcagacagtatggaacaagggagagtaaagaagttactgtggaagggagagtgcaattcgatttgcttcaggttttgttcatcgaccagttttacttaggttatttcctaacatggtttatgagaaatgtattcattgagataaaatgacaaataattattcaattgtctttcttttcttgcaggttatgcaacgggattataaattaagttcttattcactgaattctgtgtctgcacactttctttctgagcaggttagatcaaggcttctcttttgatattctgtcatatcaattttttttaacatttatatcctggaacatttttgaaggatttgagtaatatctttgatgagaagaacaatgcaaaacttccctgtattaaaattgtttcttatgggaaagagagaaccccatacatcttagttgggtcagtcatcaaacttcacatttcaatacagaagtcctagttgcaatcattagcaaacttttgtgagttattataataatctgaaatgcttacttggtgttactttgtgttccatttccttatgtggtgtatgcttgctttgtatttcaaacatgttatatggaaagctatacttgatcaagcttacatacaacctttttccttgtgttgtttagttaatatcatatcactaggagttcaagactcaccagcatgatttttactgtttttttttttttttcctttcttcattcattattgggtcaataataagaatcaaaccctatatctacctctcacccttgggtgtctttagcaccatgctatgatgcatttgggcactaggtttttgtatttcaaacatgttatatggaaagctgtacttgatcaagcttacacaacctttttccttgtgttgtttagtttatatcatatcactagaagttcaagccttaccagtgagatttttactgtctttttttttttttcccctttttcctttcttcattcattttgggtcaataataagaatcaaacactatatctaccttgcccttaggtgtctttagcaccatgctatgatgcatttgggcactagattaatgggcatttttcagtaagctctggatttcatacatcttgtgaacctcagttcactcccacttaatgtttattcgatgggctggccttgtgaacttatccttacattgatgtcatgctcattccattttcaagggcaaggttggtttggaatcccactgtttccagttcttttttccaccttttgtttctgtggaaagcttgccttttggcttacttgtacatttttggatgtaattcccaattacagtatagaattacctcttctcagtttcatcctagtgcagatgcatttatttgagaattgagatatttcatgttaaaagatatgaagtttctggtgcttgaggatgagcttagactttggtgggctcttatgataccaggatcccccaacttcactagtgtacccgtgtcaacatgagtacgggtatgagatccttattggatactccttttttgctttggatctggccatttgattttgattgattgattttttttttattttcacattccttggcaaatacttacattgaaaaaagattttactggtggtgaacttctataaagatattttgagagaaggggggcatgtagaaattggacctctttattcaattactgggtcctgaatatcaaaatggatgccaattctaggaactcacttgaacttcaattgtgaaaagatcaaataaaagttttttctaggcaaaacctctaacaaagctgggaggactgccctatccttcattgcgattcttaacctcgccctgctaagattgaatagcttcaacgttcaccaatcaatttcagttaccgtcccccattgctccacaattttcttcagatgtacctcagaccatagatgaaatggcaacccacgtaattcgatctagcctcctctgaacttcccctctatttccgaattttcctttggcgaccaccttcgcaactagactatattcctttcctcaacctttaaattcctcaaatcatggagaaagatagcttcctttgttgtttcaacgaaaaatactccttttccacctgaaatgggtacaattgttactacgcctttcttccctaagcttctcgccaccaatcttccaacctcaacccaattaacactatcatcattgctttcacacaccacggcttgcgcccacctcccaactggaacgagtccacctcttcctggtccttcaccactgactgctttcgcgtaggaccggtgcagagaacccacgtgattgtgggaacaactttcccctctaaatttccttcctttttcgattgctttcgaagaggggaccaccatcattgaagacaacgcgcttttaaaattctcccaacctctgctcttgttgccctcaggaataatcagaacagaagattttttgttggtagcgaactccgagatccttatgaaccttccgtggttgttgaagccaacttccatcaggtggacactgcattttcccctgaactttctgttgaaacccaggtggctcttcatttcgatggctttcgtcaaatgttcaatcatccacccgactgcatccttttcaaaacctaagacaaaaacagagcctctattgagctttgtaatcgagcaccactttcctccgtgctcgccttccaacctaaccaagaaggtcttcctctccatcaagaccttcaacgtcttcaaagcacttcctcttgaaatcaccattccaactgtcttcttcccttgctagacagcgaaagtgatgaagaactgcagggtcaggctccagtgattgcctaaatggatacttagtgcagctcattgtacattcttacctgaaacactacagatgcatctcattgaatgttgttacccttacccccatattcataagtgttatttccaacctgactatcacagccagacgttttggccatattcttttgctctatttaactctgttagattcattgttattgattgtggttatatatgagcttgtggcagtgaaatgatactttattctctgtttgtttggccagattattgtttttatttttttgatcagaatttggccagattattatgactttgtttttcttcttgcagaaagaggatgtccaccattcaataatatctgatcttcagaatgggaatgcggaaaccaggaggcggcttgctgtatactgtttgaaggtgcatattttgttttggtatatcttgggtttcttcttttcaacttttccaatgtctacttttttcacttcttggaatagcgtctgttttggctttaagagtgatatatccattaaaaggaagagccctattataggagtgttatatgtaagatagtagtcagcttggcttaaagcttgtgctgcataatatttaggattacattatgagcccaaatcctataagcttaaacttttaggctatgtttggttcttggaaaatttgaggaaaaatgcaaaggaaagaaaatagagaggaaaagtaaaaggaaagaaaaagtgaaggaaaataaagaatagagttaaagatgataaattatttttatttactacttcaaactcattttatttattttaactcattaatataaagattaaataattttaaaatgtataagtttctaactaattttaattatatttaattttctttggaatattttataggacaactaaacatgagaaaatcattttccttagcattttttttctttcctttgtactttccgggaaccaaacataacctaaggaaaattggtagtttaacatggtatcaaagccctgtttggtagaagctcatgttttcaaacctcactacgagcctctgtggttagagactgattgtggttgtttacctacgagcttgtgcctcttcacatgtgggtataaggctgcttgtgagggagggtgttatagcgtatgatatacattatgagtccaaatcctacaagcttaagcttttaggaaaattggtagttcaacaaggataaccaataatttagatgccattttttgtgcataagcatcctttttttttcctttcccacttttcaataacaaaagtatgatagtttgtggatacccagacacccggccccctaccctgcttccaacagaaaacatgtggatggaataagcatgtactcctacagtggtatgttccattgtagcaatggaatgactttatgttgccttggttgctaaaatcaacaagttgtcctctttttaattgtattggtgttttaattaaggtgaggcaccataaattctttcttgcccttccataaaagcttttgtcaaactaatttcatatcttgcccataatgcttgtcaaggggggcttgctcatagtcaagggtggcctccctaagcaactcttcttcaaatgtcttaacttataaggcctcttcttgatcttactcttgttctttatttctattgttatgttctcttcctattatatatagattttacaaacataaacatggtttgatatatgtcatttaggcctaggttttattttagattattctatatctggttgtcttgacagtcaaagcatcatacttcttccttcaaacctactgtatacatagtctatttattttgaaacccctctggtacattgtggaaccttcttctatatggttctattttttatttttttcccttctttttagagggcggtgaagggtttttcttaagtggtcagtactatttcgaattcttgcatatagctaacaagtctgcactatttacatcaaattactgtgattaaattattgagaggcattaatttcctaagaaatctttgactatccagtggcttcactcctctagttttgtttataacacactagggcagaccaaaaattgcaacttacatatcttgaatttggagattcactctatttcagcccaacttgacccagtcatgcttggctttgaaaattttaaccaattttgtagtatgattgaagaaatgcttgttgataaattaaaattggttctgccacaccccacaaaatgccaaactggacgaaagaatcttgtgtacatcatcatttttggtcattgactctcaatgaccttatctaaggtccaacagctagtgaatttatacaccaaaacaaatttgattaataatactaggcattatttgacgggatcccatgtttctagttttgtattctagccaatgttttaaaaggctaaaggtagtcttgaggcattttgcaaacatttattttgcaaaataaatgttttagagtgtttgcttgtttttcagtgtatttttctatagtttagctttctttggcgggaggattcctcatccttcttttgtacttatttttatctatcaataaatctttgtgttgtttccaatcaaaaaaaaaaaaaaaagaggcatgatgtaagccttgaagcagagtgaggcatatgcctcaacttaaacaaataaataattaaaattattaaaaaactcataaaattgcaataaaattgaataataaaaaagatataaacttcataatgataagattaattatttttcatagttaatagttctaatttagtttctttttctctcataaaatgtagcttatggtttatttttcattgttaatagttctgattttgttcatttttctctcataaaattcggctctctctcatggttttaccaaatggttttcaacattactgttactatcactaggatccaccaaggagtataatcatatccaattgctatattatcctccccattggtgttaatatccacccattcatctttttctttatccactaattgtagtgatatatttttttatcaatcaataattagattatatatgagatcaaccactagaacaataaacaattcccttatttttgatagataaaaagaaaaatatattacaaagggctgccaaaatagcaacccatggtatacaaaacttatataactaccaccaacaagcaccatgataaactgaagttacaaaataaaaaggaactaagccaacccaccctcaccaagggcccaactaatgtagaaaactaaaaagagttaagaggaatatcatctatacacccttttgcttccgaccaaagacaacaaacaaaagaactttttaaactttggattgagagctcttccccctcaaagggaattttgtttcttgcctttcaaactatccaaaacaagcataatggtccctctctccacaccttcttgtgtttcttgtccacaaacaaaccatgtcaccctaataaagtctctttaactgacgatggtagcacccaagatacccaaaaaagggcaaaaagcaattcctacaagattctcgtctttacgcaatggacaaggatatcgtctatggattcttcctcaacatgacacaaaaagcatctattcgccaaagaccacccccttttttggacttgatccaatgttaaggcttttccccaagtcacctcccaagcgaagaaactgactttaggtagcacccacgccttccatatgtttctctaaggaaaagggattgatgtccccagctccaaagtaatatagagcgacttcaccgtgaacttccctttcttagtcattgtccaaagcgccctatcttccttattctcaatctctctttttaccttccattttaaaagttaattgaaaattaagtaaatctttattttgtaaaaagtttaactaagtagggacccaatacaaaattcatgaaatctatattagaagtccacaaaagcatagagcatagaacccattaaaaatcattgaatattcagacttatgtctcttataaaatatataccaaaaaccccttggatatttgaggcttaggcatcaacaaccttgaggctataggctcaaagttataagccttgatagggtgaggcttaagccttagggctaatttgtagagcctttccttgaggcgagccttgaggcataagcctcaataaccttttaaaacatagactatagcaactcataattttgataggcatattaagggttggttagctatgaatctcacccacttcttttatttttttggcgattttatatttttaacatgattgatgtgaacatatgatcacatatattataactaattgtaagtttgtgttgatacctaactcatcaatattttcttgcaaacttcttactttaggcgattgttctttttgcttgacattcccttacttttgtgcttcttcatagaaaaagctttttttaagacataaacatcatttctttatttttacagtttagaattgaatattatagtagtaaacctgggaacaataagaggcaactaaaaggaagaataaacttagtagtattaagaatggttgataatgacttattttagatggatctgaaataaaattgataatactgaaatatagtccttcgacatgatatttgaacaatatacatataccaagttctaaccaccagaatcgttctcaagtactgagggtggaaaatagttatcattatcaagaaaccttttcatgttaaaatcttttaagtttttgaaaggagaccattgataatttgaataaatcctactaaggggtggggtggggatgttggtgaaggatagaataggtctcaattaaaggtaatgttagggttgtttaagcacgatatacattgtgggccctaatattaaaagcttaaatgtttaggaaaattgattacttaatatggtattcgaaagcttgtaggtctagtttaggtaacacctagggagtgaataagtgtttaaacctttttagcccaaaaattattttttaatcttgtaataatcacttttcaaattagggaaacttaaaaacaatcaaaacaagaatacaaaattttttacttggaaaaccacccacacaatgcgtggatgtataaccattggatcaaagacctctaatcaccaatccactatttgaaatcacaatacacagttttacttgatcgaaaatacccctaagtcttactccctagtatctctgcactttacgtccgcgacataacaccatgtgtgctcctagtggatctaacctaagcgcctaaggggatccaaatctctttctcaagactgagaatgtgaaaacaaacgtcttgacagtttggagtttggagaaggttgcttaggtttttccaatattcacattaaaaagcaaaaccctttttatcatagtgtttataggataaaggtacctaatggactcaaattaggatgtctaaaagaaatccaattataattgtaaaataaatccaattgcaattataaaattaatccagtaacaattgtaaaattagttcaattgcacaaaattggaattctatcatgagcgcttgagcacccttaaaagtttttaaaatcattttaaaaaacatttttgctcaatttaaaaaaccttcagtccactacatttttgccacaagagctttgtcagtgagagcatgtcatctccgacactagaaagggctctcgttgtctctcggctacactcatgcattgaacaaaaagttagtgactgaacttacaacattttttggaaatatatgctagaaaggagaccatgttatataatatagcaagataggatataataaagaaaaatatacaacgagggggtgtgctagcctttcagaaattaaagacttcgtttgtgtataatttttggggttgggctaaaatgtatattgatatggagtcgaattcccttataggtttcttggaatggatagcttccaagtaggggttgggttcttttgttttttggtttagggtttgtagccttgtatactccctgtatgctctttggctttttgccttcgttaatataatcgtttacttatcaaaaaagaaaaaaaaaagaaaaatatacaacaagaaggatgataagcaaagtgagaaaaggcggtttgtgtttataaacaaggagtaaaaaagggagaagaactctaacaaataggagagagtcagaatgggatacttcaaaaatcacatgcacaatgttcccttgatgaatttgtaggattgtcctgtttggattgattgagaagttgatttagtgaagattaaatcacactcttgggtacactcatggattcaacaagaagtttgtgacaacttacaacattttttgaaaatacatactagaaaggagaacatattatataatgtagcaagagataatataataaagaaaatatacaaaaagagtgaataaacaaagaaggatgtggaattcttccaacaagaaggacgataagcaaagtgagaaaaggctaagtactcaatttccaatctagtgaaagaataataaaaggaattcctgagaacaccttggtgacacacatctaaaagttataaggtaatttagtctttccctagcatctctgtcaatctcttcctgtcctaaaaattattgtactcctctcaagccagatagtccatcacatagcaaggatagcccctcttcacaagaatacctctctagtgctcttcctaagccaaaagacactttggaacataattcctaacacacacttttagggcacaaaataaaattcatattgttcttgattattttaagaaatatatgagttaataatctaaaaattgttgataattgttttccttgaagttgcttgttggctcactctcataattttaaaaatatatgagaaaagtaagcaaaattctgtgagcctcaatagtttcatttactcttttctatgtgaaatttgatattaaacctattacataaatatgtcttctctatgctttggaaggaagagttcaaggcaaaaaaaaaaaaaaaaaaaaaaagaatgtaactcatttgctacaccaatgaacattctttctttttcgaattgcaggatgcttatctcccacagcggcttttggacaaactgatgttcatttacaattatgttgagatggctcgtgttacaggtgtccctatctcctttcttctttccagagggcaaagcattaaggttaatcatatacttctttagaactgatgaattattgatgagcgtgcagagggatcgaacttaaggaattacaaaaatcaactattattttcaggtactttctcaacttcttaggaaggcaaaacagaagaaccttgttcttcccaatgtcaaacaagcaggatctgaacaaggaacatatgaaggtgcaactgtaagtttatcattctctctgatacttcataaactcagtcatattaacatttgggacatgacaagaaattaagtatttgggttagtttggattaatttcctataagattttaagttgtattgcagtcctaagtttctgatcctttagattatggttgtttggaaaattgttatagcacatgatgatagaacccaaaaaaaaaatagatttcctataaaaatggggtattagcttctctttttgcattttgacaacattcaattatgtacatcaacttttggttttttatagtttaggttcagtcattttgaatttgtgcttctactggtaaagagatttgctaggcttgtactcttctttccatactgagagggattgaaggctatttaggtaatcatgttgtaggtgctctattttggctactggctcctactatgaagcaaatacagactgtttttgacatttttctagtttataaggaatgctaattaattgatgaaggaaagcctgtgctatgagatctcctttagcaaacacttggagtatgtctattgctttgacattgagggaatttttctaatgtaggttttggaggcgaacgcaggattctatgaaaagccaattgcaacattggattttgcatcactatatccatctatcatgatggcatataatttatgttactgcacactggtgattcttctgatgatttcttttatttgctgcaaaaagctacttttgcacatttagatttgatttgtattatttaaatgaatattggtctgattgagaagcttttatggtaatttattttattttatttttaatggtctctgtcaaggtaacacctgaagatgttcgcaaactcaacctcccaccagaatgtgtcaacaaaactccatccggtgaaatatttgttaaatcaagtttacagaaggttaatttttttatagtaatgtggttaagcgtgtttcctatttgtaaacatgagtgaactttagtagttgttaagttttcgtgaatattatagattcacataaatgagaatatattgcaatttactttcagggcattcttccggaaattcttgaagaactattagctgcccgtaaaagagcaaaagcagatttgaaggttttttttttccatctctctcctaagtatttatgtgggttgtttcttattaaatttttatgttgctgtcagacagtgttattgctttgttatccagctttctatgttactgatggatcctttgtttatgtaattaatggtatttatcaatttttaggaagcaaaggatcctcttgagaaggctgtgctagatggtcgtcagcttgccttgaaggtaaattatgtaactgttaattgtgttatcatttcttgtatgattaattttatagttgtgatgatttagagttatatttgaactagttgtttatcactattcctattgtctcaagttcaatggtttttgttgcttttccagataagtgcgaattctgtatatgggtttacaggagccactgttggccagttaccatgtatagagatatcttctagtgttacaagctatggtaatacatactactattttttggcacttgtactgctcaatatgtaagcttttttctggataagatcagaaatactttgttttttgaatagttcttgatcttatgagttggttatccttgtcaaggtcggcagatgattgagcacacaaaaaaactggtggaagagaaatttacaacacttgggggctatgaacataatgctgaggtataccagatggacttttccttttctgatgttgtacataatttcttaaacaaggtcatgctaggtatggtcaaagttgatttgacatagatttatgggtatatttctcatgtgcataattaacatgctcagtgagcagttcaccaaatttagaacttgcctaaatcaaacaacagttttttgatcagcaagcaagaatatattataaaattcaaagggaaactgtacaccaaggtacataggacgtatacaatgggtatcaaaatgatttgctaaatggagagagaacataaaaaactactccctccctcagttggatcccacccaatctacaaaataaattgaaggcatttgcctaacctaactatgttatttgttaaaacccttccacatatttgtactccttggcatgtcaacacatgcctcacccagagatattttctatagcatagtacaagatgcttcctttagtaggtttgtgagggatcttttggtaaattgctggcctttagtaggtttgtgggaatgcaggttgaaggtcttgaaagcgaaattttggtccttttgcggagattggagtccaggaagaagggcagaagctctcaacatggaaaaaagaaagtttcaaccccaaggtctagatttgagagagaattacccaatcttcaatgttccatcaattatgatttaactacaaggaccagaaaaaggggtgggagctcttcggtgaattttgtgaactttgtggatgaagattagaatcctctcttggaatgtaaggggtttaaatgatgtggataagcagaaaatggtgagagctcttattagttctcagagggcagacctagtttgcttgcaggaaactaaggttcaacatatgtaggattttttgtgagaaagttggggataggcagattctcgaattggaaaagtgtgtattcgagaggtcaatctaggggaattttggttttttgggatgatagagttttggtgcttctagaagtggaaatgagcgtgcactctttctcttgtaggtttaagaattgtgatgaaaatttcatttgggtgttctctggggtgtataggctggtaggcaatgttgataaggaggagttgtgggcagaacttggggacatcaaggggttgtggagtgatctttggtgcattggtggggatttcaatgtggttagatttcctagagaaaggagaaataactcaagaatcccttttgcaatgaagtgttttttagaggtcattgaagagctccatttgagagatttacccctagctcgagggtgttttacctggtgtggtgacccaaatagcagatccttgtctaggagtcactttaatggtctctctcagaagttgttgcctaggcctacgtctgatcttgcccccattcttctagatggagggaggatcaaaagtggtaaatctccctttcattttgagaacatgtggctaagagtggaagggttcaaagatttggtcaagagttggtggacaggctacacttttagtggctcatttagccacatcttagtctgcaagctaaagatgctgaagtaagatctcaaaacctggaacagagaggtgattgagaatgtctcttttaacaaagaggctgccctctttcaaattggttattaggatgccaaagaaagagatttaggactatcccctgaagagtcggaagttacaaaaagggttgtggaggactttagtaagtgggcaagtatggaagagatttcatggaggcagaagtcaccatagttgtggttaagagaaggtgataaaaattcacggtttttctataagatggctaatgcttggaggaggaggaatggaagaaggttgacaagggaagatgaggtaaaggaggaggtggttaactctttctaaagagttttgttagaaattgatgagtagaggccaagtatcaatgggctgccctttttgatactagacattgaagaagcgagggccttggcgaatcctttttcagaagaagagatccttgctaccctttctagtttgtgtggggataaggcgtcgatctagataggttttccatggccttttggcaattttgttgggacattgtaaagtttgaagtgatgggacttttttctgaatttcatcatttgggtctctttgagtgaagcctgaacgctacttttattgtactcattcctaagaaagggggtgcataggatttgagggacttttagcctgttagcttggtgaggagcctttataagcttctagctaaagttttggctaataggttgaaaagagtggttgagagaatagtttcaaattcccaacatgctttcgtggagggtaaacaaatcttggatgcattcctcatagctaatgaaaccttagattcaaggcttaagagctctaaaaaggaggtcatttgcaaattggacattgaaaaggcctatgaccatgtgaattgggagttcttattagcaatcatggaaaaaatgggctttagggccaaatgagttaggtggatgaggtggtgcttttcgattttgattaatggaactcctactgggttctttgctagttttggaggtctgagatagggagacccccttaccccctttcttttcattttggcaatggagttgctttctagtatatcgaagaaggttttggaaagaggcttcattcaagggtcttaactagtgggagaggaggagtgggaaggattgtgtcctatctcctttttgccaatgatactttgattttcagtgactcaaacaaggagcatttagaggcctcgagttcggtgttcatgtggttcgaggcgatctctggcttaaaaatttatttggacaaaagcgagctaatcctagtttgagaggtctctaatatagaggatttggcaagggtgtaggagatggcaagaaggtgaggttttggaaagataggtggtgtggggaggaacctttagctatgacatttccaaagctgttttcaattgccattgataaagaagcttgggtagatcagatgtgggagtaggctagggggactggttgttggaatccaatgtttataagacagataaatgattgggggttaggtgaggtggaagggttgcttagaagattgcaaaggcatctgatcagtgaaggagtagaggatgttatagtttggcagttggggaaggggggggggggggccccttttctatcaaaactttctcccccttagtagggtgttttccgaatgggttccccccaagcttgggggggaatccctgggggccgatgaaagttggcttctttgcttgggaagcagtttgggaaaaaaattctgaccctagattagctaaaaagggggggttggggtcttcccaaaaaatattatttttgtaagggggaaaaggagtcaacaaaccctattccccttccttgtcttaagggaagcctgttatggcccccttttttggctatttttgatgttcaatgggggaggcccttctcaatcaaggatgccccccttaattgggggggggcttttttagggaaaaaaaggaaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctacttagcaagcaatgtcgatgacgagggagagccttggtgaagatatcaacaatttgtaagtcggtggaaatatgtggaagagtgataacacgagcttcaaaggcttcacggatagagtgacagtccacttcaatatgctttgtgcgctcatgatagacaggattggccgtgatctgaatagcgcttgtattatcggcatggagaggtgtaggatcggtctcagaaaaatctaactccgcaagcatacctcgaagccaaatgattttagaacaagcaagagacatcgcccgatactcagattccgtagatgacttagagactctgtcttgcttcttacttttccaagatatcaatgcatctcctaagaacacacaccaaccagtgatggagcgacgggtatccgcacaacctgcccaatcagcatcactataagcagcaaggcgagtagaattgcctgcagggaagaacaagccacgagtagaagtgccttgaacatagcgtatgatcctacgaacagcagccaaatgaagatgacgaggagtctgaaggaactggctgacttgctgtacagcaaaagaaatgtccggtctagtaatggtgagataaacaaggctacccaccaacttcctgtataaattgggatcagcaagtaagtcgccctcctctttgcgaagcttgacatttaattccatgggagtatcaacagaagtagccccttgtagaccagctgtagccaccaagtcactcgcatacttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttttattttgccatttgggaaaggggggggggggggcaccttttctatcaaatctttctactccttagtaggttgttatctgaatggtttccccacaagcttggtgtggaatccttgggtgctgatgaaagttggcttctttgcttgggaagcagtttgggaaaaaaattctgactctagattagctaaaaaggagggattggagtcttccaaagaaatattatttatgtaagggtgaagaggagtcaacaaaccatattctccttcattgtcttaaggtaagcatgttatggcaccttattttggctatttttgatgttcaatgggtgatgccgttctcaatcaaggatgccctcattagttggaggggtgcttttgtagggaagaagaggaagaaggctcggaaaactgcgcctttgtgtctattttggaccttatggaaggagagaaatcaaagagcttttgaagattctgaactaacagaccacgtgattttacgctccttcgtgtacatgtttttagagtgagttagggcacatatagagtctacttctttgtctcttttagatttcatatattggtcgggttgtaagtgaagcgagggtgttggttttttgtctatcccttttttttttggccttgtatacgtcgtgtatactttaggcgcttttaatacaattgctttacttataaaaaaaaatgcttccttggccctgtagtggcatggatgtttttgttggtagtttgaactgtaattttgaattgcgggttgagagagctaaatcccatggggttctagattaaaaatttcatgttcagcacacaccctgaaccttgaagttcatcaattgttttccaaaatcattaggttaaccctagctccctatccattttgctgcttacattattttgtcattacttgaactacatattcccaaacattgataaaaatttttatggttcttgttgtcaaaatattgtatgaatgaccgaataccttggccttgagttttgtatattatatatagactttacaaggatgctttacatggaaagcaaataaaagcaaataaattccagcatgattctagccctatacatgtaaactataatctgtcaaataatatatgctaactgtacaaaataataaatgaagaaataaggaaacaattgtgggctaaaataaggaaagaagggtgggctaaattaaggaaggaagtgtggacaacaagtgtgggctaaaataaggaaggaagtgtgacagcaaagctgtgctttgatagctgtcctttgaccagcaaaactgtcctagtgtggacagcaaagctgtcctttaacaaaagctgtcctttgacagcccccctcaaattgatgcaggttgatcaacaagcatcaatttgctacttagcaagcaatgtcgatgacgagggagagccttggtgaagatatcaacaatttgtaagtcggtggaaatatgtggaagagtgataacacgagcttcaaaggcttcacggatagagtgacagtccacttcaatatgctttgtgcgctcatgatagacaggattggccgtgatctgaatagcgcttgtattatcggcatggagaggtgtaggatcggtctcagaaaaatctaactccgcaagcatacctcgaagccaaatgattttagaacaagcaagagacatcgcccgatactcagattccgtagatgacttagagactctgtcttgcttcttacttttccaagatatcaatgcatctcctaagaacacacaccaaccagtgatggagcgacgggtatccgcacaacctgcccaatcagcatcactataagcagcaaggcgagtagaattgcctgcagggaagaacaagccacgagtagaagtgccttgaacatagcgtatgatcctacgaacagcagccaaatgaagatgacgaggagtctgaaggaactggctgacttgctgtacagcaaaagaaatgtccggtctagtaatggtgagataaacaaggctacccaccaacttcctgtataaattgggatcagcaagtaagtcgccctcctctttgcgaagcttgacatttaattccatgggagtatcaacagaagtagccccttgtagaccagctgtagccaccaagtcactcgcatacttatgttgattgagggaaataccagagggactatgatgcacctcaagaccaagaaaatatgtgagaggtccaagatctttcatatgaaaggactcggagagatgagttttgagctgaccaagtaaagcagaatcggaaccagtgatcacaatatcatcaacataaaccaaaagaacaacaatacccatgtctgatttccgaagaaacaaggaagtgtcatacttgctctgcctgaatgaaaattgtaataaagtggtgcggaatttctcaaaccaggccctcggggcctgtttgagaccataaagagagcgacgaagcttacacacatgtgaagtcggagagggaaacaatcccgggggtggcttcatataaatacactctttaagatccccatgaagaaaagcattttttacatccatctgatgtaatggccaatcactggaagcagcaagagctagaatcgtacgaacagtagtcattttagccacaggagcaaaggtctcttcataattgacaccatattcctgattattcccaagtgcaacaagccgagctttgtaacgatccaaacttccatcagatcggaccttgactgagtaaacccatttgcaacccagaggaacaatagtgggaggacagggctcaatgtcccaagtgtgattggcctctagagcggcaatttcttcctgcatagcttgtcgccaacaatcatgcttagcagcatgtgagtagcatgtgggaatatcaaatttggacaatgcagcagtaagggctgaaatagaattgccagaactggaagagggaaatccatacctattcggaggtacagacactcgagaagagcgacgtactgaaggtgctgcaactgactgcatctggagcgtggtaggatcagatatcgggtgagccaccgaaagagactgtgggcgggagcgtctcgtatacacaatacctggttgaaagcgagaactgactggatgaagatctgagaactgttgctcaaaggagggaaggaccacagtagaagaggatgacacagtagaagaggatacaggaaagaaatgttgattttcaaagaaaataacattcctagaaatacgtgtacgatgtaatgtaggatcatagcaaacaaatcccttttgacacatattatatcccaagaaagcacatcgaacagattgagcagataatttatgtcgctcatggggaggtaaatgaacaaagcatacacaaccaaaaatacgaaggtgatcataactaggttgcttagcaaataagcggaaatagggagactccatatgtaggacttgagaaggtaaacgattaatcaagtgagtagcagtttttagagcctctacccaaaacatggatggaacagaagattctaacaagagagtacgtaccacatctaaaagatgacgatttttgcgttcggcaactccattttgttgaggagtagagggacatgaacgttgatgaataatacccttagaagccaagaatgcttgaaactcagtagacaaatactcaccaccagaatctgtgcgtaatgtcttaatagaagtagaaaattgattgtcaacatacgctaaaaactcagtgaaagtacgaaagacctcagatttagagcgaagaaagtagacccaagtaaatctgctatgatcatcaatgaaagtcacataatatttaaatttctcatgtgaactaaccggggaaggtccccaaacatcactatggataagatcaaagcagtgagatgctctacttgcatgcaaagggaagggaagagttttacttttaccaagtttgcaagaatcacactcaagagataaagaataacgatccttattaccaggtaaatcagagttcaatacatgagataagatttgagtattggg

SEQ ID NO: 39 - DNA, 3714 bp, Vitis vinifera
  source 1..3714: mol_type = other DNA
cacgtgacgcaccagatcacaaaatcactccaaaaactggcgggaatccagtgttcccctccctctctccctccactctcactccgcactcttccatccacccagccatgcctgccaccacagccaccaagaaacgccctcagccgccgcagccgtctccatcgcagcctcagcccaagcaccaagcatcatcctcacccttggcggaagaagacctcgacgaggacgttttcctcgaagaaaccctcatcctcgaggatgagcacatcctccgcgacatcgaggaccgccgggcccttgcctcgcgcctctcaaagtgggctcgtccccctctctcctcagcctacctatcttcgtcccagagcatcgtttttcagcagctggagatcgattatgtgatcggggagagtcacaaggagcttatgcccaattcgtctggctccgcggcaattatccggatctttggggtcaccagggaaggacatagtgtttgctgcaatgttcatggatttgagccatatttctacatcagctgtcctccaggaatgggacccgacgatatctctcgttttcatcaggttctcgaggggaggatgagggaggtcaataggaacagtagggttcccaaatttgtccgacgtattgaaatggtgcagaaaaggagcattatgtattatcagctgcagagttcccaccctttccttaaaattgttgttgcattgccaacaatggttaccagctgccgtggtattcttgacagaggcatacaaattgaaggttttggcatgaagaattttgtgacatatgaaagcaatgttctttttgctctccgtttcatggttgattgcaacattgttggaggaaattggatagaagttcctgctggaaaatataagaaaacagcaaagaacctgtcttattgccagttagagtttgattgcttgtactcagagttgaccagccatatcccagaaggtgaattctcaaaaatggctccgttccgcatattgagttttgacattgagtgtgctggtcgtaaaggccattttcctgagccaacccatgatcctgttatccaggtggccaatcttgttactttgcaaggagaggatcagccatttgtccgtaatgttatgacccttaagtcatgttcgccaatagttggtgtcgatgtgatgtcatttgaaacagaaagagaagtcttgctagcttggagggactttatccgtgaagtggaccctgatattataattggatataacatctgcaaatttgatttgccatatctcattgagagagcttcggtcctcggaatagcagaatttccaatacttggccgcatcaggaatagtcgggtccgtgtcaaagataccactttttcttcaagacagtatggaacaagggagagtaaagaagttactgtggaagggagagtgcaattcgatttgcttcaggttatgcaacgggattataaattaagttcttattcactgaattctgtgtctgcacactttctttctgagcagaaagaggatgtccaccattcaataatatctgatcttcagaatgggaatgcggaaaccaggaggcggcttgctgtatactgtttgaaggatgcttatctcccacagcggcttttggacaaactgatgttcatttacaattatgttgagatggctcgtgttacaggtgtccctatctcctttcttctttccagagggcaaagcattaaggtactttctcaacttcttaggaaggcaaaacagaagaaccttgttcttcccaatgtcaaacaagcaggatctgaacaaggaacatatgaaggtgcaactgttttggaggcgaacgcaggattctatgaaaagccaattgcaacattggattttgcatcactatatccatctatcatgatggcatataatttatgttactgcacactggtaacacctgaagatgttcgcaaactcaacctcccaccagaatgtgtcaacaaaactccatccggtgaaatatttgttaaatcaagtttacagaagggcattcttccggaaattcttgaagaactattagctgcccgtaaaagagcaaaagcagatttgaaggaagcaaaggatcctcttgagaaggctgtgctagatggtcgtcagcttgccttgaagataagtgcgaattctgtatatgggtttacaggagccactgttggccagttaccatgtatagagatatcttctagtgttacaagctatggtcggcagatgattgagcacacaaaaaaactggtggaagagaaatttacaacacttgggggctatgaacataatgctgaggtgatatatggggacacagattcagttatggtgctttttggcgtgtctacagtggaagcagcaatgaacttgggtagagaggcagcagaatatatcagtggaactttcatgaagcccatcaaactagagttcgagaaagtttattatccatatctactgattagcaagaaaagatatgctggtttgttatggacaaacccagacaagttcgacaaaatggatacgaaaggcattgaaactgttcgaagagacaattgtctattggttaaaaatctggtaaaggaatgcctccacaaaatactaattgacagggacattcctggtgcagttcagtatgtcaagaatacaattgcagatcttctcatgaaccgcatggatttgtcacttttggttattactaagggtttgacaaagactggagatgattatgaagtaaaggcagctcatgttgaacttgctgaacgcatgcgcaagcgagatgctgcaactgctccaaatgttggggatcgagtaccatatgttattattaaagcagcaaaaggtgccaaggcttatgaaagatcagaagaccccatctatgtgctggaaaataacataccaatagaccctcaatactacctcgaaaatcaaattagcaagccacttttaagaatatttgaacccattttgaagaatgctagcaaggagcttctccatggaagtcacacaagatctatttccatctcaactccttcaaatagtggcataatgaaatttgcaaagaaacaactcagttgcattggctgcaaagctctaattagtaatacagacagaactctctgctcacactgcaagggaagagaagctgaactttattgtaaaacagtagctaatgtgtctgagttggagatgcttttcggaaggctttggacacagtgccaggagtgccaaggttccctccatcaggatgtgctttgtacaagtcgagattgtccaatattttatcggagaaagaaggcacagaaagacatggctgaagccaagctgcaattagaacggtggaacttctaagattcttctgagggtcactcattttcatcacaagtttggaaagagaaaatagatggtgcaagcttgcttatgagagaaaggtcttttgatgtctgtgcaacatttgcaggttttggtggcccagttacttgacgtatcggtgtagaacatgttgcattgcatgtttgagacgaaggccttcaaactaactgatttgtaaaaaagttggacactacaggatacagggtttgcctgaattttcttatcctgttatgggcgacgattggaggtataaatttatttttaagtggtacaagatttttgccaggaatcacttctatgctctggcttctatcaa

SEQ ID NO: 40 - DNA, 3270 bp, Vitis vinifera
  source 1..3270: mol_type = other DNA
atgcctgccaccacagccaccaagaaacgccctcagccgccgcagccgtctccatcgcagcctcagcccaagcaccaagcatcatcctcacccttggcggaagaagacctcgacgaggacgttttcctcgaagaaaccctcatcctcgaggatgagcacatcctccgcgacatcgaggaccgccgggcccttgcctcgcgcctctcaaagtgggctcgtccccctctctcctcagcctacctatcttcgtcccagagcatcgtttttcagcagctggagatcgattatgtgatcggggagagtcacaaggagcttatgcccaattcgtctggctccgcggcaattatccggatctttggggtcaccagggaaggacatagtgtttgctgcaatgttcatggatttgagccatatttctacatcagctgtcctccaggaatgggacccgacgatatctctcgttttcatcaggttctcgaggggaggatgagggaggtcaataggaacagtagggttcccaaatttgtccgacgtattgaaatggtgcagaaaaggagcattatgtattatcagctgcagagttcccaccctttccttaaaattgttgttgcattgccaacaatggttaccagctgccgtggtattcttgacagaggcatacaaattgaaggttttggcatgaagaattttgtgacatatgaaagcaatgttctttttgctctccgtttcatggttgattgcaacattgttggaggaaattggatagaagttcctgctggaaaatataagaaaacagcaaagaacctgtcttattgccagttagagtttgattgcttgtactcagagttgaccagccatatcccagaaggtgaattctcaaaaatggctccgttccgcatattgagttttgacattgagtgtgctggtcgtaaaggccattttcctgagccaacccatgatcctgttatccaggtggccaatcttgttactttgcaaggagaggatcagccatttgtccgtaatgttatgacccttaagtcatgttcgccaatagttggtgtcgatgtgatgtcatttgaaacagaaagagaagtcttgctagcttggagggactttatccgtgaagtggaccctgatattataattggatataacatctgcaaatttgatttgccatatctcattgagagagcttcggtcctcggaatagcagaatttccaatacttggccgcatcaggaatagtcgggtccgtgtcaaagataccactttttcttcaagacagtatggaacaagggagagtaaagaagttactgtggaagggagagtgcaattcgatttgcttcaggttatgcaacgggattataaattaagttcttattcactgaattctgtgtctgcacactttctttctgagcagaaagaggatgtccaccattcaataatatctgatcttcagaatgggaatgcggaaaccaggaggcggcttgctgtatactgtttgaaggatgcttatctcccacagcggcttttggacaaactgatgttcatttacaattatgttgagatggctcgtgttacaggtgtccctatctcctttcttctttccagagggcaaagcattaaggtactttctcaacttcttaggaaggcaaaacagaagaaccttgttcttcccaatgtcaaacaagcaggatctgaacaaggaacatatgaaggtgcaactgttttggaggcgaacgcaggattctatgaaaagccaattgcaacattggattttgcatcactatatccatctatcatgatggcatataatttatgttactgcacactggtaacacctgaagatgttcgcaaactcaacctcccaccagaatgtgtcaacaaaactccatccggtgaaatatttgttaaatcaagtttacagaagggcattcttccggaaattcttgaagaactattagctgcccgtaaaagagcaaaagcagatttgaaggaagcaaaggatcctcttgagaaggctgtgctagatggtcgtcagcttgccttgaagataagtgcgaattctgtatatgggtttacaggagccactgttggccagttaccatgtatagagatatcttctagtgttacaagctatggtcggcagatgattgagcacacaaaaaaactggtggaagagaaatttacaacacttgggggctatgaacataatgctgaggtgatatatggggacacagattcagttatggtgctttttggcgtgtctacagtggaagcagcaatgaacttgggtagagaggcagcagaatatatcagtggaactttcatgaagcccatcaaactagagttcgagaaagtttattatccatatctactgattagcaagaaaagatatgctggtttgttatggacaaacccagacaagttcgacaaaatggatacgaaaggcattgaaactgttcgaagagacaattgtctattggttaaaaatctggtaaaggaatgcctccacaaaatactaattgacagggacattcctggtgcagttcagtatgtcaagaatacaattgcagatcttctcatgaaccgcatggatttgtcacttttggttattactaagggtttgacaaagactggagatgattatgaagtaaaggcagctcatgttgaacttgctgaacgcatgcgcaagcgagatgctgcaactgctccaaatgttggggatcgagtaccatatgttattattaaagcagcaaaaggtgccaaggcttatgaaagatcagaagaccccatctatgtgctggaaaataacataccaatagaccctcaatactacctcgaaaatcaaattagcaagccacttttaagaatatttgaacccattttgaagaatgctagcaaggagcttctccatggaagtcacacaagatctatttccatctcaactccttcaaatagtggcataatgaaatttgcaaagaaacaactcagttgcattggctgcaaagctctaattagtaatacagacagaactctctgctcacactgcaagggaagagaagctgaactttattgtaaaacagtagctaatgtgtctgagttggagatgcttttcggaaggctttggacacagtgccaggagtgccaaggttccctccatcaggatgtgctttgtacaagtcgagattgtccaatattttatcggagaaagaaggcacagaaagacatggctgaagccaagctgcaattagaacggtggaacttctaa

SEQ ID NO: 41 - protein, 950 aa, Ipomoea batatas
MSSGNPRKRPQSAPAPSPQLQKKQQAAVEEEDVDEDIFLEETLLQYDEDSQVLRELEEQSALAERLRKWKRPQLSQAYLSQSQSVVFQQLEIDYVIGESHKELLPNSSGPAAIIRIFGVTKEGHSVCCHVHGFEPYFYISCPAGMGPDDISRFHQILEDRMREANRNNRVPKFVRRIEIVQKRSIMYYQQQGSQPFLKIVVALPTMVTTCRGILDRGIQIDGLGMKSFMTYESNVLFALRFMIDCGVVGGNWIEDFVREVDPDIIIGYNICNFDLPYLIRRAEALGMVEFPILGRVRNSRVRVKETTFSSRQLGTRESKEITLEGRVQFDLLQKEDVHHSIISDLQNGNSETRRRLAVYCLKDAYLPQRLLDKLMFIYNYVEMARVTGVPISFLLSRGQSIKVLSQLLRKAKEKNLVIPNVKQAGSEQGTYEGATVLEPRAGFYEKPIATLDFASLYPSIMMAYNLCYCTLVTPDDVRKLNLPPECVNKTPSGETFVKSNLQKGILPEILEELLAARKRAKADLKEAKDPLEKAVLDGRQLALKISANSVYGFTGATVGQLPCLEISSSVTSYGRQMIEHTKKLVEDKFKVLGGYQHNAEVIYGDTDSVMVQFGVSTVEEAMNLGREAADFISGTFIKVGAHSLLSLVFCPYHSYIVHVIVSLVLIFNLNPSRVMMVSTVLDIKAYLNTPDMLFIKSVALEEVDFVLGLTKTGDDYEVKAAHVELAERMRKRDPATAPNMGDRVPYVIVKAAKGAKAYEKSEDPIYVLENNIPIDPQYYLENQISKPLLRIFEPILKNASKELLHGSHTRSISISTPSTGGIMKFAKKQLTCIGCKALIRYNSMNQIELSANTAREEKLNFIVDQLQMVGKNYYSLCIPNLNLCLKSEMGRKVAELEKLFGRLWTQCQECQGSLHQDVLCTSRDCPIFYRRKKAQKDMAEAKIQLDRWSF

SEQ ID NO: 42 - DNA, 25115 bp, Ipomoea batatas
  source 1..25115: mol_type = genomic DNA
actccgcctctttctacttcacttgtagagatagttgaatagatgtgatgattctccaacttgaaattcttgtgatttgtttccttgatcggcttcttaatcacacatgatcttccaaataaatctgaatatcacttaataagcttatgaatatcagactgagagtttttcacttgaatagcaatatttcacgtaattcaaccttgtcaaagtgagatgggacaagggtatttataggtggaatttcagacccgttggagggagaatgaaattcaaaacctattgtctagtgtgtaatatccaatgggtagtgttttcttctttttcagaatgtcagtactttctagccgtttgtccacttttgtgcaaggacaaattgtcttttgtccctaaaaaggtgacaatcattaaatgctcattgggattgttgcataagacccaaaatgacattcagttatacccattctcaaggcaatagataagacaacttatcagagaatgtctttgccttggttataaaagagaatgattgaccttaattatcatttctcttaaactcctcgaatcaatcacactcttgattgctcggtcataagtgtttggcccttccaagttttgacctaagattctagtcttcggttcctcaaatcttcgtgtcctcgcgtcttcggctcttcggatggaacactcttcgatcgaagtcttcatgtcttcggtatctaagactcagatccttagtaaataagagatagctaaggattcggttaccttacaagactaagataaaaaacataaactattctaaagcattttcgaaagttaggggtctcttcgtatagtttggtatcatcaaaatcaacaatttggggttcctaacagtctccatcaggctcacttctacaaatttttgttctggaagacacagttggttccatttttgtacaactagatttgtatgggcttgttgatggttcaaaccattgtcatttggagttcgttgtgttgacctcgcgccacacctctggttccgaatgttgcttacacttagtggattcagcaaaaccaatccatcttgagcatgttgatttcttgtttggctgaggatgttctatacttagcaattggatttgagacctctcggttagtttggcttgccatcgaaatagccattggatcaacctcgcatgcccattcgctaagcctactgtctcagttacaaaacctacgccaaggagagtcttcatcagttgattacctagggtgagctcaaatccttgttgagcagttgcggtacaagctggacggctagtgggactcgatgagtagaatcttcacatctttagggggcagtgaccagagtaccgagctttggtgtcatccctaacaaccaaggacactcctctcactatttcacttgttgcaaatttactgaacactcatcaatatttgtttcaagatgaggagctgccaggcatgcccttgtcgccgtctatcgttactatgtcgcttgctgccatgtatgtccagactccatctactgcttggttgtctcatgagggtggttatgaacgagtatggtagcatcgcgggtagcgtagtcaaggccagcttggtggtcgcagcggtgtccgttgtcagctttgcgaggcaccggggcacattaccgtaacctactagtggtggttcattgatccaaccacaacaggagggcaattcatgtatgggcagcatgctgtgggtggtcaatttgtgccttggtagtttgcttcgggtactgttcctcgtgcaaattttgagcatcagactcctacagacaagcttcattccgcagtgttctcaaatgtgtggtatcctgacaccgatgtaaatgcccatgcctcgccacagttagatgatttgcacaatgttgagcattattcttggggcgattctcttcaggttggagatggtgctagtttgtcgatatctaatactggttatctttctatttcttctccattacggtcactgtttctaaataatattttacatgttcctcgcttgcaagcctctttactatcagttcaaaagtttatttctaataatgatgtttattttgaatttcaccataatttttgttgttgtgaaggaccgccttaccaatcaggatcttcttagtagttccagtctagggtgaatgtatcaacttcttgtgtcatctcatgttgctttccggtctgctaaagtgtctcctcaaacgtggcataataggcttggacattatcatcttcgtgctttgcgtcaagtacttcctggttgtcctgttagtgggtcttttcctttatttaataatctatgtactatgtgttagttgggaaagtctgtgcgttttcctcattcccgtgttagtcattctagtaaatcagttttaaatcttgtgtacactgatatttggggtctaaccccgattccttcttttgatgggtattgtcattttgtgatttttgttaatgatttctctcgttttatttggttttatcctatgaagctaaaaattgatgtctattctatcttcacaaattttcatgcttatgttgagaaattatttcctttttcaattaaggctgcttagacttatctcgggggtgaatatcgcaagatgcatgcggtatttgttgttgttggggttgttcatagacagtcttttcctcatactcatgaacaaaatggtcatgttgaacgtagacactaacatatcgtagaaactagacttgctttattagctcatagttatgttcttagtgttatgggtcttatgcgtttgagtctatggtttatttaatcaattgtgtaccttctttggtcacttataatgttagtccctatgcacactcctctttcataaacctccagactgttctatgttgcatgtctttggctgtgagtgatttccatgtcttcggacttataataagcataagatggattatttgtctgtttcctatgtttttcttggatattcttcttattttagtgggtgtaggtgtcttgattgtgcttctggttgtgtccatattgcctgtcatgttcattttaacgaggttgttttcctctttcttagcactgcaggtgatcatcgctctagttcttatgctctgtcgcatagttcagggctgcctttggctagtgctccgtgtactgtagctcctcctaactcttctctgtttgttccatttactgcaagggggtgttgctcaatccattgttgatacacctctgtgtttagctgtcccaccaccagatcctcctattactccatgggcgctagatctttcctctcttaatactaagtcttcgcttgtttcatctcctagatttggtgagtgtgtgcaattgcttcagcctcctcagtgtacgcatcatatagtctttcagcgtacgacttgtgctcaacgtcgtcctcctactgctcttgtggcgtctattgatgatgaggtacccacttgccattctcaagctgttaagtctccagattggcgggttgccatgaatgctgagtttaatgttcttcgtcgaaatcagacttggagattggttcctgctgatgctggtatgaatgttataggctgcaagtgggtatttcaggttaaacgtaagtcagatggtaccattgagcgttataaggcaaggttggtagctaagggttttaatcaggttactggggaggactactctaagacatttagtcctattgtcaagctaaccgccatacacctgttgttgtcctttgcagtttctcggggatggacaatgagacaaataggcgttcataatgcatttctcaatggaaaagtcaaggaggacatctatatgcggcaactgccaggttatgttcatcctactcactcaaatcatgtctataagttagagaaattgttgtatggccttcattgtttatatatgtatctagtgagtcccgtgcatatctgcttgtatatgtagatgatattttgttgttaggcattgaagttgtgtatactgattccgatgctatcttatcccaatgccagtatatgtttgaggttctacgtaaggcatgtgtggagtcttgtaaaccattagctacccctatgtctacttttgtgccaggagttaatgatgatgcatctttgctagataatcccatctcttacatgaagttggttggttccttagcttaatgtatatgtttgttactagatctgatcttttatttgtagttaatcgtttgtgtcagtttatgcatagtcccactcagcctcattatgcttctttgaagcgagttctgtgttatgttaagggtacactagatattggtttgcaaataactggttctttggccacaacagatcatgctttctccgtttctgattggggtggttgtcaaattgacaacaggtctatggatggttttgctatctttcttggttcgaattggcctcatgggtgtctaggaagcagcgcaccattgcccgttcttcaacagaagtagaatacaaaactctacctgatgttgctgcaaaggttgcatggattcaatcttcactccatgagcttagtttatctcttaattctgttcttgtgctttggtgtgataatcttggttcaacttatttgtgtgctaatccagttttcgatgctcaaacaaagcacatagagatagattatcattttgtgcgtgaacgtgtaactgctggtgaactcaaggtgaattttctttcaaccaaggaccaggtagcggatatcttcactaaacctctaccaagaccgcggtttgctgaattacgtgtcaagctaggcattgtaccttgtccatggtctacttaagggggtgtattagtatattatatacttcagaactgtatttttctaaaactattatgtttgtaatcggactctacttatatatattttctctgtaatattcctgtatttagattattgaaaaatacagagacttagtcctttatctaaactcttatcattcctaattacatcatgatccaactcttcattattattatacattagttatattgacataactatataatacaactataacactatataaacttttctagtcataataaacttggacaatctctcttggatgctttggttattggttgatcttcttgaacacctcgacagatggacctatgtgtgtgtggtgacacactgaacatttgtctaatagttcacatggttctgaacaagtttaggcaggaaattcgatgtatttaaagtattattgtgttctacaaaaaaaaaaagtattattgtgccaaattatattaaagatgcctggtttaaggaaaaaataatagtttattctattctatcatttctagcttatatgggcctgggacctgggtctaccaaaccaagcatgcatggtcaacactttacaatacgtgaactaatataactgaaaattgttacacgttaaagcggaaaaaatcgataagaaagtgatgactattaagtattaaactttagaaaaaaaaaatgttgtgggactcaaaattttaaaatctaacacaccctttataaaatttgaactttctatagtgcaattatattaaaaattcatttatcaattatctgtcaaactaaatctataattattatatatcacagatcaaggttcatcttacattgttaaccatggtctaaaaattatgtgtctacagttgacgtatttaggagggtgtattcattcatgacttttgtagacttttaacaacttttaaaatgatagattttaatacactttattgactttcataaattcgttcaaaactttttaaaactttgtaaaactctataaaagtctatatataaaacttgcataaacttttatcaacttttttattttaaaaagtctacaaaagtcattaaaattctaaattgaatacatccttacaaactttcataagcaattcataacaatattaaaccctaaaatttatagttatagaattgttcaaataaaatatttaaaaatataattactttttttttgagaaattaatattttttaaaaaatataattattactagttgcataaaataaaaaaaaattaataatatatataaaaattatatttgatatactgctaggtatcaagcaaagatctattatttgcaataacaatatgggattgctaacaacaacaaggtgcttctttttgtagcctcaaatttctattgcgaacaatagttcactattcgtaatagaatgttgttgaaaagaagaaaagaaaaaatatattacgaataaacatatgaatattatgaaatatatagtaaaaattgacggtgattgataaaaaatttactttgttcatgtatttaggagaaaaagtttttagagaaagtttagaagttgatgtgaaaaatttgtggacaaagtttagaagttagggatagaaatttcaaaagaaaaaaaatcaaaatatacaagcaacaatgaagtaaaaataatattattttgaagttttagacgaagattgtaaagtttaggagagatagaaaaattggtagagtttaggtatgaagaaaatataggataatgaagaaaaaataatttattgaatttaggtataaggtttatgaagtttaaaattttaaataaggaggaaaaaatagagtttagggagagagaaaaaaccttcagagttggtagaaagaaaattttaaaatcttggggttgggggttggatttcaactatttatatttgaaaagaaaaagtatacaaaagtctacaaaagttttaaaattaaaaagtccttataaatttataactttttaaataccacatgacttttaaaaactctttaaaagttttaattgaataccatctaacttgtaaagactctttaaaaatcttgattgaatatcgcatgacttttaaatactctctaaaaatttgaattgaatacctccaaacttttaaagttgatgaaagtctttaaaattctatgaaagtcatgattgaatacaccccctttaatatgtgtacttgcagttaacatttgatgtacctataaatagattgaatgcacataacaagattatgcaaacatataatatgttaacggcagactgaagttatcaagttaatatgttatgtgtttatgttaccatgttaaccgtcaacttcaagtatagaatatgtcaacgaactatatacataatttgaaggttattttaatttgtaccagaatctacaatacaaggtaaactttaatccatggtataatatacccttaattatcaaacaccattatattttttttaaagcttcgtcagattatattatgaacctatgacttctacgcacaattggcacaaagcgcccaacccaaattctatatttgtagcaagaatcaaactcatcctaccactaaaaataagttttaagtgagattttttattcactaaattaatatatatgaaagcaaacaccactctttttagcattatatcaagattccatcaacgaagctagatctgtttgatgcagatatgaaaataacaacaacaacaacaacaacaacaacaacaacaacaacaacaacaataataataataataataataataataataataataataataataataataataataataataataataataataataataataataatgtataattagcagttttttgttaatttgtaagtgttgtgcaccaccctagtatgtaggcagctagggtataggtaggggttatagataattaataatggttgtttctatgtgcttcgagtgatgtaaactattaggcccattccggccatcacctcgatcaccatgtactgatgaatatatattcccgacccatctctttcctttttattttatttctaaaatattcctatttttccatcacattaatgaaacatgcatgttatcatcatgtgcttcaacgttatccaatataaaatctaatatcaaagtttacatatatttgcatgtaataatgttttttgaagagttattttcgagtgaagtcgactaagactcaattaaattaattatatacaaagtactacattagacggaagtttaaaaagacaaatttaatattcaaatcattgaaatggtcgttaattattaatttattatttaagtataaaaaaataaagttaggtttttgtttctaactatagctttgggcagacataatgcagagctagcacttgccatggcggagccataaggtttcattttgggtgccaaaactatgtatttgtaaacaatgacggctattcattcagcaagtgtttcgcgacaaccaaatgtcgacttaattttttcttcacaaatataacattattttggattgtgataaattttttataagttctatgaaaatttgtattttcttatagagatataattaaatttatataattcttgatacaataaggagaagaaaaaaaaaacaaacaaacaaagctagacaattaataaacaaatgatgaaatatttatgcgaactattaaaatgttacatttattttggcacttgcattcatcctatttaattatatgccttacatagataatgacatataaccaaatctgaaagtacccgtacatatgctattgaacacttaatcatgataatcagtatcaaaacatgtcagcatattacgagatgtaatattaatacaatacattattattattattataaaaataaaaatttaaactagctagctcacccacgtactacccctatcacctcttaaaaactcaaatcttattaggaccacttaatattgtgtatagtgtcttagcttgcatctaacgagaaacccacatttaattacaattctattttaagatttatttccaccacaaatcaattatggactcatctcacaacatggatcactgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatttttgtattgaagatacattatttttgtactgtaggtacattatttgatagtatatattaaataatgtaccttcagtacaaaaataatgtaccctaaaataatgtatctacaatacaaaaataatgtaccttcaatacaaaaataatgtattttttatttttggtccacacagccatggtccatgcaataatgattgaatttggtaacttggttttagtgaatagtgagaagatacaaaagaagaagtagaatagaatcttgcggggccttggtgacaacaaaaagccacctttctctataaaagttgatcccactcttcataataaaaagatgacatttttattcagatgcagatcggatcctcctttcccactatcattattatattgtcaaaatcattaaaaagttctaatagatgagaaatgtatgtgtggagtattcaagattgtctcaatgaaatttctcataagcatgcgatgtgtttatggaatttaaaatataagttagcgaaataaaaaaatgtggattataaaaaaaattcattaaagactagcgattgatagagtactaaactttaataattctaggtttttttttaaaagcatactgagaaatataaaataagtttgtgaatattcactaaataggggcataccaatagtactctactataacatccgcaataatagagttttttgtggtttttaaagagtttttgtaggtgtgatttagaaaaaaagaatatggatgggaagagaaagaaaaaaagaagaaaaaaacattaaatttgattttaaaaaaatacaatttgtcgacgcctgtgacgcgcccgcttggcgtatttgtgcttgtcatgcaagtgttttgcgactggaatattaaagttttggatcctactttctgcaataaactcagctcttgcagatgaaacccttctcttctctctcttccttaactctccttcatgttggcattgttaccataaaaaattgtgaataatccacgatgttgagcaactcttccttaactctccttcatgttggcattgttaccataaaaagctctgaataatccacgatgtgatgctcaaggaaataaatatatgttattgtaaaaaataaaatacatttgacaaaagttccacacttcgtatcttctaaggaactcaaaataattattggtccgacaaacacaactcatcagatataaattgtgtttttagtattgctaattaaataataatcatcatcgtactaatttatatttgttttgctaagtcataattctcttaaatgatttgattcaacatctannnnnnnnaaaaagaagaaaaaaacattaaatctgattttaaaaaaaatacaatctgtcaacgcctgtgacgcgcccgcttggcatttggatcccactttctgcaataaactcagctcttgcagatgaaacccttctcttctctctcttccttaactctccttcatgttggcattgttaccataaaagactgaataatccacaatgcgatgctcaagagaataaatatatgttattgtaaaaaataaaatacatttgacaaaagttccacacttcgtatcttctaaggaactaaaaataattattggtccgacaaacacaactcatcagatataaattgtgtttttagtattgctaattaaataataatcatcatcgtactaatttatatttcttttgctaagtcataattctcttaaatgatttgattcaacatctacatgaaatggttatcattaattgtcgcgtacgtgcccttgagaaggaatactaatttataaattaatcccacgagatttattactttatttacaagatttagatgaaaaactttatctaaaaaaaatatgaaataaatttttgattcagaaaaaaatatgaagttttcaaatagtgctagaatgagattacgaatttagatttgaaaggagcgttgcctttttcgtaatatgaaagaaggcaacaagcatgttgtctttgctttcttttgaaagaatcttgactgatatttcgataaaagaaaacgttacaaattaaaatataaaagaacatgaagaaaatgacacaaatctatctatatataaaaacaatatcctcctcccaaactttcccgcccaaagttagtgatattttggtaaaatcacatattttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnattgtggccaattttatttagtccttgccacttttagtccactgttaaaaattttcgtcaaatgaccataaaaaacaagggtattttggtattttcatgctttgatcatctcctttaccttttgcagtggttttccttacacattttcatcaaatgaagaggtccggcaaaagccatgaatttgtaatgtggctcatatggctttcgtcggacctcttaatttgatgaaaatgtgtcaggatgtgtaaggaaaaccactgtaaagggtcaaggagatgaccaaagcataaatagacaaaaaaatcccttgttttggatgaccatttgacgaaaattttaacggtggactaaaagtgacaatgactaaataagactggccgcaatgtgtcaaaaattaatgaagtggactaaaattgacaatgcccaaataacaggaccaaaaatagaaatgactccaagttaaaatataaaagaacatgaagaaaatgacacacatatattataatagcaacggatcacaattaaaattatcaaaatgtatcaataatacatgtaacattattgaatatacaaatacctattaaaactaataattaatcttttctcaaaaaaaaaaagagaaaaaaactaataattaatcaatacattataaaaataacaagaaaattgcgattaatcaatgggggtgtaggcgaataatataattaaatgaaagtggtggaaacaaaatttttctagagagaatagaatgtcggtctgcttcaaattcaatgcttcgaaatccaatattttcatttttatttttatatacttaaaaaaaaaagaaaaaatgaaaagaaaaaaaaggcgaacgaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttggcgctacaaagctatgagactcatcattatcatgtatcattgtgcaacaaatttaaggggccagattcatggtttagaattctcaatttcatcaataaattaaatattctgcatgcccctgcagtgcagggaaatgttatccatcaatatgaatgttttatttgctttagtggtattactccgtatattgttgagactttttacttttagtgccaacctaatatttcactgtgtaataaacgaatagtaattttaataattacggagtatattacttataaaaattagtgtaaaataaataaataaattataaaaatattatataatattctaataatctcttctcataataaagtatttatagtaccttatatcaaataaaaagactacaaacacatctcgtgttagaataaaaaaaaaataccacaattacagagattgaattcattgtgagtatttataaacaataaccgcttacaagttagagtgcaaaatgactttggacacttaagatagagtgcaaaatgactttggatgcctaaaagttagagttgaaaaaaacaaaaaaaaagggaatgtgttgtggcaagatgcatattggattttaataaataaataaaaagaataaaaatataacttgagagccagaaataacagaaaacttgaggcacaaatgctcaaatgtaaaggttggtttactaaccctgaactttcctggttttttttataaacctaactattttagagcgcttgtaaaaatcttagaactgtacgtaatgatcaggtctcatattcagcaacccccatgtcagataatctttctacatcagtacttgagtgtatactagaagctccaccgatctaattggatcttcgcttccgccatgtctttctgcgctttcttccttcgataaaagatgggacagtctcgactgaaccaaaaaaaaaaggtttctttagagataagagcttgcaactactcggcaataataattctcagaataaccagaagttgttaacttcaagcagctaaaaatatagatgtatgcagattctataattacgcaatacgaagactgagtttaaagtagaaagtatatcaaacctggtgcacagcacgtcctgatgaagcgagccttgacactcttggcactgtgtccacaacctcccaaagagcttctctagttcagccactgtaattgattcaattcacacccactattaaacaattataacaccttttctgcccatttcactcttcaaacataggtttaaattgggtatgcatagagagtagtaattcttccctaccatttgcaactgatctacaataaagttcagcttctcttcccttgcagtgtttgcagagagttctatctgattcactggttaaaaccaaaataatcattaattaagtcttttaatctctcttttacacaaggaaaaagatagatttcccttgttaaggtacttgagttataccttattagtgctttgcatccaatgcaagttaattgttttttggcaaatttcattatgccaccggtggaaggggttgaaatggagatcgatctagtatggcttccatgcagaagctctttactggcattcttcaatatgggctcaaatatccttagaagtggctgcacaaaacccacaatcatcaattacatataataaatgagcataaagtaaaattagttatgtggcacacataccttgctaatctgattttcaaggtagtactgaggatctattggaatgttgttttctaagacatatataggatcctctgacttttcataagcctgtacatcatgaattaaccttgattataaacaatgggagggtgaatgttaaactctagtactaaattactaaaattattaccttagcacctttagcagccttaacaatgacataaggtactctatcacccatgtttggagcagtagcaggatctcgctgttaaacacaatgcatgacctttatcaaattcacacagcaggagtgacaaaagatacaaaaagatgccaagaatcttgagttggtatgataaatgggcaagtgactagcaaaggagtgatgtagatgtagacgaaaattatcattgcagacttctctaccaaatatgtttgcatataccttatagggtttctagtctagattttccatgctagtttttgggagtcatgcacatgcataattacagttttagcattcccatgtgtatatcttagtgtttttagtctaatttctccaagccaatgctttaggattaattagcagcagcacacaatagaatctcaggaagcacacaattaaatgctatttcagaagtctggaacactatatttagatgtttacaatataaagggttttaaaccaaaaatgtataaatattcatgtttcaggctaaattagtttcagcagtagtcaagaacagtagaatgttgttagcatttgggttaatcagagtgaaatttgggggactatgggaagaaagaatctaagatggcgaaatcaatgacatatttaaccaaaaagattgaaacaacaagagaggggcaatctgctatttacatttgaaataatgatcgagtagaagtacacctaaccttgcgcatccgctctgcaagttcaacatgtgctgcctttacttcataatcatctcctgtttttgtcaggccctattaaaagtaaaatttgcaattaatgacacaataaaccaattcagtagcatctattattaaaggcattttgatgaccttgtttaattcaagaaaattgcacttacctttgtaataacaagaagagacaaatccatccgattcatgagaagatcagaaatagtatttttaacatattgaacagctccaggaacatctctatcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacccataagagccacaatcagagcatttcactgattaaatacaccaccaacatatagcacgatttgctcactaaaacaaaatccacttcctcaagagccacagacttaataaaaagcatatctggagtatttaggtaggccttaatatcaagcaccgtagaaaccatcattactcttcaatcaaataaggacagggacacagaaaatagcagtggtctattcaggtggcaaaatcgacaccttgaaggattcaaattgaaaattagtacaagcgaaactatgacatggacgatataactatgatatgggcagaatactaaagaaagcaaagagtgagcaccaacttttatgaatgtgccactaatgaaatcagcagcttctctacccaagttcatggcttcttccaccgtagatacaccaaattgcaccatgactgaatctgtatctccatatataacctggtataaaataactgcttactgagacaatattgtaaagggatatctaagtaaaacttctgtactattaataaattcatctatcaagtttaatagtagtattgccaaattaagtactccatcctgtttttattggcaactttgattggacacacaacgtatgaaaatttatgtcaaagtacgcatgggatatttcactagtacccacgtttgcatttaatgcatcaatcttgttcaatgtaatagggattttgaaagattttttattaaatttcctttaaaagatgatagtaaccaattattttggggcatcccaaaaaggtaatgaggccaatacatatggcgggtgggggtgggggggaggaggagataatacctaagctgatcaatttaagtataacattagttggacaatccgtggttcaaagaggaacataatagggtgcacacgtaaaattgaattttcatctttctgcataatttacacatgcataatgctttgtgtttgcatatatggtttcctacagtatgcatagcgcagacattcaaatatgtctagcatgtatgatacatctgacgggataaatttttacaagacaggatgaactaaacaacatcctaaagtatccagtcgaattgtttcaagtcttatgaggcccctatcctctgatgcaacagaagattgacaagcaaggttcaaatgcaattcagtggactgcttccagttattatattattgaatattgataatacctataaaaagttttgtaaccagtaggcttggcaggattaggcgttgcctttgaatcagataaagatattgctaataatatacctcagcattgtgttggtagcccccaagtaccttaaacttgtcttcaacaagtttcttagtgtgttcaatcatctgccgcccttttggaaaaaagggaagatttgtaagatcaagcaaactaacagaaaacatttttcaattgttttataggaatattgtgcaaatcccaatggaaaagactattaaaaatatagtaccatagcttgtaacacttgaagatatttccagacatggtaattgaccaacggtagctcccgtaaacccatatacagaatttgcactaatctgaaaccagaagacatcagatggatcaaactatgtgccactagtttgaatatcagaagtcagattaataattgatgagccagacaaaggaaaataaatagtactagctgatataaataactaaaattgacatggtttagtaggtttaccttcaaagccagttgacgtccatctaaaacagctttctcaagaggatccttcgcttcctgaaatcaaactgtattggttaccttcatggggaaagatgcgagtttgaacaggctaaaagtcttattgcatccaatgaacaatatagtaccactttttcctttttaaaaaaccatatataagtatatgaaacttaaggtcactttttaaaaacttatactgtttaactataaaaattataattataatttgagagctatagagacaagaaaagcattagcaaatatgcatcattatacatattctgttgctgtgcaaaaagcaaacatgcctgccataacaatagaagacagctcattgtttcttaagctgtctccctttacctattttcatagcaagtagcagaatctaccatttctgatttaacaaaactccaatgattatttcatttactctgattacacactattatcattagaagatttcaaagctgtaaatccaataacccagcatatgcagaattttgagcctccatcaaaatttaattttagacctggtcctatgtacatatgctattcaatgctaagcagtgaagttccataagaggttaagaagtagctgtttggacagtaatgatggagttaagttagattccaagggcaatacaaaaaataacaaaattgggatttgcataaaaccatacatggaactcctgcagacaagtaatacagggagtaaaataagccacaaagatagttaacacctttaaatccgctttggcccttttacgagctgccagtagttcttcaagaatttccgggagtattccctgaataagagaacttaagtaattatgctcagataatagtagcttccttatgaatggcaatagaattataaacataaaagaaacaatgattttataagtcctcgagaacaaaataaaaagaaaattttggattcaaagttaaccttctgtaaatttgacttgacaaatgtttcaccagatggagtcttgtttacacactccggggggaggttgagtttgcggacatcatcaggagttacctagacagagattatagggaaaaggaaaataattaagcttcaaaacatctagaatcattctattgtacattaatgcttatagcccaaccatcatatggagcaatttgctaggcactgtttagataaaaagtctgaccaacttattgtattgatgaaattattaaagtaatggctgacttaaaatcttcaatcttgtgaagaacatttaattttactatcacacaataattattttggcattcataatggcaaggaactatttagttagagaatgttttcagttcttcatttctaattttccaaaatgttttatgtttccaatttataaaatacaaagccatttgaaatccaattgtttcttcatatagaaatacgaataacagttgtaagtgataattgtatcgagtggtggggtgtaggaagctaggagtggaggtacatcagtaggtgggtggtaaggaaaatatgcaggtagtggtggatggaaggaagcaatgatggtaatgaaggtgaaggtggtgggtgagggtacgtagatagggggttgaaagttcataagtaatgataatggtgatgatgataagtaaaataatgtatgtttttgtatttgggaactgatttcaaattcctttgttgattttggaaagaaaactctgtttttcatttttcttgaaaactagagattttttccaagtactcattagaccctaaatattgggtaaagcaaggaggggaactacagcccagagaaaaatattttaccagtgtgcagtaacacaaattatatgccatcatgattgatggatatagagatgcaaaatccagagtagcaatgggtttctcatagaacccagcacggggctccaaaacctgtgacataaagatagatatcactgttataaaaactcgccaacaaaaaaggtatacaacgttagaggaacattcttttctattggggctatgtttcacattcaagaagtctagtgatatgtgtatctaaagctttacacaactagatcaatgttgagggtaaggtttgtggattaaacaaaatatatatataattaccgttgcaccctcatatgttccttgctcagatccagcttgtttgacatttgggatgacaagatttttttcttttgccttcctcaaaagttgagaaagcacctgaaaatagtggtttacaattcaaatctccaacacagccacatctttgatctatcaccacaaaagaatactaggaaaagtgagttcataagaaaataaccttaatgctctgcccccgagacaggaggaatgaaataggaacgcctgtgactctagccatttccacatagttgtaaatgaacattaatttatccaaaagccgctgagggagataagcatcctgcaaattaatgaggcattggcaaaccatagtactatacacacacttaagtagattaagctcatcaacatctcagatcaaagtaacttaaaacttcaaaggaaaactaaaccttaccctgcaaaactcattaaagagtaaaagtacaactgaatttacagatcatacttacctttaaacaatatacggcaagtcgcctccttgtttctgagttaccattttgaagatcagatattatagaatgatgtacatcctctttctgcaagcaaaataatagctttagatcacaaatgaagccaacaggaagaagattttttgagagatgtcagtgaaaaataaacctaaggagtataagaatattaaactgaaactgttgcaagctaagcagttggagcagaagtagaagttcaactgtgtcaatttttgaattgttaatggcaaagagtaaacagcaagggagcaatacaaggaaaatagccctcccaaaaccaaaatatacaaatataaaacctctagaatatatcaaaaatatataaagcgaagaacaggcaaattggacgaaaatgaagcaaaattcatgtcagatagttttattatcgttgtcccaattatggtcagaatggtagttactttctagaactctcgatcaaatccacatccaaaattgtgaaaagaacaattagtaaaaaggcaaaaagcacaagccttgcctgttcattaagaaaatgtgctgagacagaattaagtgaataagagctcaatttataatccctctgtacaacctgcaagggaagcaaaatatatcactcagatatctatgatattataaaacagggaaaactatgaatcagaaactcttccaatgaatttgtgtgtgtaaacctgaagtaaatcaaattgaaccctcccttccaaagtaatttctttactttcccttgttccgagttgcctgaaatagtatgctcatgaatcagtctagttagcaagcttcacagtcaaaatctctgagaaaaatacatgatatcatattcatcagcttaaaaaaaatacggagtatcaaaaatatccatgcaggaataccatttgggatttacaaactgaggaggtgttaaggttacatgttgcattcagagcatttcaagtacagaaatagtaacaaacaaacctctacattcatataaaaatgccatacattcataaaaatagtgatttcacattgcaatgagcatttcaagtatagaaatggtaacaaaaagacctctaccattcacaaggtcatatagatgtaagaatgtcacaagactgattaataaaaataatggtctaaatatgggattatatgtgggtcttgctgtatataggtaccactgaattatgtatctccaactcatctaaaagcttgaaatgtcacataacaccagggaatttttactgtctttatatatgatagaccaagcctattatgcccctccagtagttgcacaggattgatctcagtgaatcacaattacatgcaagtaatggccctgtaggtttaaagaaggtgagccccttataacaagatatataattatatatgagtaaatattgaatcccaggatcaatatctaattggagattgtagtaactcattttaatttcagtatgaaaatatattgggtggtgtatgatcaagatgctagatgtcaacgcgaggaaataacagcacaaaatatcagagaagttaaataaagttgggcattgcaaaggaaatttttgaacagtgaacaatcagcatgaaaatataatgggaccttgaagaaaatgttgtttctttcacacggaccctgctgtttcgtactcgtccaagaataggaaactctaccatccctaaggcctcagctctctacaacccagataatgcatctcagtagatcaataaaatataaaatataatttaaaaaacacattgaaattaatggaaacattgacaaggtggaaaattaaaacattatccactaacccgaatgagatatggcaaatcaaagttgcagatgttatacccaattataatatcagggtcaacttcccgtacaaagtcctacagttcaggcaaattttgagtcaaaaacaaaaacaaaagagacaaaaacagtacacaaataagcattccttgttagcaatgtttatgaatacttatcacaacgacaacaacaacagaaaaaaaagcatatcaacttttaagccacagcatttaataattccttataaccggaacaaatttgtgaaataatcaagagagacacattagcacatcacagtgctgacgcacaggcaggtagaaattagatcagattttccaactgcttgagtttaattggaaagcacacaaaagcaaaacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaaagtcagaatagctaaagaacaatcataaaggaaaacaaattaaaaacatgaccaaatgtcaacaaaagaaagcaggaacatatttggattttatgaaggctcatgcatgaggatacaggcaatcaaactctagttgggaataggagatattctttgatgatttcttgtactttccagctggaacttcaatccaatttccaccaacaacgccacaatcaatcataaagcgaagagcgaagagaacattactttcatatgtcataaaactcttcataccaagtccatcaatttgaatacccctatcaagtatacctgccaatttgttatgcagacaaactaaaattaatacaagttttacatttatatattgtggaaaaataaaggggaaaaacctgatgatcattataaattgcaacacaagtttagtgaacccaatgatgtgtgtaaacaattatgaattatccaacttaattgaacccacttcttttaattagggctacaccctataaggctataaataacaaagtatctgaccaatgtaacacagatgagaacgatttggaaatctcttatgaagaatttgcactagtcgcataacatttgacctactatatatttttagtaatgagctcccttgtcattgaatccttgagccaaccaacctatggaatactattccttctattttatctatatattaaccaaacaatggaatagcaattattttccattctttatctattccatcctttatggaaccatattcctatttcctttgttttcattccatgaaccaaatgtgccatgaatgatcaaccacagctatttcaataacttgctatttccacttgtggcagagaggcacatcatcccacggaattcttgtctccatcgtgatcattttctctgctgccacagtgcaaaagtcagctctcgccctcttagagattcatcttactaagagtttaagaaaagtacaaaaaataaaataaaataaaaggacttatgctatggttgtactagggtcacttaaaatgaatgagtgttgagaacaaataactttaatggataaccatttggactccgaaagtacaagttaagaagttattcatttaccacagggatgtttaagttccagaaaaaaaacaagaggtttacagtagagacaaataaataggaaagtccaaatcatgcataccacggcatgtggtaaccattgttggcaatgccactacaattttaagaaaaggttgagaaccctgctgctggtaatacataatacttctcttctggactatttcaatgcgacggacaaacttaggaaccctattgttcctatttgcctctctcattctatcctgcagtaatagcataaagatataaccaaatattaaatccaagagaggaaaaaaatgattggaagaaggtataaccacataaatatgtatgacaaaaaaataaatgtgcatacctcaagaatttgatgaaaacgagatatgtcatcaggacccattcctgcagggcaactgatatagaaatatggttcaaatccatgtacatgacaacaaacactgtgacctgaccaacaccaacaagagatattccttagaagttagaaagatagaaaggacatagagagtcatcagtcatgcactggatgcagaaaatcttcagttctattcaggttccttttagactcaattaccagacaaaaccccatggacatctagagattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttattacaattacttttagaaatgcatcttatattgatgaattagtgttctcagccagatggataaagtcttaacacatagagtacagaagtttctatctttctccatgaatgaatacaaaattattacattaccaaattacaacccaaacaacaaaattagctgcattttgtttcgttacttatgagcatgcattttatgagtgtaagcaacacagagagttgcacaaaccttccttagtaactccaaatattcttataatggcagcaggaccagaagaattaggcagcaattctttatggctctccccaatcacgtagtcaatttccaattgctgaaatactatgaaagacacaaatcacatcaatttcagaaaactggccattttaaaacacaatacaatgtgcaaatttattacaataattcactcaccaaacaccacacgttaaatggataattttatgaaaaaaaaaaaaaaaagttttaaaaatcttattttgacatgaaaactgaaattaacaaattgatcattttcagagctttcgaaaccctaggaatcaatggtataaaaccataaaatcaatgaattgtaacaaaaattgatcaaacatggaaatgctagacgcatttcaaatagaagaaaggaaccaagctacgaaactataacactcctaaagttgacttcaactttcactcacctacgctttgcgactgagagaggtaagcttgcgaaagctgaggccgcttccatttgcggagacgctcggcgagggcggattgctcctcgagctcgcgtaggacctgggagtcctcgtcgtactgaagcaaggtttcttcgaggaagatgtcttcatcgacgtcctcctcttccacggcagcctgttgtttcttctgcagctgcggcgacggtgccggcgccgactgtggtcgcttccgaggattgccgctgctcat

SEQ ID NO: 43 - DNA, 2853 bp, Ipomoea batatas
  source 1..2853: mol_type = other DNA
atgagcagcggcaatcctcggaagcgaccacagtcggcgccggcaccgtcgccgcagctgcagaagaaacaacaggctgccgtggaagaggaggacgtcgatgaagacatcttcctcgaagaaaccttgcttcagtacgacgaggactcccaggtcctacgcgagctcgaggagcaatccgccctcgccgagcgtctccgcaaatggaagcggcctcagctttcgcaagcttacctctctcagtcgcaaagcgtagtatttcagcaattggaaattgactacgtgattggggagagccataaagaattgctgcctaattcttctggtcctgctgccattataagaatatttggagttactaaggaaggtcacagtgtttgttgtcatgtacatggatttgaaccatatttctatatcagttgccctgcaggaatgggtcctgatgacatatctcgttttcatcaaattcttgaggatagaatgagagaggcaaataggaacaatagggttcctaagtttgtccgtcgcattgaaatagtccagaagagaagtattatgtattaccagcagcagggttctcaaccttttcttaaaattgtagtggcattgccaacaatggttaccacatgccgtggtatacttgataggggtattcaaattgatggacttggtatgaagagttttatgacatatgaaagtaatgttctcttcgctcttcgctttatgattgattgtggcgttgttggtggaaattggattgaagactttgtacgggaagttgaccctgatattataattgggtataacatctgcaactttgatttgccatatctcattcggagagctgaggccttagggatggtagagtttcctattcttggacgagtacgaaacagcagggtccgtgtgaaagaaacaacattttcttcaaggcaactcggaacaagggaaagtaaagaaattactttggaagggagggttcaatttgatttacttcagaaagaggatgtacatcattctataatatctgatcttcaaaatggtaactcagaaacaaggaggcgacttgccgtatattgtttaaaggatgcttatctccctcagcggcttttggataaattaatgttcatttacaactatgtggaaatggctagagtcacaggcgttcctatttcattcctcctgtctcgggggcagagcattaaggtgctttctcaacttttgaggaaggcaaaagaaaaaaatcttgtcatcccaaatgtcaaacaagctggatctgagcaaggaacatatgagggtgcaacggttttggagccccgtgctgggttctatgagaaacccattgctactctggattttgcatctctatatccatcaatcatgatggcatataatttgtgttactgcacactggtaactcctgatgatgtccgcaaactcaacctccccccggagtgtgtaaacaagactccatctggtgaaacatttgtcaagtcaaatttacagaagggaatactcccggaaattcttgaagaactactggcagctcgtaaaagggccaaagcggatttaaaggaagcgaaggatcctcttgagaaagctgttttagatggacgtcaactggctttgaagattagtgcaaattctgtatatgggtttacgggagctaccgttggtcaattaccatgtctggaaatatcttcaagtgttacaagctatgggcggcagatgattgaacacactaagaaacttgttgaagacaagtttaaggtacttgggggctaccaacacaatgctgaggttatatatggagatacagattcagtcatggtgcaatttggtgtatctacggtggaagaagccatgaacttgggtagagaagctgctgatttcattagtggcacattcataaaagttggtgctcactctttgctttctttagtattctgcccatatcatagttatatcgtccatgtcatagtttcgcttgtactaattttcaatttgaatccttcaagagtaatgatggtttctacggtgcttgatattaaggcctacctaaatactccagatatgctttttattaagtctgtggctcttgaggaagtggattttgttttaggcctgacaaaaacaggagatgattatgaagtaaaggcagcacatgttgaacttgcagagcggatgcgcaagcgagatcctgctactgctccaaacatgggtgatagagtaccttatgtcattgttaaggctgctaaaggtgctaaggcttatgaaaagtcagaggatcctatatatgtcttagaaaacaacattccaatagatcctcagtactaccttgaaaatcagattagcaagccacttctaaggatatttgagcccatattgaagaatgccagtaaagagcttctgcatggaagccatactagatcgatctccatttcaaccccttccaccggtggcataatgaaatttgccaaaaaacaattaacttgcattggatgcaaagcactaataaggtataactcaatgaatcagatagaactctctgcaaacactgcaagggaagagaagctgaactttattgtagatcagttgcaaatggtagggaagaattactactctctatgcatacccaatttaaacctatgtttgaagagtgaaatgggcagaaaagtggctgaactagagaagctctttgggaggttgtggacacagtgccaagagtgtcaaggctcgcttcatcaggacgtgctgtgcaccagtcgagactgtcccatcttttatcgaaggaagaaagcgcagaaagacatggcggaagcgaagatccaattagatcggtggagcttctag

SEQ ID NO: 44 - DNA, 2853 bp, Ipomoea batatas; identical to SEQ ID NO: 43
  source 1..2853: mol_type = other DNA

SEQ ID NO: 45 - protein, 1086 aa, Solanum chilense
MNSGNSKKRSAAATPSPQQQQKHHAAMEEEDLDEDVFLEETLLQYEEDSQVLRELEESNSIKERLQKWKRPSLSQAYIAQTQSISFQQLEIDYIIAESHKELLPNSSGQAAILRIFGVTKEGNSVCCHVHGFEPYFYISCPPGMGPDDISRFHQILEGRMREANRNSNAPKFVRRIEVVQKRSIMYYQQQASQPFLKIVVALPTMVTSCRGILDRGIQIDGLGMKSFLTYESNVLFALRFMIDCNIVGGNWIEVPVGKYKKAARNLSYCQLEFDCLYSDLISHSPEGEFSKMAPFRILSFDIECAGRKGHFPEPTQDPVIQVANLVTLQGQDQPLIRNVMTLKSCSPIVGVDIMSFDTEKEVLRAWRDLIREVDPDIIIGYNICNFDLPYLIRRAEVLGMAEFPVLGRIRNSRVRVKDATFSSRQHGTRESKEITIEGRVQFDLFQAVQRDYKLSSYSLNSVSAHFLNEQKEDVHHSIISDLQNGNAETRRRLAVYCLKDAYLPQRLLDKLMFIYNYVEMARVTGVPISFLLSRGQSIKVLSQLLRKAKQKDLVIPNAKQAGSEQGTFEGATVLEARAGFYEKPIATLDFASLYPSIMMAYNLCYCTLVTPEEVRKLNLPPDCVNKTPSGETFVKSTLQKGILPEILEELLTARKRAKADLKEATDPRVKAVLDGRQLALKISANSVYGFTGATVGQLPCLEISSSVTSYGRQMIEHTKKLVEDKFTVLGGYEHNAEVIYGDTDSVMVQFGVPTVEEAMKLGREAADYISGTFIKPIKLEFEKIYYPYLLISKKRYAGLLWTNPDKHDKMDAKGIETVRRDNCLLVKNLVTECLYKILVDRDVPGAVQYVKNTISDLLLNRMDLSLLVITKGLTKTGDDYEVKTAHVELAERMRKRDAATAPSIGDRVPYVIIKAAKGAKAYEKSEDPIYVLENNIPIDPQYYLENQISKPLLRIFEPILKNASKELLHGDHTRSIAVPTPSNSGIMRFAKKQLTCIGCKTPLSGSDRTICKHCKGREAELYCRTVANVAELENLFGKLWTQCQECQGSLHQDVLCTSRDCPIFYRRKKAQKDMAEAKTQLDRWNF

SEQ ID NO: 46 - DNA, 20827 bp, Solanum chilense
  source 1..20827: mol_type = genomic DNA
ttaaaaattccaccggtctaattgagttttagcttcagccatgtctttctgagccttcttcctccgatagaagattgggcagtctcgactggtcaaaatcaagataatttagaagaatgaagggggagaggaataactagcaatagccacgcatctatcagtaaaagtagaaagcaactcgaacctagtgcatagcacatcctgatggagagagccttggcattcttggcattgtgtccatagcttcccaaatagattctctagttcagccactgcatttaatacagttataaacaattagcaagcaacgttcaagcagttcatagctctgagtccttccctagagacatcatctaaatagatatgacacgccatctcctccttacgaaattaaaaaggagatgtacaaaagaacatgagatgaattcctagcataccattagccacggttctgcaataaagttcggcctctcttcctttgcagtgcttgcagattgttcgatcagagccactggatgaaggagcaaataagttaataagctgttctgcaacctttactatagcttcttccaaaaaagaagaaaacaatacatccgctcatcatctcttgtcaaagagagatgcacaaacagagacagagaggcgcaaaattgttagcaagcttgtgggagccttcacactgctatgatctcctaggcccacatcacttttaaccttttttgcccaagaaattaaaaaaaaaataaaaaattgggacagatgtttatttcataaagcaagttcattgatttggaggtttaataaaaattggtaaaaaaaggcccatcaacaagaagcttttatagaaaatggcaccatatccattttgaaacatattaactactgcctaactctaaggtagtcttttatctcaacattcctacttactattcatgtcagtgacaaagcatgaaattcaagtatagctcctacaatggctcttccaagataaattacaatttcaacagaaacttaaaactaacaaccaatatacagcttagagaaggtacactgatgctcctaggttggaagtacaaatcctatgtcagtaactcacaggtcgaggagaattttgagaggaaggagggattctccttagaaatgaaaaacaatcttatcttgtaaaacatatagggacaagaaagacaatagagcacctgagtggtgttttgcatccaatgcaagtgagttgcttcttggcaaatctcataataccactgtttgaaggggtcggaactgcgatggatcttgtgtgatctccatgaagaagctctttacttgcattcttcagtattggctcaaaaatcctcagaagtggctgatataaaacacttccgttaaaacagttacaatgagcatgttaattaaacgaaaaaatcgagctatactgaccttactaatttgattttcaagatagtattgaggatctataggtatgttgttctctaacacgtatatagggtcttctgacttttcatatgcctgcttgtcataatataaccatgatttgacacaagcacacaaaatgatgagattataatcacatactaaatcagttggatgattaccttggccccttttgcagctttaatgattacgtaaggtactctatcaccaatgctaggtgctgtagcagcatctcgctgtcattgaaaagaaaggttttcatcaatttcattttttatggtgcgctaaagccataagaatctcagaaatgtggtttctgcaatagttaaatgggcagctcattctgccacttgaatacaatgttacaagagagcacgcaaaacacgacactatataaaatgtaccttaaaatgaatgtcaattagacaacctgttccttttaccttctattgtctttgggttgaacaaattatatcttataaaatcacatgaaaccattacaggaaagaattaacaagagggaaaaggaaagtaagccaacaaaacaaaaggatagctatgagccttttagtaactttatattctgatcaacctttttgacgagtggaactatatattctaataaacgtgcttctcttattcttttcttcgataaagagtgaggtctgggccagtttgcacactcttcgactaattccacaaggtactgcataactttgtcaaacaagcttggacagaggtaagaatagggcttttgcctccactgagatttaaacatgataccttgtgattctcaacccacttcattgacctctagggcacacccttgagggtacacttttctatttccttctgagtacaatgaaccttctaatagtcaggaaatcctcctatcgtgagttataaggttgccctcactttatatggtcttgaaaattctcccctcgtgcgctttaaggttgtggtctccttatataggcctccagaatcctcatgagctaacttttggtattgagctaaccccaaaacgttcatttcttcacaattatattaatgagaaacaattgtgaaaccgcgcttacaagtctgactcggatgctatgtaaaggagtctgaacacctaatccaaatccaatgaaggtcaagcatatgttctaacatagctcatagggtaacaaaatgatggagctattaatcttccaacacatttgattaacagcaaatatgaggaatactggaatccttggcacaaaaaccagcacagaacttcagaaaagttgagctactgacaacaagataacttaaatatagtgacgtctggtttatccttatttagagtacttgaaaattaaaccatcaaaaatttggatgacagcagccttgccaccagaaataaggaacaaattcaatacataatttagatgggaaactcaacagcttaatcgaacaaatatcaagcagcatatacttgtgtgcaagatgccacgcattgatcttcacagacaaaggacaaaagtaactgaacccctcttgaaggaatctacttatatttttaaaaagatatgcaaataaattgagtttaaccaaccttgcgcattcgctctgcaagttcaacatgtgctgtctttacttcataatcatctccagtttttgtcagaccctattaaccctcagaagttagacaatgctaatcacataaactaacaattccaaaatattcaaacaagttgcaagaaataaatgccccacattttcctgaataagagtattctcaccttagtaataactaaaagagacagatccatgcggttcagaagaagatctgagatagtattcttgacgtactgaacagcaccaggaacatctctgtctactagtattttataaagacactcggttacaaggttcttgactaagagacaattatccctccgaacagtttcaatccctggtgaaaaagggtagaaatgtaacaatttatcaaacacaatatgcacctaagaaatgaacaatttgaatacagtaagaatcattaaatttgaggtaacacatttgatttgctaatttttcaaggataatatgaaaaaaattaaaggttggcattgaagggtgagagaagttccaagaatatgctgaaagatgaaaaggataggcctcgacagagctcacctttagcatccattttgtcatgtttgtccggatttgtccaaagaaggccagcatatctcttcttgctaatcagcaaatatgggtaataaatcttctcaaattctagttttattggctgaaaatcaaatatgccaccagcagcagaaggaaaaaatgaaaaataaattttagtagaagagattaaaaaaatacataggtacactgctgagaacacatctcaatatttaaacattttaggattctctttattagcaaggatctcacaagttagcaagtttcctcagtagaataccataaaccatgataaatttacaaaattgcatcaagtggaataagtatatcaatactacttatcaagaagaagttgaactgatcgagcacagtaagtttactagtttctgatgctttttcacttcttcctcttcatgatatataccctaataacaaatatgctataaccatgaaaggccaaaaccacctaatgggcatcatcaaatagtaataactgaccaactgattagagaatataaaaacttcatgaataactgtagttgtggcatgtaagcaggctcatacttgtctcaagagaaagtgatagtcaaagaagaacaaagaaactgcactgaagtgatcatctgaagaagtgtttagatgctactggttttttaagaaaaggttggtttagatacaattaaatctatcgaaggttaggacgcactttgataaatgtcccgctaatgtaatcagcagcttctctgcccaatttcatagcctcttccactgtaggcacaccaaactgcaccatcacggaatctgtatctccatatataacctggcatcacaaaacaatcacaattagaggacaattttttgataaagttaataatctagtgctaaccaaccagtgacactctgacaacacccataagatattaattgggaatctataacatctataaaaacttcagataatgtacctcagcgttgtgttcatagcccccaagtactgtaaacttgtcttccacaagcttttttgtgtgttcaatcatctgtcgacctaggaagaacaaacaagttcaaaaagcaagtacttgaattggctttatatacaacaaagttccaatagttcagtgtcatgtaataaagtagctagtaataattgcattaccatagctagtaacacttgaagatatttctaaacatggtaattgaccaacagtagctccagtaaacccatatactgaatttgcactgatctgaaaaccagaagacatcagtagttattgacctgactaaattctttatagttctaaaatcattgtagtaccaataataaagagaaccaaattcatatatctgccaagaaaaagtgaaagaacacagttataaagttggatattgactaaaaatgcatgaattaccttcaaagctagctgacggccatctaagacagctttgactcgagggtctgtggcttcctgaaacccaaaatatattgtcataattcaaagttcagaggataactcaaatatttatctacacatcatctgttagatatgcaaaaaatggtgagtagacaaatactcgagtaaaaacatcgggcttctgataggcagttcaccagcttcccacaacttacttgggcatgtcattggggcacatacacaaggagcaagaaatttggaatggcataatttaaaagacaaaaaatcggccaattggaaagctcagtatatctctttgggagggacagtcacttcaataaactctgtgttggactctcttccaactttttttttgaagaaggtaacttgtattataaacaaaaaggaatactcagaaagtattccagtaatacttacaacaaaattactgggaacgtcctcttcttcttgaagatatccgtgtttacaccaaaaaaagaaaaggactagacagttcatcttcagtttctgcagagaacaactcttgttttctaaacatctttggttccttcctattcatatagtccaccaaatgcaagctgggacaatattccaactcatgttatgtctttatttcctatgcttgccaaagtggaagaaagacttgacaaactgagaagagatttcttgtggatagggaataaagaagggaaggatttgcatttagtcaaatggcagactgcgcagttacataaacaatccggtggacttggaatcagaatcttggggttgcaaaatagatgcctttggtcaaaatggatatggaggttcaacactgaggaacaagccctgtggagggaggtgatagtcaacaagtacgggttgaatggaaattgggtctccagctctgtgaatagcacttatggggtgtcaagcttggagaaaaatcagaaatttttggcctgacctaagtagacaaatagtctacaagatgggaaatgggactaaaatcatattctgtaaggacaattggaatggaaatgagacactgatggtcttatttccagatcagtttccttgtgtaccaattggtctactcatggatggaatattgtgtttagaacgcctttaaatgactggaagattgggagaatggctgagctattacatgttctgaacgggttcaatggggttactggcagaaaaaaattcaaccatatggaaacactctagatatggaagcctttctgttaacaagctattattaaggaggtgaaggagcattcaggtggtaagcttggcccgtggaagcagatttggaggaacaaggtgccaaccaagataaagtgtctcttgtttggttgccagaagagcatgctccacacatgagaaattgaaaaggaggagtttctatattgccttttggtgctctgagactgaggaaaccaacaaccacttgttcctccattgcataaagacaacacaattgtggaaccttattctcagactaacacgcacagaatgggttatgcctgagcatacaacagatctacttagttgctggatcagcagaggggggagaaaatctaagaaaaaattggtggagtacaataccatcatgcatttggtggtctatttggaagaaagaaatagcagatgttttgaaaacaaggctaattcaatagagaaagtcaaatggaactgcctgactactttatatttttggtgtaaagaggaagggatagaagaagctgcacaaattttggatattattggcacttcgtaatcaattagcctttttgttttttctcttgttgggtacaaatttttggaggtctccactctccagcatatccttaatgctgaggaatacaacaatatcttctcaaaaagataggcagttcacttcaagaaactggattctttatactaatttgatgcatcagaaaattaagaaacaagatgcagttgacagaagctggatatggagccatggtggtggcaaattgagaactctcatggatcaaacgacaatttaaagaaccgaagtttagagagattggtaaaatggaacttttgtatgctagaatcaagctacataacatattgcatcaaatattgataaatatgacataacatgagaggaacatgttggtttaataatttaatacagatgcacatctattcaaatgcaaggcataaggataaaaagaatctacatagcatgaaattacactgtgtccctcagaagctctttgcaagtagttatggattttacaatcttgccataacatatcaggttacaagagggtccagagccactttaaaagtttccctgacaatcaaacttctgtagaaaaaatgatttgtccattttaacggcaagaaaaaaactagtcatctgcattgtccaatagcatctaatactagtgctgtggaaataagatgctcagtgctaggatgtgaatatgtaaaaatatacaccaagtgcttggttgcagtagtgtaatactgacaaatgaaaacactacaaattccttttatcacttccttaaaaaggaaacttttagagccattaaatctagtgaaaggagggcagtattgagtgtaactatatacaaacaaaaggaaatctcaagtaaacagttaaaagccctacttcacaatcaacaatcaagtatcctgatttaaccaaaaaaaaaacagattttggattctctatgcttagttcttgtctcttacccatagaattaaaacatttaagatatatgcatatgccgactgcatattatcatagatgtggaggattgatgtccttgattaaacccaaggctagttggatcaaaacccaataaatccagaatgattctttcaaacattagatggaaccaaattccaaagactgaacacaacaacaacatacccaatgtaatcccataagtagggtctggagagggtaaggtgtaacttaccccgacctttgtggggtagaggttgtatccgatagatcctcggttcaaaaggaaagttaatcagagaagggttctaagaaaaaaaatgacaacaaaaaacaagatacaaagcaaaatgaggcaacagatagtagaaaaatataaaagaacaaaaatatagtgaacacctttaaatctgcttttgctcttttacgagcagtcaatagttcttcaagaatttccggaagtattccctgacaaaaaacaattaaattatcataatcgaaacaagaacataaataccgggattaaaccagcagttatatttaccttctgtaaggttgacttgacaaacgtttcaccagaaggggtcttgttgacacaatctggaggaaggttcagtttgcgaacctcttcaggagttacctagacatgagggaagaaaaccaaaacttagaaccggaagcattaacgtgaatatcttaatgctcagcacttaattctaaggtttcaccaacatcaattgaacactccggtaaaaaagatatatcatggtcaagactcaaacaagaacacttactagcgtgcagtaacataaattatatgccatcatgattgatggatatagagatgcaaaatccaatgttgcaattggcttctcatagaaaccagcccgtgcctccaaaacctgtgaccagatattttgaaaggaaaaatacaaaagaatcaagacttacagttattacctctaacctaagaaatcaacatcaaaggatataaaaggtgtgtgatagtcagatttgatatcctccaaacatacatgttcattttttataactatggtgtccagggccaaacttgagcgcacctcaactaatactcgaaacatatgtttaacaagagtaatataccaaatataaagcttggcacaacaggagctttacaagtgctaacttttctttacaagataaaatatccagctatatgatgggtgttacttattttcttaccgtagccccctcaaacgttccttgttcagatccagcctgttttgcatttggtatgacaagatccttttgcttcgccttccttaaaagttgagaaagcacctgaagatagaggagattagaataattaagattaagaccttcattaattgcaaataaatgagaccttacttaattttccactagaggaaaatgaagcaagtataggactaaccttaatgctttgccctcttgagagaagaaaagatataggtacaccagtgactcgagccatctccacatagttataaatgaacattaatttgtccaaaagccgttgtgggagataagcatcctgtaacggaactaaagcattcagttttcttgctaagtgagactgaatattcacatcagaaaattaaatttcatttgaactcatctagaataccagttactatgttctataactcattggccaaaactactcattgctcgttatctacatgaaacaatcattacgtccaactgtatttgggaaaggatgacagaagtgtatcaacatttggcataataaaaatcaaacaatattctatctttattgaccactagatcacacccttaatgttgtaacagtaacttctttgatgaaaacaatgaagttatatttggtttctttacatgcaaacatctttcgagtggaacattctacaccaaaaggctgtctctataagcataaagaaatcaaaactaaatattcttggagcagaattttacatatatcccctatctaaagaaacttgtatgcatgtctaaatctaaacaaaactaaaagaattaactattccatgaaataagcaacactcagcacacaatcaaatccaattcaataatttccttcaaccggaacagaactatacacctttagacaatatacagcaagccggcgcctcgtttctgcattgccattttgaagatctgatataattgaatgatggacatcctctttctgcaggcatagcagtagtattagaaaaaaattacttgctctaaacaagacaagtatattcaataggatataaggataggggaaacaagacaaatgaacagattatgggcataaaagaacataaaggaaaacaaaggtataaaagaccctccatctcaattttcagatagcaacaaatacaatgatatgtttataataaaggtcaaaacaatatgattaagatgtcttcctttctgccccactggataaagatctgaaattaaacttcacgagtaatttaataaaaataatagtgcaccggcgcagagaacttaattaaagaatgcactggagctagaatgaccaactatgttgtatggggcagagtgtttccagtcaagaattctcacattagaagactcaagttgtggaaatgaggacactaagatgatgtgtgggcttactaggagagataaaattagaaatgatgatatttgggtcaaggtggaggagaagacagattcgagactaagatgattcaaacatgtgaagaggagatgtacggatgccccaatgcagaggtgtgagaagttgggtttgaatggtttcaagagaggtagaactagaccaaagaatacgggggagagatgattagactggacatgaaataacttcaacttaccgaggacatgaccttagataggagtctatggaagacaaaaattagtgtagaaggctattaagtacttgagttttgtctcacttgtgcttgagtactagtagttgtagcagtattcttatagtttcttgttctccgatttctatcagtatatgttatttcttgtactttgattatagtattgtcttgttatagatattatttctttctttttttcctccaaaatgttttgtcatgctatctttagtatttttgccgtggcttcttcacttcagttattttttttccccagattgcgttgttatgtttttttcttgagccgagagtatcggaattaacttctctacctcccaagaaaggagtaaggtatgtgtaacagtaccattcacagaccccacttgtggggcatgttgttgttgttccacttgagctagaaagacggtgaacaatgggcaattaatgaaaatcctcacttcttcgatgctatgtgtgcttctcgttgctgttcatggctactatgaacaataatttaattgaataggatctcctcatttgatctttgattcctggctctccctattttattcctaaaagagttcttccatttgacattaccattttccaaaaaaaaaagagctcttccatttgtttttctccatatcaacacgagacataaataggggcgagttcccccaactcctccttgctcactggaatcttctctctccaactggtcatgcttccatagcatctaggcagccccgaatagacactaaagcagaaataagatacttaaaattattaaatttaagatgttttgttcaatggtacttgacatttttatgtgatagctggtagcgagaccatgttgaagttacctttttttctacataaatgtgtgttgtggtgactggaaaaggatcagagtttagcaagtgcacttgtatttaaaaataaatcacaactttttacacatgtttcttgcacaaaattgagtatttcttctgttctttactgaactcaaggtcgcaatcacagattttttctttacgaactcatgtagatcaaatacagctcaagccattaatttcacaattaacatgcccaaatcaaaatagaaagctagcaagtagactcgtaatccttcaagctaacaaaccctgtacacaaagaaactacaaagcattaccgcaagagctactaatgttatacttaaatatacatgagcaatggccctaatggaatcactcaacttagttatcaacataagtagagacgtgatatggatggcatccctccaactttaatcagaggtcacagaaactcctggtaggtaacgcttctcctatggtgggtcaactcatgactctgattaaatagaccagtgagttttaagatatcgggtacataaagcaaaaataaagagcattgaagtgtttgaacataaacaagagtgcatgtagggtcatcgctcaaaatcatgtaaaaatttattggcagaaggtacatggttgtgggcagatgtagcagtatgtgagttgtgacaagaaactatggatagaagagcaaaactaacctgctcattcagaaaatgtgctgagacagaatttagtgagtaagaacttagcttataatccctctgcacagcctgcaatacaagagaataacagatttactagcattcaactgttttcctaagtaaagttctttccacagcaattagaacagcttattgatacagccataatgaagaaaacctgaaataaatcaaattgaactctcccttctattgttatttctttactttcccttgtcccatgctgtctgcaacacaaaacgcatacatcagtccacagcttctcaagtttttttttttataaagtcagcttcactaaacgcaaaacttaattgtacttgtcacccttgggatatttttttcttttagactttgatgatcacaaataggagtggcaaaattagcacatgaaaagcacgacactcccaagttgacccactcattattaactcagaccatcaaaaaagttggttgatagcccaaattgacccatgaaacattttgtcaaaaaatattctcataacatttttttctgacatatatatagctctagtaaagggaaaaacagtttaataggtaatcttttttaataaaagtacaaacaaggaaattaaatcttagcaagaattgggtgggttgggttatgaaccgctgtttagtccatttcagcgcaaataaatattgggctagttatttcattaacccaggccattttgacccatccatttgacaaccctaatcacacttagatgaagattaaatcaatttcagcatagcctcagggaaatttccttttacgatcacaatcaactagttgaagactgatctttgagtttcaaccgagtgaatttttgggaaatgtttgaattaagaagtgctgcaactccacctttggagatgaaacaatgctctaggaggaaaaatatgggtgataccaattactccattttactgtcctattacaaggagaaacattacaaaggttctgataagcagcggctaattggaacatgttttagattagtatataatcccatatttcagaaaccacatacagtgatggaacataaatccaaaactgaacaggccagtggttagatacaaatatagtcaacatacaccagacggaaaaaaatgccattttgtattacctacagataaagtagaaacctaaaagtacttctaaacaaacagaagataacaaacaaaaagatataatatgaccttgaagaaaaggttgcatcctttacacgaaccctgctgtttctgatgcgtccaagcacaggaaattctgccatccctaagacttcagctctctgataagaagataaaatgccttcagtagttcaacattacacatggaaaaacaaacatgacctagcctttcccaatctcttttgggaagggaagaaacatttttttccacatcagttccaccatcagcatcacagctaaaccacaatttattttaattttttattaaatgtggtgtccgcgagcttgcacacatctcaactaattctatgggatacctgtcacatcccactagcaacatgaaccaagtaactctgtccactaaggctagaacaaatggaaagaaatcaccttatgtttttgtctctgctgagacctcatagttctttcccacttcattgaccacaatggtgtagctaaacaacgatttatcatgtagtgctttctttcacttctgcctacggactttttatagtcctcaactcttaatccagttaataattaagaaacaatacttttggctactatattaacaaacaaattgttaagcttgcccagcaaatcttttttttttttttttgatgaagttgcccagcaaatcttattcactttagctctcatattattgttttggagccattatttaaacatataactacataagaagcttgctagtcaaaatacaataccttttaccaatcaaaaaagggaaaacagaagctcattacccggatgagatatggcaaatcaaagttgcagatgttatatccaattattatatcagggtccacctcccggataaggtcctgcaagtcaaaagaaagttaaaagaaaacatgaattggtcattggtattactaatagtagagacatctttttcttacaaaatattgttttcaggaaagtattctggaatagttttatgcaattgtgcaaactaatgtatgagactgaaatggcatatccaaacactctatgttcatattaactatgagaaagtcccaccacttcctcagcaccaatcaaccaagattcatatgccaagtaagggaagtgtaaatagcccatgaaagtttatttccgtcattatttctactcctttttccataatatttaacttctaacttgtccaaattagtctgttcagcctgcttcactcttcggcttgtgcattacaatctggagcataaaacaacagggacaatgtgaaacactcacgctaaaagaaaccgtgtaaaagggtaagtgccatcacatgaagccaaattgaaagaatcaactgtcacacacaaaatctctgctcgtctgtccaacaaacccccattccgccaccacacaaacgaagagaaaaggtggaaactgtatccaattaaaatacaaatttcatgcaaaaaatattaccctccaagcccgcaatacttctttttctgtgtcaaaggacataatatcaaccccaactatcggtgagcatgacttaagggtcatcacattccgaattaatggctgatcctgtccctgcaaggtaaccagattggccacctagaatgaagttaaaatcttgtcagcaacaggaaagtaaacaacatatattttccagatatatagaataaattgggactaacttgaataacaggatcttgtgtaggctccggaaagtggcctttacgaccagcacattcaatatcgaaactcaatatgcgaaatggagccatcttcgaaaattctccttcaggagaatggcttatcaagtcagaatagctaaaagaaaatgttaaggaagttccagatgatgatccctaatggatagaaagaatgcactcttaggtcatatattcatcatgcattaggatacagacaatcaaactctaattgacaataggagagatttcttgcagctttcttgtatttcccaactggaacttcaatccagttgccaccaacaatattgcagtcaatcatgaagcgaagagcaaaaagaacattactttcataggtaagaaagctcttcataccaagcccgtcaatttgaatacctctatcgagaatacctgccccattttacatttcaatcaggtgggaaaagcaggaaaaggaatcggtaatagaatcaattctataaaatcaatcagaagttaaacattttatcgcaattctgttgcatccatatgttgaaaaacataacccaaattctgtcgaacatattgttctccagatttcagcaagtaaactcaagagagaatattaaggagaaatttcaccagaagcataagatgcatatatattattttatatgactcaatgtcaaggaatacattaaattatcatggcaagactggcaagcaaaatgctatattaaatatagcacaagataaatgggtaatgccaaggacagtctaagaacttattagagctggactggatgtaaaatgagaaatgaacagagggcgttggagtacctttcagctatgttaggtggtttggaaggaaaagccagaacttcaagatctaaactgcagctgtttgcagtttgaaacatgaatgacttagtctgttaatttctgttgtaatcttttccttcttttaaatgaaataagaaggataccattaacaaaaagcatcaagaagatgcaagcattacaaagagattatctgctcccaggaaggcaaaaaaagctgtcacaagttcacaactatcaacatagatctacggagctgataaattctaggaaagactctacagtaattacagagaactggttgactcaactaagaagactgatcaaatacctagcttcagagaagaaatgggagttgggatgccatcaataaaccttttgttcctctctatccaatccgtaagcaccacaaaatacaagtaggaatcaaagacatatctttttaatggacttcccaactttctaggttctgttgcaatcttaaaatatattatatatatatgaggacgcaatgatagagttcactagttgctaaatacaggggtaaaattaacccaactgaacaacataagaaaacatctactttccagcactgtcttacatcttgatatttctatcaataacacttgtagtgtgtgtgctgtgtccgaggcgtgtgccacaagcacaaaagataagacaaaatcaaaaaagcaccttcatcagaaagatacagtataattgaataagtgaaacagataatataccacggcaactggtaaccattgttggcaatgccactacaatcttgagaaaaggctgagaagcctgctgttggtaatacatgatgcttcttttctgaactacttcaatgcgacggacaaacttaggggcattgctgttcctattcgcctctctcatcctaccctacaccaaccaacaagaataatatagcaactgcagcatttgccattaataaaccaaactggtgaagaattcaatcatagcatgcttatgcaaacaagcaggtcaacatacctcaagaatttggtgaaaacgggagatatcgtcagggcccattccaggagggcagctgatatagaagtatggttcaaatccatgtacatgacaacagacgctatttcctgaccagggcataaacagacatttcttatatgtggaagacaagctacgtcaacctcaataaatacattaatgatggattccaaggtattgtgttccttctgaactcactcctcaccctaccaagtacaacctccacttaagcttttcattttcagttctaatatctggtgtaatatgatagcatagaaaatactaaaaaagaagctataattttcttctataaaggaaatggcacaagaacctctttaaacaaatagaacagaaacacaaaaggaaaatattacaactagagaatccactaagatgcaacttcaactcaaatttgtaccaatacattatacacagtcaagagaaaatctatgtttgaaactgtggttggtatcagcgtgcctcacaagcttgcagccctcagaacgctctaattgtttttacatttacgaaaaaacagcttcaccgattaaaaaaaacaactcccatttcatgacgtgcatttatacctctaggagactactatattacttggaatgccagaataaatcatctttctagatcaatagcaccatatctacaacacaacaacaagaattgcaaatgcatacgacaacagcattaaaaattgacaaaccttcctttgtgaccccaaagattcttaaaatggcagcctgaccagaagaattaggtagcaattctttatggctctctgcaataatgtagtcaatttccagctgctgaaacgctgagaaaggaccaccaaatttcaacagaactttcagaatgtgattgtgaaagttagagctaaatgaagtaattgatgtatttcgaaacatttttgttttacctaaaaatccaacttcttgttgttcactaaattattataagatccacatgtacacattgtcctcaactcaataaacaaacacttttcttggataagtcaaaattattcattaataaacacctgaatagttatcgaaagataataagtacaagacgtgcattgtgactctacttgagtcatacattgtccaccaccaagtctgacaatatataaacttgcttaataggattccatttgagcacaggaattaaaataatttgttgctcataccaagagcacgaaaagacttcaaaatttttggtttctttttgcttggtaaagagaaggcttcaaaattttcggagcaacaacaggtttctattttttgtcaatacctacacggcatcagacttatgtcaatatgagcaatccaaaagtaggtacttttcttcctattctgccttcaaagttcaaaattttattttaggttttctgataattaataaatatgagagtcataaaacaatgaaaacaaaaaaaaatttgaattatactccttaattgagtacagaacaatttatttacatgaagacaaaccaaggaaagaataaaatataagaatattccaaattaattaaacataaataaaaagaactaccaaaataagaatcttgaacttaaactttaaagtaaaatggacttcgaattcctataataaacgaaaatggcttccacatatacacacttagattgtgtttggtatggaggaaaatattttctatttttccatgtcatgttggtcaaaatttatggaaaatattttcccattcctaaaatatgaagaaattgaatttcgtagtgggcataagaaaacaagttccacaaataagaaacccacatattttcttggaaaacaattttcaaaaaatattttccttaccaaatacacatttagactagcttagcttaactgtttaaaaaaaaataaaaagtacataaaagggactaaatgtcaattgcaaaaaacccgagttaaagagcattgcccagatgtagcttttagacaagtgtaggctcactcatttctttttaagtcaaaactatatacaatgtaaagagagagggacttaaatttagtgttcgttgaccttgagaagcacacgacaaagtcctaagcgaagttctatggagatgttgggaggtgagaggtgtccatgtagttaaccttagagtgattaagaacatgtacggtggaaccaagactcgggtaaggacagtgagaggggactcaaagcacttctctaaaatgattgactgaaacgcaaagcagagttaacgtgaagctggagtttcggagacggaccctggagtctaaagatttgaagttgagtaggaataagacagaattcatggactgcaagttcaatgatgtgattaatgaagcagatggggatgtaaggcttgatactaaagtcatccccaagagaggaagctacaagtacttggggttagtaatccaaggcagtggggagattaacgatggtgtcacacatcgtatcaaagtgggatggatgaaatggaggctcacatttggtgttttgtgtgataagtatatgccgttaagactaaaaggtaagttctacagagtggtggttaggtcaactatgttgtttggccagagtgttggctaattaagaaagtttgtccataagatgaaagtagtacatatgaggatgctgagatgaatgtgtaaggacatcatgagagataatattaagaataaaaagattcaggacaacaacatgatgggtgtgaaccccgtggtggataagatgtagaaagggaggttgagatagttcagacatatatagaggaaaaacacagttgcctcggtatggaggtgagaggtttggtcatgatggcatgaggaggagaagaggtaggactaagaaattttgaggaaaggtaattagacaagacatattgcaactttaacttagcaaagacaggactcctgataggagggtgtggaggttgagtatcatggtagagggatagtaggttgccaagcaattttctttttcatccttcagtattactctcatttttaagtttgttttatcataaatctctagtatttcttgtttgtttcttccatcgtgtttattttactatcttgttgttattattgtttacttgtcatctcttcttgaaccgagggtctataggaaacagtctccctacccttacaaggatttgtgtagactcgaccctctccccagacccaccttgcgagattatattgggtattttgttgttgttatatatacttatatactgaagttcgtaaactatattataattacatcaaatgcacgctctaatatcaaattaaatcaaataaaacatctgcacacattgttgaaaagagcagccggtccgtgctactccgcaccagctgacctgaaatcctttgtaagaaaagcttaacaccaaatgaattaccttcagaaacctaaacattcaacaccataattgagatagactccaccaaattcaccaaataatttaaaaaactagggaaacaagaaaaaagagcttccagaaaaagaaatgaacaatacattaaacaagcagaaacaaaaaaaacgaaggcatgaagtcgaaaattaagagcaaaatgtttctcaattaaaattttcttacatatgctttgtgtctgagcgatgtaagcttgagatagagaaggacgtttccacttctgaagcctctcctttatggaattgctctcttcgagctcgcgaaggacctgtgaatcttcttcgtactgaagcaatgtctcttcgagaaatacatcctcgtctaggtcctcttcttccatggctgcgtggtgtttctgctgctgttgaggcgacggcgttgctgccgccgatcgcttcttagagttgccggagttcat

SEQ ID NO: 47 - DNA, 3261 bp, Solanum chilense
  source 1..3261: mol_type = other DNA
atgaactccggcaactctaagaagcgatcggcggcagcaacgccgtcgcctcaacagcagcagaaacaccacgcagccatggaagaagaggacctagacgaggatgtatttctcgaagagacattgcttcagtacgaagaagattcacaggtccttcgcgagctcgaagagagcaattccataaaggagaggcttcagaagtggaaacgtccttctctatctcaagcttacatcgctcagacacaaagcatatcgtttcagcagctggaaattgactacattattgcagagagccataaagaattgctacctaattcttctggtcaggctgccattttaagaatctttggggtcacaaaggaaggaaatagcgtctgttgtcatgtacatggatttgaaccatacttctatatcagctgccctcctggaatgggccctgacgatatctcccgttttcaccaaattcttgagggtaggatgagagaggcgaataggaacagcaatgcccctaagtttgtccgtcgcattgaagtagttcagaaaagaagcatcatgtattaccaacagcaggcttctcagccttttctcaagattgtagtggcattgccaacaatggttaccagttgccgtggtattctcgatagaggtattcaaattgacgggcttggtatgaagagctttcttacctatgaaagtaatgttctttttgctcttcgcttcatgattgactgcaatattgttggtggcaactggattgaagttccagttgggaaatacaagaaagctgcaagaaatctctcctattgtcaattagagtttgattgtctctattctgacttgataagccattctcctgaaggagaattttcgaagatggctccatttcgcatattgagtttcgatattgaatgtgctggtcgtaaaggccactttccggagcctacacaagatcctgttattcaagtggccaatctggttaccttgcagggacaggatcagccattaattcggaatgtgatgacccttaagtcatgctcaccgatagttggggttgatattatgtcctttgacacagaaaaagaagtattgcgggcttggagggaccttatccgggaggtggaccctgatataataattggatataacatctgcaactttgatttgccatatctcatccggagagctgaagtcttagggatggcagaatttcctgtgcttggacgcatcagaaacagcagggttcgtgtaaaggatgcaaccttttcttcaagacagcatgggacaagggaaagtaaagaaataacaatagaagggagagttcaatttgatttatttcaggctgtgcagagggattataagctaagttcttactcactaaattctgtctcagcacattttctgaatgagcagaaagaggatgtccatcattcaattatatcagatcttcaaaatggcaatgcagaaacgaggcgccggcttgctgtatattgtctaaaggatgcttatctcccacaacggcttttggacaaattaatgttcatttataactatgtggagatggctcgagtcactggtgtacctatatcttttcttctctcaagagggcaaagcattaaggtgctttctcaacttttaaggaaggcgaagcaaaaggatcttgtcataccaaatgcaaaacaggctggatctgaacaaggaacgtttgagggggctacggttttggaggcacgggctggtttctatgagaagccaattgcaacattggattttgcatctctatatccatcaatcatgatggcatataatttatgttactgcacgctagtaactcctgaagaggttcgcaaactgaaccttcctccagattgtgtcaacaagaccccttctggtgaaacgtttgtcaagtcaaccttacagaagggaatacttccggaaattcttgaagaactattgactgctcgtaaaagagcaaaagcagatttaaaggaagccacagaccctcgagtcaaagctgtcttagatggccgtcagctagctttgaagatcagtgcaaattcagtatatgggtttactggagctactgttggtcaattaccatgtttagaaatatcttcaagtgttactagctatggtcgacagatgattgaacacacaaaaaagcttgtggaagacaagtttacagtacttgggggctatgaacacaacgctgaggttatatatggagatacagattccgtgatggtgcagtttggtgtgcctacagtggaagaggctatgaaattgggcagagaagctgctgattacattagcgggacatttatcaaaccaataaaactagaatttgagaagatttattacccatatttgctgattagcaagaagagatatgctggccttctttggacaaatccggacaaacatgacaaaatggatgctaaagggattgaaactgttcggagggataattgtctcttagtcaagaaccttgtaaccgagtgtctttataaaatactagtagacagagatgttcctggtgctgttcagtacgtcaagaatactatctcagatcttcttctgaaccgcatggatctgtctcttttagttattactaagggtctgacaaaaactggagatgattatgaagtaaagacagcacatgttgaacttgcagagcgaatgcgcaagcgagatgctgctacagcacctagcattggtgatagagtaccttacgtaatcattaaagctgcaaaaggggccaaggcatatgaaaagtcagaagaccctatatacgtgttagagaacaacatacctatagatcctcaatactatcttgaaaatcaaattagtaagccacttctgaggatttttgagccaatactgaagaatgcaagtaaagagcttcttcatggagatcacacaagatccatcgcagttccgaccccttcaaacagtggtattatgagatttgccaagaagcaactcacttgcattggatgcaaaacaccactcagtggctctgatcgaacaatctgcaagcactgcaaaggaagagaggccgaactttattgcagaaccgtggctaatgtggctgaactagagaatctatttgggaagctatggacacaatgccaagaatgccaaggctctctccatcaggatgtgctatgcactagtcgagactgcccaatcttctatcggaggaagaaggctcagaaagacatggctgaagctaaaactcaattagaccggtggaatttttaa

SEQ ID NO: 48 - DNA, 3261 bp, Solanum chilense; identical to SEQ ID NO: 47
  source 1..3261: mol_type = other DNA

SEQ ID NO: 49 - protein, 1091 aa, Manihot esculenta
MNRNGNTRKRPPPTSQPTPSPPAPKHQATTPAVDDEFVDEDVFLDETLIAEEENLILRDLEERQALASRLAKWSRPPLSHEYTSQSRSIIFQQLEIDYVIGESNKELLPDRSGVAAIIRIFGVTREGHSVCCLVHGFEPYFYISCPPGMGPDDISRFQQILEGRMKEMNRNSQVPKFVRRIEMVQKRSIMYYQQQPSHPFLKIVVALPTMVASCRGILDKGIHIDGLGMKSFMTYESNVLFALRFMIDCNVVGGNWIEVPAGKYKKTSKNLSYGQLEFDCLYSELISHAPEGEFSKMAPFRILSFDIECAGRKGHFPEPTHDPVIQVANLVTMQGEDQPFIRNVMTLNSCSPIVGVDVMSFDTEREVLLAWRDFIREVDPDIIIGYNICKFDLPYLIERAETLGIAEFPVFGRVKNSRVRVKDTTFSSRQYGTRESKEVTLEGRVQFDLLQAMQRDYKLSSYSLNSVSAHFLSEQKEDVHHSIISDLQNGNAETRRRLAVYCLKDAYLPQRLLDKLMFIYNYVEMARLTGVPLSFLLSRGQSIKVLSQLLRKAKQKNLVIPNVKQAGSEQGTYEGATVLEARAGFYEKPIATLDFASLYPSIMMAYNLCYCTLVTPEDVRKLNIPPECVNKTPSGETFVKSNLQKGILPEILEELLAARRRAKADLKEAKDPLVKAVLDGRQLALKVSANSVYGFTGATIGQLPCIEISSSVTSYGRQMIEHTKKLVEEKFTITGGYEHNAEVIYGDTDSVMVQFGVPTVEEAMKLGREAAECISGTFIKPIKLEFEKVYYPYLLISKKRYAGLFWTNPNKFDKMDTKGIETVRRDNCLLVKNLVNECLHKILIDRDVPGAVQYVKNTISDLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNSDRTLCTHCKGREAELYCKTVSQVSELELLFGRLWTQCQECQGSLHQDVLCTSRDCPIFYRRKKAQKDMAEAKRQLDRWNF

SEQ ID NO: 50 - DNA, 22400 bp, Manihot esculenta
  source 1..22400: mol_type = genomic DNA
ctatctctcattctcactctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattagtgagtaacttcggtgtttcctaattatgggtattattgttgtggattacttttattttcttttatctttcttttattgtgaaattcgatggatttattattttctttacggtcagtatttcagcagctggaaattgattatgtgattggagagagtaataaggagctgctgcctgataggtcaggggtcgctgcgattatcagaatttttggggtcacaagggaaggttaggtcattattgtaatttagaaaataatccctataaaactgagaacatgctatagaaaagtgaaattttttactttttttttttaataaatttgttaaatttgtggtgtagttgtattgtatatgtgtagaagcatacttttacttacactatctatgggttttttactcggtgaagcgaagggtgtagttttcaatttcaaaggtttctaattggcaaatttgataacatattcatggaattcattggtaaacgagtgtactaattatgttttatgcatgagtagttaaatatggcaatctattcatgtgcctggaaatgcaacatctatttattctgtaacattcttatttcagtatattttaatttggtttccaattatgctccatattttgcctctctcctctgatttccaaaccaaataaacaaaaatattgtacacatttgctttttcgactttggatcagtggtttcattctgatttcaacccagtttgtctatttcatcaaatttagatttgatacatacatgtaacacaatatccatattttttaaatttctttctctaattgacattggcacacacttgcactgatgaattctttgaataggtagatgattagtttctatatcatttaagattttttttttcttgttgaatttgaatcagcgacatattgctctttgagaatctcagttacttttttatcattttattctcttgccaatttcaacgttgtgttatataataaactttatagctatggatatggttaaagcatcttgaactacatacagtttgaaacttgtaattgtttctacctcacacgaatatatttacatactgactttttgttaggacatagtgtttgctgccttgttcacggatttgagccatatttttacatcagttgccctcctgggatgggtcctgatgatatttctcgttttcaacaaattcttgaggtatttcatctacgaatttctctgcctatatatttatagataactacatgcatacacacatatgtatatagagctaattgattgcttaattatctcaatatcatctttgttttgggtgtttaaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtaagcatcttcaattttttaatagatgtaactgtgatgcaaacatgtataatctcttttaagttgtttacagcataatgattttgatatctgtattgaattttgagtctcttgacaattcccaatcatttgcaggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtatccttatgcatctcgatcattctttccttgagagaatacttcaattcttattgtccatacttggggctgttttcttgaaaacaattttctgtttttcatttttttttttttttggtaaggaaactctccattttgcattttctataaagtaagggaaaacgtggagatcccattatagaaaacaattttctaattcaattttttaaaatattcatatcattcataataataaaaaaaaatcattataaattatatataaaattttgtttaacttttaaattttttaatatttgttattcatttaatgtataatagtatgattaatttttatcattgtaaattaatattttcctaaataattattcaattttagttatgacaaatttatggtgtaattttagatatagaaaagtcattattttctatgtcaaaaaatttggagacgaagattttttcgtaaaggaaaatagtaaaaactaacttatcactattttcctaaccttagttaaattttcaaggcaaccggaagctagttttccaatccttcatttggaaaattggcataagtgaatgaaactctgttttccagttctcttagctaacttttctagaaaactactccagttttccacagtgaacatttccgtagtgtttttcaagattaattccttgaccaatgttcataggtattcagaattgataagccatgccccagaaggggaattctcaaagatggctccattccgcattttaagttttgatattgaatgtgctggtcggaaaggtcattttcctgagcctacccatgatcctgttattcaggtttttccctagtttttatgcttgttttacttcagtcaagcagtctggttggtggatggatgataattcttttttcactgttgctgaaatcaaaatttccatttcattttaggtggcaaatctagttactatgcagggggaggaccaaccatttatccgtaatgttatgactttaaactcatgctctccaatagttggtgtagatgtgatgtcttttgatacggagagagaagtcctgctggcttggagggtatgtttgcttttctctatagttctcttttgctttgaagaaagctaggttgcatgtttataatttgaataactcttctatacaattattttaacatgttagatttggtgaggaaaatttctgtaagtaatataataaaaaactcataagataatagcaacctgagacttttgaatggcatagttgttctaaagcaaaaatattttagcaaatttaatagtatggtaatagatgcatctgtcatatactcctatcttcctgggtttgctgcttgaatccagtttgatctatagctgtagtggcccatgtgtctcctgcagttataatgttcatgtcttattggaaccaccatcttgaatgtgtccgtcaatggaagttggaagttgaagctttgagctatctgcatttgatcaatgggatggactgtttttttttttttttatcttgtgaaatgccatcttatttgcttgtttcagaaaataatggcttccacagttatttatttattactctggataaaaaaaatttcattcatagtgtttgattgaaaacctagcttaaagtacatacagccaggtgaaattgctcctctttacctctcactccatcctttatctcctcctcctcctttttatttatttatttctttatttttagatttcggtaaatctatcttattaaacaaaattctcagttcatagtcaacaatctcatatactgagcatgacctagaggtgtgcaaaatctgctttggcagttaaaaggattttggatatcaagaagaaaccacaatttacagtttgtgaatttctcaaaccaatttagactatctttgagtacagaaatcaaactaatcaaaatgttagttcacaatttggtttggcatggaaatcccttttggctgaagaagccattcttttagattctcctgtcacaactatgcttatcaaacacttggccatggttgcactaaaatccactttcttccatatatttgctgtgtaagcatgaatcttcatgcacccttagctttgccttctttgataccctaatctttgtgcagtgcttaaatataggactctagcttaacaatgtgtatcttaactgtactatcagttgatgggttgatcttgtattagttttatgatcaggtgctgcataaaggttattaataatttcacgtagtaggatatagtgcattttatgatgtagtaagcaggttttgcatgcaggattttatccgtgaagtggacccagatattataattggatataatatctgcaaatttgacttgccgtatctcattgaggtaattagaagtcttctatattctctttatgcatttttatagtgaagtttttttctttaattttgttttgcagtttaataaatttgaaatgctatttttcttggtttacagagagctgaaactttgggtatagcagagtttcctgtgttcggccgtgtcaagaatagcagggttcgggtcaaagacacaacattttcatcgaggtagtatttttgtttgttacaacaattatcatgtcttttgtgtgcgtgtctttcgtgtgcgtgcttacatattcttgattggagggcattgctttataaactttttttttcttctctcgctttttataatcttcttgttgtagttgtatttggagctctttaggaggattccttgcttactatgctgtatttgttgagttctatttgattccttctgtaccttgttttattggtcaagttctgtagaagtattccattccatgtctaccatgtagcactctcctttctttctcaataaaatattttcgtatatattgaaaaaatattgatgccgtcataagctttaagttgaagattagaatacttgtgcacagtgcatgttatacttttgcttcctgtctaagatttttttttcccatgaaatttgatgagaagagttactgccttcccgagaatttcagagatttaatgagaagtttaatgagaagttcagagaatttcatggaatatgagtgatgtgcttttatttgtctataaacccaaaatgaacttttatgtccttttggtttcataaatttcagactctcaggtgaaatccctagtagtcgtaaaaatagcaatataattgggaagatcgtattgtaatttgatttcaaaaatctgggtcatccttaatgtgagagtaagctttaaagaatgtgtgtatggtgttccttacctggatttagtgcttaaacattggcaccttaatccttgttgctgtttgcagacaatatggtacaagggaaagtaaagaagttacattggaaggaagggttcaatttgatttgcttcaggttttgtgcaataaaactttttctttccacaatctcatttttgtttctgagataaaagttcatttaggcatgttgttatccttccatccaggcaatgcaacgagattataaattaagttcttactcattaaattctgtctcggcgcacttcctctctgagcaggtaagatatagaattgtttggctcaagtgtcatttcttcttgaagtttcagtacactatgattgacctgctttgttttttattatctaattgttaatatacaccatagaagtagtcttagctggatcaaaatatgagtaacttttcctccctaactttctccatttgctttctgtaattacttttgtactacgtctttgtaagtttctgccttgaattattgttcatactggtaatgaaagcattggttgcaataggaagttagcaattttctcagaaatttacgttgcacattaactattttggttctttttgggtctcaatcaatgtatgtgcagtttttctctctcttctctcagccattttctatttaaccttcttcattatggaaaaaagatatttggtgctgatgttgctgacatgttatttgtcttgttcagaaagaggatgtccaccattcaataatatctgatcttcagaatggtaatgcagaaactaggaggcggcttgccgtgtattgtttaaaggtgcatagttttgtttatcttctgtggccctttttcctaaaatttgccttactttgcttctctcttttgctggcaattctttcattattgaactatatttgatgcattaaaatttgacaatttatttcttctttgaaacaaggatttgcatcgactagtctatcttgatattatagattgtgaacttgatgtaatgaaatgcagtgagtctgctacctgtttataaatctttagtgttactggatattgattgaaatcgtgtggtactttacttttggaattgcaggatgcttatctcccacagcggctcttggacaagttgatgtttatttataattatgtagaaatggcccgtgtcacaggcgtccctttatctttccttctttccaggggacaaagtatcaaggttagtgacctgtcatcgcacatcagttgattgaatcttacttgatcaagctaatcaaatcataggatgttgacctcttgttttcaggtactttctcaactcctgaggaaggctaaacagaagaaccttgttattcccaatgtcaaacaggcaggatctgaacaaggaacatatgaaggagccactgtgagtaaattttatctgcatggtcatgtgtactgttttaagaattacaatggctttgagtcgatagttccatctactggtttagccttattttatgtaaatatatcttgctagaaagtaaaagtatatggcttgcaatgttatacatcagtttctgactattaatttgtagttagaactgcaagtgccactatttcaaaatgttacacatcagtttctgactattgatttatagtaagagctgcaagtaccactgcttcaaaatatttggtttttgcgtaccctgcattgtttaggcatgtgggtaatctgtctcttataaagacttgagaaatcttcccccttccaaattcttaatggtaataataaaaaatttacatcacataactgattcactaacttgaatactaccatagttgatgaagttgttgtattctttcttttgtgagtacgtgtgttcatgtaggtgtggacatggatgtgagtcctccctcgtgtttctctcttctttcacctcttcttttgtgccccttttttgggttttttgtgagttgggggggtgggtttgggtgtggtttaagtcttgtatcttggtaattgtcttttacttccccctttacaaagctgaatcatctttgccaatgatttggatctaacaaaatttcaggttttggaggctagagcaggattctacgaaaagccaattgcaacactagattttgcatctttgtatccttcaataatgatggcatataatctatgttactgcaccttggtaataatcttgaccatttattctttttgttggcataacgattatggcttgtaatattggcacaaactttgtttatttctatgttaaacttttgtcttttgcttgtcaaggtgactcctgaagatgttcgtaaactcaatattccacctgaatgtgtcaacaaaactccgtctggtgaaacatttgtcaaatcaaatttacaaaaggttaaatcatgaattttttttataatcatttttattggcgtcatcatcatcttgattaccaggattaattagaattatctttcagggaattcttcctgaaattcttgaagaactattagctgctcgaagaagagcaaaagcagatttgaaggtaattatctattgttttggctgctttattaaatgtttgtatttattattcctttgtgcattgcttattattcctttgtgcgttgccaattttttactctgttgtaatgttactgctaggtttctaggcctttgtgctttttatgattttatctaataattttaggaagctaaggatccgcttgtgaaggctgttctagatggtcgacaactggccttgaaggtaagcttatagatacattgatcattcgatgctcatttttagtgttcacaatctatttactatctagtgaggccaaagcttatagaaagttatacgccatcaagtggatggcctcaaatgcttttaaaatattatctcttttctctttaaaattgattgaaatattgaatttttgttactaaaaaataaatataagtgattagttaaaatttaatgaatacgttgaatgaattatttcattatatcccagaatatctaactttgagttaaagctaacttttgtacttcccatatttctttttgttaagattcaataattagaaaatatattttaaaaataatgttattcttagatcaactattattgcttgaaatttgtattctttactattttatattcatgaaagaatatttagcatttccataaagtgtatggtcttatcatcttggaaattatttatttttttgtgattaatataatattttatgaagtgtctttaaaattttccctcccttggcttccctttttggttatgttaatgtttaatgagaaaatgacttgtttatcattctgttatatacaagtaaatacttgttggtttttcagtaatccaaatatttaattttatgaattttattgataaatttttgtgtttatcaaagtttgactatgtatatttaacattttcaaactaattgttttatattatttagtgaacttagatcactcattttgtagacgatgcaaaaagtgcagtgagaagactagtataataagataaagaataattgggcaatttcagtgtgttttctctgttaaagcaaatgtgtatatatacagattacaagggcaatttctaagaattctttattaatcaattagcctatgattatataatctcctttaattatgtatagattatgttcacaatctaatactctcccttaagttggagcatagatgtttatcatgtccaacttgttagaaaaatattctattcaaggctcatttaaagttttggtgagtagatcacccaattgctctcctatcttcacataactactcacccagttgctctcctgtcttcacataactagtcacataaccagtggataactactcacccagttgctctcctgtcttcacataactagtcacataaccagtggagattaaattttcttgaatcttctaatggatgaaatgacaatcaatttcaatatgcttagtccattcgtggtatacttgattgaaagcaatgtgaagagtagcctgattattacactaaagtttcacgggtgacgcaacatccataccaatttagaatctcacaagtgaatcattgagccatggctctgtattctgactaggcactgaatttggatatcacattttgtttcttactttttcaaaacactaggtttcctccaacaaatacacagtagtctatagtcgaccttttatcacttttgaatctcgcccaatcaacgtcaggaaaacttaagtgcagtatgcccatgatgactcaatcctttttagataatatagaatttgttctagagcagtataatgtttcaccataaatgcagacatgaactggcttacaacgcttactgctaggggtgagcagtattcatttcaaactgaaaaaaccgaccgaaccgaattaatttcaaaattcggttcggttttttattcaattcggttcggtttggtttttaatttcagaaattttggttatttcggtttggtttggttcggttttaatcagaaaaaaattgaaaaaaaccgaaccaaaccgaccgaatgctcacccctacttactgcaaaagcaatagctgactaagtcattaccgtaaagtagttcagctttccaaccaacttcctgtacctctttggatcatcacaagggtctctgtcatcctttgttagatatatatattaggaatcattggtgtgctagagagtttagctcttattttcccaatttctgtaagtaagtcaacgatatacttcttttgagatagaaaaattctctttttacccctcaagacttcgactcctaagaaatatttaacttgactcaagtttttagattgaaaacttgcatgcaagtagtttttgagaggagatcttatactatcattctctataatgataatgtcattaacatacacaacaagaagaataatactagtatctgaatttttataaaagaatgaatggtcacacttgctttttttcaatccaaatttttgaataacttcactgaacttgtcaaaccatgcacggagactctgcttcaaaccatataaagattttttcaaatggctgacttttccatactcccttgagcaacaatggttgcttcatatacactttctcttggaagtcgccatgtaaaagtgcattcttgatatctaactggtgtaaaggacaatgctgagaagttgttagggagatgaacaagcaaattgagatcatttttgcctcaggcgaaaaagtgtcagaatagtcaacgccataggtttgtacataacctttagcgacatgaaggctatagaaccatctggattgactttgatgacaaaaatccatttagagttcacaacctttttgttcgagggtagattaactagttctcaaatatgattttcatctagacccttaatctcctcaaacattgtatcatgccatccagaatgagtcaaggcctctttgactgtcttaaacaaagaaatggaatctagagagacaattaaggagatagaagaagacaggtaattataagacagaaagttagcaacagagtatgtagactttcattgtcgtttacctttacaaaagctaatgtgaaggtcaaggtcactcggtggtgaatccgatggcaaagaagattctggtgcaggacatgtatcatcaggtacttggtctccgagagtaaacttgaacaactgccggtttaataggaggctaagtattaggaggaatgttacaatcatattgtacaacaaaaggcatactcaaagagttttcaccatcagatatgattttataaataagtcgcacatcgtcctcctttcaactagtagaggtttgtgtaacaggataaaaaggaatttgctcgaaaaaggctatatctattgagactagatatttgttgaggtttggagagtaacactgataccccttttgaaggggatcaagtttagtcacatgaggtttgatattccgaacatagcactaaaaagtcgtggtttcagagaaaataatagcttcttaagaaagggcatgcattaatgagaaagcattcagtagagacaatatcagtccaaaattgcttagaaacttgcatttgaaacaacagagctcaggcagtcttaaggagatgtcgattcttcctttcagctaccccattttgagcgggtgtatcaacacatgactattggtaaagaatactatgttgagtcatataagtctgaaatgattttgatatatattttttagcattgtcctttcgtaaaatttgcacaaaaacattaaattgagtcttgatttcagcataaaaggcaaaaatataagaaaacatctctgaatgacactttataaaataaatcaaagtcgttctagagtaatcatccacaaaagtgataaaatatatgaatctggtcttagatccaatcggacatgggtcctaaacttctgaatggactaattcaataactctaggacttaaagagtttcaatgatgttttgtaaaatgacatgactcacattttagtgaagatacctcatgaaattgagggcatagcttcttcaaaactaaagaaggatgtctcaaccgacaatgtgctttgaacggagacacgacactggagcagacaacgcattgaggcacccatgcatccaaaatgtaaagacccaagatacatgtcttttactaataatctgttttgttataagatcttgaatgagacaatgattaggaaaaaaaaaacacaataatttaggtctttggtaagtttacttacagagattagattaaaggaaaattaggtaagcttaacacacatgataggataataggagtaggtttaacagtctcagcctcagaatccaaaatatccaaaatattataggttgatctatcagctatagtaaaaagagaatgtgttttatgagatcgaaagctggtaaaaatatgagaattacatgtcatgtgatctgtgacattagaatcaatgatccatttgtatgaagaggaaataagacaagtttacttatctcagcgtctaccggagacgttgagataattttaataatatgggttttactattagaaaccatttcaaccaaaactttatatttctatgccttcaaatgaattaaaggaaaacaacagaacctaatcacgtgaacagtaacccagtgaacagacagtaggctaccactgaaggacactttccaatcaatgtcggatggctggagttctactagagacgtgctggaaatgaccgggcgattgttggggaagaaaccggaagtcaaacggaggccacacatgctcccgcgcgctgtcgcataaaactagagctgaaatttggatcggcgcatgagcaccactcgcggaagctccagtcactgttttctaaggaacagttggaaataatgttgcgtctctccttgggcctttggtgagatcaaacagacacccaaacttgatgacccaacaactagtttctcaacccgaaaaattcaaaaaaattagacatccaaaattagctcaaaggctatgatacaatataacaagacaaggaataattgggaaatctcagtgtgttttctctattaaagtaagtgtgtatatatatagattacaaggccttattaaggcaattcctaagaaccctctatcaattaattagcctataattatgtaattttctttaattatgtacggattatattcacactcgaacaactaacttagatgcatgttggttcaggatttatacaatcttccattggtttgagctctttgattatcttatgttattgtaggtaagtgcaaattctgtatatggatttacaggagctactattggccagttgccatgtatagaaatatcctctagcgttacaagctacggtatgctaatacaagattctccctaattaatatggtgtttttttattcaatttgatctaacatgttgttggttgcaaaggtcgacagatgattgaacacactaaaaagcttgtagaagaaaaattcaccataacaggaggatatgaacacaatgcagaggtatgcaatctggaatttagatgagtattataatgtagtattttgaaaatgtttattggttaaaatgatgtagttattttaaaatgttgctccatttagaagtattttggtttgtttttaagagtttcacatcataaaattcataaatttaacctttctttgcattaaacacaccattttgttttattgtttaatccagcatcacacactaatttattatctttttgtccttgttttatacctctctgtttgatttgctgtcactcacactctctttgttttgtgcattaatcaatttgttcttattatagatgtgactttagtttcgtttccaatgctctcagtcctaatgccattttagtatcattatgcgtctttcaaaatcaaataatcttccaaattgaattttgaaatttccaatttgtggttttggatttacacaatatcttagttcaattcaaacagaagactttaggtttgagaactgattgattttcactggaaaactaaattgaaccaattcaatttcagtttttgatttggtttaggagagagcattcggccggtttggttctgaaccgaactgaatttgaaaaactgaaaaccaaagtctttaaaattataaaccgaattaaattgatattatagaaaaatcgaaccaaatcaaactgataaatatcagtttggtttgggtcaacaaaaaccgaaattcccaaattatagcaacaagcctaagaatggcctcctcaaattgcaacaaccaacaagagaatgacacttaatattaatttgttccaaaatcaatatataacatttgtaaatagacaaggaataattgtgtaatttcagtgtgttttctctattgaagtaagtgtgtatatgtacagattacaagaccttgttaagtcaacttctaagaattctctatcaatcaattaacctatgattatgtaatctcctttaattatgtataaattatattcacactctaacactccccctcaagttggagcatagatgtttattatacccaacttgttaggaaaatattctattcgagactcatttaaagttttggtgagtagatcacctagttgctctcctttcttcacataactagtgaagattaaattttcttgaatctactcacggatgaaatgacaataaactttaatatgcttagtctatttgtgatatactggattagaagccatgtaaagagcagtctgattatcacaccaaagtttcgtgggtgacacaacatccataccaatttctttcagcagatgatttatccatgccttggtcttgtattctgactcggcattagatttggacactacattttgcttcttacttttttaaaacacgaggttttcttcaacaaatacacagtagcctgtagtcgatcttttatcacttttggttcccactcgatcagcattagaaaaacactcgagtgcagtatctcgagtgcagcatgcccataatcaccgtagactatactgagtcatttagttatgataaatactatacatttagttatgatatttatgaattatataaacactataaatttagttaaaattgcatataaaaatatatgcattttgttagttatttcattacattttgatattttattacactattactattattattgagcgaaaatcactattaaaatgcattacattatttttttaattaaatataatattaaaaatgagaattttaattatctaatgaattaaaattttatatggtactttatatttaattttcagattatttaaatgaattttattaatttattatgaaccatatatttaatataaaaataattttaacatttaatttataaaaaaaaattagaaatacatgaaaagtgagaatttattaatagttacagcaggttaaacacacacttctgcatgtttttatattatatgtggcaactatgcatgttaagattagtacaaaagcaaagaaaaatcctcacttgtagacaacccaaaaagcagaggaaaccacagcatcaaagaagaaaaaaacagggtgggcagattatcttcaatgagtgggagggcaacaagagaaggaagaaagaagaaagagagaaggagaaaataaaaaaaatccaaatgatgttcttcaagcaaaagcagaaaagcaaggtgagaagaaggaggagaaaagatgaaaaaataaaagaaagaacctggatttgcatctatccattcatgagtttggatttcctcttcttttttttttcttcttccgacaactagaaccgccttgtgtaactaggacgtgccttgtctgcgcctgcatgagggcgcctcccgcctgacaccactcaaggcgccagggctggcgcttggtgcgccttgcgcctcaagcgcgcctttcacaacactggtaataatataataaattatcttgttattaatattatgaaaataaaattttagtattggtgttagtaatatataataaatattatttatttatttataaatattgtctatttatagaaaattaatattaattttagttatatttatatattttttattttttattatatatatatatattatattattattctatgttctataatctatattgtaattatattctatatttatattttatgaaatagtgttttgaaataataatattatttattgtataaattgttatattatatagtatatataatttgtataatttagaaatttatatattatttttggttaatattattatttaattataaatatattatctataattatagttatatatatatgtataaatttatttatatatattaagaaattaatatttatcttatatattatataaacataatttatcaccaactatattttatatattttaatataattgaagaaattaatatctagttagattaaaatatataaattgtagttagtgatgcattttatgcatatatacatgtacatttaaaatatataaaaaaatttttaactttgtccgcttttttaaaaaggctttgccttgtgccttgcctcttgcctcttgcctaaggggatagaaggctccatcgccttagtgttgcctcgcgtgcaaaagccctagttatgaataattcgcctgagaacctgcatatgcgatggtgatgcttcggcaccaagacatgcacagctttgacttggcgagaacatatggtgattggcaagaatggacggtgatagaaggctctattgccttggtttttcagttttgttcaaaactgaatcagttgattgcacacctagaagaaatacagaatttggttattttggtttttacaatttttttatcaaactgaccaaatgctattccctcatgcaaaagcagtcaattcttggtgccaacttaaatcatattctcctgccgagatcattagtaacttccgcaaattggctggcttctgtttctgtggagtttatgattggagagaaggctagatatgttggtggaattggaagttaaataattttatgaatggagagaaagctagaacttttgatggaattgaagtttgctaagtgatgtgggattttaaattgattttgctgttctgctggttgtcttatttattcaccatatagatttattgagtatactttattaatgtattagactaggctatccttttctttcggtgcactttgttcctcgtttgttctaaaatccacaaagcttttggcatttcatatcaataacacattgtggtaagctttatctatagggaaaaagcgtcgtagattaccacatgaaaaatattatgcatttttgtctatttggaaatttgaatttcaaatcatgaacataagaatgcaaaaatgagtttcatttacggaatatgtggatgtagtaagctaatcctctctattttacatttctatatgcttggtattgattaaaattttcttacatacctgttttgagtacaatataggtcaaaagcagtcctactttttatggtaatagcttgacctgatcgaagtgataatttcaggttatatatggggacacagattcagttatggtacaatttggtgtgcctactgttgaagaagcaatgaaattgggaagagaagctgctgaatgcatcagtgggactttcataaaagtaatttttatcttgaattctctgatttagaatttaactatttgatagtctttgcaaattcatttttagttaaatcagccaggatagctaatgaagagcctttgaaatacagcaactatgataagctttgaatggtaattttgaactccaattaaatgaattggcagtgtactgtgatttttcgcaaaaaaggaaaggcagggttgaattactcaattgagatattatttcctgaatatgaacttctcaaatatcaaaattgttagtttaattgttgttcttttttcttttttggtctcaatatagttgcctctgtctcacacatgcatttttttgtgtgattgttttagtgtagttttcttcatatgacttatggatttcttaacttttagcaaatgagtaaaataaaccaaatctaagtctgttattttccacgagattcctcttatacagctgaaattgaacacagccaaagttactatactggttttatgttcaatatttttatacctttaattgagtcaacttcacaaggttactcgtgtgagttggtgacatttaataattttttctgtgcaaatgttgctaggaagttattgtcggataatgctacagcgcatcatgatacataagttagaatgatgtctgaataaatcagtcatttatattttaaaaatagtcgtttctattttaaaaagataatcactgggggcttgaagactatctagtctggcaacagtgggaagagacatgggaattctcattatgaaatttgttgaacttggtttctgtttgctatagctatatgattttttcagatatggatacacctgtagtgttcatccaacttgaattctaattaaatgctaaggtttggaagctgtcaaattagctttatgtgattcaacttttggcctttttttttggccttttttttctatcaacttttttaatggttatcatttaactcactcatttcatttttgtatttgggtttttgaatgtgaagcctatcaagctagaatttgagaaggtttattatccatatctgttgattagcaagaagagatatgccggtttattctggactaatcccaacaaatttgacaaaatggatactaaaggtgaatacaacctctcaaacatctgagcttctggatattgtttgggaataatctattgacttttgtcatatatcttgggcaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaaggtgtgaaattgtattggttagggtcctacatactgtccttaaactgctgtataacaacatctaaatatcttgtgtcatcaacatggatattttcttcctgtcctaatttattttgtttaacagggtctgacaaagactggagacgattacgaagttaaggcagctcatgttgaacttgctgaacgcatgcgcaaggtgctttttttttagtgttcttttttcctcctttgtagaagggcttgttcttttgctagaattctggagagaggaattaaaagtctgggaccttaataatcttgtttttatttagataagttgagattaggctcaaaattctataggaatttaaaatcagataggatgggatttttagatttttaaagattttatttaaacttaattattctggtgggcaaaacataaagtactactaatctacgttgaaacaaggaaaatagcaaagaaaagaaactactgatgccatctgtaagaacctggatagattcttcgatggattttggagattaggtgagagagaaagtagatgtagatgagaagagaagggaggagaaaatagagtagatgagaagagaattaggagagtagagaggaggattctgttattggttattcttcaatgatcaatacagatagttgtttcttatttatagcagacccacaactaatttctgttgcaaccaattttccctcgaaaaccaacagctcacacacacagctgtactaagctcctctgtttttcctcctaaaatacataacaccatcttatcattttgaatgatattcaccaacatgaaacgctccaaggcacaagccataaagaagccaagaaaactatcttactctgcaggatgtgggaaaacaaagtttaataatctataatacagcataagatcttattctgtttgttattgacagagagatgctgccactgctccaaatgttggggatcgagtaccttatgttattataaaagctgcaaaaggtgccaaggtagttttctgggatttggttttgtttaatacctttttctccaaattacttggtacggtcctctgattgttcaatttttatatgcaactgataatcaggcttatgagagatcagaagatcctatctatgttctggaaaataatataccaatagatcctcagtattatcttgaaaaccaaatcagcaaggttagtttggtatatgttttgtacaaagccaaatagtgattctttttctaatattcattaattataaatgttgatattgcagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattaggtatgttttatttgtattccactttatctctgccttagcactacccgtattggctgcaaggcactaattaggtacttccaaggtctctgtggatggtgggaggccctagattacttgctatagcttttcggtttcttgtacttttaattggtaaaaggtactttgtaatttgtaaatcaactgaaattgagatctctatttcagcgcctctatgacccaaagatttccttaaacactcatatggttgcattcatttactttcccgagttatataatatatttgagatatctgaggttttagaggagaaggcaaatttggcaccatttatttatttatgatttatcaagttcaatgattgcatgatttttcactctttttctcattctgcttagtgaacttctgttcatcacagactaagagtgcaaagtaggattctgtattctttctcacttacaataataccaattatttaagatgttttactatctccttaattgatcagatctattgtaatagagtgaaaatcaaatgcacctttatctctagatattttgtgcattcaaaattccatatgcgttttttatgtaataattagtgctccttttttctgcttttgctgtgggtatttcctcattgattatgggattctatccttctttgctgccatacgaatacaggcattatatttatttgaagagcctaatactattaatttggttttggaccagcaattcagatcgaactctctgcacacactgtaaaggaagagaagcagaattgtactgcaaaacggtttctcaaggtagacatctcttctgaattctgaatcatgataatttgtgtaattgtaatagagatggtgtttattaagcagaattcatggattgattgcagtgtccgaactggagttgctttttgggaggctgtggacacagtgccaagagtgccagggctctctccatcaggatgttctttgcactaggttttattttctttccattctgttaccattaaataatattctttgaaaaaaatgtttgcagggtttcctggtagctaaattattagctcaaattgtaactaaatattttatttttctttaataaaaagaaagaaagaagaatcacagagcacatattctacgatactttgtaatcactgatttttgttatgcttcatatgtccacagtcgggattgcccaatattctataggagaaagaaggcgcaaaaggacatggctgaagccaaacggcagttggataggtggaacttctaatctgataacaaggttagtatcttttattctccgagtcattgtttatcttttacatattcgtgaattaacaaatctgatgttaactcactagggaaaattcggaagtcagtttgtcacagaggcctgcaatagttgagggtccaactgacttgaaaggttgcatatctgattggctagtattaaggcagaagctgtgtcgagacatgagttctaaaatggcagattttcaggttgattgacctttcgcaggctttgatagtcgggatgttgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttattttggtctatcttggattacttgtacatctctggccatttttggctgtagcttgaatgtcattacgata

SEQ ID NO: 51 - DNA, 3863 bp, Manihot esculenta
  source 1..3863: mol_type = other DNA
tctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtctcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaatctgataacaagggaaaattcggaagtcagtttgtcacagaggcctgcaatagttgagggtccaactgacttgaaaggttgcatatctgattggctagtattaaggcagaagctgtgtcgagacatgagttctaaaatggcagattttcaggttgattgacctttcgcaggctttgatagtcgggatgttgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttattttggtctatcttggattacttgtacatctctggccatttttggctgtagcttgaatgtcattacgata

SEQ ID NO: 52 - DNA, 3276 bp, Manihot esculenta
  source 1..3276: mol_type = other DNA
atgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtctcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaa

SEQ ID NO: 53 - protein, 1091 aa, Manihot esculenta
MNRNGNTRKRPPPTSQPTPSPPAPKHQATTPAVDDEFVDEDVFLDETLIAEEENLILRDLEERQALASRLAKWSRPPLSHEYTSQSRSIIFQQLEIDYVIGESNKELLPDRSGVAAIIRIFGVTREGHSVCCLVHGFEPYFYISCPPGMGPDDISRFQQILEGRMKEMNRNSQVPKFVRRIEMVQKRSIMYYQQQPSHPFLKIVVALPTMVASCRGILDKGIHIDGLGMKSFMTYESNVLFALRFMIDCNVVGGNWIEVPAGKYKKTSKNLSYGQLEFDCLYSELISHAPEGEFSKMAPFRILSFDIECAGRKGHFPEPTHDPVIQVANLVTMQGEDQPFIRNVMTLNSCSPIVGVDVMSFDTEREVLLAWRDFIREVDPDIIIGYNICKFDLPYLIERAETLGIAEFPVFGRVKNSRVRVKDTTFSSRQYGTRESKEVTLEGRVQFDLLQAMQRDYKLSSYSLNSVSAHFLSEQKEDVHHSIISDLQNGNAETRRRLAVYCLKDAYLPQRLLDKLMFIYNYVEMARVTGVPLSFLLSRGQSIKVLSQLLRKAKQKNLVIPNVKQAGSEQGTYEGATVLEARAGFYEKPIATLDFASLYPSIMMAYNLCYCTLVTPEDVRKLNIPPECVNKTPSGETFVKSNLQKGILPEILEELLAARRRAKADLKEAKDPLVKAVLDVRQLALKVSANSVYGFTGATIGQLPCIEISSSVTSYGRQMIEHTKKLVEEKFTITGGYEHNAEVIYGDTDSVMVQFGVPTVEEAMKLGREAAECISGTFIKPIKLEFEKVYYPYLLISKKRYAGLFWTNPNKFDKMDTKGIETVRRDNCLLVKNLVNECLHKILIDRDVPGAVQYVKNTISDLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNSDRTLCTHCKGREAELYCKTVSQVSELELLFGRLWTQCQECQGSLHQDVLCTSRDCPIFYRRKKAQKDMAEAKRQLDRWNF

SEQ ID NO: 54 - DNA, 22384 bp, Manihot esculenta
  source 1..22384: mol_type = genomic DNA
tatcgtaatgacattcaagctacagccaaaaatggccagagatgtacaagtaatccaagatagaccaaaataacacattcaagtgtcggcctaaacacacgcggggatagagatagattacaaggattagagatttatccattaaattaactctgaagatgtattacttacactcttaattacctgagcaaaccttagctacaaaactgagacgcaagtattcacaagtatcagcgatacaagaagcatagggaagttgcaaccactgtaagcttgttccccagttcaacatcccgactatcaaagcctgcgaaaggtcaatcaacctgaaaatctgccattttagaactcatgtctcgacacagcttctgccttaatactagccaatcagatatgcaacctttcaagtcagttggaccctcaactattgcaggcctctgtgacaaactgacttccgaattttccctagtgagttaacatcagatttgttaattcacgaatatgtaaaagataaacaatgactcggagaataaaagatactaaccttgttatcagattagaagttccacctatccaactgccgtttggcttcagccatgtccttttgcgccttctttctcctatagaatattgggcaatcccgactgtggacatatgaagcataacaaaaatcagtgattacaaagtatcgtagaatatgtgctctgtgattcttctttctttctttttattaaagaaaaataaaatatttagttacaatttgagctaataatttagctaccaggaaaccctgcaaacatttttttcaaagaatattatttaatggtaacagaatggaaagaaaataaaacctagtgcaaagaacatcctgatggagagagccctggcactcttggcactgtgtccacagcctcccaaaaagcaactccagttcggacactgcaatcaatccatgaattctgcttaataaacaccatctctattacaattacacaaattatcatgattcagaattcagaagagatgtctaccttgagaaaccgttttgcagtacaattctgcttctcttcctttacagtgtgtgcagagagttcgatctgaattgctggtccaaaaccaaattaatagtattaggctcttcaaataaatataatgcctgtattcgtatggcagcaaagaaggatagaatcccataatcaatgaggaaatacccacagcaaaagcagaaaaaaggagcactaattattacataaaaaacgcatatggaattttgaatgcacaaaatatctagagataaaggtgcatttgattttcactctattacaatagatctgatcaattaaggagatagtaaaacatcttaaataattggtattattgtaagtgagaaagaatacagaatcctactttgcactcttagtctgtgatgaacagaagttcactaagcagaatgagaaaaagagtgaaaaatcatgcaatcattgaacttgataaatcataaataaataaatggtgccaaatttgccttctcctctaaaacctcagatatctcaaatatattatataactcgggaaagtaaatgaatgcaaccatatgagtgtttaaggaaatctttgggtcatagaggcgctgaaatagagatctcaatttcagttgatttacaaattacaaagtaccttttaccaattaaaagtacaagaaaccgaaaagctatagcaagtaatctagggcctcccaccatccacagagaccttggaagtacctaattagtgccttgcagccaatacgggtagtgctaaggcagagataaagtggaatacaaataaaacatacctaattagtgccttgcagccaatacaactgagttgtttcttagcaaatttcataatgccactatttgaaggagtggagatggaaatagatcttgtatgacttccatggaggagttccttgctggcattcttcaatatgggctcaaaaattcttaaaagtggctgcaatatcaacatttataattaatgaatattagaaaaagaatcactatttggctttgtacaaaacatataccaaactaaccttgctgatttggttttcaagataatactgaggatctattggtatattattttccagaacatagataggatcttctgatctctcataagcctgattatcagttgcatataaaaattgaacaatcagaggaccgtaccaagtaatttggagaaaaaggtattaaacaaaaccaaatcccagaaaactaccttggcaccttttgcagcttttataataacataaggtactcgatccccaacatttggagcagtggcagcatctctctgtcaataacaaacagaataagatcttatgctgtattatagattattaaactttgttttcccacatcctgcagagtaagatagttttcttggcttctttatggcttgtgccttggagcgtttcatgttggtgaatatcattcaaaatgataagatggtgttatgtattttaggaggaaaaacagaggagcttagtacagctgtgtgtgtgagctgttggttttcgagggaaaattggttgcaacagaaattagttgtgggtctgctataaataagaaacaactatctgtattgatcattgaagaataaccaataacagaatcctcctctctactctcctaattctcttctcatctactctattttctcctcccttctcttctcatctacatctactttctctctcacctaatctccaaaatccatcgaagaatctatccaggttcttacagatggcatcagtagtttcttttctttgctattttccttgtttcaacgtagattagtagtactttatgttttgcccaccagaataattaagtttaaataaaatctttaaaaatctaaaaatcccatcctatctgattttaaattcctatagaattttgagcctaatctcaacttatctaaataaaaacaagattattaaggtcccagacttttaattcctctctccagaattctagcaaaagaacaagcccttctacaaaggaggaaaaaagaacactaaaaaaaaagcaccttgcgcatgcgttcagcaagttcaacatgagctgccttaacttcgtaatcgtctccagtctttgtcagaccctgttaaacaaaataaattaggacaggaagaaaatatccatgttgatgacacaagatatttagatgttgttatacagcagtttaaggacagtatgtaggaccctaaccaatacaatttcacacctttgtaataaccagaagtgacaaatccatacgattcatgagaagatcagagatggtattcttgacatactgaactgcaccaggcacatctctgtctattagtatcttgtgaagacactcatttaccaggtttttgaccaataaacagttgtcccttctaactgtttcaattcctgcccaagatatatgacaaaagtcaatagattattcccaaacaatatccagaagctcagatgtttgagaggttgtattcacctttagtatccattttgtcaaatttgttgggattagtccagaataaaccggcatatctcttcttgctaatcaacagatatggataataaaccttctcaaattctagcttgataggcttcacattcaaaaacccaaatacaaaaatgaaatgagtgagttaaatgataaccattaaaaaagttgatagaaaaaaaaggccaaaaaaaaaggccaaaagttgaatcacataaagctaatttgacagcttccaaaccttagcatttaattagaattcaagttggatgaacactacaggtgtatccatatctgaaaaaatcatatagctatagcaaacagaaaccaagttcaacaaatttcataatgagaattcccatgtctcttcccactgttgccagactagatagtcttcaagcccccagtgattatctttttaaaatagaaacgactatttttaaaatataaatgactgatttattcagacatcattctaacttatgtatcatgatgcgctgtagcattatccgacaataacttcctagcaacatttgcacagaaaaaattattaaatgtcaccaactcacacgagtaaccttgtgaagttgactcaattaaaggtataaaaatattgaacataaaaccagtatagtaactttggctgtgttcaatttcagctgtataagaggaatctcgtggaaaataacagacttagatttggtttattttactcatttgctaaaagttaagaaatccataagtcatatgaagaaaactacactaaaacaatcacacaaaaaaatgcatgtgtgagacagaggcaactatattgagaccaaaaaagaaaaaagaacaacaattaaactaacaattttgatatttgagaagttcatattcaggaaataatatctcaattgagtaattcaaccctgcctttccttttttgcgaaaaatcacagtacactgccaattcatttaattggagttcaaaattaccattcaaagcttatcatagttgctgtatttcaaaggctcttcattagctatcctggctgatttaactaaaaatgaatttgcaaagactatcaaatagttaaattctaaatcagagaattcaagataaaaattacttttatgaaagtcccactgatgcattcagcagcttctcttcccaatttcattgcttcttcaacagtaggcacaccaaattgtaccataactgaatctgtgtccccatatataacctgaaattatcacttcgatcaggtcaagctattaccataaaaagtaggactgcttttgacctatattgtactcaaaacaggtatgtaagaaaattttaatcaataccaagcatatagaaatgtaaaatagagaggattagcttactacatccacatattccgtaaatgaaactcatttttgcattcttatgttcatgatttgaaattcaaatttccaaatagacaaaaatgcataatatttttcatgtggtaatctacgacgctttttccctatagataaagcttaccacaatgtgttattgatatgaaatgccaaaagctttgtggattttagaacaaacgaggaacaaagtgcaccgaaagaaaaggatagcctagtctaatacattaataaagtatactcaataaatctatatggtgaataaataagacaaccagcagaacagcaaaatcaatttaaaatcccacatcacttagcaaacttcaattccatcaaaagttctagctttctctccattcataaaattatttaacttccaattccaccaacatatctagccttctctccaatcataaactccacagaaacagaagccagccaatttgcggaagttactaatgatctcggcaggagaatatgatttaagttggcaccaagaattgactgcttttgcatgagggaatagcatttggtcagtttgataaaaaaattgtaaaaaccaaaataaccaaattctgtatttcttctaggtgtgcaatcaactgattcagttttgaacaaaactgaaaaaccaaggcaatagagccttctatcaccgtccattcttgccaatcaccatatgttctcgccaagtcaaagctgtgcatgtcttggtgccgaagcatcaccatcgcatatgcaggttctcaggcgaattattcataactagggcttttgcacgcgaggcaacactaaggcgatggagccttctatccccttaggcaagaggcaagaggcaaggcacaaggcaaagcctttttaaaaaagcggacaaagttaaaaatttttttatatattttaaatgtacatgtatatatgcataaaatgcatcactaactacaatttatatattttaatctaactagatattaatttcttcaattatattaaaatatataaaatatagttggtgataaattatgtttatataatatataagataaatattaatttcttaatatatataaataaatttatacatatatatataactataattatagataatatatttataattaaataataatattaaccaaaaataatatataaatttctaaattatacaaattatatatactatataatataacaatttatacaataaataatattattatttcaaaacactatttcataaaatataaatatagaatataattacaatatagattatagaacatagaataataatataatatatatatatataataaaaaataaaaaatatataaatataactaaaattaatattaattttctataaatagacaatatttataaataaataaataatatttattatatattactaacaccaatactaaaattttattttcataatattaataacaagataatttattatattattaccagtgttgtgaaaggcgcgcttgaggcgcaaggcgcaccaagcgccagccctggcgccttgagtggtgtcaggcgggaggcgccctcatgcaggcgcagacaaggcacgtcctagttacacaaggcggttctagttgtcggaagaagaaaaaaaaaagaagaggaaatccaaactcatgaatggatagatgcaaatccaggttctttcttttattttttcatcttttctcctccttcttctcaccttgcttttctgcttttgcttgaagaacatcatttggattttttttattttctccttctctctttcttctttcttccttctcttgttgccctcccactcattgaagataatctgcccaccctgtttttttcttctttgatgctgtggtttcctctgctttttgggttgtctacaagtgaggatttttctttgcttttgtactaatcttaacatgcatagttgccacatataatataaaaacatgcagaagtgtgtgtttaacctgctgtaactattaataaattctcacttttcatgtatttctaatttttttttataaattaaatgttaaaattatttttatattaaatatatggttcataataaattaataaaattcatttaaataatctgaaaattaaatataaagtaccatataaaattttaattcattagataattaaaattctcatttttaatattatatttaattaaaaaaataatgtaatgcattttaatagtgattttcgctcaataataatagtaatagtgtaataaaatatcaaaatgtaatgaaataactaacaaaatgcatatatttttatatgcaattttaactaaatttatagtgtttatataattcataaatatcataactaaatgtatagtatttatcataactaaatgactcagtatagtctacggtgattatgggcatgctgcactcgagatactgcactcgagtgtttttctaatgctgatcgagtgggaaccaaaagtgataaaagatcgactacaggctactgtgtatttgttgaagaaaacctcgtgttttaaaaaagtaagaagcaaaatgtagtgtccaaatctaatgccgagtcagaatacaagaccaaggcatggataaatcatctgctgaaagaaattggtatggatgttgtgtcacccacgaaactttggtgtgataatcagactgctctttacatggcttctaatccagtatatcacaaatagactaagcatattaaagtttattgtcatttcatccgtgagtagattcaagaaaatttaatcttcactagttatgtgaagaaaggagagcaactaggtgatctactcaccaaaactttaaatgagtctcgaatagaatattttcctaacaagttgggtataataaacatctatgctccaacttgagggggagtgttagagtgtgaatataatttatacataattaaaggagattacataatcataggttaattgattgatagagaattcttagaagttgacttaacaaggtcttgtaatctgtacatatacacacttacttcaatagagaaaacacactgaaattacacaattattccttgtctatttacaaatgttatatattgattttggaacaaattaatattaagtgtcattctcttgttggttgttgcaatttgaggaggccattcttaggcttgttgctataatttgggaatttcggtttttgttgacccaaaccaaactgatatttatcagtttgatttggttcgatttttctataatatcaatttaattcggtttataattttaaagactttggttttcagtttttcaaattcagttcggttcagaaccaaaccggccgaatgctctctcctaaaccaaatcaaaaactgaaattgaattggttcaatttagttttccagtgaaaatcaatcagttctcaaacctaaagtcttctgtttgaattgaactaagatattgtgtaaatccaaaaccacaaattggaaatttcaaaattcaatttggaagattatttgattttgaaagacgcataatgatactaaaatggcattaggactgagagcattggaaacgaaactaaagtcacatctataataagaacaaattgattaatgcacaaaacaaagagagtgtgagtgacagcaaatcaaacagagaggtataaaacaaggacaaaaagataataaattagtgtgtgatgctggattaaacaataaaacaaaatggtgtgtttaatgcaaagaaaggttaaatttatgaattttatgatgtgaaactcttaaaaacaaaccaaaatacttctaaatggagcaacattttaaaataactacatcattttaaccaataaacattttcaaaatactacattataatactcatctaaattccagattgcatacctctgcattgtgttcatatcctcctgttatggtgaatttttcttctacaagctttttagtgtgttcaatcatctgtcgacctttgcaaccaacaacatgttagatcaaattgaataaaaaaacaccatattaattagggagaatcttgtattagcataccgtagcttgtaacgctagaggatatttctatacatggcaactggccaatagtagctcctgtaaatccatatacagaatttgcacttacctacaataacataagataatcaaagagctcaaaccaatggaagattgtataaatcctgaaccaacatgcatctaagttagttgttcgagtgtgaatataatccgtacataattaaagaaaattacataattataggctaattaattgatagagggttcttaggaattgccttaataaggccttgtaatctatatatatacacacttactttaatagagaaaacacactgagatttcccaattattccttgtcttgttatattgtatcatagcctttgagctaattttggatgtctaatttttttgaatttttcgggttgagaaactagttgttgggtcatcaagtttgggtgtctgtttgatctcaccaaaggcccaaggagagacgcaacattatttccaactgttccttagaaaacagtgactggagcttccgcgagtggtgctcatgcgccgatccaaatttcagctctagttttatgcgacagcgcgcgggagcatgtgtggcctccgtttgacttccggtttcttccccaacaatcgcccggtcatttccagcacgtctctagtagaactccagccatccgacattgattggaaagtgtccttcagtggtagcctactgtctgttcactgggttactgttcacgtgattaggttctgttgttttcctttaattcatttgaaggcatagaaatataaagttttggttgaaatggtttctaatagtaaaacccatattattaaaattatctcaacgtctccggtagacgctgagataagtaaacttgtcttatttcctcttcatacaaatggatcattgattctaatgtcacagatcacatgacatgtaattctcatatttttaccagctttcgatctcataaaacacattctctttttactatagctgatagatcaacctataatattttggatattttggattctgaggctgagactgttaaacctactcctattatcctatcatgtgtgttaagcttacctaattttcctttaatctaatctctgtaagtaaacttaccaaagacctaaattattgtgttttttttttcctaatcattgtctcattcaagatcttataacaaaacagattattagtaaaagacatgtatcttgggtctttacattttggatgcatgggtgcctcaatgcgttgtctgctccagtgtcgtgtctccgttcaaagcacattgtcggttgagacatccttctttagttttgaagaagctatgccctcaatttcatgaggtatcttcactaaaatgtgagtcatgtcattttacaaaacatcattgaaactctttaagtcctagagttattgaattagtccattcagaagtttaggacccatgtccgattggatctaagaccagattcatatattttatcacttttgtggatgattactctagaacgactttgatttattttataaagtgtcattcagagatgttttcttatatttttgccttttatgctgaaatcaagactcaatttaatgtttttgtgcaaattttacgaaaggacaatgctaaaaaatatatatcaaaatcatttcagacttatatgactcaacatagtattctttaccaatagtcatgtgttgatacacccgctcaaaatggggtagctgaaaggaagaatcgacatctccttaagactgcctgagctctgttgtttcaaatgcaagtttctaagcaattttggactgatattgtctctactgaatgctttctcattaatgcatgccctttcttaagaagctattattttctctgaaaccacgactttttagtgctatgttcggaatatcaaacctcatgtgactaaacttgatccccttcaaaaggggtatcagtgttactctccaaacctcaacaaatatctagtctcaatagatatagcctttttcgagcaaattcctttttatcctgttacacaaacctctactagttgaaaggaggacgatgtgcgacttatttataaaatcatatctgatggtgaaaactctttgagtatgccttttgttgtacaatatgattgtaacattcctcctaatacttagcctcctattaaaccggcagttgttcaagtttactctcggagaccaagtacctgatgatacatgtcctgcaccagaatcttctttgccatcggattcaccaccgagtgaccttgaccttcacattagcttttgtaaaggtaaacgacaatgaaagtctacatactctgttgctaactttctgtcttataattacctgtcttcttctatctccttaattgtctctctagattccatttctttgtttaagacagtcaaagaggccttgactcattctggatggcatgatacaatgtttgaggagattaagggtctagatgaaaatcatatttgagaactagttaatctaccctcgaacaaaaaggttgtgaactctaaatggatttttgtcatcaaagtcaatccagatggttctatagccttcatgtcgctaaaggttatgtacaaacctatggcgttgactattctgacactttttcgcctgaggcaaaaatgatctcaatttgcttgttcatctccctaacaacttctcagcattgtcctttacaccagttagatatcaagaatgcacttttacatggcgacttccaagagaaagtgtatatgaagcaaccattgttgctcaagggagtatggaaaagtcagccatttgaaaaaatctttatatggtttgaagcagagtctccgtgcatggtttgacaagttcagtgaagttattcaaaaatttggattgaaaaaaagcaagtgtgaccattcattcttttataaaaattcagatactagtattattcttcttgttgtgtatgttaatgacattatcattatagagaatgatagtataagatctcctctcaaaaactacttgcatgcaagttttcaatctaaaaacttgagtcaagttaaatatttcttaggagtcgaagtcttgaggggtaaaaagagaatttttctatctcaaaagaagtatatcgttgacttacttacagaaattgggaaaataagagctaaactctctagcacaccaatgattcctaatatatatatctaacaaaggatgacagagacccttgtgatgatccaaagaggtacaggaagttggttggaaagctgaactactttacggtaatgacttagtcagctattgcttttgcagtaagtaggggtgagcattcggtcggtttggttcggtttttttcaatttttttctgattaaaaccgaaccaaaccaaaccgaaataaccaaaatttctgaaattaaaaaccaaaccgaaccgaattgaataaaaaaccgaaccgaattttgaaattaattcggttcggtcggttttttcagtttgaaatgaatactgctcacccctagcagtaagcgttgtaagccagttcatgtctgcatttatggtgaaacattatactgctctagaacaaattctatattatctaaaaaggattgagtcatcatgggcatactgcacttaagttttcctgacgttgattgggcgagattcaaaagtgataaaaggtcgactatagactactgtgtatttgttggaggaaacctagtgttttgaaaaagtaagaaacaaaatgtgatatccaaattcagtgcctagtcagaatacagagccatggctcaatgattcacttgtgagattctaaattggtatggatgttgcgtcacccgtgaaactttagtgtaataatcaggctactcttcacattgctttcaatcaagtataccacgaatggactaagcatattgaaattgattgtcatttcatccattagaagattcaagaaaatttaatctccactggttatgtgactagttatgtgaagacaggagagcaactgggtgagtagttatccactggttatgtgactagttatgtgaagacaggagagcaactgggtgagtagttatgtgaagataggagagcaattgggtgatctactcaccaaaactttaaatgagccttgaatagaatatttttctaacaagttggacatgataaacatctatgctccaacttaagggagagtattagattgtgaacataatctatacataattaaaggagattatataatcataggctaattgattaataaagaattcttagaaattgcccttgtaatctgtatatatacacatttgctttaacagagaaaacacactgaaattgcccaattattctttatcttattatactagtcttctcactgcactttttgcatcgtctacaaaatgagtgatctaagttcactaaataatataaaacaattagtttgaaaatgttaaatatacatagtcaaactttgataaacacaaaaatttatcaataaaattcataaaattaaatatttggattactgaaaaaccaacaagtatttacttgtatataacagaatgataaacaagtcattttctcattaaacattaacataaccaaaaagggaagccaagggagggaaaattttaaagacacttcataaaatattatattaatcacaaaaaataaataatttccaagatgataagaccatacactttatggaaatgctaaatattctttcatgaatataaaatagtaaagaatacaaatttcaagcaataatagttgatctaagaataacattattttttaaaatatattttctaattattgaatcttaacaaaaagaaatatgggaagtacaaaagttagctttaactccaaagttagatattctgggatataatgaaataattcattcaacgtattcattaaattttaactaatcacttatatttattttttagtaacaaaaattcaatatttcaatcaattttaaagagaaaagagataatattttaaaagcatttgaggccatccacttgatggcgtataactttctataagctttggcctcactagatagtaaatagattgtgaacactaaaaatgagcatcgaatgatcaatgtatctataagcttaccttcaaggccagttgtcgaacatctagaacagccttcacaagcggatccttagcttcctaaaattattagataaaatcataaaaagcacaaaggcctagaaacctagcagtaacattacaacagagtaaaaaattggcaacgcacaaaggaataataagcaatgcacaaaggaataataaatacaaacatttaataaagcagccaaaacaatagataattaccttcaaatctgcttttgctcttcttcgagcagctaatagttcttcaagaatttcaggaagaattccctgaaagataattctaattaatcctggtaatcaagatgatgatgacgccaataaaaatgattataaaaaaaattcatgatttaaccttttgtaaatttgatttgacaaatgtttcaccagacggagttttgttgacacattcaggtggaatattgagtttacgaacatcttcaggagtcaccttgacaagcaaaagacaaaagtttaacatagaaataaacaaagtttgtgccaatattacaagccataatcgttatgccaacaaaaagaataaatggtcaagattattaccaaggtgcagtaacatagattatatgccatcattattgaaggatacaaagatgcaaaatctagtgttgcaattggcttttcgtagaatcctgctctagcctccaaaacctgaaattttgttagatccaaatcattggcaaagatgattcagctttgtaaagggggaagtaaaagacaattaccaagatacaagacttaaaccacacccaaacccacccccccaactcacaaaaaacccaaaaaaggggcacaaaagaagaggtgaaagaagagagaaacacgagggaggactcacatccatgtccacacctacatgaacacacgtactcacaaaagaaagaatacaacaacttcatcaactatggtagtattcaagttagtgaatcagttatgtgatgtaaattttttattattaccattaagaatttggaagggggaagatttctcaagtctttataagagacagattacccacatgcctaaacaatgcagggtacgcaaaaaccaaatattttgaagcagtggtacttgcagctcttactataaatcaatagtcagaaactgatgtgtaacattttgaaatagtggcacttgcagttctaactacaaattaatagtcagaaactgatgtataacattgcaagccatatacttttactttctagcaagatatatttacataaaataaggctaaaccagtagatggaactatcgactcaaagccattgtaattcttaaaacagtacacatgaccatgcagataaaatttactcacagtggctccttcatatgttccttgttcagatcctgcctgtttgacattgggaataacaaggttcttctgtttagccttcctcaggagttgagaaagtacctgaaaacaagaggtcaacatcctatgatttgattagcttgatcaagtaagattcaatcaactgatgtgcgatgacaggtcactaaccttgatactttgtcccctggaaagaaggaaagataaagggacgcctgtgacacgggccatttctacataattataaataaacatcaacttgtccaagagccgctgtgggagataagcatcctgcaattccaaaagtaaagtaccacacgatttcaatcaatatccagtaacactaaagatttataaacaggtagcagactcactgcatttcattacatcaagttcacaatctataatatcaagatagactagtcgatgcaaatccttgtttcaaagaagaaataaattgtcaaattttaatgcatcaaatatagttcaataatgaaagaattgccagcaaaagagagaagcaaagtaaggcaaattttaggaaaaagggccacagaagataaacaaaactatgcacctttaaacaatacacggcaagccgcctcctagtttctgcattaccattctgaagatcagatattattgaatggtggacatcctctttctgaacaagacaaataacatgtcagcaacatcagcaccaaatatcttttttccataatgaagaaggttaaatagaaaatggctgagagaagagagagaaaaactgcacatacattgattgagacccaaaaagaaccaaaatagttaatgtgcaacgtaaatttctgagaaaattgctaacttcctattgcaaccaatgctttcattaccagtatgaacaataattcaaggcagaaacttacaaagacgtagtacaaaagtaattacagaaagcaaatggagaaagttagggaggaaaagttactcatattttgatccagctaagactacttctatggtgtatattaacaattagataataaaaaacaaagcaggtcaatcatagtgtactgaaacttcaagaagaaatgacacttgagccaaacaattctatatcttacctgctcagagaggaagtgcgccgagacagaatttaatgagtaagaacttaatttataatctcgttgcattgcctggatggaaggataacaacatgcctaaatgaacttttatctcagaaacaaaaatgagattgtggaaagaaaaagttttattgcacaaaacctgaagcaaatcaaattgaacccttccttccaatgtaacttctttactttcccttgtaccatattgtctgcaaacagcaacaaggattaaggtgccaatgtttaagcactaaatccaggtaaggaacaccatacacacattctttaaagcttactctcacattaaggatgacccagatttttgaaatcaaattacaatacgatcttcccaattatattgctatttttacgactactagggatttcacctgagagtctgaaatttatgaaaccaaaaggacataaaagttcattttgggtttatagacaaataaaagcacatcactcatattccatgaaattctctgaacttctcattaaacttctcattaaatctctgaaattctcgggaaggcagtaactcttctcatcaaatttcatgggaaaaaaaaatcttagacaggaagcaaaagtataacatgcactgtgcacaagtattctaatcttcaacttaaagcttatgacggcatcaatattttttcaatatatacgaaaatattttattgagaaagaaaggagagtgctacatggtagacatggaatggaatacttctacagaacttgaccaataaaacaaggtacagaaggaatcaaatagaactcaacaaatacagcatagtaagcaaggaatcctcctaaagagctccaaatacaactacaacaagaagattataaaaagcgagagaagaaaaaaaaagtttataaagcaatgccctccaatcaagaatatgtaagcacgcacacgaaagacacgcacacaaaagacatgataattgttgtaacaaacaaaaatactacctcgatgaaaatgttgtgtctttgacccgaaccctgctattcttgacacggccgaacacaggaaactctgctatacccaaagtttcagctctctgtaaaccaagaaaaatagcatttcaaatttattaaactgcaaaacaaaattaaagaaaaaaacttcactataaaaatgcataaagagaatatagaagacttctaattacctcaatgagatacggcaagtcaaatttgcagatattatatccaattataatatctgggtccacttcacggataaaatcctgcatgcaaaacctgcttactacatcataaaatgcactatatcctactacgtgaaattattaataacctttatgcagcacctgatcataaaactaatacaagatcaacccatcaactgatagtacagttaagatacacattgttaagctagagtcctatatttaagcactgcacaaagattagggtatcaaagaaggcaaagctaagggtgcatgaagattcatgcttacacagcaaatatatggaagaaagtggattttagtgcaaccatggccaagtgtttgataagcatagttgtgacaggagaatctaaaagaatggcttcttcagccaaaagggatttccatgccaaaccaaattgtgaactaacattttgattagtttgatttctgtactcaaagatagtctaaattggtttgagaaattcacaaactgtaaattgtggtttcttcttgatatccaaaatccttttaactgccaaagcagattttgcacacctctaggtcatgctcagtatatgagattgttgactatgaactgagaattttgtttaataagatagatttaccgaaatctaaaaataaagaaataaataaataaaaaggaggaggaggagataaaggatggagtgagaggtaaagaggagcaatttcacctggctgtatgtactttaagctaggttttcaatcaaacactatgaatgaaattttttttatccagagtaataaataaataactgtggaagccattattttctgaaacaagcaaataagatggcatttcacaagataaaaaaaaaaaaaaacagtccatcccattgatcaaatgcagatagctcaaagcttcaacttccaacttccattgacggacacattcaagatggtggttccaataagacatgaacattataactgcaggagacacatgggccactacagctatagatcaaactggattcaagcagcaaacccaggaagataggagtatatgacagatgcatctattaccatactattaaatttgctaaaatatttttgctttagaacaactatgccattcaaaagtctcaggttgctattatcttatgagttttttattatattacttacagaaattttcctcaccaaatctaacatgttaaaataattgtatagaagagttattcaaattataaacatgcaacctagctttcttcaaagcaaaagagaactatagagaaaagcaaacataccctccaagccagcaggacttctctctccgtatcaaaagacatcacatctacaccaactattggagagcatgagtttaaagtcataacattacggataaatggttggtcctccccctgcatagtaactagatttgccacctaaaatgaaatggaaattttgatttcagcaacagtgaaaaaagaattatcatccatccaccaaccagactgcttgactgaagtaaaacaagcataaaaactagggaaaaacctgaataacaggatcatgggtaggctcaggaaaatgacctttccgaccagcacattcaatatcaaaacttaaaatgcggaatggagccatctttgagaattccccttctggggcatggcttatcaattctgaatacctatgaacattggtcaaggaattaatcttgaaaaacactacggaaatgttcactgtggaaaactggagtagttttctagaaaagttagctaagagaactggaaaacagagtttcattcacttatgccaattttccaaatgaaggattggaaaactagcttccggttgccttgaaaatttaactaaggttaggaaaatagtgataagttagtttttactattttcctttacgaaaaaatcttcgtctccaaattttttgacatagaaaataatgacttttctatatctaaaattacaccataaatttgtcataactaaaattgaataattatttaggaaaatattaatttacaatgataaaaattaatcatactattatacattaaatgaataacaaatattaaaaaatttaaaagttaaacaaaattttatatataatttataatgatttttttttattattatgaatgatatgaatattttaaaaaattgaattagaaaattgttttctataatgggatctccacgttttcccttactttatagaaaatgcaaaatggagagtttccttaccaaaaaaaaaaaaaaatgaaaaacagaaaattgttttcaagaaaacagccccaagtatggacaataagaattgaagtattctctcaaggaaagaatgatcgagatgcataaggatacaggcaatcaaactcaagttggccatatgacagattctttgatgtcttcttatattttccagctggtacttcaatccaatttccaccaacaacattacagtcaatcatgaagcggagggcaaagagaacattgctttcatatgtcataaagctcttcataccaaggccatcaatgtgtatgcccttatcaagaatacctgcaaatgattgggaattgtcaagagactcaaaattcaatacagatatcaaaatcattatgctgtaaacaacttaaaagagattatacatgtttgcatcacagttacatctattaaaaaattgaagatgcttaccacgacagctggctaccattgttggcaatgcaactacaatcttcagaaaaggatgtgatggctgttgctggtaatacataatgctccttttttgaaccatttctattcgccgtacaaatttgggcacttgactgttcctattcatctccttcatcctccccttaaacacccaaaacaaagatgatattgagataattaagcaatcaattagctctatatacatatgtgtgtatgcatgtagttatctataaatatataggcagagaaattcgtagatgaaatacctcaagaatttgttgaaaacgagaaatatcatcaggacccatcccaggagggcaactgatgtaaaaatatggctcaaatccgtgaacaaggcagcaaacactatgtcctaacaaaaagtcagtatgtaaatatattcgtgtgaggtagaaacaattacaagtttcaaactgtatgtagttcaagatgctttaaccatatccatagctataaagtttattatataacacaacgttgaaattggcaagagaataaaatgataaaaaagtaactgagattctcaaagagcaatatgtcgctgattcaaattcaacaagaaaaaaaaaatcttaaatgatatagaaactaatcatctacctattcaaagaattcatcagtgcaagtgtgtgccaatgtcaattagagaaagaaatttaaaaaatatggatattgtgttacatgtatgtatcaaatctaaatttgatgaaatagacaaactgggttgaaatcagaatgaaaccactgatccaaagtcgaaaaagcaaatgtgtacaatatttttgtttatttggtttggaaatcagaggagagaggcaaaatatggagcataattggaaaccaaattaaaatatactgaaataagaatgttacagaataaatagatgttgcatttccaggcacatgaatagattgccatatttaactactcatgcataaaacataattagtacactcgtttaccaatgaattccatgaatatgttatcaaatttgccaattagaaacctttgaaattgaaaactacacccttcgcttcaccgagtaaaaaacccatagatagtgtaagtaaaagtatgcttctacacatatacaatacaactacaccacaaatttaacaaatttattaaaaaaaaaaagtaaaaaatttcacttttctatagcatgttctcagttttatagggattattttctaaattacaataatgacctaaccttcccttgtgaccccaaaaattctgataatcgcagcgacccctgacctatcaggcagcagctccttattactctctccaatcacataatcaatttccagctgctgaaatactgaccgtaaagaaaataataaatccatcgaatttcacaataaaagaaagataaaagaaaataaaagtaatccacaacaataatacccataattaggaaacaccgaagttactcactaatgctacgagattgggaagtgtactcgtgagagaggggcggccgagaccacttggcaaggcgggatgcaagggcctggcgctcctcgaggtcacggaggatgagattctcctcttccgcaataagggtttcatcaaggaaaacatcctcatccacgaattcatcgtctacggctggtgtcgtggcttgatgcttcggtgctggtggcgatggcgtgggctgtgatgtcggcggggggcgcttcctggtgttgccatttcggttcatggccgtcgttttggcgggagatttcagttcgctgtaccgtgcttagaaaaggagggaaagtagatgaaaagaaagcgagggaaatagaaataatgagagtgagagtgaga

SEQ ID NO: 55 - DNA, 3863 bp, Manihot esculenta
  source 1..3863: mol_type = other DNA
tctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatgttcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaatctgataacaagggaaaattcggaagtcagtttgtcacagaggcctgcaatagttgagggtccaactgacttgaaaggttgcatatctgattggctagtattaaggcagaagctgtgtcgagacatgagttctaaaatggcagattttcaggttgattgacctttcgcaggctttgatagtcgggatgttgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttattttggtctatcttggattacttgtacatctctggccatttttggctgtagcttgaatgtcattacgata

SEQ ID NO: 56 - DNA, 3276 bp, Manihot esculenta
  source 1..3276: mol_type = other DNA
atgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatgttcgacaactggccttgaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaa

SEQ ID NO: 57 - protein, 1091 aa, Manihot esculenta
MNRNGNTRKRPPPTSQPTPSPPAPKHQATTPAVDDEFVDEDVFLDETLIAEEENLILRDLEERQALASRLAKWSRPPLSHEYTSQSRSIIFQQLEIDYVIGESNKELLPDRSGVAAIIRIFGVTREGHSVCCLVHGFEPYFYISCPPGMGPDDISRFQQILEGRMKEMNRNSQVPKFVRRIEMVQKRSIMYYQQQPSHPFLKIVVALPTMVASCRGILDKGIHIDGLGMKSFMTYESNVLFALRFMIDCNVVGGNWIEVPAGKYKKTSKNLSYGQLEFDCLYSELISHAPEGEFSKMAPFRILSFDIECAGRKGHFPEPTHDPVIQVANLVTMQGEDQPFIRNVMTLNSCSPIVGVDVMSFDTEREVLLAWRDFIREVDPDIIIGYNICKFDLPYLIERAETLGIAEFPVFGRVKNSRVRVKDTTFSSRQYGTRESKEVTLEGRVQFDLLQAMQRDYKLSSYSLNSVSAHFLSEQKEDVHHSIISDLQNGNAETRRRLAVYCLKDAYLPQRLLDKLMFIYNYVEMARVTGVPLSFLLSRGQSIKVLSQLLRKAKQKNLVIPNVKQAGSEQGTYEGATVLEARAGFYEKPIATLDFASLYPSIMMAYNLCYCTLVTPEDVRKLNIPPECVNKTPSGETFVKSNLQKGILPEILEELLAARRRAKADLKEAKDPLVKAVLDGRQLAFKVSANSVYGFTGATIGQLPCIEISSSVTSYGRQMIEHTKKLVEEKFTITGGYEHNAEVIYGDTDSVMVQFGVPTVEEAMKLGREAAECISGTFIKPIKLEFEKVYYPYLLISKKRYAGLFWTNPNKFDKMDTKGIETVRRDNCLLVKNLVNECLHKILIDRDVPGAVQYVKNTISDLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNSDRTLCTHCKGREAELYCKTVSQVSELELLFGRLWTQCQECQGSLHQDVLCTSRDCPIFYRRKKAQKDMAEAKRQLDRWNF

SEQ ID NO: 58 - DNA, 22400 bp, Manihot esculenta
  source 1..22400: mol_type = genomic DNA
ctatctctcattctcactctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattagtgagtaacttcggtgtttcctaattatgggtattattgttgtggattacttttattttcttttatctttcttttattgtgaaattcgatggatttattattttctttacggtcagtatttcagcagctggaaattgattatgtgattggagagagtaataaggagctgctgcctgataggtcaggggtcgctgcgattatcagaatttttggggtcacaagggaaggttaggtcattattgtaatttagaaaataatccctataaaactgagaacatgctatagaaaagtgaaattttttactttttttttttaataaatttgttaaatttgtggtgtagttgtattgtatatgtgtagaagcatacttttacttacactatctatgggttttttactcggtgaagcgaagggtgtagttttcaatttcaaaggtttctaattggcaaatttgataacatattcatggaattcattggtaaacgagtgtactaattatgttttatgcatgagtagttaaatatggcaatctattcatgtgcctggaaatgcaacatctatttattctgtaacattcttatttcagtatattttaatttggtttccaattatgctccatattttgcctctctcctctgatttccaaaccaaataaacaaaaatattgtacacatttgctttttcgactttggatcagtggtttcattctgatttcaacccagtttgtctatttcatcaaatttagatttgatacatacatgtaacacaatatccatattttttaaatttctttctctaattgacattggcacacacttgcactgatgaattctttgaataggtagatgattagtttctatatcatttaagattttttttttcttgttgaatttgaatcagcgacatattgctctttgagaatctcagttacttttttatcattttattctcttgccaatttcaacgttgtgttatataataaactttatagctatggatatggttaaagcatcttgaactacatacagtttgaaacttgtaattgtttctacctcacacgaatatatttacatactgactttttgttaggacatagtgtttgctgccttgttcacggatttgagccatatttttacatcagttgccctcctgggatgggtcctgatgatatttctcgttttcaacaaattcttgaggtatttcatctacgaatttctctgcctatatatttatagataactacatgcatacacacatatgtatatagagctaattgattgcttaattatctcaatatcatctttgttttgggtgtttaaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtaagcatcttcaattttttaatagatgtaactgtgatgcaaacatgtataatctcttttaagttgtttacagcataatgattttgatatctgtattgaattttgagtctcttgacaattcccaatcatttgcaggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtatccttatgcatctcgatcattctttccttgagagaatacttcaattcttattgtccatacttggggctgttttcttgaaaacaattttctgtttttcatttttttttttttttggtaaggaaactctccattttgcattttctataaagtaagggaaaacgtggagatcccattatagaaaacaattttctaattcaattttttaaaatattcatatcattcataataataaaaaaaaatcattataaattatatataaaattttgtttaacttttaaattttttaatatttgttattcatttaatgtataatagtatgattaatttttatcattgtaaattaatattttcctaaataattattcaattttagttatgacaaatttatggtgtaattttagatatagaaaagtcattattttctatgtcaaaaaatttggagacgaagattttttcgtaaaggaaaatagtaaaaactaacttatcactattttcctaaccttagttaaattttcaaggcaaccggaagctagttttccaatccttcatttggaaaattggcataagtgaatgaaactctgttttccagttctcttagctaacttttctagaaaactactccagttttccacagtgaacatttccgtagtgtttttcaagattaattccttgaccaatgttcataggtattcagaattgataagccatgccccagaaggggaattctcaaagatggctccattccgcattttaagttttgatattgaatgtgctggtcggaaaggtcattttcctgagcctacccatgatcctgttattcaggtttttccctagtttttatgcttgttttacttcagtcaagcagtctggttggtggatggatgataattcttttttcactgttgctgaaatcaaaatttccatttcattttaggtggcaaatctagttactatgcagggggaggaccaaccatttatccgtaatgttatgactttaaactcatgctctccaatagttggtgtagatgtgatgtcttttgatacggagagagaagtcctgctggcttggagggtatgtttgcttttctctatagttctcttttgctttgaagaaagctaggttgcatgtttataatttgaataactcttctatacaattattttaacatgttagatttggtgaggaaaatttctgtaagtaatataataaaaaactcataagataatagcaacctgagacttttgaatggcatagttgttctaaagcaaaaatattttagcaaatttaatagtatggtaatagatgcatctgtcatatactcctatcttcctgggtttgctgcttgaatccagtttgatctatagctgtagtggcccatgtgtctcctgcagttataatgttcatgtcttattggaaccaccatcttgaatgtgtccgtcaatggaagttggaagttgaagctttgagctatctgcatttgatcaatgggatggactgtttttttttttttttatcttgtgaaatgccatcttatttgcttgtttcagaaaataatggcttccacagttatttatttattactctggataaaaaaaatttcattcatagtgtttgattgaaaacctagcttaaagtacatacagccaggtgaaattgctcctctttacctctcactccatcctttatctcctcctcctcctttttatttatttatttctttatttttagatttcggtaaatctatcttattaaacaaaattctcagttcatagtcaacaatctcatatactgagcatgacctagaggtgtgcaaaatctgctttggcagttaaaaggattttggatatcaagaagaaaccacaatttacagtttgtgaatttctcaaaccaatttagactatctttgagtacagaaatcaaactaatcaaaatgttagttcacaatttggtttggcatggaaatcccttttggctgaagaagccattcttttagattctcctgtcacaactatgcttatcaaacacttggccatggttgcactaaaatccactttcttccatatatttgctgtgtaagcatgaatcttcatgcacccttagctttgccttctttgataccctaatctttgtgcagtgcttaaatataggactctagcttaacaatgtgtatcttaactgtactatcagttgatgggttgatcttgtattagttttatgatcaggtgctgcataaaggttattaataatttcacgtagtaggatatagtgcattttatgatgtagtaagcaggttttgcatgcaggattttatccgtgaagtggacccagatattataattggatataatatctgcaaatttgacttgccgtatctcattgaggtaattagaagtcttctatattctctttatgcatttttatagtgaagtttttttctttaattttgttttgcagtttaataaatttgaaatgctatttttcttggtttacagagagctgaaactttgggtatagcagagtttcctgtgttcggccgtgtcaagaatagcagggttcgggtcaaagacacaacattttcatcgaggtagtatttttgtttgttacaacaattatcatgtcttttgtgtgcgtgtctttcgtgtgcgtgcttacatattcttgattggagggcattgctttataaactttttttttcttctctcgctttttataatcttcttgttgtagttgtatttggagctctttaggaggattccttgcttactatgctgtatttgttgagttctatttgattccttctgtaccttgttttattggtcaagttctgtagaagtattccattccatgtctaccatgtagcactctcctttctttctcaataaaatattttcgtatatattgaaaaaatattgatgccgtcataagctttaagttgaagattagaatacttgtgcacagtgcatgttatacttttgcttcctgtctaagatttttttttcccatgaaatttgatgagaagagttactgccttcccgagaatttcagagatttaatgagaagtttaatgagaagttcagagaatttcatggaatatgagtgatgtgcttttatttgtctataaacccaaaatgaacttttatgtccttttggtttcataaatttcagactctcaggtgaaatccctagtagtcgtaaaaatagcaatataattgggaagatcgtattgtaatttgatttcaaaaatctgggtcatccttaatgtgagagtaagctttaaagaatgtgtgtatggtgttccttacctggatttagtgcttaaacattggcaccttaatccttgttgctgtttgcagacaatatggtacaagggaaagtaaagaagttacattggaaggaagggttcaatttgatttgcttcaggttttgtgcaataaaactttttctttccacaatctcatttttgtttctgagataaaagttcatttaggcatgttgttatccttccatccaggcaatgcaacgagattataaattaagttcttactcattaaattctgtctcggcgcacttcctctctgagcaggtaagatatagaattgtttggctcaagtgtcatttcttcttgaagtttcagtacactatgattgacctgctttgttttttattatctaattgttaatatacaccatagaagtagtcttagctggatcaaaatatgagtaacttttcctccctaactttctccatttgctttctgtaattacttttgtactacgtctttgtaagtttctgccttgaattattgttcatactggtaatgaaagcattggttgcaataggaagttagcaattttctcagaaatttacgttgcacattaactattttggttctttttgggtctcaatcaatgtatgtgcagtttttctctctcttctctcagccattttctatttaaccttcttcattatggaaaaaagatatttggtgctgatgttgctgacatgttatttgtcttgttcagaaagaggatgtccaccattcaataatatctgatcttcagaatggtaatgcagaaactaggaggcggcttgccgtgtattgtttaaaggtgcatagttttgtttatcttctgtggccctttttcctaaaatttgccttactttgcttctctcttttgctggcaattctttcattattgaactatatttgatgcattaaaatttgacaatttatttcttctttgaaacaaggatttgcatcgactagtctatcttgatattatagattgtgaacttgatgtaatgaaatgcagtgagtctgctacctgtttataaatctttagtgttactggatattgattgaaatcgtgtggtactttacttttggaattgcaggatgcttatctcccacagcggctcttggacaagttgatgtttatttataattatgtagaaatggcccgtgtcacaggcgtccctttatctttccttctttccaggggacaaagtatcaaggttagtgacctgtcatcgcacatcagttgattgaatcttacttgatcaagctaatcaaatcataggatgttgacctcttgttttcaggtactttctcaactcctgaggaaggctaaacagaagaaccttgttattcccaatgtcaaacaggcaggatctgaacaaggaacatatgaaggagccactgtgagtaaattttatctgcatggtcatgtgtactgttttaagaattacaatggctttgagtcgatagttccatctactggtttagccttattttatgtaaatatatcttgctagaaagtaaaagtatatggcttgcaatgttatacatcagtttctgactattaatttgtagttagaactgcaagtgccactatttcaaaatgttacacatcagtttctgactattgatttatagtaagagctgcaagtaccactgcttcaaaatatttggtttttgcgtaccctgcattgtttaggcatgtgggtaatctgtctcttataaagacttgagaaatcttcccccttccaaattcttaatggtaataataaaaaatttacatcacataactgattcactaacttgaatactaccatagttgatgaagttgttgtattctttcttttgtgagtacgtgtgttcatgtaggtgtggacatggatgtgagtcctccctcgtgtttctctcttctttcacctcttcttttgtgccccttttttgggttttttgtgagttgggggggtgggtttgggtgtggtttaagtcttgtatcttggtaattgtcttttacttccccctttacaaagctgaatcatctttgccaatgatttggatctaacaaaatttcaggttttggaggctagagcaggattctacgaaaagccaattgcaacactagattttgcatctttgtatccttcaataatgatggcatataatctatgttactgcaccttggtaataatcttgaccatttattctttttgttggcataacgattatggcttgtaatattggcacaaactttgtttatttctatgttaaacttttgtcttttgcttgtcaaggtgactcctgaagatgttcgtaaactcaatattccacctgaatgtgtcaacaaaactccgtctggtgaaacatttgtcaaatcaaatttacaaaaggttaaatcatgaattttttttataatcatttttattggcgtcatcatcatcttgattaccaggattaattagaattatctttcagggaattcttcctgaaattcttgaagaactattagctgctcgaagaagagcaaaagcagatttgaaggtaattatctattgttttggctgctttattaaatgtttgtatttattattcctttgtgcattgcttattattcctttgtgcgttgccaattttttactctgttgtaatgttactgctaggtttctaggcctttgtgctttttatgattttatctaataattttaggaagctaaggatccgcttgtgaaggctgttctagatggtcgacaactggccttcaaggtaagcttatagatacattgatcattcgatgctcatttttagtgttcacaatctatttactatctagtgaggccaaagcttatagaaagttatacgccatcaagtggatggcctcaaatgcttttaaaatattatctcttttctctttaaaattgattgaaatattgaatttttgttactaaaaaataaatataagtgattagttaaaatttaatgaatacgttgaatgaattatttcattatatcccagaatatctaactttgagttaaagctaacttttgtacttcccatatttctttttgttaagattcaataattagaaaatatattttaaaaataatgttattcttagatcaactattattgcttgaaatttgtattctttactattttatattcatgaaagaatatttagcatttccataaagtgtatggtcttatcatcttggaaattatttatttttttgtgattaatataatattttatgaagtgtctttaaaattttccctcccttggcttccctttttggttatgttaatgtttaatgagaaaatgacttgtttatcattctgttatatacaagtaaatacttgttggtttttcagtaatccaaatatttaattttatgaattttattgataaatttttgtgtttatcaaagtttgactatgtatatttaacattttcaaactaattgttttatattatttagtgaacttagatcactcattttgtagacgatgcaaaaagtgcagtgagaagactagtataataagataaagaataattgggcaatttcagtgtgttttctctgttaaagcaaatgtgtatatatacagattacaagggcaatttctaagaattctttattaatcaattagcctatgattatataatctcctttaattatgtatagattatgttcacaatctaatactctcccttaagttggagcatagatgtttatcatgtccaacttgttagaaaaatattctattcaaggctcatttaaagttttggtgagtagatcacccaattgctctcctatcttcacataactactcacccagttgctctcctgtcttcacataactagtcacataaccagtggataactactcacccagttgctctcctgtcttcacataactagtcacataaccagtggagattaaattttcttgaatcttctaatggatgaaatgacaatcaatttcaatatgcttagtccattcgtggtatacttgattgaaagcaatgtgaagagtagcctgattattacactaaagtttcacgggtgacgcaacatccataccaatttagaatctcacaagtgaatcattgagccatggctctgtattctgactaggcactgaatttggatatcacattttgtttcttactttttcaaaacactaggtttcctccaacaaatacacagtagtctatagtcgaccttttatcacttttgaatctcgcccaatcaacgtcaggaaaacttaagtgcagtatgcccatgatgactcaatcctttttagataatatagaatttgttctagagcagtataatgtttcaccataaatgcagacatgaactggcttacaacgcttactgctaggggtgagcagtattcatttcaaactgaaaaaaccgaccgaaccgaattaatttcaaaattcggttcggttttttattcaattcggttcggtttggtttttaatttcagaaattttggttatttcggtttggtttggttcggttttaatcagaaaaaaattgaaaaaaaccgaaccaaaccgaccgaatgctcacccctacttactgcaaaagcaatagctgactaagtcattaccgtaaagtagttcagctttccaaccaacttcctgtacctctttggatcatcacaagggtctctgtcatcctttgttagatatatatattaggaatcattggtgtgctagagagtttagctcttattttcccaatttctgtaagtaagtcaacgatatacttcttttgagatagaaaaattctctttttacccctcaagacttcgactcctaagaaatatttaacttgactcaagtttttagattgaaaacttgcatgcaagtagtttttgagaggagatcttatactatcattctctataatgataatgtcattaacatacacaacaagaagaataatactagtatctgaatttttataaaagaatgaatggtcacacttgctttttttcaatccaaatttttgaataacttcactgaacttgtcaaaccatgcacggagactctgcttcaaaccatataaagattttttcaaatggctgacttttccatactcccttgagcaacaatggttgcttcatatacactttctcttggaagtcgccatgtaaaagtgcattcttgatatctaactggtgtaaaggacaatgctgagaagttgttagggagatgaacaagcaaattgagatcatttttgcctcaggcgaaaaagtgtcagaatagtcaacgccataggtttgtacataacctttagcgacatgaaggctatagaaccatctggattgactttgatgacaaaaatccatttagagttcacaacctttttgttcgagggtagattaactagttctcaaatatgattttcatctagacccttaatctcctcaaacattgtatcatgccatccagaatgagtcaaggcctctttgactgtcttaaacaaagaaatggaatctagagagacaattaaggagatagaagaagacaggtaattataagacagaaagttagcaacagagtatgtagactttcattgtcgtttacctttacaaaagctaatgtgaaggtcaaggtcactcggtggtgaatccgatggcaaagaagattctggtgcaggacatgtatcatcaggtacttggtctccgagagtaaacttgaacaactgccggtttaataggaggctaagtattaggaggaatgttacaatcatattgtacaacaaaaggcatactcaaagagttttcaccatcagatatgattttataaataagtcgcacatcgtcctcctttcaactagtagaggtttgtgtaacaggataaaaaggaatttgctcgaaaaaggctatatctattgagactagatatttgttgaggtttggagagtaacactgataccccttttgaaggggatcaagtttagtcacatgaggtttgatattccgaacatagcactaaaaagtcgtggtttcagagaaaataatagcttcttaagaaagggcatgcattaatgagaaagcattcagtagagacaatatcagtccaaaattgcttagaaacttgcatttgaaacaacagagctcaggcagtcttaaggagatgtcgattcttcctttcagctaccccattttgagcgggtgtatcaacacatgactattggtaaagaatactatgttgagtcatataagtctgaaatgattttgatatatattttttagcattgtcctttcgtaaaatttgcacaaaaacattaaattgagtcttgatttcagcataaaaggcaaaaatataagaaaacatctctgaatgacactttataaaataaatcaaagtcgttctagagtaatcatccacaaaagtgataaaatatatgaatctggtcttagatccaatcggacatgggtcctaaacttctgaatggactaattcaataactctaggacttaaagagtttcaatgatgttttgtaaaatgacatgactcacattttagtgaagatacctcatgaaattgagggcatagcttcttcaaaactaaagaaggatgtctcaaccgacaatgtgctttgaacggagacacgacactggagcagacaacgcattgaggcacccatgcatccaaaatgtaaagacccaagatacatgtcttttactaataatctgttttgttataagatcttgaatgagacaatgattaggaaaaaaaaaacacaataatttaggtctttggtaagtttacttacagagattagattaaaggaaaattaggtaagcttaacacacatgataggataataggagtaggtttaacagtctcagcctcagaatccaaaatatccaaaatattataggttgatctatcagctatagtaaaaagagaatgtgttttatgagatcgaaagctggtaaaaatatgagaattacatgtcatgtgatctgtgacattagaatcaatgatccatttgtatgaagaggaaataagacaagtttacttatctcagcgtctaccggagacgttgagataattttaataatatgggttttactattagaaaccatttcaaccaaaactttatatttctatgccttcaaatgaattaaaggaaaacaacagaacctaatcacgtgaacagtaacccagtgaacagacagtaggctaccactgaaggacactttccaatcaatgtcggatggctggagttctactagagacgtgctggaaatgaccgggcgattgttggggaagaaaccggaagtcaaacggaggccacacatgctcccgcgcgctgtcgcataaaactagagctgaaatttggatcggcgcatgagcaccactcgcggaagctccagtcactgttttctaaggaacagttggaaataatgttgcgtctctccttgggcctttggtgagatcaaacagacacccaaacttgatgacccaacaactagtttctcaacccgaaaaattcaaaaaaattagacatccaaaattagctcaaaggctatgatacaatataacaagacaaggaataattgggaaatctcagtgtgttttctctattaaagtaagtgtgtatatatatagattacaaggccttattaaggcaattcctaagaaccctctatcaattaattagcctataattatgtaattttctttaattatgtacggattatattcacactcgaacaactaacttagatgcatgttggttcaggatttatacaatcttccattggtttgagctctttgattatcttatgttattgtaggtaagtgcaaattctgtatatggatttacaggagctactattggccagttgccatgtatagaaatatcctctagcgttacaagctacggtatgctaatacaagattctccctaattaatatggtgtttttttattcaatttgatctaacatgttgttggttgcaaaggtcgacagatgattgaacacactaaaaagcttgtagaagaaaaattcaccataacaggaggatatgaacacaatgcagaggtatgcaatctggaatttagatgagtattataatgtagtattttgaaaatgtttattggttaaaatgatgtagttattttaaaatgttgctccatttagaagtattttggtttgtttttaagagtttcacatcataaaattcataaatttaacctttctttgcattaaacacaccattttgttttattgtttaatccagcatcacacactaatttattatctttttgtccttgttttatacctctctgtttgatttgctgtcactcacactctctttgttttgtgcattaatcaatttgttcttattatagatgtgactttagtttcgtttccaatgctctcagtcctaatgccattttagtatcattatgcgtctttcaaaatcaaataatcttccaaattgaattttgaaatttccaatttgtggttttggatttacacaatatcttagttcaattcaaacagaagactttaggtttgagaactgattgattttcactggaaaactaaattgaaccaattcaatttcagtttttgatttggtttaggagagagcattcggccggtttggttctgaaccgaactgaatttgaaaaactgaaaaccaaagtctttaaaattataaaccgaattaaattgatattatagaaaaatcgaaccaaatcaaactgataaatatcagtttggtttgggtcaacaaaaaccgaaattcccaaattatagcaacaagcctaagaatggcctcctcaaattgcaacaaccaacaagagaatgacacttaatattaatttgttccaaaatcaatatataacatttgtaaatagacaaggaataattgtgtaatttcagtgtgttttctctattgaagtaagtgtgtatatgtacagattacaagaccttgttaagtcaacttctaagaattctctatcaatcaattaacctatgattatgtaatctcctttaattatgtataaattatattcacactctaacactccccctcaagttggagcatagatgtttattatacccaacttgttaggaaaatattctattcgagactcatttaaagttttggtgagtagatcacctagttgctctcctttcttcacataactagtgaagattaaattttcttgaatctactcacggatgaaatgacaataaactttaatatgcttagtctatttgtgatatactggattagaagccatgtaaagagcagtctgattatcacaccaaagtttcgtgggtgacacaacatccataccaatttctttcagcagatgatttatccatgccttggtcttgtattctgactcggcattagatttggacactacattttgcttcttacttttttaaaacacgaggttttcttcaacaaatacacagtagcctgtagtcgatcttttatcacttttggttcccactcgatcagcattagaaaaacactcgagtgcagtatctcgagtgcagcatgcccataatcaccgtagactatactgagtcatttagttatgataaatactatacatttagttatgatatttatgaattatataaacactataaatttagttaaaattgcatataaaaatatatgcattttgttagttatttcattacattttgatattttattacactattactattattattgagcgaaaatcactattaaaatgcattacattatttttttaattaaatataatattaaaaatgagaattttaattatctaatgaattaaaattttatatggtactttatatttaattttcagattatttaaatgaattttattaatttattatgaaccatatatttaatataaaaataattttaacatttaatttataaaaaaaaattagaaatacatgaaaagtgagaatttattaatagttacagcaggttaaacacacacttctgcatgtttttatattatatgtggcaactatgcatgttaagattagtacaaaagcaaagaaaaatcctcacttgtagacaacccaaaaagcagaggaaaccacagcatcaaagaagaaaaaaacagggtgggcagattatcttcaatgagtgggagggcaacaagagaaggaagaaagaagaaagagagaaggagaaaataaaaaaaatccaaatgatgttcttcaagcaaaagcagaaaagcaaggtgagaagaaggaggagaaaagatgaaaaaataaaagaaagaacctggatttgcatctatccattcatgagtttggatttcctcttcttttttttttcttcttccgacaactagaaccgccttgtgtaactaggacgtgccttgtctgcgcctgcatgagggcgcctcccgcctgacaccactcaaggcgccagggctggcgcttggtgcgccttgcgcctcaagcgcgcctttcacaacactggtaataatataataaattatcttgttattaatattatgaaaataaaattttagtattggtgttagtaatatataataaatattatttatttatttataaatattgtctatttatagaaaattaatattaattttagttatatttatatattttttattttttattatatatatatatattatattattattctatgttctataatctatattgtaattatattctatatttatattttatgaaatagtgttttgaaataataatattatttattgtataaattgttatattatatagtatatataatttgtataatttagaaatttatatattatttttggttaatattattatttaattataaatatattatctataattatagttatatatatatgtataaatttatttatatatattaagaaattaatatttatcttatatattatataaacataatttatcaccaactatattttatatattttaatataattgaagaaattaatatctagttagattaaaatatataaattgtagttagtgatgcattttatgcatatatacatgtacatttaaaatatataaaaaaatttttaactttgtccgcttttttaaaaaggctttgccttgtgccttgcctcttgcctcttgcctaaggggatagaaggctccatcgccttagtgttgcctcgcgtgcaaaagccctagttatgaataattcgcctgagaacctgcatatgcgatggtgatgcttcggcaccaagacatgcacagctttgacttggcgagaacatatggtgattggcaagaatggacggtgatagaaggctctattgccttggtttttcagttttgttcaaaactgaatcagttgattgcacacctagaagaaatacagaatttggttattttggtttttacaatttttttatcaaactgaccaaatgctattccctcatgcaaaagcagtcaattcttggtgccaacttaaatcatattctcctgccgagatcattagtaacttccgcaaattggctggcttctgtttctgtggagtttatgattggagagaaggctagatatgttggtggaattggaagttaaataattttatgaatggagagaaagctagaacttttgatggaattgaagtttgctaagtgatgtgggattttaaattgattttgctgttctgctggttgtcttatttattcaccatatagatttattgagtatactttattaatgtattagactaggctatccttttctttcggtgcactttgttcctcgtttgttctaaaatccacaaagcttttggcatttcatatcaataacacattgtggtaagctttatctatagggaaaaagcgtcgtagattaccacatgaaaaatattatgcatttttgtctatttggaaatttgaatttcaaatcatgaacataagaatgcaaaaatgagtttcatttacggaatatgtggatgtagtaagctaatcctctctattttacatttctatatgcttggtattgattaaaattttcttacatacctgttttgagtacaatataggtcaaaagcagtcctactttttatggtaatagcttgacctgatcgaagtgataatttcaggttatatatggggacacagattcagttatggtacaatttggtgtgcctactgttgaagaagcaatgaaattgggaagagaagctgctgaatgcatcagtgggactttcataaaagtaatttttatcttgaattctctgatttagaatttaactatttgatagtctttgcaaattcatttttagttaaatcagccaggatagctaatgaagagcctttgaaatacagcaactatgataagctttgaatggtaattttgaactccaattaaatgaattggcagtgtactgtgatttttcgcaaaaaaggaaaggcagggttgaattactcaattgagatattatttcctgaatatgaacttctcaaatatcaaaattgttagtttaattgttgttcttttttcttttttggtctcaatatagttgcctctgtctcacacatgcatttttttgtgtgattgttttagtgtagttttcttcatatgacttatggatttcttaacttttagcaaatgagtaaaataaaccaaatctaagtctgttattttccacgagattcctcttatacagctgaaattgaacacagccaaagttactatactggttttatgttcaatatttttatacctttaattgagtcaacttcacaaggttactcgtgtgagttggtgacatttaataattttttctgtgcaaatgttgctaggaagttattgtcggataatgctacagcgcatcatgatacataagttagaatgatgtctgaataaatcagtcatttatattttaaaaatagtcgtttctattttaaaaagataatcactgggggcttgaagactatctagtctggcaacagtgggaagagacatgggaattctcattatgaaatttgttgaacttggtttctgtttgctatagctatatgattttttcagatatggatacacctgtagtgttcatccaacttgaattctaattaaatgctaaggtttggaagctgtcaaattagctttatgtgattcaacttttggcctttttttttggccttttttttctatcaacttttttaatggttatcatttaactcactcatttcatttttgtatttgggtttttgaatgtgaagcctatcaagctagaatttgagaaggtttattatccatatctgttgattagcaagaagagatatgccggtttattctggactaatcccaacaaatttgacaaaatggatactaaaggtgaatacaacctctcaaacatctgagcttctggatattgtttgggaataatctattgacttttgtcatatatcttgggcaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaaggtgtgaaattgtattggttagggtcctacatactgtccttaaactgctgtataacaacatctaaatatcttgtgtcatcaacatggatattttcttcctgtcctaatttattttgtttaacagggtctgacaaagactggagacgattacgaagttaaggcagctcatgttgaacttgctgaacgcatgcgcaaggtgctttttttttagtgttcttttttcctcctttgtagaagggcttgttcttttgctagaattctggagagaggaattaaaagtctgggaccttaataatcttgtttttatttagataagttgagattaggctcaaaattctataggaatttaaaatcagataggatgggatttttagatttttaaagattttatttaaacttaattattctggtgggcaaaacataaagtactactaatctacgttgaaacaaggaaaatagcaaagaaaagaaactactgatgccatctgtaagaacctggatagattcttcgatggattttggagattaggtgagagagaaagtagatgtagatgagaagagaagggaggagaaaatagagtagatgagaagagaattaggagagtagagaggaggattctgttattggttattcttcaatgatcaatacagatagttgtttcttatttatagcagacccacaactaatttctgttgcaaccaattttccctcgaaaaccaacagctcacacacacagctgtactaagctcctctgtttttcctcctaaaatacataacaccatcttatcattttgaatgatattcaccaacatgaaacgctccaaggcacaagccataaagaagccaagaaaactatcttactctgcaggatgtgggaaaacaaagtttaataatctataatacagcataagatcttattctgtttgttattgacagagagatgctgccactgctccaaatgttggggatcgagtaccttatgttattataaaagctgcaaaaggtgccaaggtagttttctgggatttggttttgtttaatacctttttctccaaattacttggtacggtcctctgattgttcaatttttatatgcaactgataatcaggcttatgagagatcagaagatcctatctatgttctggaaaataatataccaatagatcctcagtattatcttgaaaaccaaatcagcaaggttagtttggtatatgttttgtacaaagccaaatagtgattctttttctaatattcattaattataaatgttgatattgcagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattaggtatgttttatttgtattccactttatctctgccttagcactacccgtattggctgcaaggcactaattaggtacttccaaggtctctgtggatggtgggaggccctagattacttgctatagcttttcggtttcttgtacttttaattggtaaaaggtactttgtaatttgtaaatcaactgaaattgagatctctatttcagcgcctctatgacccaaagatttccttaaacactcatatggttgcattcatttactttcccgagttatataatatatttgagatatctgaggttttagaggagaaggcaaatttggcaccatttatttatttatgatttatcaagttcaatgattgcatgatttttcactctttttctcattctgcttagtgaacttctgttcatcacagactaagagtgcaaagtaggattctgtattctttctcacttacaataataccaattatttaagatgttttactatctccttaattgatcagatctattgtaatagagtgaaaatcaaatgcacctttatctctagatattttgtgcattcaaaattccatatgcgttttttatgtaataattagtgctccttttttctgcttttgctgtgggtatttcctcattgattatgggattctatccttctttgctgccatacgaatacaggcattatatttatttgaagagcctaatactattaatttggttttggaccagcaattcagatcgaactctctgcacacactgtaaaggaagagaagcagaattgtactgcaaaacggtttctcaaggtagacatctcttctgaattctgaatcatgataatttgtgtaattgtaatagagatggtgtttattaagcagaattcatggattgattgcagtgtccgaactggagttgctttttgggaggctgtggacacagtgccaagagtgccagggctctctccatcaggatgttctttgcactaggttttattttctttccattctgttaccattaaataatattctttgaaaaaaatgtttgcagggtttcctggtagctaaattattagctcaaattgtaactaaatattttatttttctttaataaaaagaaagaaagaagaatcacagagcacatattctacgatactttgtaatcactgatttttgttatgcttcatatgtccacagtcgggattgcccaatattctataggagaaagaaggcgcaaaaggacatggctgaagccaaacggcagttggataggtggaacttctaatctgataacaaggttagtatcttttattctccgagtcattgtttatcttttacatattcgtgaattaacaaatctgatgttaactcactagggaaaattcggaagtcagtttgtcacagaggcctgcaatagttgagggtccaactgacttgaaaggttgcatatctgattggctagtattaaggcagaagctgtgtcgagacatgagttctaaaatggcagattttcaggttgattgacctttcgcaggctttgatagtcgggatgttgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttattttggtctatcttggattacttgtacatctctggccatttttggctgtagcttgaatgtcattacgata

SEQ ID NO: 59 - DNA, 3863 bp, Manihot esculenta
  source 1..3863: mol_type = other DNA
tctcactctcactctcattatttctatttccctcgctttcttttcatctactttccctccttttctaagcacggtacagcgaactgaaatctcccgccaaaacgacggccatgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttcaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaatctgataacaagggaaaattcggaagtcagtttgtcacagaggcctgcaatagttgagggtccaactgacttgaaaggttgcatatctgattggctagtattaaggcagaagctgtgtcgagacatgagttctaaaatggcagattttcaggttgattgacctttcgcaggctttgatagtcgggatgttgaactggggaacaagcttacagtggttgcaacttccctatgcttcttgtatcgctgatacttgtgaatacttgcgtctcagttttgtagctaaggtttgctcaggtaattaagagtgtaagtaatacatcttcagagttaatttaatggataaatctctaatccttgtaatctatctctatccccgcgtgtgtttaggccgacacttgaatgtgttattttggtctatcttggattacttgtacatctctggccatttttggctgtagcttgaatgtcattacgata

SEQ ID NO: 60 - DNA, 3276 bp, Manihot esculenta
  source 1..3276: mol_type = other DNA
atgaaccgaaatggcaacaccaggaagcgccccccgccgacatcacagcccacgccatcgccaccagcaccgaagcatcaagccacgacaccagccgtagacgatgaattcgtggatgaggatgttttccttgatgaaacccttattgcggaagaggagaatctcatcctccgtgacctcgaggagcgccaggcccttgcatcccgccttgccaagtggtctcggccgcccctctctcacgagtacacttcccaatctcgtagcattatatttcagcagctggaaattgattatgtgattggagagagtaataaggagctgctgcctgataggtcaggggtcgctgcgattatcagaatttttggggtcacaagggaaggacatagtgtttgctgccttgttcacggatttgagccatatttttacatcagttgccctcctgggatgggtcctgatgatatttctcgttttcaacaaattcttgaggggaggatgaaggagatgaataggaacagtcaagtgcccaaatttgtacggcgaatagaaatggttcaaaaaaggagcattatgtattaccagcaacagccatcacatccttttctgaagattgtagttgcattgccaacaatggtagccagctgtcgtggtattcttgataagggcatacacattgatggccttggtatgaagagctttatgacatatgaaagcaatgttctctttgccctccgcttcatgattgactgtaatgttgttggtggaaattggattgaagtaccagctggaaaatataagaagacatcaaagaatctgtcatatggccaacttgagtttgattgcctgtattcagaattgataagccatgccccagaaggggaattctcaaagatggctccattccgcattttaagttttgatattgaatgtgctggtcggaaaggtcattttcctgagcctacccatgatcctgttattcaggtggcaaatctagttactatgcagggggaggaccaaccatttatccgtaatgttatgactttaaactcatgctctccaatagttggtgtagatgtgatgtcttttgatacggagagagaagtcctgctggcttggagggattttatccgtgaagtggacccagatattataattggatataatatctgcaaatttgacttgccgtatctcattgagagagctgaaactttgggtatagcagagtttcctgtgttcggccgtgtcaagaatagcagggttcgggtcaaagacacaacattttcatcgagacaatatggtacaagggaaagtaaagaagttacattggaaggaagggttcaatttgatttgcttcaggcaatgcaacgagattataaattaagttcttactcattaaattctgtctcggcgcacttcctctctgagcagaaagaggatgtccaccattcaataatatctgatcttcagaatggtaatgcagaaactaggaggcggcttgccgtgtattgtttaaaggatgcttatctcccacagcggctcttggacaagttgatgtttatttataattatgtagaaatggcccgtgtcacaggcgtccctttatctttccttctttccaggggacaaagtatcaaggtactttctcaactcctgaggaaggctaaacagaagaaccttgttattcccaatgtcaaacaggcaggatctgaacaaggaacatatgaaggagccactgttttggaggctagagcaggattctacgaaaagccaattgcaacactagattttgcatctttgtatccttcaataatgatggcatataatctatgttactgcaccttggtgactcctgaagatgttcgtaaactcaatattccacctgaatgtgtcaacaaaactccgtctggtgaaacatttgtcaaatcaaatttacaaaagggaattcttcctgaaattcttgaagaactattagctgctcgaagaagagcaaaagcagatttgaaggaagctaaggatccgcttgtgaaggctgttctagatggtcgacaactggccttcaaggtaagtgcaaattctgtatatggatttacaggagctactattggccagttgccatgtatagaaatatcctctagcgttacaagctacggtcgacagatgattgaacacactaaaaagcttgtagaagaaaaattcaccataacaggaggatatgaacacaatgcagaggttatatatggggacacagattcagttatggtacaatttggtgtgcctactgttgaagaagcaatgaaattgggaagagaagctgctgaatgcatcagtgggactttcataaaacctatcaagctagaatttgagaaggtttattatccatatctgttgattagcaagaagagatatgccggtttattctggactaatcccaacaaatttgacaaaatggatactaaaggaattgaaacagttagaagggacaactgtttattggtcaaaaacctggtaaatgagtgtcttcacaagatactaatagacagagatgtgcctggtgcagttcagtatgtcaagaataccatctctgatcttctcatgaatcgtatggatttgtcacttctggttattacaaagggtctgacaaagactggagacgattacgaagttaaggcagctcatgttgaacttgctgaacgcatgcgcaagagagatgctgccactgctccaaatgttggggatcgagtaccttatgttattataaaagctgcaaaaggtgccaaggcttatgagagatcagaagatcctatctatgttctggaaaataatataccaatagatcctcagtattatcttgaaaaccaaatcagcaagccacttttaagaatttttgagcccatattgaagaatgccagcaaggaactcctccatggaagtcatacaagatctatttccatctccactccttcaaatagtggcattatgaaatttgctaagaaacaactcagttgtattggctgcaaggcactaattagcaattcagatcgaactctctgcacacactgtaaaggaagagaagcagaattgtactgcaaaacggtttctcaagtgtccgaactggagttgctttttgggaggctgtggacacagtgccaagagtgccagggctctctccatcaggatgttctttgcactagtcgggattgcccaatattctataggagaaagaaggcgcaaaaggacatggctgaagccaaacggcagttggataggtggaacttctaa

SEQ ID NO: 61 - protein, 21 aa, Manihot esculenta
FIYNYVEMARVTGVPLSFLLS

SEQ ID NO: 62 - protein, 21 aa, Manihot esculenta
FIYNYVEMARLTGVPLSFLLS

SEQ ID NO: 63 - protein, 9 aa, Manihot esculenta
EMARLTGVP

SEQ ID NO: 64 - protein, 9 aa, Manihot esculenta
EMARVTGVP

SEQ ID NO: 65 - protein, 28 aa, Manihot esculenta
DPLVKAVLDGRQLALKVSANSVYGFTGA

SEQ ID NO: 66 - protein, 28 aa, Manihot esculenta
DPLVKAVLDVRQLALKVSANSVYGFTGA

SEQ ID NO: 67 - protein, 28 aa, Manihot esculenta
DPLVKAVLDGRQLAFKVSANSVYGFTGA

SEQ ID NO: 68 - DNA, 39 bp, Manihot esculenta
  source 1..39: mol_type = genomic DNA
atctgaacaaggaacatatgaaggagccactgttttgga

SEQ ID NO: 69 - DNA, 39 bp, Manihot esculenta
  source 1..39: mol_type = genomic DNA
atctgaacaaggaacatattaaggagccactgttttgga

SEQ ID NO: 70 - protein, 1097 aa, Saccharomyces cerevisiae
MSEKRSLPMVDVKIDDEDTPQLEKKIKRQSIDHGVGSEPVSTIEIIPSDSFRKYNSQGFKAKDTDLMGTQLESTFEQELSQMEHDMADQEEHDLSSFERKKLPTDFDPSLYDISFQQIDAEQSVLNGIKDENTSTVVRFFGVTSEGHSVLCNVTGFKNYLYVPAPNSSDANDQEQINKFVHYLNETFDHAIDSIEVVSKQSIWGYSGDTKLPFWKIYVTYPHMVNKLRTAFERGHLSFNSWFSNGTTTYDNIAYTLRLMVDCGIVGMSWITLPKGKYSMIEPNNRVSSCQLEVSINYRNLIAHPAEGDWSHTAPLRIMSFDIECAGRIGVFPEPEYDPVIQIANVVSIAGAKKPFIRNVFTLNTCSPITGSMIFSHATEEEMLSNWRNFIIKVDPDVIIGYNTTNFDIPYLLNRAKALKVNDFPYFGRLKTVKQEIKESVFSSKAYGTRETKNVNIDGRLQLDLLQFIQREYKLRSYTLNAVSAHFLGEQKEDVHYSIISDLQNGDSETRRRLAVYCLKDAYLPLRLMEKLMALVNYTEMARVTGVPFSYLLARGQQIKVVSQLFRKCLEIDTVIPNMQSQASDDQYEGATVIEPIRGYYDVPIATLDFNSLYPSIMMAHNLCYTTLCNKATVERLNLKIDEDYVITPNGDYFVTTKRRRGILPIILDELISARKRAKKDLRDEKDPFKRDVLNGRQLALKISANSVYGFTGATVGKLPCLAISSSVTAYGRTMILKTKTAVQEKYCIKNGYKHDAVVVYGDTDSVMVKFGTTDLKEAMDLGTEAAKYVSTLFKHPINLEFEKAYFPYLLINKKRYAGLFWTNPDKFDKLDQKGLASVRRDSCSLVSIVMNKVLKKILIERNVDGALAFVRETINDILHNRVDISKLIISKTLAPNYTNPQPHAVLAERMKRREGVGPNVGDRVDYVIIGGNDKLYNRAEDPLFVLENNIQVDSRYYLTNQLQNPIISIVAPIIGDKQANGMFVVKSIKINTGSQKGGLMSFIKKVEACKSCKGPLRKGEGPLCSNCLARSGELYIKALYDVRDLEEKYSRLWTQCQRCAGNLHSEVLCSNKNCDIFYMRVKVKKELQEKVEQLSKW

SEQ ID NO: 71 - protein, 1091 aa, Manihot esculenta
MNRNGNTRKRPPPTSQPTPSPPAPKHQATTPAVDDEFVDEDVFLDETLIAEEENLILRDLEERQALASRLAKWSRPSLCHEYTSQSRSIIFQQLEIDYVIGESHKELLPDRSGVAAIIRIFGVTREGHSVCCLVHGFEPYFYISCPPGMGPDDISRFQQILEGRMKEMNRNSQVPKFVRRIEMVQKRSIMYYQQQPSHPFLKIVVALPTMVASCRGILDKGIHIDGLGMKSFMTYESNVLFALRFMIDCNVVGGNWIEVPAGKYKKTSKNLSYGQLEFDCLYSELISHAPEGEFSKMAPFRILSFDIECAGRKGHFPEPTHDPVIQVANLVTMQGEDQPFIRNVMTLNSCSPIVGVDVMSFDTEREVLLAWRDFIREVDPDIIIGYNICKFDLPYLIERAETLGIAEFPVFGRVKNSRVRVKDTTFSSRQYGTRESKEVTLEGRVQFDLLQAMQRDYKLSSYSLNSVSAHFLSEQKEDVHHSIISDLQNGNAETRRRLAVYCLKDAYLPQRLLDKLMFIYNYVEMARVTGVPLSFLLSRGQSIKVLSQLLRKAKQKNLVIPNVKQAGSEQGTYEGATVLEARAGFYEKPIATLDFASLYPSIMMAYNLCYCTLVTPEDVRKLNIPPECVNKTPSGETFVKSNLQKGILPEILEELLAARRRAKADLKEAKDPLVKAVLDGRQLALKVSANSVYGFTGATIGQLPCIEISSSVTSYGRQMIEHTKKLVEEKFTITGGYEHNAEVIYGDTDSVMVQFGVPTVEEAMKLGREAAECISGTFIKPIKLEFEKVYYPYLLISKKRYAGLFWTNPNKFDKMDTKGIETVRRDNCLLVKNLVNECLHKILIDRDVPGAVQYVKNTISDLLMNRMDLSLLVITKGLTKTGDDYEVKAAHVELAERMRKRDAATAPNVGDRVPYVIIKAAKGAKAYERSEDPIYVLENNIPIDPQYYLENQISKPLLRIFEPILKNASKELLHGSHTRSISISTPSNSGIMKFAKKQLSCIGCKALISNSDRTLCTHCKGREAELYCKTVSQVSELELLFGRLWTQCQECQGSLHQDVLCTSRDCPIFYRRKKAQKDMAEAKRQLDRWNF

(19) United States
(12) Patent Application Publication
(10) Pub. No.: US 2025/0361522 A1
(43) Pub. Date: Nov. 27, 2025

(54) GEMINIVIRUS RESISTANT PLANTS

(71) Applicants: WILHELM GRUISSEM, Forch (CH); YI-WEN LIM, Zurich (CH); PASCAL SCHLÄPFER, Baden (CH); JOEL KUON, Bern (CH); Donald Danforth Plant Science Center, ST. LOUIS, MO (US)

(72) Inventors: REBECCA BART, ST. LOUIS, MO (US); NIGEL TAYLOR, ST. LOUIS, MO (US); BEN MANSFELD, ST. LOUIS, MO (US); WILHELM GRUISSEM, FORCH (CH); YI-WEN LIM, ZURICH (CH); PASCAL SCHLÄPFER, BADEN (CH); KERRIGAN GILBERT, ST. LOUIS, MO (US); JOEL KUON, BERN (CH)

(21) Appl. No.: 18/848,668

(22) PCT Filed: Mar. 20, 2023
(86) PCT No.: PCT/US2023/064691

§ 371 Date: Sep. 19, 2024

Related U.S. Application Data
(60) Provisional application No. 63/269,685, filed on Mar. 21, 2022.
(60) Provisional application No. 63/362,477, filed on Apr. 5, 2022.

(51) Int. Cl. C12N 15/82; C07K 14/415; C12N 9/12

(52) U.S. Cl. CPC C12N 15/8283; C07K 14/415; C12N 9/1252

(57) ABSTRACT

The disclosure relates to DNA polymerase delta subunit 1 (POLD1) polypeptides that mediate resistance to geminiviruses in plants. Also disclosed are plants comprising polynucleotides encoding the POLD1 polypeptides along with related methods of using polynucleotides that encode the POLD1 polypeptides to enhance resistance of a plant to infection by a geminivirus.

### CROSS-REFERENCE TO RELATED APPLICATIONS

This application is a U.S. National Phase of PCT/US2023/064691, filed Mar. 20, 2023, which claims priority to provisional applications U.S. Ser. No. 63/362,477, filed Apr. 5, 2022 and U.S. Ser. No. 63/269,685, filed Mar. 21, 2022, which are incorporated herein by reference in their entireties.

### SEQUENCE LISTING XML

The instant application contains a sequence listing, which has been submitted in XML file format by electronic submission and is hereby incorporated by reference in its entirety. Said XML file, created on May 13, 2025, is named P13774US02.xml and is 508,851 bytes in size.

### BACKGROUND

Geminiviruses cause disease in major staple food and cash crops including cassava, cotton, tomato, maize, soybean, sweet potato, beets, peppers, and okra. Combined losses run into many billion US dollars per year. Inherent resistance to geminivirus is rare and its molecular mechanism largely unknown. Existing control methods include spraying insecticides to control insect vectors, and in some cases, conventional breeding.

Cassava (Manihot esculenta Crantz) is a highly heterozygous staple root crop that feeds nearly a billion people worldwide. Cassava yields are suppressed by infections with cassava mosaic geminiviruses (CMG, Family Geminiviridae: Genus Begomovirus) which collectively cause cassava mosaic disease (CMD). Eleven species of CMG are known to infect cassava across sub-Saharan Africa, the Indian subcontinent, and recently in serval countries of South-East Asia. CMGs possess two circular single-stranded DNA genomes that are transmitted by the whitefly Bemisia tabaci and spread by farmers who plant infected stem cuttings to establish the next cropping cycle. Three types of resistance to CMGs have been described in cassava as CMD1, CMD2, and CMD3. In all cases the genes responsible for resistance and their modes of action remain unknown. Understanding genetic sources for resistance to geminiviruses is critical to securing yields for cassava farmers.

### SUMMARY

Transgenic plants with enhanced resistance to at least one geminivirus are provided, the plants comprising a transgene encoding a DNA polymerase delta subunit 1 (POLD1) polypeptide, wherein the POLD1 polypeptide comprises a mutation of at least one amino acid in or near the active center of the POLD1 polypeptide.

Plants with enhanced resistance to at least one geminivirus are provided, the plant comprising a polynucleotide encoding a POLD1 polypeptide, wherein the POLD1 polypeptide comprises a mutation of at least one amino acid at a position corresponding to position 528, 598, 680, 694, or 685 of SEQ ID NO: 1. In certain embodiments, the plant is not a cassava plant.

Plant cells and plant parts from any of the plants of the present disclosure are also provided. In certain embodiments, the plant part is a seed.

Methods of enhancing resistance of a plant to infection by a geminivirus are provided, the methods comprising: modifying at least one plant cell to comprise a polynucleotide encoding a POLD1 polypeptide, wherein the POLD1 polypeptide comprises a mutation of at least one amino acid in or near the active center of the POLD1 polypeptide. In certain embodiments, the modifying comprises transforming at least one plant cell with a polynucleotide encoding the POLD1 polypeptide. In certain embodiments, the modifying comprises using genome editing to modify the nucleotide sequence of a native gene in the genome of the plant cell.

Methods of limiting a disease caused by a geminivirus in agricultural crop production are provided, the methods comprising: planting a seedling, cutting, tuber, or seed of any of the plants of the present disclosure; and growing the seedling, cutting, tuber, or seed under conditions favorable for the growth and development of a plant resulting therefrom. In certain embodiments, the plant is subjected to geminivirus infection.

Methods for selecting a plant with resistance to a disease caused by a geminivirus are provided, the methods comprising: detecting the presence of (i) a POLD1 polypeptide or (ii) a polynucleotide encoding the POLD1 polypeptide, wherein the POLD1 polypeptide comprises a mutation of at least one amino acid in or near the active center of the POLD1 polypeptide in a plant, or in at least one part or cell thereof; and selecting the plant comprising the POLD1 polypeptide or the polynucleotide encoding the POLD1 polypeptide. In certain embodiments, the plant is within a mixed population of plants comprising other plants which lack the POLD1 polypeptide comprising the mutation.

Methods for introducing resistance to a disease caused by a geminivirus into a plant are provided, the methods comprising: (a) crossing a first plant comprising in its genome a polynucleotide encoding a POLD1 polypeptide with a second plant lacking in its genome the polynucleotide, wherein the POLD1 polypeptide comprises a mutation of at least one amino acid in or near the active center of the POLD1 polypeptide, whereby at least one progeny plant is produced; (b) genotyping at least one progeny plant for the presence of the mutation; and (c) selecting at least one progeny plant comprising in its genome the polynucleotide encoding the POLD1 polypeptide comprising the mutation.

Polynucleotides encoding a POLD1 polypeptide, wherein the POLD1 polypeptide comprises a mutation of at least one amino acid in or near the active center of the POLD1 polypeptide, and wherein the polynucleotide is operably linked to a polynucleotide comprising a heterologous promoter are provided. Also provided are vectors and host cells comprising any of the polynucleotides of the present disclosure.

Methods of producing a commodity plant product are provided, the methods comprising: (i) processing any of the plants of the present disclosure, or a part thereof; and (ii) recovering the commodity plant product from the processed plant or part thereof.

Biological samples comprising a detectable amount of a polynucleotide comprising a transgene encoding a POLD1 polypeptide, wherein the POLD1 polypeptide comprises a mutation of at least one amino acid in or near the active center of the POLD1 polypeptide are also provided.

### BRIEF DESCRIPTION OF THE DRAWINGS

FIG. 1A-C show CMD2 type cassava varieties lose resistance upon de novo morphogenesis. FIG. 1A shows that TME204-WT CMD2-type plants challenged with cassava mosaic geminivirus remains symptom free (left), embryogenic structures arise from tissue culture induced de novo morphogenesis (middle), and regenerated plant shows classic mosaic symptoms after virus challenge (right) FIG. 1B shows F1 populations derived from heterozygous resistant parents (NASE14, NASE19, TME14) crossed with susceptible loss-of-CMD2-resistance (LCR) line. Plants were grown and phenotyped in the field in Uganda and scored for disease over two years on a 1-5 disease score. FIG. 1C shows the disease rating distribution across all populations segregates at 1:1. χ²=0.59. In each population, ˜15% of lines with consistent phenotypes over the two years were selected for bulk segregant analysis (BSA) mapping (solid circles).

FIG. 2A-D shows full genome bisulfite sequencing of cassava varieties before and after tissue culture induced de novo morphogenesis. FIG. 2A is analysis strategy cartoon. FIG. 2B shows a single peak on chromosome 13 was identified as differentially methylated between resistant and susceptible lines. FIG. 2C shows differential methylation across two haplotypes for CG, CHG and CHH methylation. FIG. 2D shows MePOLD1 does not display differential methylation between resistant and susceptible cassava lines.

FIG. 3A-B shows CMD2-mediated resistance remains stable in cassava progeny lines generated through sexual crosses. TME204-LCR lines were crossed with TME14 which carries functional CMD2-type resistance. FIG. 3A illustrates three progeny lines passed through successive cycles of somatic embryogenesis. Plants were regenerated after each cycle and inoculated with the virulent EACMV isolate K201. FIG. 3B shows CMD leaf symptoms assessed visually on a 0-5 scale over 44 days, after which plants were ratooned and new growth scored until 90 days after inoculation. TME14 wildtype plants displayed the recovery phenotype typical for this landrace, while TME14 (LCR) lines passed through embryogenesis became highly CMD susceptible, displaying the highest disease severity (5) and no recovery (arrowed). Plants regenerated from TME14xLCR F1 progenies remained resistant to CMD at a level not significantly different from the original F1 progeny line from which they were derived. OES: organized embryogenic structures.

FIG. 4 shows a cartoon model. In the model, L2 contains the resistant allele while L3 does not (1). L3 gives rise to embryogenetic tissue (Loss of CMD2 Resistance-LCR) through de novo morphogenesis that is non chimeric and lacks the resistance allele (2). A similar scenario is possible if the L1 is −/− for CMD2 and gives rise to embryogenic tissue. When an LCR plant is crossed with a chimeric wildtype (WT) CMD2-type cassava variety, F1 progeny match the genotype of the parental L2 layer and therefore segregate resistance 1:1 and are not chimeric (4). Consequently, when resistant and susceptible F1 progeny are passaged through tissue culture induced de novo morphogenesis, the maintain their resistant or susceptible phenotypes, respectively.

FIG. 5A-F shows whole genome sequencing and genome variant analysis (WGS-GVA) and fine mapping reveal nonsynonymous SNPs in MePOLD1 that segregate with resistance. FIG. 5A shows TME204-WT and F1 progeny, TME419 WT, 60444 WT and TME204, TME419 and 60444 plants regenerated from tissue culture were tested for resistance and susceptibility. TME204 WT, F1-3, F1-7, F1-8, and TME419-WT plants had CMD2 resistance while all other plants were susceptible to ACMV infections. A haplotype-restricted G to C transversion in the MePOLD1 gene at location 9,081,215 bp causes a heterozygous V528L mutation in MePOLD1 (SEQ ID NOs: 61-62). FIG. 5B shows tricube-smoothed allele frequency enrichment (ΔSNP-index) across the TME204 hap1 assembly. Line denotes the 95% confidence interval. The highlighted region on Chr12 defines the significantly linked CMD2 region. FIG. 5C shows enlargement of the CMD2 locus mapping results. The dashed lines indicate the borders of the mapped locus between ˜5-13 Mb. The previously reported associated marker from Rabbi et al., 2020 is indicated by arrow. FIG. 5D shows SNP data from genotyping by sequencing improves the mapping resolution to ˜300 kb using individual recombinants within the broader CMD2 locus. The genotype at each SNP is indicated. The resistance phenotype is indicated on the left bar. Genotypes are extended downstream until the next SNP called. Two homozygous resistant and susceptible lines are added as a control (top and bottom). Based on the location of the mapped locus, and the previously identified GWAS marker, KASP markers (M1-8) were developed for fine mapping (positions denoted by dot-dash lines in B and C). FIG. 5E shows recombinants from a fine-mapping population within the region place CMD2 in the 190 Kb interval between markers M3 and M7. Lines P1581 and P1561 are non-recombinant susceptible and resistant controls, respectively. FIG. 5F shows genomic rearrangements within the fine mapped CMD2 locus introduce new gene candidates.

FIG. 6A-C shows genotyping using Kompetitive Allele Specific PCR (KASP) markers. FIG. 6A shows the entire ˜1000 F1 population was screened using 4 KASP markers, M1, M2, M6, and M8. Dot plot of the raw relative fluorescence units (RFU) for the two allele specific primers. Each point is an individual F1 progeny and the allele call made for each marker is indicated. FIG. 6B shows the distribution of genotype calls for each of the two phenotypic states (R-Resistant, S-Susceptible). FIG. 6C shows a view of the all the calls of the entire ˜1000 individual population. The markers on the x-axis are ordered by their genomic position allowing to visualize recombinants between the markers. The resistance phenotype is indicated on the left bar.

FIG. 7A-B shows gene expression analysis of genes within the fine-mapped CMD2 locus. FIG. 7A shows resistant TME204 wildtype plants were compared to susceptible TME204-LCR plants regenerated through embryogenesis during production of friable embryonic callus (FEC). FIG. 7B shows a comparison resistant and susceptible F1 plants derived from a TME204-WT self-cross. Of the 8 genes defined within the 190 Kb locus, only 6 are detected as expressed in our datasets. BRIX1—RIBOSOME BIOGENESIS PROTEIN BRIX; PER3—PEROXIDASE 3-RELATED; POLD1—DNA POLYMERASE DELTA; ZINCF—ZINC FINGER, CCCH-type.

FIG. 8A-D shows VIGS silencing of MePOLD1. CMD-susceptible cassava 60444 recovers from ACMV infection when MePOLD1 is downregulated by VIGS. FIG. 8A and FIG. 8B show the percentage of symptomatic 60444 plants and CMD symptom severity, respectively, 18 weeks post-inoculation: ACMV (n=15), GUS-VIGS (n=30), MePOLD1-VIGS (n=40), and Mock (n=15). Bars show standard error. FIG. 8C shows quantification of ACMV titre post-onset of CMD symptoms after inoculation with ACMV (n=3), GUS-VIGS (n=10), MePOLD1-VIGS (n=10), and Mock (n=3) (Mann-Whitney U test, ***=P<0.001). Week 0 is the first onset of symptoms detected on individual plants. FIG. 8D shows CMD symptoms on cassava leaves after ACMV-VIGS inoculation of 60444 plants with week 0 being when first symptoms were detected on individual plants.

FIG. 9 shows MePOLD1 expression relative to expression of the gene encoding Tubulin 1 β chain (MeTUB1, Manes.08G061700) after ACMV-VIGS inoculation (non-modified ACMV, GUS-VIGS, MePOLD1-VIGS and mock) of CMD-susceptible cassava 60444. Week 0 is the first onset of symptoms of individual plants and week 2 is two weeks after that and so on. Number of biological replicates at Week 0, 2 and 4 respectively: ACMV (n=3, 3, 3), GUS-VIGS (n=10, 10, 8), MePOLD1-VIGS (n=9, 10, 9), and mock (n=3, 3, 3).

FIG. 10 illustrates a hypothesis to explain the observed MePOLD1 expression pattern after ACMV-VIGS inoculation and reduction of ACMV load in CMD-susceptible 60444. The hypothetical MePOLD1 expression starts off under normal conditions. Once the plant has been inoculated with the MePOLD1 VIGS construct which is a modified ACMV clone (VIGS/ACMV), the rise of VIGS/ACMV leads to the reduction (increase in MePOLD1 siRNA as well as siRNA of ACMV) of MePOLD1 expression. Since ACMV needs MePOLD1 to replicate, as MePOLD1 expression drops and ACMV siRNA increases, the quantity of VIGS/ACMV will also decrease. The reduction in VIGS/ACMV then leads to lower production of siRNA against MePOLD1 thereby allowing the expression of MePOLD1 to return to approximately normal quantities. Since MePOLD1 level returns close to normal, VIGS/ACMV will also begin increase in quantity. However, since there are residual amounts of ACMV siRNA, VIGS/ACMV will never be able to establish itself thus leading to a cyclic equilibrium where VIGS/ACMV is maintained at a low quantity and MePOLD1 expression remains almost unchanged.

FIG. 11A-D shows nonsynonymous SNPs in POLD1. FIG. 11A is a dendrogram of samples used to perform whole genome sequencing of Manihot esculenta. Non-synonymous SNPs in various cultivars lead to amino acid changes in MePOLD1 that segregate with CMD2 resistance. Plants that were identified to be susceptible by infection assay are indicated. Resistant plants in Clade 1 harbor V528L mutations. Plants of clade 2 harbor G680V mutations TMS-9102324 WT harbors L685F mutation. FIG. 11B shows average CMD severity across a diverse set of cassava cultivars from the HapMapII population (Ramu et al., 2017) that either have one of the three mutations or an unknown SNP in MePOLD1 (“Other”). FIG. 11C shows identity of all nonsynonymous SNPs in varieties from the “Other” category in FIG. 11B; varieties are split into either CMD severity score below 2 or CMD severity score above 2. SNPs found only in cultivars with CMD severity scores below 2.0 are in dark gray, all other SNPs are in light gray. FIG. 11D is a three-dimensional structure of S. cerevisiae POLD1 (PDB: 3IAY) with corresponding MePOLD1 nonsynonymous mutations highlighted; V528L, G680V, and L685F. Additional residues from (c), L685F and L598W, are also indicated. Residue identity and position in the yeast protein are noted and corresponding positions for MePOLD1 are in parentheses. POLD1 functional domains are indicated: N-terminal domain, exonuclease domain, and structural motifs of the polymerase domain are individually indicated: palm, fingers, and thumb. A zoomed in view of the 3D structure centered on the mutated residues found in cassava is also shown (bottom).

FIG. 12A is a schematic diagram of the POLD1 protein from cassava. Lollipop flags indicate locations of resistance alleles. Active site motifs in the exonuclease and pol are indicated by outlined boxes. FIG. 12B shows a protein alignment of POLD1 sequences (SEQ ID NOs: 63-67). Sequences from three varieties containing a non-synonymous SNP are included. Affected amino acid is noted by an arrowhead; position of the last amino acid in alignment is indicated in paratheses. Manes. 12G77400: Manihot esculenta AM560-2 v6.1; Athaliana: Arabidopsis thaliana; Hsapiens: Homo sapiens; Scerevisiae: Saccharomyces cerevisiae.

FIG. 13A-B shows the premature stop codon within the resistant haplotype of MePOLD1 in susceptible line 5001-NASE14- #41. 5001-NASE14- #41 is a transgenic line from resistant NASE14. FIG. 13A is a schematic diagram showing the gene structure of the resistant haplotype of MePOLD1. The exons are indicated as solid boxes, and the introns are indicated as lines. The mutation site in the resistant haplotype of MePOLD1 in line 5001-NASE14- #41 is indicated. FIG. 13B shows that Sanger sequencing analysis identified a mutation in the resistant haplotype of MePOLD1 in line 5001-NASE14- #41 (SEQ ID NOs: 68-69). The full-length cDNA sequence was amplified by a pair of primers which specifically worked for resistant haplotype of MePOLD1 in resistant NASE14 and its derived lines.

FIG. 14A-C shows the identification of amino acid regions predicted to cause POLD1 to mediate resistance to geminiviruses based on the mutations identified in cassava. FIG. 14A-B is an alignment of yeast and cassava POLD1 sequences (SEQ ID NOs: 1, 49, 70, and 71). Mutations found in cassava are shown in bold and underlined. Twenty-one regions of interest (amino acids shown in bold) were identified. Regions were merged if two regions were spaced by no more than five amino acids (shown in bold italics). FIG. 14C shows the crystal structure of yeast POLD1. The arithmetic mid-point of all atoms belonging to the five amino acids was used to define the center of a sphere of influence (sphere). This sphere then was defined to have a radius to the outermost atom of the five amino acids (H10 of V543), involved in mutations, including its van der Waal radius of 1.2 Å.

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, flakes, syrup, oil, starch, and cereals manufactured in whole or in part to contain crop plant by-products. In certain embodiments, the biological sample is “non-regenerable” (i.e., incapable of being regenerated into a plant or plant part).

As used herein, the term “elite germplasm” or “elite plant” refers to any germplasm or plant, respectively, that has resulted from breeding and selection for superior agronomic performance.

As used herein, the terms “include,” “includes,” and “including” are to be construed as at least having the features to which they refer while not excluding any additional unspecified features.

As used herein, the term “introducing” is intended presenting to the plant a polynucleotide in such a manner that the polynucleotide gains access to the interior of a cell of the plant. The methods of the disclosure do not depend on a particular method for introducing a polynucleotide to a plant, only that the polynucleotide gains access to the interior of at least one cell of the plant.

As used herein, the term “heterologous” refers to a polynucleotide that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

As used herein, a “native gene” is intended to mean a gene that is a naturally-occurring gene in its natural or native position in the genome of a plant. Such a native gene has not been genetically engineered or otherwise modified in nucleotide sequence and/or position in the genome the plant through human intervention, nor has such a native gene been introduced into the genome of the plant via artificial methods such as, for example, plant transformation.

As used herein, the term “operably linked” is intended to mean a functional linkage between two or more elements. For example, an operable linkage between a polynucleotide or gene of interest and a regulatory sequence (i.e. a promoter) is a functional link that allows for expression of the polynucleotide of interest. Operably linked elements may be contiguous or non-contiguous. When used to refer to the joining of two protein coding regions, by operably linked is intended that the coding regions are in the same reading frame.

As used herein, the terms “orthologous” or “ortholog” are used to describe genes or proteins encoded by those genes that are from different species but which have the same function (e.g., encode enzymes that catalyze the same reactions). Orthologous genes will typically encode proteins with some degree of sequence identity (e.g., at least 40%, 50%, 60%, 70%, 80%, 90%, or 95% sequence identity, conservation of sequence motifs, and/or conservation of structural features).

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the term “polynucleotide” is not intended to limit the present disclosure to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotides of the disclosure also encompass all forms of polynucleotides including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

As used herein, the term “stable transformation” is intended that a polynucleotide introduced into a plant integrates into the genome of the plant and is capable of being inherited by progeny thereof. As used herein, the term “transient transformation” is intended that a polynucleotide introduced into a plant does not integrate into the genome of the plant.

As used herein, the terms “transgenic plant” and “transformed plant” are equivalent terms that refer to a “plant” as described above, wherein the plant comprises polynucleotide that is introduced into a plant (i.e., a transgene) by, for example, any of the stable and transient transformation methods disclosed elsewhere herein or otherwise known in the art. Such transgenic plants and transformed plants also refer, for example, the plant into which the polynucleotide was first introduced and also any of its progeny plants that comprise the polynucleotide.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure relates to the identification of DNA polymerase delta subunit 1 (POLD1) genes that mediate resistance to geminiviruses, including cassava mosaic geminiviruses. The present disclosure provides nucleotide sequences of cassava POLD1 (MePOLD1) genes, orthologs thereof, and other naturally occurring variants of such POLD1 genes and synthetic or artificial (i.e. non-naturally occurring) variants thereof. POLD1 nucleotide sequences include, but not limited to, the nucleotide sequences of MePOLD1 set forth in SEQ ID NOs: 2-4.

In certain embodiments, the POLD1 polypeptides encoded by the polynucleotides of the disclosure are functional POLD1 polypeptides, or part(s), or domain(s) thereof, which are capable of conferring on a plant enhanced resistance to at least one geminivirus. Such POLD1 polypeptides of the present disclosure include, but are not limited to, the POLD1 polypeptide comprising the amino acid sequence set forth in SEQ ID NO: 1 and that comprise a mutation of at least one amino acid in or near the active center of the POLD1 polypeptide. In certain embodiments, the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1. In certain embodiments, the POLD1 polypeptide comprises a mutation of at least one amino acid at a position corresponding to position 520, 528, 598, 606, 617, 627, 680, 684, 685, 714, or 758 of SEQ ID NO: 1. In certain embodiments, the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1. In certain embodiments, the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

The POLD1 polynucleotides of the disclosure can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present disclosure. Such sequences include sequences that are orthologs of the disclosed sequences. Genes found in different species are considered orthologs when their nucleotide sequences and/or their encoded amino acid sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species.

In certain embodiment, the orthologs of the present disclosure have a nucleotide sequence having at least 80%, 85%, 90%, 91%, 92%, 93%, 94%, 95%, 96%, 97%, 98%, 99%, or greater nucleotide sequence identity to at least one nucleotide sequence set forth in SEQ ID NOs: 2-4 and/or encode a polypeptide having least 80%, 85%, 90%, 91%, 92%, 93%, 94%, 95%, 96%, 97%, 98%, 99%, or greater amino acid sequence identity to the amino acid sequence set forth in SEQ ID NO: 1.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e. genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the disclosure. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, New York). An extensive guide to the hybridization of nucleic acids is found in Tijssen (1993) Laboratory Techniques in Biochemistry and Molecular Biology Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Examples of orthologs of MePOLD1 are provided in Table 1. In certain embodiments, the polynucleotide encoding the POLD1 polypeptide has at least about 80%, 85%, 90%, 91%, 92%, 93%, 94%, 95%, 96%, 97%, 98%, 99% or more sequence identity across the entire nucleotide sequence set forth in at least one of SEQ ID NOs: 2-4, 6-8, 10-12, 14-16, 18-20, 22-24, 26-28, 30-32, 34-36, 38-40, or 42-44 and encodes a POLD1 polypeptide comprising a mutation of at least one amino acid in or near the active center of the POLD1 polypeptide. In certain embodiments, the polynucleotide encode a POLD1 polypeptide having at least about 75%, 80%, 85%, 90%, 91%, 92%, 93%, 94%, 95%, 96%, 97%, 98%, 99% or more sequence identity to the entire amino acid sequence set forth in at least one of SEQ ID NOs: 1, 5, 9, 13, 17, 21, 25, 29, 33, 37, or 41 and includes a mutation of at least one amino acid in or near the active center of the POLD1 polypeptide.

TABLE 1

| | Protein | Genomic | Transcript | CDS |
|---|---|---|---|---|
| Cassava | SEQ ID NO: 1 | SEQ ID NO: 2 | SEQ ID NO: 3 | SEQ ID NO: 4 |
| (Manihot esculenta) | | | | |
| Tomato | SEQ ID NO: 5 | SEQ ID NO: 6 | SEQ ID NO: 7 | SEQ ID NO: 8 |
| (Solanum lycopersicum) | | | | |
| Cotton | SEQ ID NO: 9 | SEQ ID NO: 10 | SEQ ID NO: 11 | SEQ ID NO: 12 |
| (Gossypium hirsutum) | | | | |
| Bean | SEQ ID NO: 13 | SEQ ID NO: 14 | SEQ ID NO: 15 | SEQ ID NO: 16 |
| (Phaseolus vulgaris) | | | | |
| Soybean | SEQ ID NO: 17 | SEQ ID NO: 18 | SEQ ID NO: 19 | SEQ ID NO: 20 |
| (Glycine max) | | | | |
| Maize | SEQ ID NO: 21 | SEQ ID NO: 22 | SEQ ID NO: 23 | SEQ ID NO: 24 |
| (Zea mays) | | | | |
| Beet | SEQ ID NO: 25 | SEQ ID NO: 26 | SEQ ID NO: 27 | SEQ ID NO: 28 |
| (Beta vulgaris) | | | | |
| Pepper | SEQ ID NO: 29 | SEQ ID NO: 30 | SEQ ID NO: 31 | SEQ ID NO: 32 |
| (Capsicum annuum) | | | | |
| Vegetable Marrow | SEQ ID NO: 33 | SEQ ID NO: 34 | SEQ ID NO: 35 | SEQ ID NO: 36 |
| (Cucurbita pepo | | | | |
| subsp. pepo) | | | | |
| Grape | SEQ ID NO: 37 | SEQ ID NO: 38 | SEQ ID NO: 39 | SEQ ID NO: 40 |
| (Vitis vinifera) | | | | |
| Sweet Potato | SEQ ID NO: 41 | SEQ ID NO: 42 | SEQ ID NO: 43 | SEQ ID NO: 44 |
| (Ipomoea batatas) | | | | |

“Variants” is intended to mean substantially similar sequences. For polynucleotides, a variant comprises a polynucleotide having deletions (i.e. truncations) at the 5′ and/or 3′ end; deletion and/or addition of one or more nucleotides at one or more internal sites in the polynucleotide; and/or substitution of one or more nucleotides at one or more sites in the polynucleotide. For polynucleotides, conservative variants include those sequences that, because of the degeneracy of the genetic code, encode the amino acid sequence of one of the POLD1 polypeptides of the disclosure. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a POLD1 polypeptide of the disclosure. Generally, variants of a particular polynucleotide of the disclosure will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments, variants of a particular polynucleotide of the disclosure will have at least about 75%, 80%, 85%, 90%, 91%, 92%, 93%, 94%, 95%, 96%, 97%, 98%, 99% or more sequence identity to at least one nucleotide sequence selected from SEQ ID NOs: 2-4, 6-8, 10-12, 14-16, 18-20, 22-24, 26-28, 30-32, 34-36, 38-40, or 42-44, and optionally comprise a non-naturally occurring nucleotide sequence that differs from the nucleotide sequence set forth in SEQ ID NOs: 2-4, 6-8, 10-12, 14-16, 18-20, 22-24, 26-28, 30-32, 34-36, 38-40, or 42-44 by at least one nucleotide modification, wherein the at least one nucleotide modification comprises the substitution of at least one nucleotide, the addition of at least one nucleotide, or the deletion of at least one nucleotide. It is understood that the addition of at least one nucleotide can be the addition of one or more nucleotides within a nucleotide sequence of the present disclosure, the addition of one or more nucleotides to the 5′ end of a nucleotide sequence of the present disclosure, and/or the addition of one or more nucleotides to the 3′ end of a nucleotide sequence of the present disclosure.

Variants of a particular polynucleotide of the disclosure can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to at least one polypeptide having the amino acid sequence selected from SEQ ID NOs: 1, 5, 9, 13, 17, 21, 25, 29, 33, 37, and 41 is disclosed. Percent sequence identity between any two polypeptides can be calculated using sequence alignment programs and parameters described elsewhere herein. Where any given pair of polynucleotides of the disclosure is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity. In certain embodiments, variants of a particular polypeptide of the disclosure will have at least about 60%, 65%, 70%, 75%, 80%, 85%, 90%, 91%, 92%, 93%, 94%, 95%, 96%, 97%, 98%, 99% or more sequence identity to at least one of the amino acid sequences set forth in SEQ ID NOs: 1, 5, 9, 13, 17, 21, 25, 29, 33, 37, or 41, and optionally comprises a non-naturally occurring amino acid sequence that differs from at least one amino acid sequence selected from SEQ ID NOs: 1, 5, 9, 13, 17, 21, 25, 29, 33, 37 and 41 by at least one amino acid modification, wherein the at least one amino acid modification comprises the substitution of at least one amino acid, the addition of at least one amino acid, or the deletion of at least one amino acid. It is understood that the addition of at least one amino acid can be the addition of one or more amino acids within an amino acid sequence of the present disclosure, the addition of one or more amino acids to the N-terminal end of an amino acid sequence of the present disclosure, and/or the addition of one or more amino acids to the C-terminal end of an amino acid sequence of the present disclosure. “Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation.

The POLD1 polypeptides of the disclosure may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymology. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington. D.C.), herein incorporated by reference. Conservative substitutions, such as exchanging one amino acid with another having similar properties, may be optimal.

Thus, the polynucleotides of the disclosure include both the naturally occurring sequences as well as mutant and other variant forms. Likewise, the polypeptide of the disclosure encompass naturally occurring polypeptides as well as variations and modified forms thereof. In certain embodiments, such variants confer to a plant or part thereof enhanced resistance at least one geminivirus. In certain embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

Variant polynucleotides and polypeptide also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

To determine the percent identity of two amino acid sequences or of two nucleic acids, the sequences are aligned for optimal comparison purposes. The percent identity between the two sequences is a function of the number of identical positions shared by the sequences (i.e. percent identity=number of identical positions/total number of positions (e.g. overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotides of the disclosure. BLAST protein searches can be performed with the XBLAST program, score=50, wordlength=3, to obtain amino acid sequences homologous to polypeptides of the disclosure. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g. XBLAST and NBLAST) can be used. BLAST, Gapped BLAST, and PSI-Blast, XBLAST and NBLAST are available on the World Wide Web at ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM 120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences and using multiple alignment by means of the algorithm ClustalW (Nucleic Acid Research, 22(22): 4673-4680, 1994) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the World Wide Web at ebi.ac.uk/Tools/clustalw/index).

Fragments of the disclosed polynucleotides and polypeptides encoded thereby are also encompassed by the present disclosure. By “fragment” is intended a portion of the polynucleotide or a portion of the amino acid sequence and hence polypeptide encoded thereby. Fragments of polynucleotides comprising coding sequences may encode polypeptide fragments that retain biological activity of the full-length polypeptide. Alternatively, fragments of a polynucleotide that are useful as hybridization probes generally do not encode proteins that retain biological activity. Thus, fragments of a nucleotide sequence may range from at least about 20 nucleotides, about 50 nucleotides, about 100 nucleotides, and up to the full-length polynucleotide of the disclosure. In certain embodiments, the fragments of the disclosed polynucleotides and polypeptide encoded thereby are those that are capable of conferring to a plant resistance to a geminivirus.

The present disclosure further provides methods of enhancing resistance of a plant to infection by a geminivirus. The methods comprise modifying at least one plant cell to comprise a polynucleotide encoding a POLD1 polypeptide of the disclosure, and optionally regenerating a plant from the modified plant cell comprising the polynucleotide. In a first aspect, the methods of enhancing resistance of a plant to infection by a geminivirus comprise transforming at least one plant cell with a polynucleotide encoding a POLD1 polypeptide of the disclosure. In certain embodiments, the polynucleotide is stably incorporated into the genome of the plant cell. In a second aspect, the methods of enhancing resistance of a plant to infection by a geminivirus involve the use of a genome-editing method to modify the nucleotide sequences of a native gene in the genome of the plant cell to comprise a polynucleotide encoding a POLD1 polypeptide of the present disclosure. Thus, the methods of the disclosure also encompass gene replacement to produce a polynucleotide encoding a POLD1 polypeptide of the disclosure in the genome of a plant cell. If desired, the methods of the first and/or second aspect can further comprise regenerating the plant cell into a plant comprising in its genome the polynucleotide. In certain embodiments, such a regenerated plant comprises enhanced resistance of a plant to infection by a geminivirus.

The polynucleotide encoding a POLD1 polypeptide can be provided in a polynucleotide construct (e.g., an expression cassette) for expression in the plant. The polynucleotide construct can include in the 5′-3′ direction of transcription, a transcriptional and translational initiation region (i.e., a promoter), a POLD1 coding region, and a transcriptional and translational termination region (i.e. termination region) functional in a plant. The regulatory regions (i.e. promoters, transcriptional regulatory regions, and translational termination regions) and/or the POLD1 coding region may be native/analogous to the cell or to each other. Alternatively, the regulatory regions and/or the POLD1 coding region may be heterologous to the cell or to each other.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The polynucleotide constructs may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); poty virus leaders, for example, TEV leader (Tobacco Etch Virus) (Gallie et al. (1995) Gene 165 (2): 233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the polynucleotide construct, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions, may be involved.

A number of promoters can be used. The choice of heterologous promoter can depend on a number of factors such as, for example, the desired timing, localization, and pattern of expression as well as responsiveness to particular biotic or abiotic stimulus. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those described in U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the POLD1 polypeptide within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12 (2): 255-265; Kawamata et al. (1997) Plant Cell Physiol. 38 (7): 792-803; Hansen et al. (1997) Mol. Gen Genet. 254 (3): 337-343; Russell et al. (1997) Transgenic Res. 6 (2): 157-168; Rinehart et al. (1996) Plant Physiol. 112 (3): 1331-1341; Van Camp et al. (1996) Plant Physiol. 112 (2): 525-535; Canevascini et al. (1996) Plant Physiol. 112 (2): 513-524; Yamamoto et al. (1994) Plant Cell Physiol. 35 (5): 773-778; Lam (1994) Results Probl. Cell Differ. 20:181-196; Orozco et al. (1993) Plant Mol Biol. 23 (6): 1129-1138; Matsuoka et al. (1993) Proc Natl. Acad. Sci. USA 90 (20): 9586-9590; and Guevara-Garcia et al. (1993) Plant J. 4 (3): 495-505. Such promoters can be modified, if necessary, for weak expression.

In certain embodiments, it will be beneficial to express the gene from an inducible promoter, particularly from a pathogen-inducible promoter. Such promoters include those from pathogenesis-related proteins (PR proteins), which are induced following infection by a pathogen; e.g. PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Such inducible promoters include the maize PRms gene promoter, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used with the polynucleotides of the disclosure. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2): 141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

While it may be optimal to express the POLD1 polypeptide using heterologous promoters, the native promoter of the corresponding POLD1 gene may be used.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) (ell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

The polynucleotide construct can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004). J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not intended to be limiting. Any selectable marker gene can be used.

Any methods known in the art for modifying DNA in the genome of a plant can be used to modify genomic nucleotide sequences in planta, for example, to create or insert a POLD1 gene or even to replace or modify an endogenous POLD1 gene or allele thereof. Such methods include, but are not limited to, genome-editing (or gene-editing) techniques, such as, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Amould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34: e149; U.S. Pat. App. Pub. No. 2009/0133152; and U.S. Pat. App. Pub. No. 2007/0117128; all of which are herein incorporated in their entirety by reference.

Unless stated otherwise or apparent from the context of a use, the term “gene replacement” is intended to mean the replacement of any portion of a first polynucleotide molecule (e.g. a chromosome) that involves homologous recombination with a second polynucleotide molecule using a genome-editing technique as disclosed herein, whereby at least a part of the nucleotide sequence of the first polynucleotide molecule is replaced with the nucleotide sequence of the second polynucleotide molecule. It is recognized that such gene replacement can result in additions, deletions, and/or modifications in the nucleotide sequence of the first polynucleotide molecule and can involve the replacement of an entire gene or genes, the replacement of any part or parts of one gene, or the replacement of non-gene sequences in the first polynucleotide molecule.

The CRISPR/Cas nuclease system can also be used to make double-strand breaks at specific recognition sequences in the genome of a plant for gene modification or gene replacement through homologous recombination. The CRISPR/Cas nuclease is an RNA-guided DNA endonuclease system performing sequence-specific double-stranded breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In certain embodiments, the CRISPR/Cas system is a prime editing system. Prime editing of a target sequence enables the incorporation of a nucleotide change including a single-nucleotide change (e.g., any transition or any transversion), an insertion of one or more nucleotides, or a deletion of one or more nucleotides. A Cas nuclease fused with a reverse transcriptase is guided to a specific DNA sequence by a modified guide RNA, named a prime editing guide RNA (pegRNA). The pegRNA is altered (relative to a standard guide RNA) to comprise an extended portion that provides a DNA synthesis template sequence which encodes a single strand DNA flap, which is homologous to a strand of the targeted endogenous DNA sequence to be edited, but which contains the desired one or more nucleotide changes and which, following synthesis by the reverse transcriptase, becomes incorporated into the target DNA molecule. The Cas polypeptide may be modified such that it has nickase activity. Prime editing is disclosed in, for example, PCT Publication WO/2020/191248, the entire contents of which is hereby incorporated by reference.

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas. 1013133107; Scholze & Boch (2010) Virulence 1:428-432; Christian et al. Genetics (2010) 186:757-761; Li el al. (2010) Nuc. Acids Res. (2010) doi: 10.1093/nar/gkq704; and Miller et al. (2011) Nature Biotechnology 29:143-148; all of which are herein incorporated by reference.

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The methods of the present disclosure find use in producing plants with enhanced resistance to at least one geminivirus. Typically, the methods of the present disclosure will enhance or increase the resistance of the subject plant to the disease by at least 25%, 50%, 75%, 100%, 150%, 200%, 250%, 500% or more when compared to the resistance of a control plant to the same geminivirus. Unless stated otherwise or apparent from the context of a use, a control plant is a plant that does not comprise the polynucleotide encoding a POLD1 polypeptide the present disclosure. In certain embodiments, the control plant is essentially identical (e.g. same species, subspecies, and variety) to the plant comprising the polynucleotide encoding a POLD1 polypeptide of the present disclosure except the control does not comprise the polynucleotide. In certain embodiments, the control will comprise a polynucleotide encoding a POLD1 polypeptide but not comprise a mutation of at least one amino acid in or near the active center of the POLD1 polypeptide.

The methods of the disclosure involve introducing a polynucleotide or polynucleotide construct into a plant. Methods for introducing polynucleotides or polynucleotide constructs into plants are known in the art including, but not limited to, stable transformation methods, transient transformation methods, and virus-mediated methods.

Depending on the desired outcome, the polynucleotides of the disclosure can be stably incorporated into the genome of the plant cell or not stably incorporated into genome of the plant cell. If, for example, the desired outcome is to produce a stably transformed plant with enhanced resistance to a plant disease caused by at least one geminivirus, then the polynucleotide can be, for example, fused into a plant transformation vector suitable for the stable incorporation of the polynucleotide into the genome of the plant cell. Typically, the stably transformed plant cell will be regenerated into a transformed plant that comprises in its genome the polynucleotide. Such a stably transformed plant is capable of transmitting the polynucleotide to progeny plants in subsequent generations via sexual and/or asexual reproduction. Plant transformation vectors, methods for stably transforming plants with an introduced polynucleotide and methods for plant regeneration from transformed plant cells and tissues are generally known in the art for both monocotyledonous and dicotyledonous plants or described elsewhere herein.

In other embodiments in which it is not desired to stably incorporate the polynucleotide in the genome of the plant, transient transformation methods can be utilized to introduce the polynucleotide into one or more plant cells of a plant. Such transient transformation methods include, for example, viral-based methods which involve the use of viral particles or at least viral nucleic acids. Generally, such viral-based methods involve constructing a modified viral nucleic acid comprising a heterologous polynucleotide of the disclosure operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by means of spraying, atomizing, dusting, spreading or pouring. It is recognized that it may be desirable to prepare formulations comprising the modified virus and/or modified viral nucleic acids before applying to the plant or part or parts thereof. Methods for making pesticidal formulations are generally known in the art or described elsewhere herein.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block. M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P: 119-124; Davies, et al. (1993) Plant Cell Rep. 12:180-183; Dong, J. A. and Mchughen, A. (1993) Plant Sci. 91:139-148; Franklin. C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5:17-27; Eapen et al. (1994) Plant Cell Rep. 13:582-586; Hartman, et al. (1994) Nature Biotechnology 12:919-923; Ritala, et al. (1994) Plant. Mol. Biol. 24:317-325; and Wan, Y. C. and Lemaux, P. G. (1994) Plant Physiol. 104:3748.

For the transformation of plants and plant cells, the nucleotide sequences of the disclosure can be inserted using standard techniques into any vector known in the art that is suitable for expression of the nucleotide sequences in a plant or plant cell. The selection of the vector depends on the transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100:247-250; Scheid et al., (1991) Mol. Gen. Genet., 228:104-112; Guerche et al., (1987) Plant Science 52:111-116; Neuhause et al., (1987) Theor. Appl Genet. 75:30-36; Klein et al., (1987) Nature 327:70-73; Howell et al., (1980) Science 208:1265; Horsch et al., (1985) Science 227:1229-1231; DeBlock et al., (1989) Plant Physiology 91:694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the disclosure may be introduced into plants by contacting plants with a virus or viral nucleic acids. Generally, such methods involve incorporating a polynucleotide or polynucleotide construct of the disclosure within a viral DNA or RNA molecule. Further, it is recognized that promoters of the disclosure also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48. Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In certain embodiments, the polynucleotides of the disclosure can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91:2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example. McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure stably incorporated into their genome.

Unless expressly stated or apparent from the context of usage, the methods and compositions of the present disclosure can be used with any plant species including, for example, monocotyledonous plants and dicotyledonous plants. Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), triticale (×Triticosecale or Triticum×Secale) sorghum (Sorghum bicolor, Sorghum vulgare), teff (Eragrostis tef), millet (e.g. pearl millet (Pennisetum glaucum), proso millet (Panicum miliaceum), foxtail millet (Setaria italica), finger millet (Eleusine coracana), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanut (Arachis hypogaea), cotton (Gossypium barbadense, Gossypium hirsutum), strawberry (e.g. Fragaria×ananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachwya, D. bulbifera, D. esculenta, D. dumetorum, D. trifida), cassava (Manihot esculenta), coffee (Coffea spp.), coconut (Cocos nucifera), okra (Abelmoschus esculentus), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), grape (Vitis vinifera), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), Cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon, tomato (Solanum lycopersicum), eggplant (Solanum melongena), pepper (Capsicum annuum), lettuce (e.g. Lactuca sativa), bean (Phaseolus vulgaris), lima bean (Phaseolus limensis), pea (Lathyrus spp.), chickpea (Cicer arietinum), and members of the genus Cucumis such as cucumber (C. sativus), cantaloupe (C. cantalupensis), and musk melon (C. melo), and ornamentals. Ornamentals include azalea (Rhododendron spp.), Hydrangea (Macrophylla hydrangea), Hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. In certain embodiments, the plant is a cassava, tomato, cotton, bean, soybean, maize, beet, pepper, grape, sweet potato, cucurbit, or okra plant. In certain embodiments, the plant is a cassava plant. In certain other embodiments, the plant is any plant except for a cassava plant.

The composition and methods of the present disclosure find use in producing plants with enhanced resistance to at least one geminivirus. Geminiviruses causing damage to plants include, for example, African cassava mosaic virus (ACMV), Indian cassava mosaic virus (ICMV), Bean calico mosaic virus, Bean dwarf mosaic virus, Bean golden mosaic virus (BGMV), Bhendi yellow vein mosaic virus (BYVMV), Chilli leaf curl virus (ChiLCV), Pepper golden mosaic virus, Pepper huasteco yellow vein virus (PHV), Pepper leaf curl virus (PepLCV), Potato yellow mosaic virus (PYMV), Sweet potato leaf curl virus, Tomato yellow leaf curl virus (TYLCV), Tomato leaf curl virus (TLCV), Cotton leaf curl virus (CLCV), Beet curly top virus (BCTV), Grapevine red blotch virus, Maize streak virus (MSV), and Wheat dwarf virus (WDV).

The present disclosure provides transformed plants, seeds, and plant cells produced by the methods of present disclosure and/or comprising a polynucleotide encoding a POLD1 polypeptide of the present disclosure. Also provided are progeny plants and seeds thereof comprising a polynucleotide of the present disclosure. The present disclosure also provides fruits, seeds, tubers, leaves, stems, roots, and other plant parts produced by the transformed plants and/or progeny plants of the disclosure as well as biological samples comprising, or produced or derived from, the plants or any part or parts thereof including, but not limited to, fruits, tubers, leaves, stems, roots, and seed. In certain embodiments, the biological sample is a commodity plant product. Commodity plant product include, for example, cassava flour or cassava meal products produced from cassava roots. It is recognized that such commodity plant products can be consumed or used by humans and other animals including, but not limited to, pets (e.g. dogs and cats), livestock (e.g. pigs, cows, chickens, turkeys, and ducks), and animals produced in freshwater and marine aquaculture systems (e.g. fish, shrimp, prawns, crayfish, and lobsters).

The plants disclosed herein find use in methods for limiting disease caused by a geminivirus in agricultural crop production, particularly in regions where such a disease is prevalent and is known to negatively impact, or at least has the potential to negatively impact, agricultural yield. The methods of the disclosure comprise planting a seedling, cutting, tuber, or seed of the present disclosure, wherein the seedling, tuber, or seed comprises a polynucleotide encoding a POLD1 polypeptide of the present disclosure. The methods further comprise growing the plant that is derived from the seedling, cutting, tuber, or seed under conditions favorable for the growth and development of the plant, and optionally harvesting at least one fruit, tuber, leaf, root, or seed from the plant.

The present disclosure additionally provides methods for identifying and/or selecting a plant with resistance to a disease caused by a geminivirus. The methods find use in breeding plants for resistance to diseases caused by geminiviruses such as, for example, cassava mosaic disease. The methods comprise detecting in a plant, or in at least one part or cell thereof, the presence of a polynucleotide encoding a POLD1 polypeptide of the present disclosure. In certain embodiments, detecting the presence of the polynucleotide comprises detecting the entire POLD1 nucleotide sequence in genomic DNA isolated from a plant. In certain embodiments, however, detecting the presence of the polynucleotide comprises detecting the presence of at least one marker within the POLD1 nucleotide sequence, optionally wherein the marker encodes an amino acid mutation in or near the active center of the POLD1 polypeptide. In other embodiments, detecting the presence of the polynucleotide comprises detecting the presence of the POLD1 polypeptide encoded by the polynucleotide using, for example, immunological detection methods involving antibodies specific to the POLD1 polypeptide.

In the methods for identifying and/or selecting a plant with resistance to a disease caused by a geminivirus, detecting the presence of the POLD1 polynucleotide in the plant can involve one or more of the following molecular biology techniques that are disclosed elsewhere herein or otherwise known in the art including, but not limited to, isolating genomic DNA and/or RNA from the plant, amplifying a nucleic acid molecule comprising the POLD1 polynucleotide and/or marker therein by PCR amplification, sequencing a nucleic acid molecule comprising the POLD1 polynucleotide and/or marker, identifying the POLD1 polynucleotide, the marker, or a transcript of the POLD1 polynucleotide by nucleic acid hybridization, and conducting an immunological assay for the detection of the POLD1 polypeptide encoded by the polynucleotide. It is recognized that oligonucleotide probes and PCR primers can be designed to identity the POLD1 polynucleotides of the present disclosure and that such probes and PCR primers can be utilized in methods disclosed elsewhere herein or otherwise known in the art to rapidly identify one or more plants comprising the presence of a POLD1 polynucleotide of the present disclosure in a population of plants.

Additionally provided are methods for introducing resistance to a disease caused by a geminivirus into a plant. The methods comprise crossing (i.e. cross-pollinating) a first plant comprising in its genome a polynucleotide encoding a POLD1 polypeptide of present disclosure with a second plant lacking in its genome the polynucleotide. Either the first plant or the second plant can be the pollen donor plant. For example, if the first plant is the pollen donor plant, then the second plant is the pollen-recipient plant. Likewise, if the second plant is the pollen donor plant, then the first plant is the pollen-recipient plant. Following the crossing, the pollen-recipient plant is grown under conditions favorable for the growth and development of the plant and for a sufficient period of time for seed to mature or to achieve an otherwise desirable growth stage for use. The seed can then be harvested and those seed comprising the polynucleotide identified by any method known in the art including, for example, the methods for identifying a plant with resistance to a disease caused by a geminivirus that are described elsewhere herein.

### EMBODIMENTS

The following numbered embodiments also form part of the present disclosure:

1. A transgenic plant, or a plant cell thereof, with enhanced resistance to at least one geminivirus, the plant comprising a transgene encoding a DNA polymerase delta subunit 1 (POLD1) polypeptide, wherein the POLD1 polypeptide comprises a mutation of at least one amino acid in or near the active center of the POLD1 polypeptide.

2. The transgenic plant of embodiment 1, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

3. The transgenic plant of embodiment 1 or embodiment 2, wherein the POLD1 polypeptide comprises a mutation of at least one amino acid at a position corresponding to position 520, 528, 598, 606, 617, 627, 680, 684, 685, 714, or 758 of SEQ ID NO: 1.

4. The transgenic plant of embodiment 1, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

5. The transgenic plant of any one of embodiments 1 to 4, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

6. The transgenic plant of any one of embodiments 1 to 5, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

7. The transgenic plant of any one of embodiments 1 to 6, wherein the transgene encoding the POLD1 polypeptide has at least 80%, at least 90%, at least 95%, at least 98%, or at least 99% sequence identity to at least one of the nucleotide sequences set forth in SEQ ID NOS: 2-4, 6-8, 10-12, 14-16, 18-20, 22-24, 26-28, 30-32, 34-36, 38-40, 42-44, 46-48, 50-52, 54-56, or 58-60.

8. The transgenic plant of any one of embodiments 1 to 7, wherein the plant is heterozygous for the mutation.

9. The transgenic plant of any one of embodiments 1 to 8, wherein the transgene is operably linked to a heterologous promoter functional in a plant cell.

10. The transgenic plant of any one of embodiments 1 to 9, wherein the geminivirus is a Begomovirus.

11. The transgenic plant of any one of embodiments 1 to 10, wherein the geminivirus is a Cassava mosaic virus.

12. The transgenic plant of any one of embodiments 1 to 11, wherein the plant is a cassava, tomato, cotton, bean, soybean, maize, beet, pepper, grape, sweet potato, cucurbit, or okra plant.

13. The transgenic plant of any one of embodiments 1 to 12, wherein the plant is a cassava plant.

14. A plant, or a plant cell thereof, with enhanced resistance to at least one geminivirus, the plant comprising a polynucleotide encoding a POLD1 polypeptide, wherein the POLD1 polypeptide comprises (a) a mutation of at least one amino acid at a position corresponding to position 528, 598, 680, 694, or 685 of SEQ ID NO: 1, wherein the plant is not a cassava plant; (b) a mutation of at least one amino acid at a position corresponding to position 627 of SEQ ID NO: 1, wherein the plant is not a tomato plant; (c) a mutation of at least one amino acid at a position corresponding to position 606, 714, or 758 of SEQ ID NO: 1, wherein the plant is not a cotton plant; or (d) a mutation of at least one amino acid at a position corresponding to position 520 or 617, wherein the plant is not a soybean plant.

15. The plant of embodiment 14, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

16. The plant of embodiment 14 or embodiment 15, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

17. The plant of any one of embodiments 14 to 16, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

18. The plant of any one of embodiments 14 to 17, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

19. The plant of any one of embodiments 14 to 18, wherein the plant is heterozygous for the mutation.

20. The plant of any one of embodiments 14 to 19, wherein the geminivirus is a Begomovirus.

21. The plant of any one of embodiments 14 to 20, wherein the plant is a cassava, tomato, cotton, bean, soybean, maize, beet, pepper, grape, sweet potato, cucurbit, or okra plant.

22. A plant part obtained from the plant of any one of embodiments 1 to 21, optionally wherein the plant part is a seed.

23. A method of enhancing resistance of a plant to infection by a geminivirus, the method comprising: modifying at least one plant cell to comprise a polynucleotide encoding a POLD1 polypeptide, wherein the POLD1 polypeptide comprises a mutation of at least one amino acid in or near the active center of the POLD1 polypeptide.

24. The method of embodiment 23, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

25. The method of embodiment 23 or embodiment 24, wherein the POLD1 polypeptide comprises a mutation of at least one amino acid at a position corresponding to position 520, 528, 598, 606, 617, 627, 680, 684, 685, 714, or 758 of SEQ ID NO: 1.

26. The method of any one of embodiments 23 to 25, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

27. The method of any one of embodiments 23 to 26, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

28. The method of any one of embodiments 23 to 27, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

29. The method of any one of embodiments 23 to 28, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

30. The method of any one of embodiments 23 to 29, wherein the modifying comprises transforming at least one plant cell with a polynucleotide encoding the POLD1 polypeptide.

31. The method of any one of embodiments 23 to 30, wherein the polynucleotide is operably linked to a promoter functional in a plant cell.

32. The method of any one of embodiments 23 to 29, wherein the modifying comprises using genome editing to modify the nucleotide sequence of a native gene in the genome of the plant cell.

33. The method of embodiment 32, wherein the genome editing comprises using a zinc-finger nuclease (ZFN), a TAL (transcription activator-like) effector nuclease (TALEN), or a Clustered Regularly Interspaced Short Palindromic Repeats/CRISPR-associated nuclease (CRISPR/Cas nuclease) system.

34. The method of any one of embodiments 23 to 33, wherein the plant cell is regenerated into a plant comprising in its genome the polynucleotide.

35. The method of any one of embodiments 23 to 34, further comprising selecting for a plant or a plant cell having enhanced resistance to the geminivirus as compared to a corresponding control plant or plant cell without the polynucleotide.

36. The method of any one of embodiments 23 to 35, wherein the geminivirus is a Begomovirus.

37. The method of any one of embodiments 23 to 36, wherein the geminivirus is a Cassava mosaic virus.

38. The method of any one of embodiments 23 to 37, wherein the plant is a cassava, tomato, cotton, bean, soybean, maize, beet, pepper, grape, sweet potato, cucurbit, or okra plant.

39. The method of any one of embodiments 23 to 38, wherein the plant is a cassava plant.

40. A method of limiting a disease caused by a geminivirus in agricultural crop production, the method comprising: planting a seedling, cutting, tuber, or seed of the plant of any one of embodiments 1 to 21; and growing the seedling, cutting, tuber, or seed under conditions favorable for the growth and development of a plant resulting therefrom, optionally wherein the plant is subjected to geminivirus infection.

41. The method of embodiment 40, further comprising harvesting at least one fruit, tuber, leaf, root, and/or seed from the plant.

42. The method of embodiment 40 or embodiment 41, wherein the plant is a cassava plant, and wherein the disease is cassava mosaic disease.

43. A method for selecting a plant with resistance to a disease caused by a geminivirus, the method comprising: detecting the presence of (i) a POLD1 polypeptide or (ii) a polynucleotide encoding the POLD1 polypeptide, wherein the POLD1 polypeptide comprises a mutation of at least one amino acid in or near the active center of the POLD1 polypeptide in a plant, or in at least one part or cell thereof, wherein the plant is within a mixed population of plants comprising other plants which lack the POLD1 polypeptide comprising the mutation; and selecting the plant comprising the POLD1 polypeptide or the polynucleotide encoding the POLD1 polypeptide.

44. The method of embodiment 43, further comprising the step of selfing or crossing the selected plant.

45. The method of embodiment 43 or embodiment 44, wherein the crossing is to a plant lacking the polynucleotide encoding the POLD1 polypeptide comprising the mutation, optionally wherein progeny of the cross are subjected to the detecting and selecting steps.

46. The method of any one of embodiments 43 to 45, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

47. The method of any one of embodiments 43 to 46, wherein the POLD1 polypeptide comprises a mutation of at least one amino acid at a position corresponding to position 520, 528, 598, 606, 617, 627, 680, 684, 685, 714, or 758 of SEQ ID NO: 1.

48. The method of any one of embodiments 43 to 45, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

49. The method of any one of embodiments 43 to 48, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

50. The method of embodiment 43, wherein the plant is a cassava, tomato, cotton, bean, soybean, maize, beet, pepper, grape, sweet potato, cucurbit, or okra plant.

51. The method of embodiment 43, wherein the plant is a cassava plant, and wherein the disease is cassava mosaic disease.

52. The method of any one of embodiments 43 to 51, wherein the detecting comprises PCR amplification, nucleic acid sequencing, nucleic acid hybridization, or an immunological assay.

53. A method for introducing resistance to a disease caused by a geminivirus into a plant, the method comprising: (a) crossing a first plant comprising in its genome a polynucleotide encoding a POLD1 polypeptide with a second plant lacking in its genome the polynucleotide, wherein the POLD1 polypeptide comprises a mutation of at least one amino acid in or near the active center of the POLD1 polypeptide, whereby at least one progeny plant is produced; (b) genotyping at least one progeny plant for the presence of the mutation; and (c) selecting at least one progeny plant comprising in its genome the polynucleotide encoding the POLD1 polypeptide comprising the mutation.

54. The method of embodiment 53, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

55. The method of embodiment 53, wherein the POLD1 polypeptide comprises a mutation of at least one amino acid at a position corresponding to position 520, 528, 598, 606, 617, 627, 680, 684, 685, 714, or 758 of SEQ ID NO: 1.

56. The method of embodiment 53, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

57. The method of embodiment 53, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

58. The method of embodiment 53, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

59. The method of embodiment 53, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

60. The method of any one of embodiments 53 to 59, further comprising (i) backcrossing at least one selected progeny plant of (c) to a plant that is of the genotype as the second plant, whereby at least one progeny plant is produced from the backcrossing, optionally wherein progeny of the backcross are subjected to the genotyping; and (ii) selecting at least one progeny plant comprising in its genome the polynucleotide that is produced from the backcrossing of (i).

61. A polynucleotide encoding a POLD1 polypeptide, wherein the POLD1 polypeptide comprises a mutation of at least one amino acid in or near the active center of the POLD1 polypeptide, and wherein the polynucleotide is operably linked to a polynucleotide comprising a heterologous promoter.

62. The polynucleotide of embodiment 61, wherein the polynucleotide is capable of conferring resistance to a disease caused by a geminivirus to a plant comprising the polynucleotide.

63. The polynucleotide of embodiment 61, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

64. The polynucleotide of embodiment 61, wherein the POLD1 polypeptide comprises a mutation of at least one amino acid at a position corresponding to position 520, 528, 598, 606, 617, 627, 680, 684, 685, 714, or 758 of SEQ ID NO: 1.

65. The polynucleotide of embodiment 61, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

66. The polynucleotide of embodiment 61, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

67. The polynucleotide of embodiment 61, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

68. The polynucleotide of embodiment 61, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

69. A vector comprising the polynucleotide of any one of embodiments 61 to 68.

70. A host cell comprising the polynucleotide of any one of embodiments 61 to 68.

71. The host cell of embodiment 70, wherein the host cell is a plant cell, optionally wherein the plant cell is a cassava cell.

72. A method of producing a commodity plant product, the method comprising: (i) processing the plant of any one of embodiments 1 to 21, or a part thereof; and (ii) recovering the commodity plant product from the processed plant or part thereof.

73. The method of embodiment 72, wherein the commodity plant product is cassava flour or cassava meal.

74. The method of embodiment 72, wherein the commodity plant product comprises a detectable amount of the polynucleotide encoding the POLD1 polypeptide.

75. A biological sample comprising a detectable amount of a polynucleotide comprising a transgene encoding a POLD1 polypeptide, wherein the POLD1 polypeptide comprises a mutation of at least one amino acid in or near the active center of the POLD1 polypeptide.

76. The biological sample of embodiment 75, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

77. The biological sample of embodiment 75, wherein the POLD1 polypeptide comprises a mutation of at least one amino acid at a position corresponding to position 520, 528, 598, 606, 617, 627, 680, 684, 685, 714, or 758 of SEQ ID NO: 1.

78. The biological sample of embodiment 75, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

79. The biological sample of embodiment 75, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

80. The biological sample of embodiment 75, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57.

81. The biological sample of embodiment 75, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

82. The biological sample of embodiment 75, wherein the polynucleotide is operably linked to a heterologous promoter functional in a plant cell.

83. The biological sample of any one of embodiments 75 to 82, wherein the biological sample is non-regenerable.

84. The biological sample of any one of embodiments 75 to 82, wherein the biological sample comprises cassava flour or cassava meal.

85. A tomato plant, or a plant cell thereof, with enhanced resistance to at least one geminivirus, the plant comprising a polynucleotide encoding a POLD1 polypeptide, wherein the POLD1 polypeptide comprises a mutation of at least one amino acid at a position corresponding to position 515, 523, 593, 601, 612, 675, 679, 680, 622, 709, or 753 of SEQ ID NO: 5.

86. The tomato plant of embodiment 85, wherein the POLD1 polypeptide comprises a serine at the position corresponding to position 515 of SEQ ID NO: 5, a leucine at the position corresponding to position 523 of SEQ ID NO: 5, a tryptophan at the position corresponding to position 593 of SEQ ID NO: 5, a cysteine at the position corresponding to position 601 of SEQ ID NO: 5, a lysine at the position corresponding to position 612 of SEQ ID NO: 5, an aspartic acid at the position corresponding to position 622 of SEQ ID NO: 5, a valine or an arginine at the position corresponding to position 675 of SEQ ID NO: 5, a glycine at the position corresponding to position 679 of SEQ ID NO: 5, a phenylalanine at the position corresponding to position 680 of SEQ ID NO: 5, an arginine at the position corresponding to position 709 of SEQ ID NO: 5, or a phenylalanine at the position corresponding to position 753 of SEQ ID NO: 5.

87. The tomato plant of embodiment 85 or embodiment 86, wherein the POLD1 polypeptide has at least 80%, at least 90%, at least 95%, at least 98%, or at least 99% sequence identity to the amino acid sequence set forth in SEQ ID NO: 5 or 45.

88. The tomato plant of any one of embodiments 85-87, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 6-8 or 46-48.

89. The tomato plant of any one of embodiments 85-88, wherein the tomato plant is Solanum lycopersicum.

90. The tomato plant of any one of embodiments 85-89, wherein the tomato plant comprises elite germplasm.

91. The method of any one of embodiments 23-36, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

92. The method of embodiment 91, wherein the POLD1 polypeptide comprises a serine at the position corresponding to position 515 of SEQ ID NO: 5, a leucine at the position corresponding to position 523 of SEQ ID NO: 5, a tryptophan at the position corresponding to position 593 of SEQ ID NO: 5, a cysteine at the position corresponding to position 601 of SEQ ID NO: 5, a lysine at the position corresponding to position 612 of SEQ ID NO: 5, an aspartic acid at the position corresponding to position 622 of SEQ ID NO: 5, a valine or an arginine at the position corresponding to position 675 of SEQ ID NO: 5, a glycine at the position corresponding to position 679 of SEQ ID NO: 5, a phenylalanine at the position corresponding to position 680 of SEQ ID NO: 5, an arginine at the position corresponding to position 709 of SEQ ID NO: 5, or a phenylalanine at the position corresponding to position 753 of SEQ ID NO: 5.

93. The method of embodiment 91 or embodiment 92, wherein the POLD1 polypeptide has at least 80%, at least 90%, at least 95%, at least 98%, or at least 99% sequence identity to the amino acid sequence set forth in SEQ ID NO: 5 or 45.

94. The method of any one of embodiments 91-93, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 6-8 or 46-48.

95. The method of any one of embodiments 91-94, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

96. The method of any one of embodiments 43-52, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

97. The method of embodiment 96, wherein the POLD1 polypeptide comprises a serine at the position corresponding to position 515 of SEQ ID NO: 5, a leucine at the position corresponding to position 523 of SEQ ID NO: 5, a tryptophan at the position corresponding to position 593 of SEQ ID NO: 5, a cysteine at the position corresponding to position 601 of SEQ ID NO: 5, a lysine at the position corresponding to position 612 of SEQ ID NO: 5, an aspartic acid at the position corresponding to position 622 of SEQ ID NO: 5, a valine or an arginine at the position corresponding to position 675 of SEQ ID NO: 5, a glycine at the position corresponding to position 679 of SEQ ID NO: 5, a phenylalanine at the position corresponding to position 680 of SEQ ID NO: 5, an arginine at the position corresponding to position 709 of SEQ ID NO: 5, or a phenylalanine at the position corresponding to position 753 of SEQ ID NO: 5.

98. The method of embodiment 96 or embodiment 97, wherein the POLD1 polypeptide has at least 80%, at least 90%, at least 95%, at least 98%, or at least 99% sequence identity to the amino acid sequence set forth in SEQ ID NO: 5 or 45.

99. The method of any one of embodiments 96-98, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 6-8 or 46-48.

100. The method of any one of embodiments 96-99, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

101. The method of any one of embodiments 53-60, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

102. The method of embodiment 101, wherein the POLD1 polypeptide comprises a serine at the position corresponding to position 515 of SEQ ID NO: 5, a leucine at the position corresponding to position 523 of SEQ ID NO: 5, a tryptophan at the position corresponding to position 593 of SEQ ID NO: 5, a cysteine at the position corresponding to position 601 of SEQ ID NO: 5, a lysine at the position corresponding to position 612 of SEQ ID NO: 5, an aspartic acid at the position corresponding to position 622 of SEQ ID NO: 5, a valine or an arginine at the position corresponding to position 675 of SEQ ID NO: 5, a glycine at the position corresponding to position 679 of SEQ ID NO: 5, a phenylalanine at the position corresponding to position 680 of SEQ ID NO: 5, an arginine at the position corresponding to position 709 of SEQ ID NO: 5, or a phenylalanine at the position corresponding to position 753 of SEQ ID NO: 5.

103. The method of embodiment 101 or embodiment 102, wherein the POLD1 polypeptide has at least 80%, at least 90%, at least 95%, at least 98%, or at least 99% sequence identity to the amino acid sequence set forth in SEQ ID NO: 5 or 45.

104. The method of any one of embodiments 101-103, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 6-8 or 46-48.

105. The method of any one of embodiments 101-104, wherein the plant is tomato, and wherein the POLD1 polypeptide comprises a mutation of at least one amino acid at a position corresponding to position 515, 523, 593, 601, 612, 675, 679, 680, 622, 709, or 753 of SEQ ID NO: 5.

106. A cotton plant, or a plant cell thereof, with enhanced resistance to at least one geminivirus, the plant comprising a polynucleotide encoding a POLD1 polypeptide, wherein the POLD1 polypeptide comprises a mutation of at least one amino acid at a position corresponding to position 526, 534, 604, 612, 623, 633, 686, 690, 691, 720, or 764 of SEQ ID NO: 9.

107. The cotton plant of embodiment 106, wherein the POLD1 polypeptide comprises a serine at the position corresponding to position 526 of SEQ ID NO: 9, a leucine at the position corresponding to position 534 of SEQ ID NO: 9, a tryptophan at the position corresponding to position 604 of SEQ ID NO: 9, a cysteine at the position corresponding to position 612 of SEQ ID NO: 9, a lysine at the position corresponding to position 623 of SEQ ID NO: 9, an aspartic acid at the position corresponding to position 633 of SEQ ID NO: 9, a valine or an arginine at the position corresponding to position 686 of SEQ ID NO: 9, a glycine at the position corresponding to position 690 of SEQ ID NO: 9, a phenylalanine at the position corresponding to position 691 of SEQ ID NO: 9, an arginine at the position corresponding to position 720 of SEQ ID NO: 9, or a phenylalanine at the position corresponding to position 764 of SEQ ID NO: 9.

108. The cotton plant of embodiment 106 or embodiment 107, wherein the POLD1 polypeptide has at least 80%, at least 90%, at least 95%, at least 98%, or at least 99% sequence identity to the amino acid sequence set forth in SEQ ID NO: 9.

109. The cotton plant of any one of embodiments 106-108, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 10-12.

110. The cotton plant of any one of embodiments 106-109, wherein the cotton plant comprises elite germplasm.

111. The method of any one of embodiments 23-36, wherein the plant is cotton, and wherein the POLD1 polypeptide comprises a mutation of at least one amino acid at a position corresponding to position 526, 534, 604, 612, 623, 633, 686, 690, 691, 720, or 764 of SEQ ID NO: 9.

112. The method of embodiment 111, wherein the POLD1 polypeptide comprises a serine at the position corresponding to position 526 of SEQ ID NO: 9, a leucine at the position corresponding to position 534 of SEQ ID NO: 9, a tryptophan at the position corresponding to position 604 of SEQ ID NO: 9, a cysteine at the position corresponding to position 612 of SEQ ID NO: 9, a lysine at the position corresponding to position 623 of SEQ ID NO: 9, an aspartic acid at the position corresponding to position 633 of SEQ ID NO: 9, a valine or an arginine at the position corresponding to position 686 of SEQ ID NO: 9, a glycine at the position corresponding to position 690 of SEQ ID NO: 9, a phenylalanine at the position corresponding to position 691 of SEQ ID NO: 9, an arginine at the position corresponding to position 720 of SEQ ID NO: 9, or a phenylalanine at the position corresponding to position 764 of SEQ ID NO: 9.

113. The method of embodiment 111 or embodiment 112, wherein the POLD1 polypeptide has at least 80%, at least 90%, at least 95%, at least 98%, or at least 99% sequence identity to the amino acid sequence set forth in SEQ ID NO: 9.

114. The method of any one of embodiments 111-113, wherein the polynucleotide encoding the POLD1 polypeptide has at least 80%, at least 90%, at least 95%, at least 98%, or at least 99% sequence identity to at least one of the nucleotide sequences set forth in SEQ ID NOS: 10-12.

115. The method any one of embodiments 43-52, wherein the plant is cotton, and wherein the POLD1 polypeptide comprises a mutation of at least one amino acid at a position corresponding to position 526, 534, 604, 612, 623, 633, 686, 690, 691, 720, or 764 of SEQ ID NO: 9.

116. The method of embodiment 115, wherein the POLD1 polypeptide comprises a serine at the position corresponding to position 526 of SEQ ID NO: 9, a leucine at the position corresponding to position 534 of SEQ ID NO: 9, a tryptophan at the position corresponding to position 604 of SEQ ID NO: 9, a cysteine at the position corresponding to position 612 of SEQ ID NO: 9, a lysine at the position corresponding to position 623 of SEQ ID NO: 9, an aspartic acid at the position corresponding to position 633 of SEQ ID NO: 9, a valine or an arginine at the position corresponding to position 686 of SEQ ID NO: 9, a glycine at the position corresponding to position 690 of SEQ ID NO: 9, a phenylalanine at the position corresponding to position 691 of SEQ ID NO: 9, an arginine at the position corresponding to position 720 of SEQ ID NO: 9, or a phenylalanine at the position corresponding to position 764 of SEQ ID NO: 9.

117. The method of embodiment 115 or embodiment 116, wherein the POLD1 polypeptide has at least 80%, at least 90%, at least 95%, at least 98%, or at least 99% sequence identity to the amino acid sequence set forth in SEQ ID NO: 9.

118. The method of any one of embodiments 115-117, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 10-12.

119. The method of any one of embodiments 53-60, wherein the plant is cotton, and wherein the POLD1 polypeptide comprises a mutation of at least one amino acid at a position corresponding to position 526, 534, 604, 612, 623, 633, 686, 690, 691, 720, or 764 of SEQ ID NO: 9.

120. The method of embodiment 119, wherein the POLD1 polypeptide comprises a serine at the position corresponding to position 526 of SEQ ID NO: 9, a leucine at the position corresponding to position 534 of SEQ ID NO: 9, a tryptophan at the position corresponding to position 604 of SEQ ID NO: 9, a cysteine at the position corresponding to position 612 of SEQ ID NO: 9, a lysine at the position corresponding to position 623 of SEQ ID NO: 9, an aspartic acid at the position corresponding to position 633 of SEQ ID NO: 9, a valine or an arginine at the position corresponding to position 686 of SEQ ID NO: 9, a glycine at the position corresponding to position 690 of SEQ ID NO: 9, a phenylalanine at the position corresponding to position 691 of SEQ ID NO: 9, an arginine at the position corresponding to position 720 of SEQ ID NO: 9, or a phenylalanine at the position corresponding to position 764 of SEQ ID NO: 9.

121. The method of embodiment 119 or embodiment 120, wherein the POLD1 polypeptide has at least 80%, at least 90%, at least 95%, at least 98%, or at least 99% sequence identity to the amino acid sequence set forth in SEQ ID NO: 9.

122. The method of any one of embodiments 119-121, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 10-12.

123. A soybean plant, or a plant cell thereof, with enhanced resistance to at least one geminivirus, the plant comprising a polynucleotide encoding a POLD1 polypeptide, wherein the POLD1 polypeptide comprises a mutation of at least one amino acid at a position corresponding to position 514, 522, 592, 600, 611, 621, 674, 678, 679, 708, or 752 of SEQ ID NO: 17.

124. The soybean plant of embodiment 123, wherein the POLD1 polypeptide comprises a serine at the position corresponding to position 514 of SEQ ID NO: 17, a leucine at the position corresponding to position 522 of SEQ ID NO: 17, a tryptophan at the position corresponding to position 592 of SEQ ID NO: 17, a cysteine at the position corresponding to position 600 of SEQ ID NO: 17, a lysine at the position corresponding to position 611 of SEQ ID NO: 17, an aspartic acid at the position corresponding to position 621 of SEQ ID NO: 17, a valine or an arginine at the position corresponding to position 674 of SEQ ID NO: 17, a glycine at the position corresponding to position 678 of SEQ ID NO: 17, a phenylalanine at the position corresponding to position 679 of SEQ ID NO: 17, an arginine at the position corresponding to position 708 of SEQ ID NO: 17, or a phenylalanine at the position corresponding to position 752 of SEQ ID NO: 17.

125. The soybean plant of embodiment 123 or embodiment 124, wherein the POLD1 polypeptide has at least 80%, at least 90%, at least 95%, at least 98%, or at least 99% sequence identity to the amino acid sequence set forth in SEQ ID NO: 17.

126. The soybean plant of any one of embodiments 123-125, wherein the polynucleotide encoding the POLD1 polypeptide has at least 80%, at least 90%, at least 95%, at least 98%, or at least 99% sequence identity to at least one of the nucleotide sequences set forth in SEQ ID NOS: 18-20.

127. The soybean plant any one of embodiments 123-126, wherein the soybean plant comprises elite germplasm.

128. The method of any one of embodiments 23-36, wherein the plant is soybean, and wherein the POLD1 polypeptide comprises a mutation of at least one amino acid at a position corresponding to position 514, 522, 592, 600, 611, 621, 674, 678, 679, 708, or 752 of SEQ ID NO: 17.

129. The method of embodiment 128, wherein the POLD1 polypeptide comprises a serine at the position corresponding to position 514 of SEQ ID NO: 17, a leucine at the position corresponding to position 522 of SEQ ID NO: 17, a tryptophan at the position corresponding to position 592 of SEQ ID NO: 17, a cysteine at the position corresponding to position 600 of SEQ ID NO: 17, a lysine at the position corresponding to position 611 of SEQ ID NO: 17, an aspartic acid at the position corresponding to position 621 of SEQ ID NO: 17, a valine or an arginine at the position corresponding to position 674 of SEQ ID NO: 17, a glycine at the position corresponding to position 678 of SEQ ID NO: 17, a phenylalanine at the position corresponding to position 679 of SEQ ID NO: 17, an arginine at the position corresponding to position 708 of SEQ ID NO: 17, or a phenylalanine at the position corresponding to position 752 of SEQ ID NO: 17.

130. The method of embodiment 128 or embodiment 129, wherein the POLD1 polypeptide has at least 80%, at least 90%, at least 95%, at least 98%, or at least 99% sequence identity to the amino acid sequence set forth in SEQ ID NO: 17.

131. The method of any one of embodiments 128-130, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 18-20.

132. The method any one of embodiments 43-52, wherein the plant is soybean, and wherein the POLD1 polypeptide comprises a mutation of at least one amino acid at a position corresponding to position 514, 522, 592, 600, 611, 621, 674, 678, 679, 708, or 752 of SEQ ID NO: 17.

133. The method of embodiment 132, wherein the POLD1 polypeptide comprises a serine at the position corresponding to position 514 of SEQ ID NO: 17, a leucine at the position corresponding to position 522 of SEQ ID NO: 17, a tryptophan at the position corresponding to position 592 of SEQ ID NO: 17, a cysteine at the position corresponding to position 600 of SEQ ID NO: 17, a lysine at the position corresponding to position 611 of SEQ ID NO: 17, an aspartic acid at the position corresponding to position 621 of SEQ ID NO: 17, a valine or an arginine at the position corresponding to position 674 of SEQ ID NO: 17, a glycine at the position corresponding to position 678 of SEQ ID NO: 17, a phenylalanine at the position corresponding to position 679 of SEQ ID NO: 17, an arginine at the position corresponding to position 708 of SEQ ID NO: 17, or a phenylalanine at the position corresponding to position 752 of SEQ ID NO: 17.

134. The method of embodiment 132 or embodiment 133, wherein the POLD1 polypeptide has at least 80%, at least 90%, at least 95%, at least 98%, or at least 99% sequence identity to the amino acid sequence set forth in SEQ ID NO: 17.

135. The method of any one of embodiments 132-134, wherein the polynucleotide encoding the POLD1 polypeptide has at least 80%, at least 90%, at least 95%, at least 98%, or at least 99% sequence identity to at least one of the nucleotide sequences set forth in SEQ ID NOS: 18-20.

136. The method of any one of embodiments 53-60, wherein the plant is soybean, and wherein the POLD1 polypeptide comprises a mutation of at least one amino acid at a position corresponding to position 514, 522, 592, 600, 611, 621, 674, 678, 679, 708, or 752 of SEQ ID NO: 17.

137. The method of embodiment 136, wherein the POLD1 polypeptide comprises a serine at the position corresponding to position 514 of SEQ ID NO: 17, a leucine at the position corresponding to position 522 of SEQ ID NO: 17, a tryptophan at the position corresponding to position 592 of SEQ ID NO: 17, a cysteine at the position corresponding to position 600 of SEQ ID NO: 17, a lysine at the position corresponding to position 611 of SEQ ID NO: 17, an aspartic acid at the position corresponding to position 621 of SEQ ID NO: 17, a valine or an arginine at the position corresponding to position 674 of SEQ ID NO: 17, a glycine at the position corresponding to position 678 of SEQ ID NO: 17, a phenylalanine at the position corresponding to position 679 of SEQ ID NO: 17, an arginine at the position corresponding to position 708 of SEQ ID NO: 17, or a phenylalanine at the position corresponding to position 752 of SEQ ID NO: 17.

138. The method of embodiment 136 or embodiment 137, wherein the POLD1 polypeptide has at least 80%, at least 90%, at least 95%, at least 98%, or at least 99% sequence identity to the amino acid sequence set forth in SEQ ID NO: 17.

139. The method of any one of embodiments 136-138, wherein the polynucleotide encoding the POLD1 polypeptide has at least 80%, at least 90%, at least 95%, at least 98%, or at least 99% sequence identity to at least one of the nucleotide sequences set forth in SEQ ID NOs: 18-20.

### EXAMPLES

### Example 1

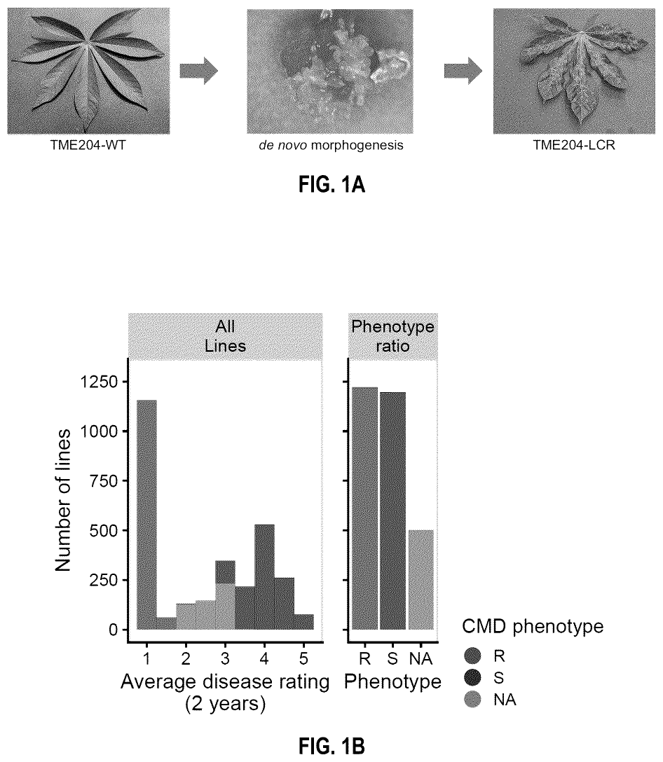

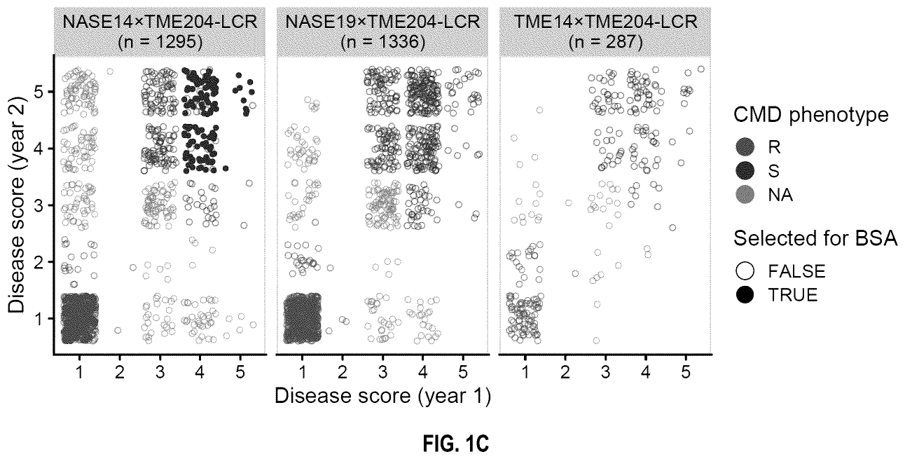

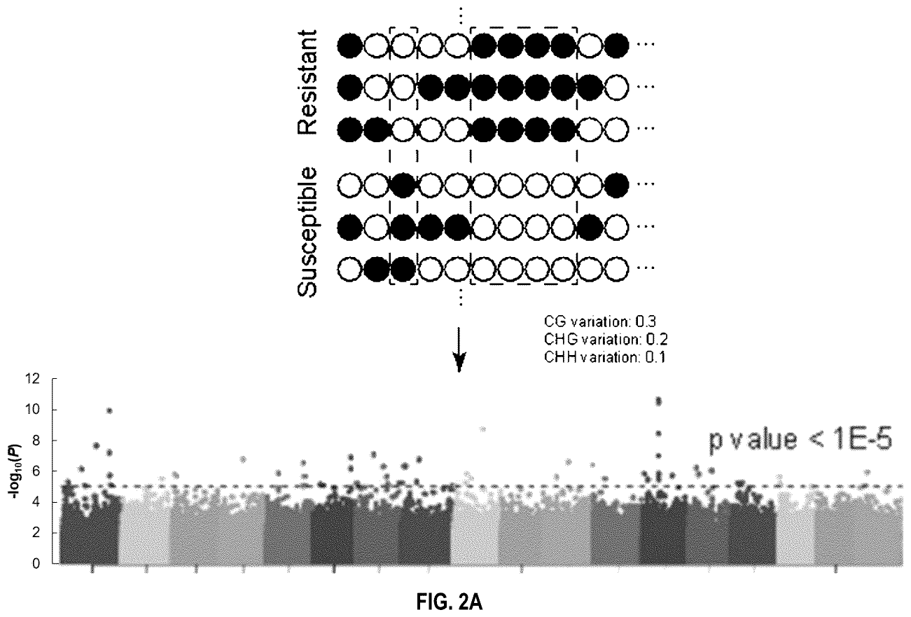

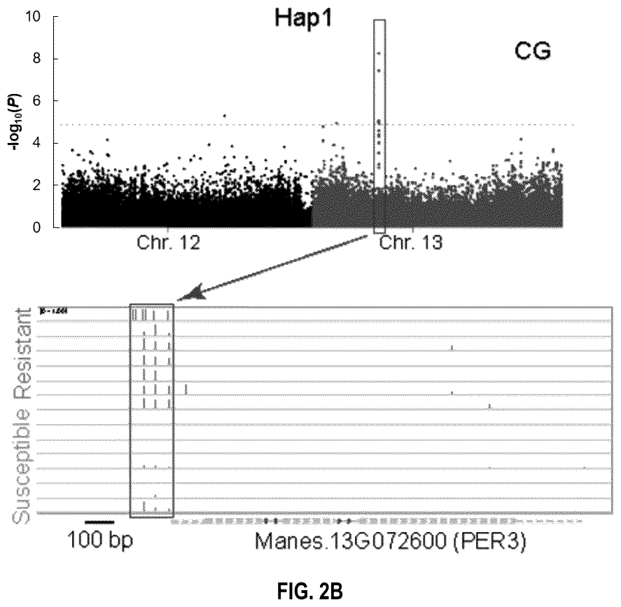

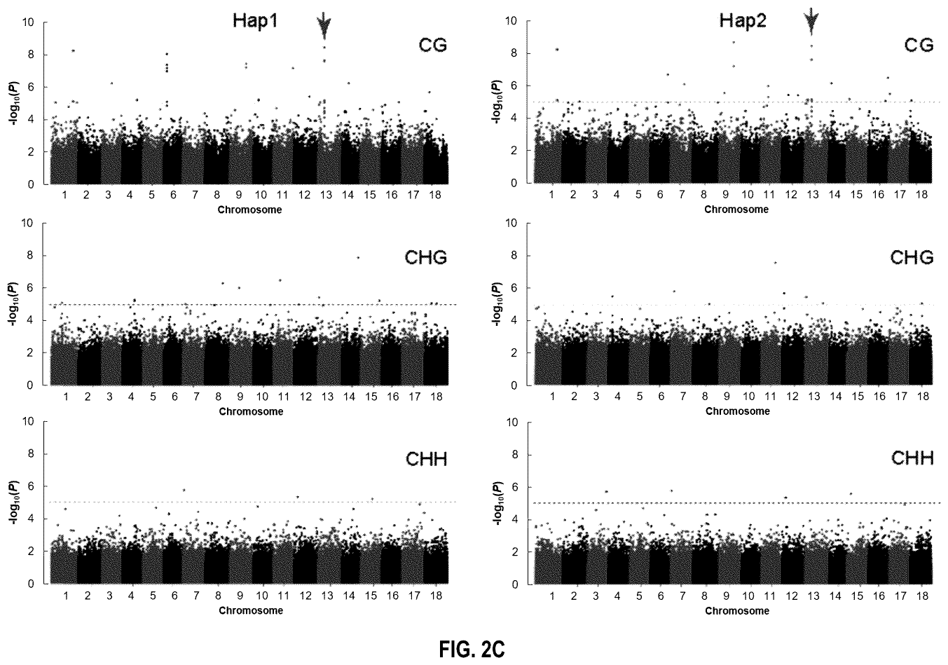

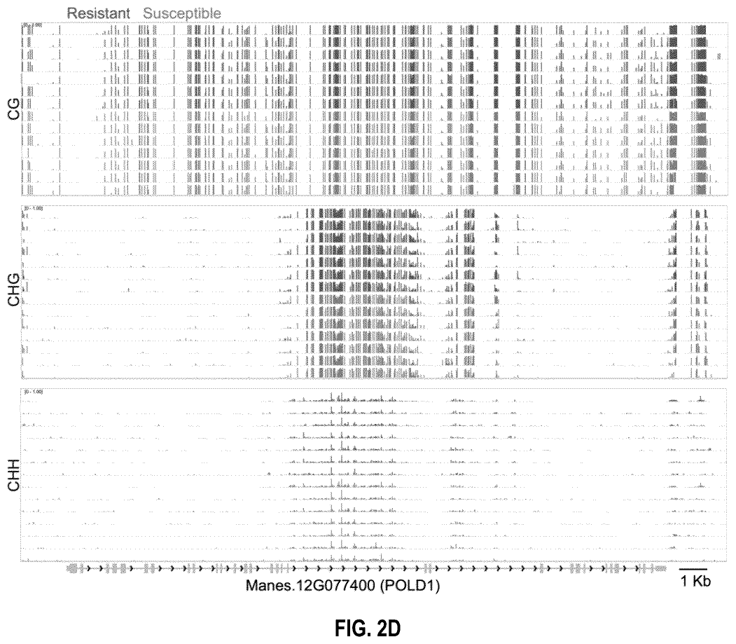

CMD2-associated resistance, which was discovered in landraces collected across West Africa, is a dominant single genetic locus located on Chromosome 12. CMD2-type resistance is lost when plants are regenerated through de novo morphogenesis in tissue culture (FIG. 1A). While loss of CMD2 resistance (LCR) occurs consistently in multiple landraces it was not observed in varieties generated through breeding programs. Epigenetic somaclonal variation is well known to produce phenotypic changes in plants regenerated from in vitro cultures. Therefore, it was hypothesised that the LCR phenotype is caused by culture-induced epigenetic changes at the CMD2 locus. Single-cytosine resolution epigenome-wide association studies (EWAS) were performed on multiple cassava plant lines, before and after in vitro morphogenesis. While methylation changes were found across the genome, no consistent methylation changes were observed within the CMD2 locus (FIG. 2). Table 2 shows the genotypes of plants used for EWAS analysis.

TABLE 2

| Name | Genotype | ID | Phenotype | Reads No. | Coverage* | Conversion rate** |
|---|---|---|---|---|---|---|
| TME 7 WT | TME7 | cBS14p | Resistant to CMD | 846983521 | 114 | 99.40% |
| TME 7 WT | TME7 | cBS8p | | 859059800 | 115.62 | 99.42% |
| TME 7 WT | TME7 | cBS15p | | 844574034 | 113.67 | 99.40% |
| TME7 OES | TME7 | cBS10p | Susceptible to | 853329597 | 114.85 | 99.41% |
| TME7 OES | TME7 | cBS11p | CMD | 774679128 | 104.27 | 99.41% |
| TME7 OES | TME7 | cBS12p | | 775731790 | 104.41 | 99.49% |
| TME7 | TME7 | cBS13p | Resistant to CMD | 726885049 | 97.83 | 99.55% |
| Organogenesis | | | | | | |
| TME7 | TME7 | cBS7p | | 832384407 | 112.03 | 99.52% |
| Organogenesis | | | | | | |
| TME7 | TME7 | cBS9p | | 760693488 | 102.38 | 99.53% |
| Organogenesis | | | | | | |
| TME7 | TME7 | cBS16p | Susceptible to | 794495267 | 106.93 | 99.39% |
| Organogenesis | | | CMD | | | |
| TME7 | TME7 | cBS17p | | 754538912 | 101.56 | 99.41% |
| Organogenesis | | | | | | |
| TME7 | TME7 | cBS18p | | 744368702 | 100.19 | 99.43% |
| Organogenesis | | | | | | |
| TME204 WT | TME204 | TME204 | Resistant to CMD | 1606930158 | 223.43 | 99.70% |
| TME204 FEC | TME204 | FEC | Susceptible to | 1659955923 | 233.43 | 99.76% |
| | | | CMD | | | |

WT: wildtype plant, OES: plant regenerated from organized embryogenic structure, Organogenesis: plant
regenerated via caulogenesis, FEC: plant regenerated from friable embryogenic callus.
*Estimated with genome size,
**Estimated with chloroplast

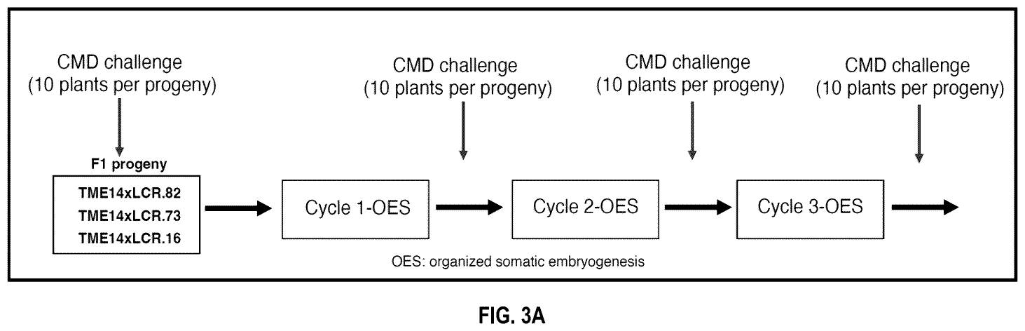

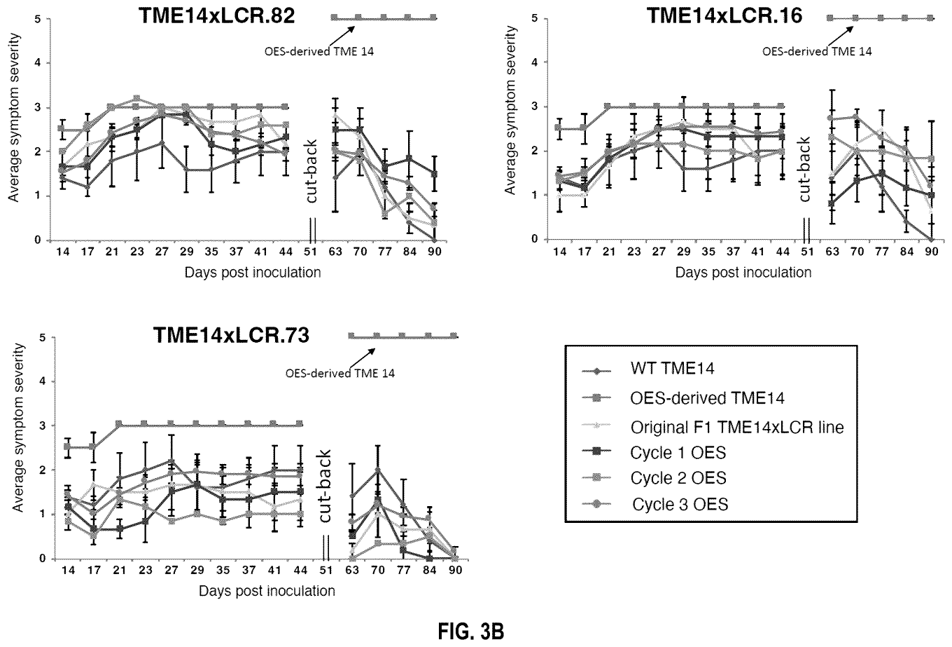

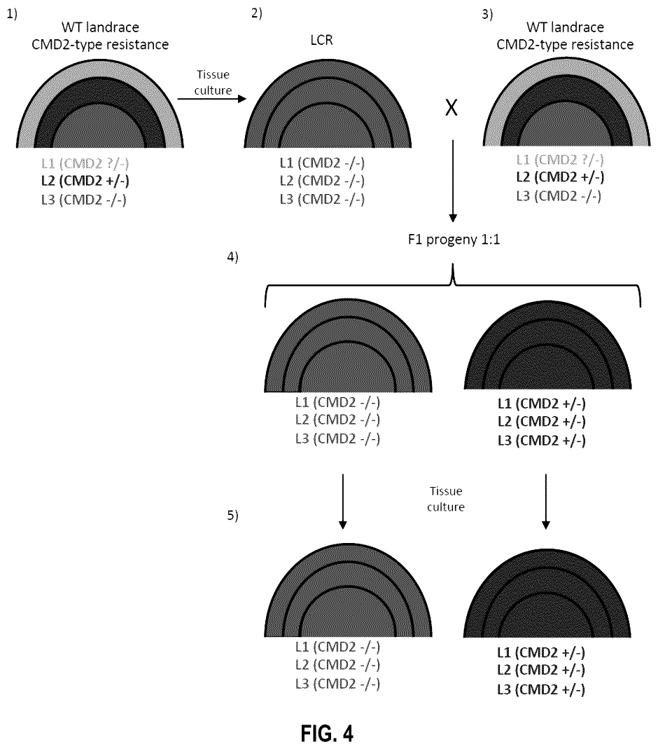

The relationship between the CMD2 and the LCR phenotypes was investigated by generating three large mapping populations derived from tissue culture regenerated, CMD susceptible plants (TME204-LCR) crossed with resistant varieties heterozygous for CMD2 (NASE14, NASE19, TME14). Field phenotyping was performed over two years at a high CMD pressure location in Uganda, and progeny lines assessed for resistance or susceptibility to CMD (FIG. 1B). Resistance segregated at 1:1 ratio (across all populations, χ² p-value=0.59), indicating that the dominant wildtype allele of CMD2 is sufficient to restore resistance, and that the CMD2 and LCR phenotypes are caused by a single genetic locus. If LCR results from a somaclonal epiallele, then passage of CMD resistant F₁ progeny through morphogenesis would result in the LCR phenotype. However, three independent, resistant F₁ progeny retained resistance through three consecutive cycles of somatic embryogenesis and plant regeneration, indicating that meiosis stabilises CMD2-derived resistance and prevents the LCR phenotype (FIG. 3). These results indicate that the CMD2 and LCR traits have a genomic basis. We postulate that spontaneous mutation(s) occurred in the West African landraces and became fixed as periclinal chimeras that were selected by farmers and maintained through clonal propagation, as observed previously in other plant species. Loss of resistance to CMD would be explained if de novo morphogenesis occurs from cell layers that do not carry the resistance allele. Gametes are typically derived from cells within the L2 layer of the meristem, thus if L2 cells carried the dominant CMD2 mutation it would be transmitted to the next generation in a Mendelian manner. Resulting progeny plants would not be chimeric for the resistance allele and, as reported here, would not lose resistance to CMD after morphogenesis (FIG. 4).

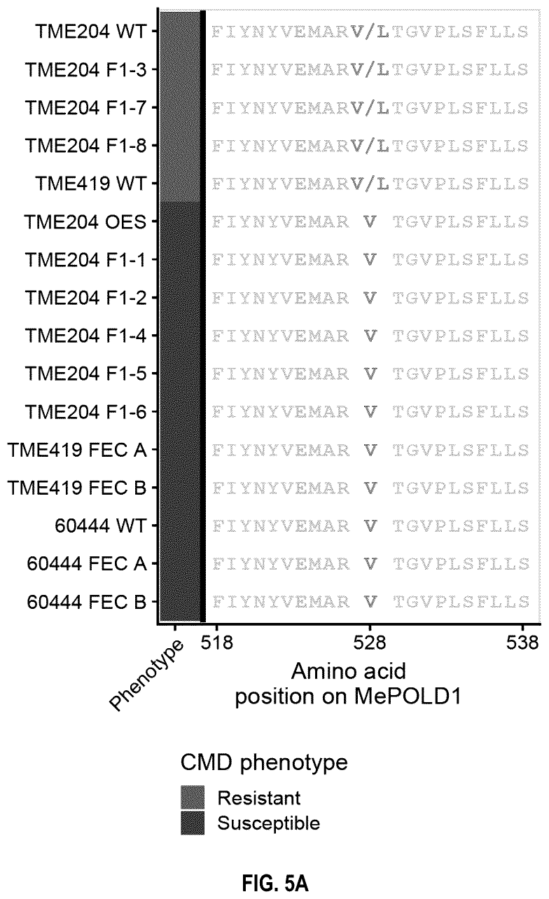

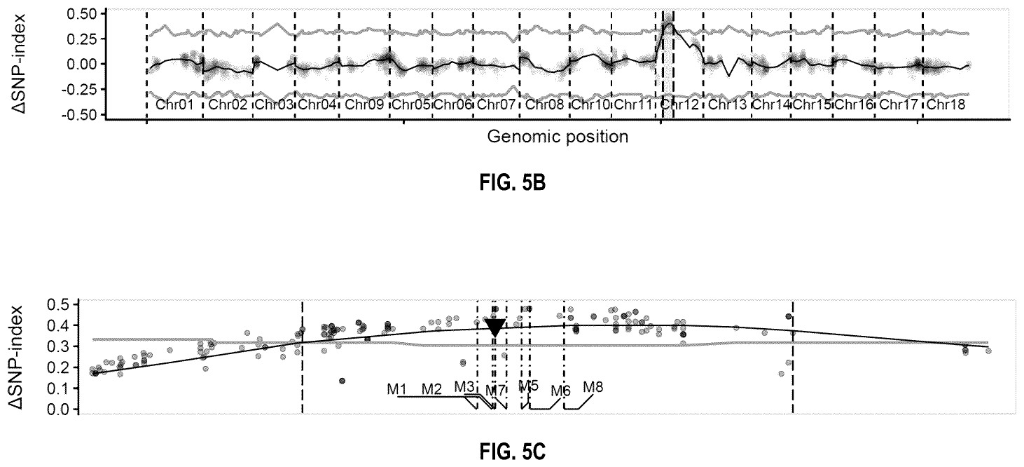

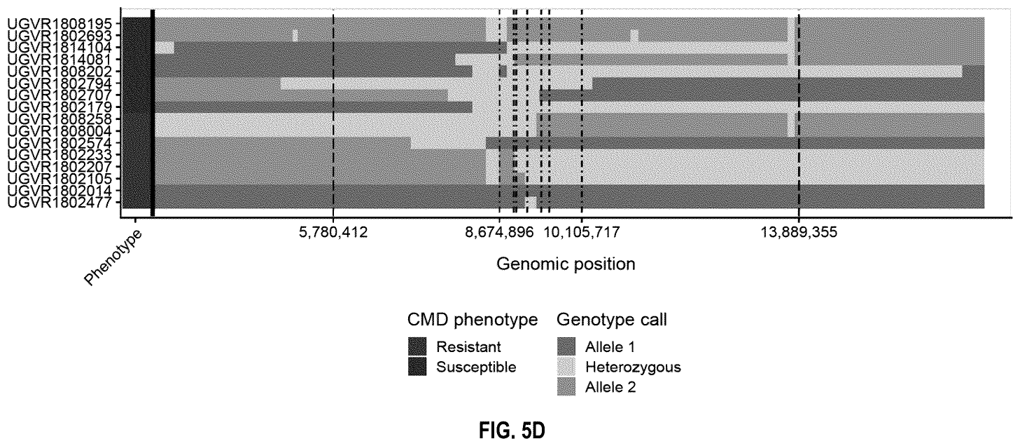

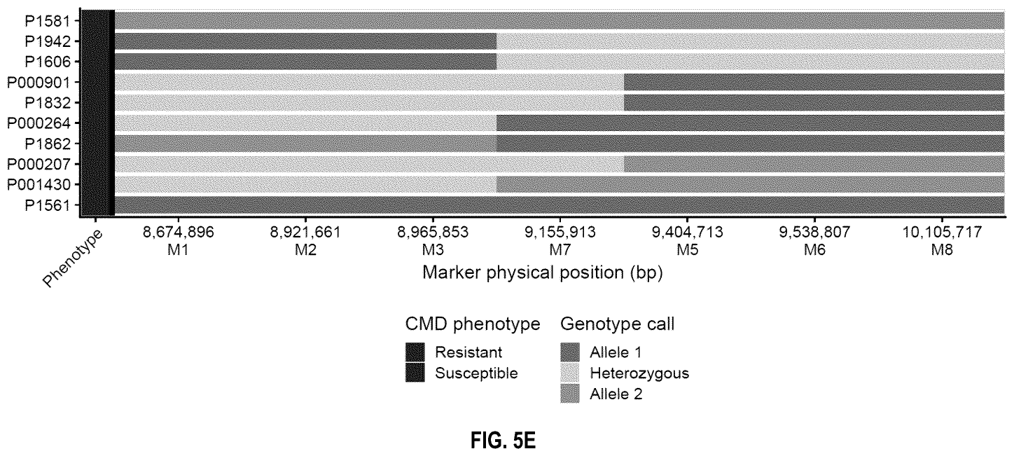

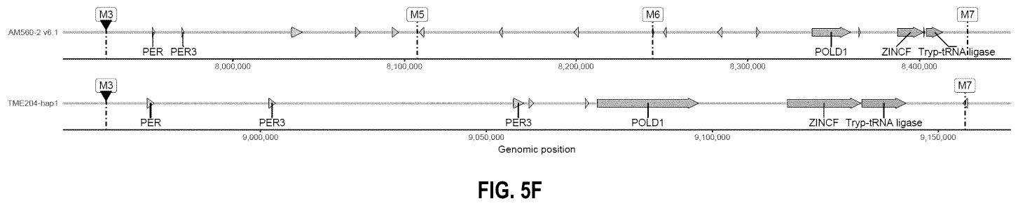

Whole genome sequencing and genetic variant analysis (WGS-GVA) were combined with fine-mapping to identify CMD2 and further understand the LCR trait. WGS-GVA has been used to understand the genetics behind rare human diseases. Causal variants shared by multiple individuals or families are revealed by comparison of WGS from sick and healthy individuals. WGS-GVA was performed to identify genetic changes in three CMD resistant and five susceptible F₁ plants. A filtering approach identified 405 SNPs segregating with the resistance phenotype in these individuals. If the LCR phenotype is indeed caused by a mutation within CMD2, then susceptible LCR lines should share variants with susceptible F₁ individuals, while wildtype resistant TME204 would not. Of the 405 SNPs identified in the resistant F₁ progeny, only one nonsynonymous SNP is heterozygous in the genome of resistant TME204 and absent in the genome of susceptible TME204-LCR plants. This observation is consistent with the hypothesis that CMD resistance is a chimeric trait in landraces and that passage through culture-induced embryogenesis leads to loss of chimerism. The SNP is located in the coding sequence of MePOLD1 (Manes.12G077400) and changes valine to leucine (V528L) (FIG. 5A). The amino acid sequence of MePOLD1 having the V528L substitution is set forth in SEQ ID NO: 49; genomic, transcript, and coding sequences which encode SEQ ID NO: 49 are set forth in SEQ ID NOs: 50, 51, and 52, respectively. EWAS confirmed that MePOLD1 has no DNA methylation differences in resistant and susceptible genotypes (FIG. 2D).

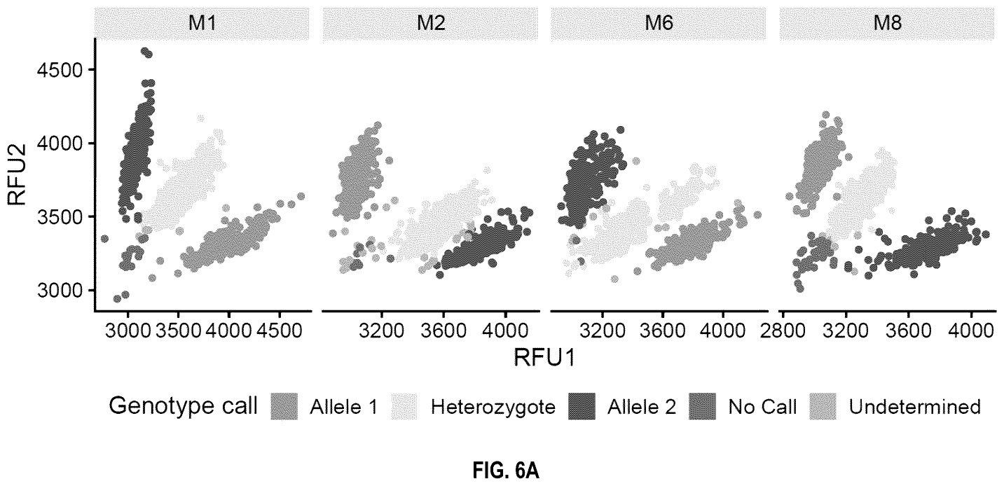

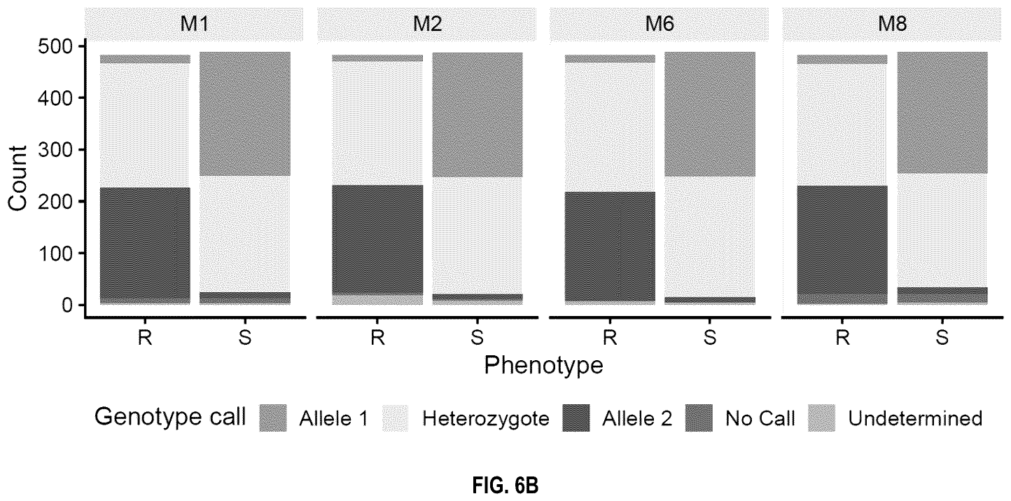

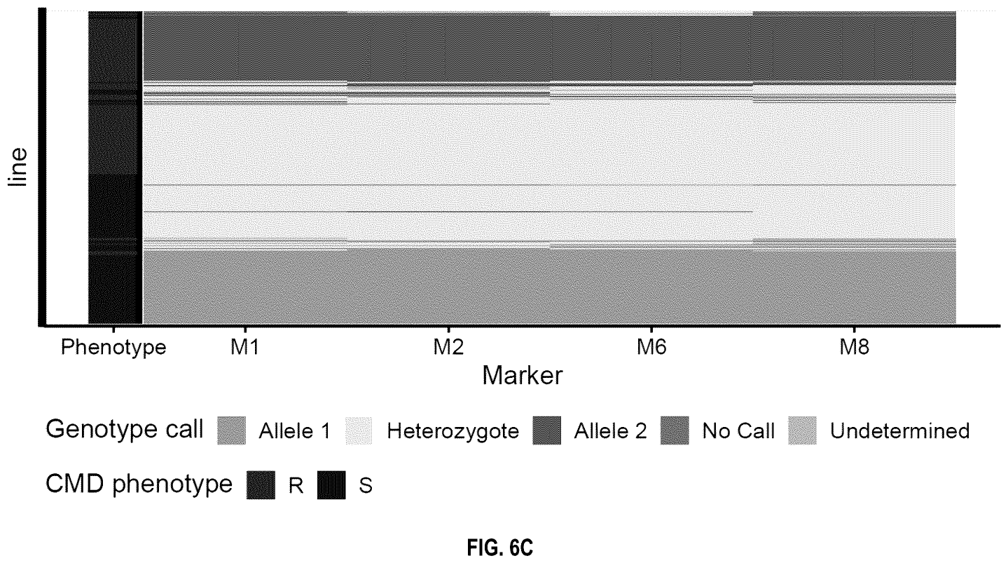

Fine-mapping was also pursued to pinpoint the CMD2/LCR genomic location. The recently released haplotype resolved genome assemblies of CMD2-resistant African cultivars TME7 and TME204 were leveraged to perform in silico bulk segregant analysis (BSA) to map CMD2 resistance. First, F₁ progeny were screened in the field in Uganda and genotyped with GBS (FIG. 1B). These data co-localize the CMD2/LCR locus with the previously identified CMD2 locus, placing it on Chromosome12 between 5 and 13 Mb of the TME204 haplotype 1 assembly (FIG. 5B). Recombinants within this region were identified using SNP calls from individual samples, thus narrowing the CMD2/LCR-locus to roughly 300 kb (FIG. 5C-D). To more accurately fine-map the locus, kompetitive allele specific PCR (KASP) markers were developed bracketing this region (FIG. 5C-F, FIG. 6). Approximately 1,000 F₁ individuals derived from a NASE14×TME204-LCR cross were genotyped and then phenotyped in the greenhouse using a previously described virus induced gene silencing (VIGS)-based infection assay. Sixty four (˜6.57 cM) recombinants were identified between markers M1 and M8 and those individuals were further screened using three additional markers (M3, M5, M7). This allowed the identification of recombinants which narrowed the CMD2/LCR locus to 190 kb, between M3 (8,965,853 bp) and M7 (9,155,913 bp) in the TME204-hap1 assembly (FIG. 5E-F).

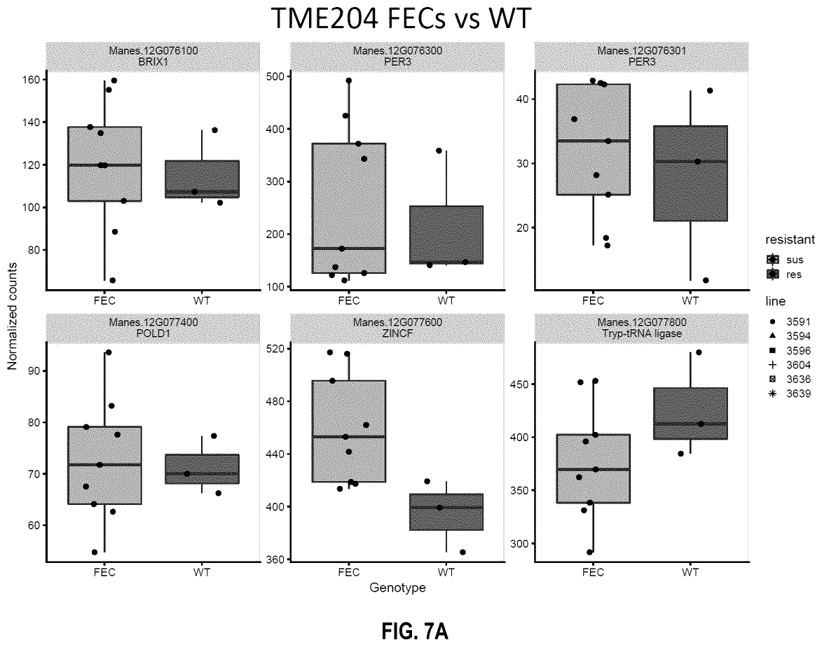

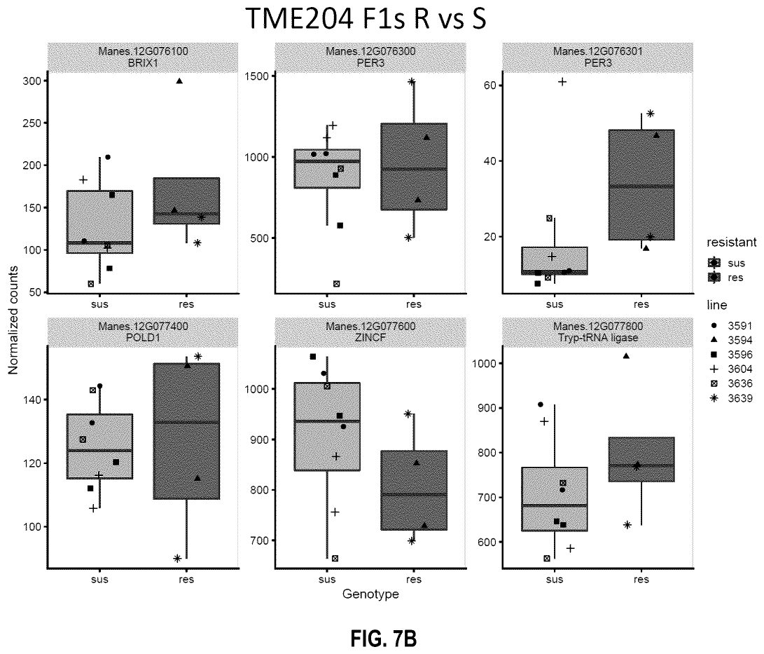

The marker order in both TME7 and TME204 assemblies is different than in the AM560-2 v6.1 assembly, suggesting a translocation or assembly error in the region which may have complicated previous efforts to find CMD2 (FIG. 5F). The newly defined fine-mapped locus consists of eight annotated genes, including several peroxidase genes that were previously proposed as CMD2 candidate genes and MePOLD1 (FIG. 5F). Differential gene expression analyses between susceptible and resistant individuals revealed no significant differences for genes found within this region (FIG. 7). Nucleotide level comparison of WGS data revealed that the V528L SNP in MePOLD1 was the only genetic change between these recombinant lines.

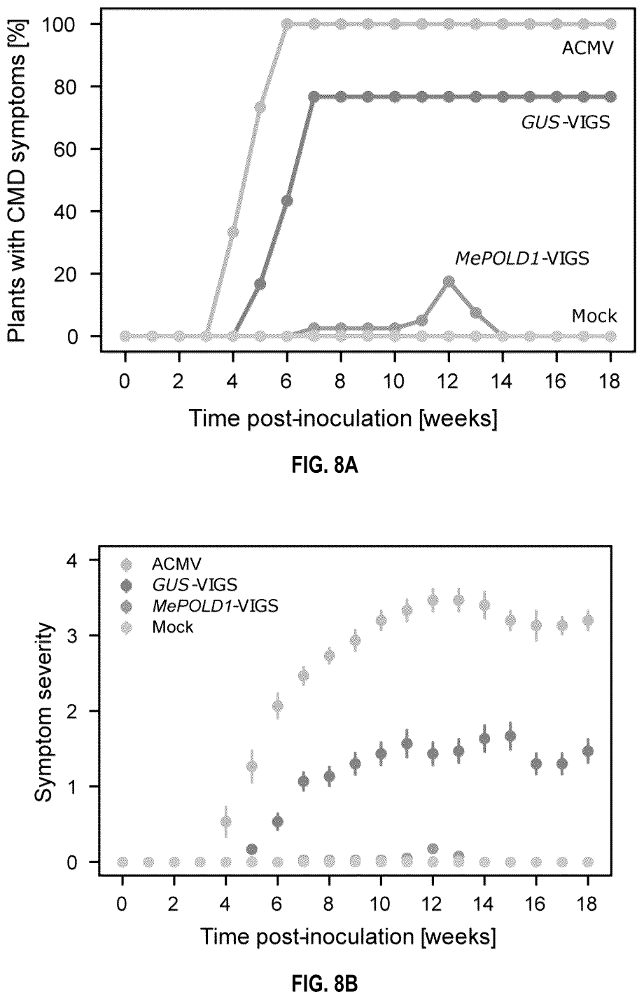

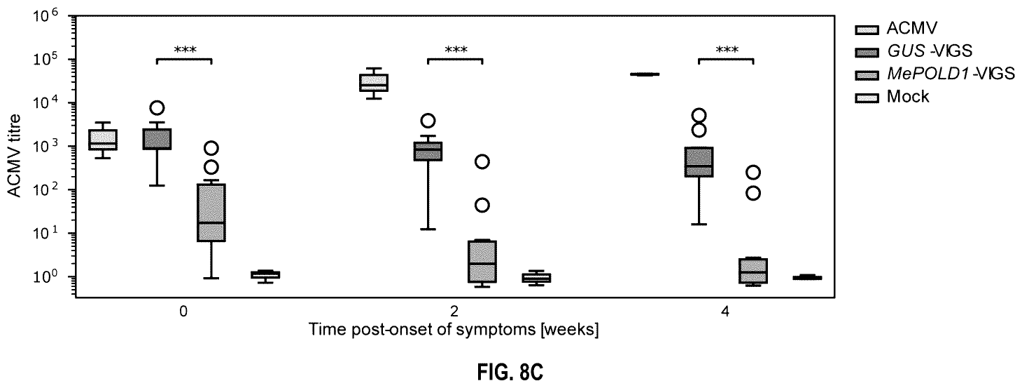

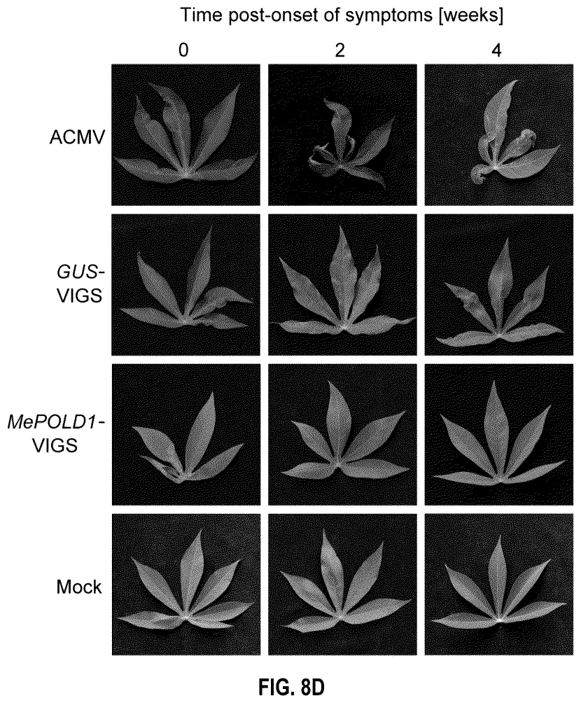

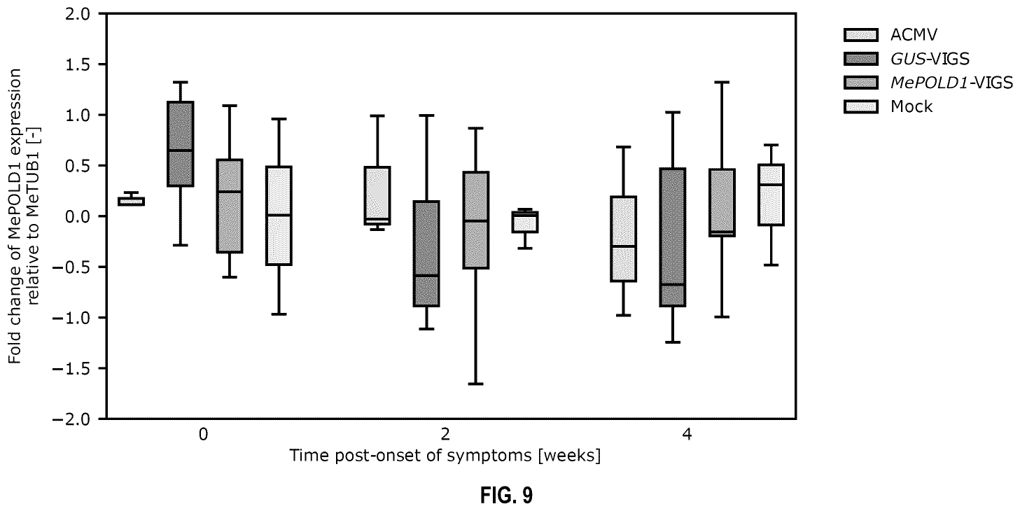

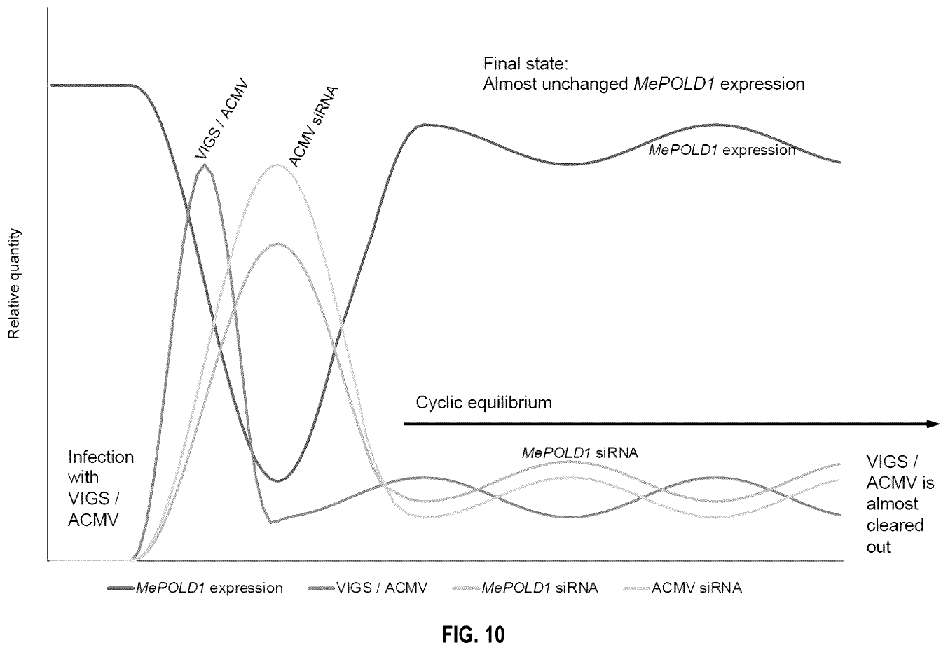

Taken together, these data suggest that variation within the MePOLD1 CDS underlie CMD2-type resistance. Finding a nonsynonymous SNP by WGS-GVA in the precisely mapped CMD2 locus by chance is statistically improbable (P=6.1×10⁻⁴, Monte Carlo simulation, n=100,000). Components of the DNA polymerase complex have been reported previously as required for susceptibility to geminiviruses. To understand if this holds true for cassava, MePOLD1 was targeted for downregulation in the CMD-susceptible cassava variety 60444 using VIGS (MePOLD1-VIGS). After inoculation with MePOLD1-VIGS, only 25% (n=40) of 60444 plants showed symptoms of infection compared to plants infected with GUS-VIGS (76.7%, n=30) and African cassava mosaic virus (ACMV) (100%, n=15). CMD symptom severity after MePOLD1-VIGS was also reduced in infected plants of 60444 (Hypergeometric Test, P<0.05, n=40, FIGS. 8A-B) and virus titre was significantly lower when compared to plants inoculated with control VIGS constructs or unmodified ACMV (FIG. 8C). Importantly, plants of 60444 that displayed CMD symptoms after inoculation with MePOLD1-VIGS underwent a recovery phenotype typical of CMD2 resistance and atypical for this highly CMD-susceptible variety (FIG. 8D). Together, the results demonstrate that MePOLD1-VIGS is sufficient to provide CMD resistance. While the phenotypic result of silencing MePOLD1 was clear, we did not observe a significant downregulation of MePOLD1 mRNA levels in 60444 inoculated with MePOLD1-VIGS vectors (FIG. 9). This may be because MePOLD1 is already expressed at very low levels in leaf tissues, or reflects inherent complexity associated with using a viral vector to down-regulate a gene required for virus replication (FIG. 10).

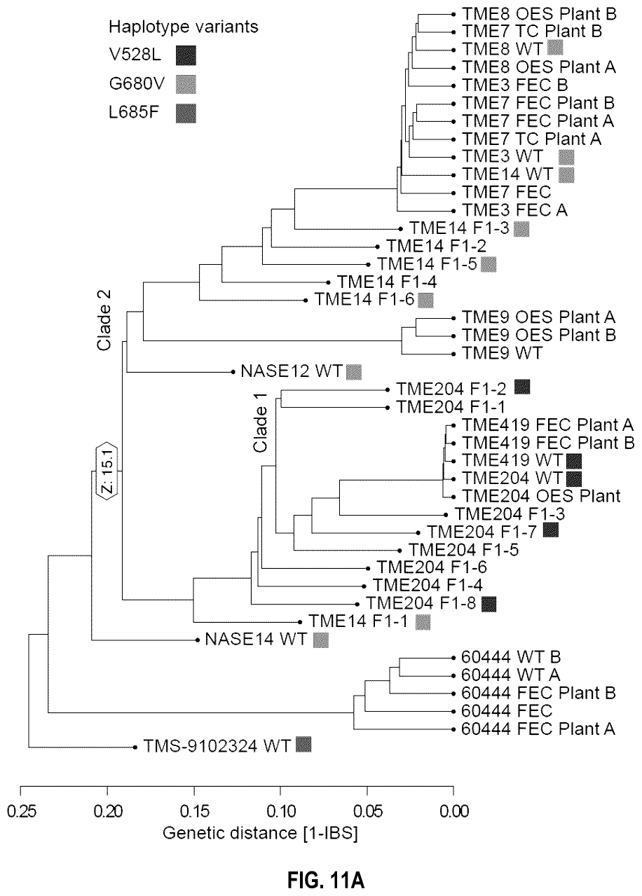

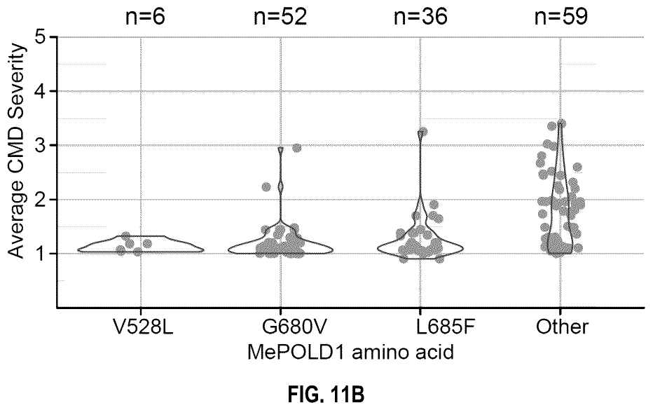

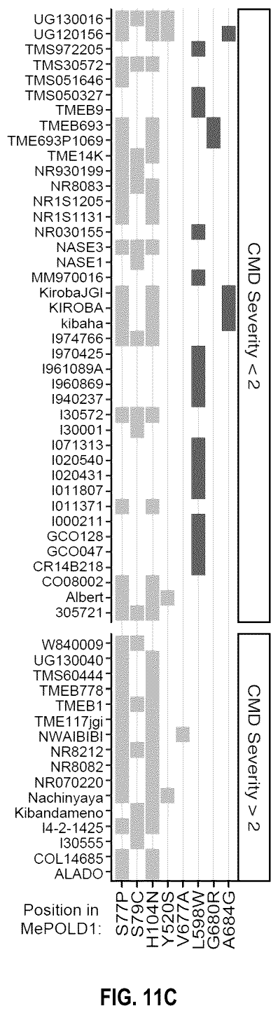

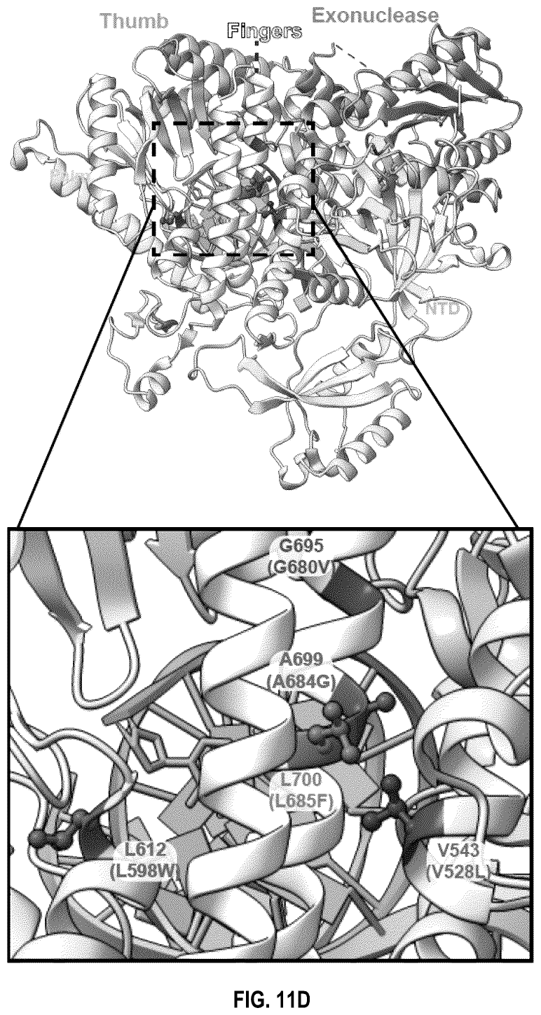

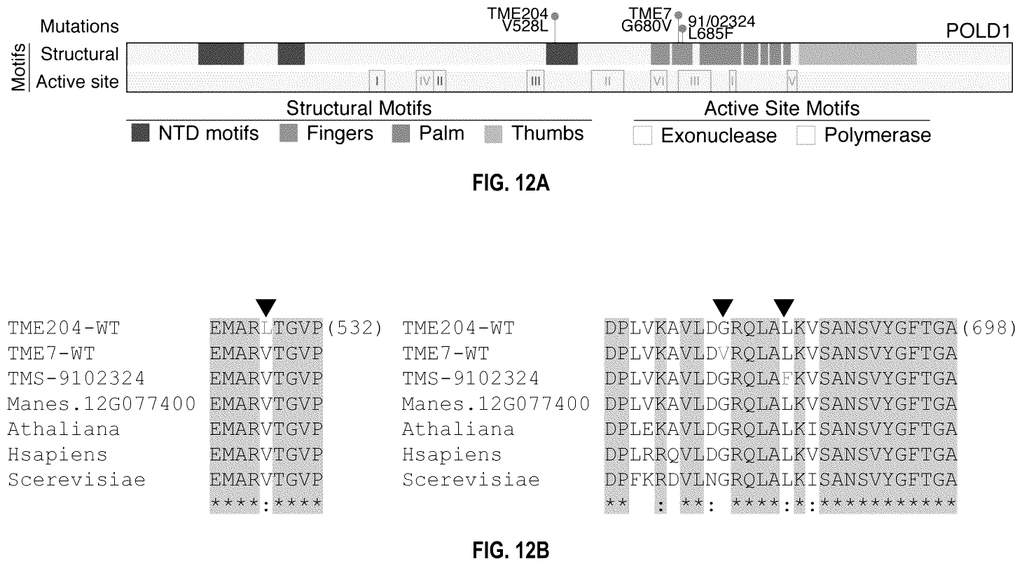

The MePOLD1 coding sequence of additional CMD-resistant cultivars was investigated using WGS-GVA and/or Sanger sequencing (FIG. 11). The V528L allele present in TME204 was also observed in TME419, consistent with these landraces being closely related, and both collected from farmers' fields in Togo/Benin. While other resistant varieties did not contain the V528L allele, two additional nonsynonymous SNPs were identified within MePOLD1 (G680V in TME3, TME8, TME14, NASE12 and NASE14 and L685F in TMS-9102324) (FIG. 11, FIG. 12). The amino acid sequence of MePOLD1 having the G680V substitution is set forth in SEQ ID NO: 53; genomic, transcript, and coding sequences which encode SEQ ID NO: 53 are set forth in SEQ ID NOs: 54, 54, and 56, respectively. The amino acid sequence of MePOLD1 having the L685F substitution is set forth in SEQ ID NO: 57; genomic, transcript, and coding sequences which encode SEQ ID NO: 57 are set forth in SEQ ID NOs: 58, 59, and 60, respectively. These results suggest that several distinct MePOLD1 alleles may explain CMD2 resistance. Publicly available re-sequencing data of diverse cassava germplasm were also queried and these varieties were cross referenced for CMD severity phenotype data available at CassavaBase. Of the 241 accessions with re-sequencing data, 153 have associated CMD susceptibility scores. MePOLD1 SNPs were identified in 94 of the resistant accessions (CMD score of less than 2 out of 5). Specifically, 6, 52, and 36 accessions harbour V528L, G680V, or L685F, respectively. (FIG. 11B). Analysis of the remaining 59 varieties identified three additional nonsynonymous SNPs in MePOLD1, unique to accessions with CMD severity scores below 2: L598W, G680R, and A684G; found in 17, 2, and 4 samples, respectively (FIG. 11C). In every case, across 117 samples in which POLD1 variants were identified, the putative resistance allele is observed in the heterozygous context, suggesting that these amino acid changes might be deleterious if homozygous. Five of the six mutations identified in MePOLD1 (V528L, G680V, G680R, A684G, L685F) are immediately adjacent to the R696-E539 (MePOLD1: R681-E524) salt bridge between the finger and N-terminal domains described in yeast POLD (FIG. 11D). Mutations disrupting this salt bridge have been shown to result in decreased polymerase activity and fidelity. Furthermore, a homozygous R696W mutation is lethal in yeast and is associated with oncogenesis in humans.

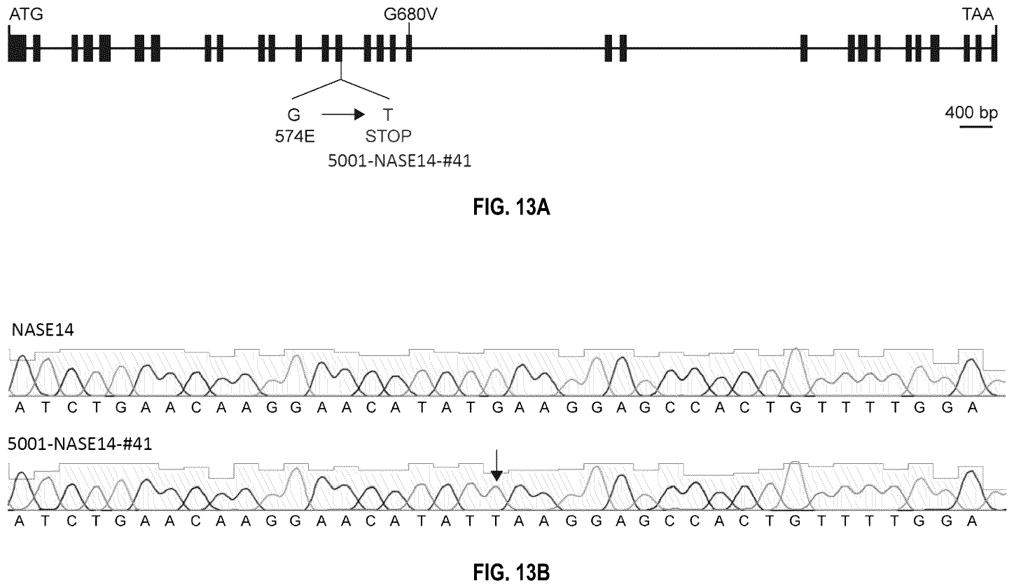

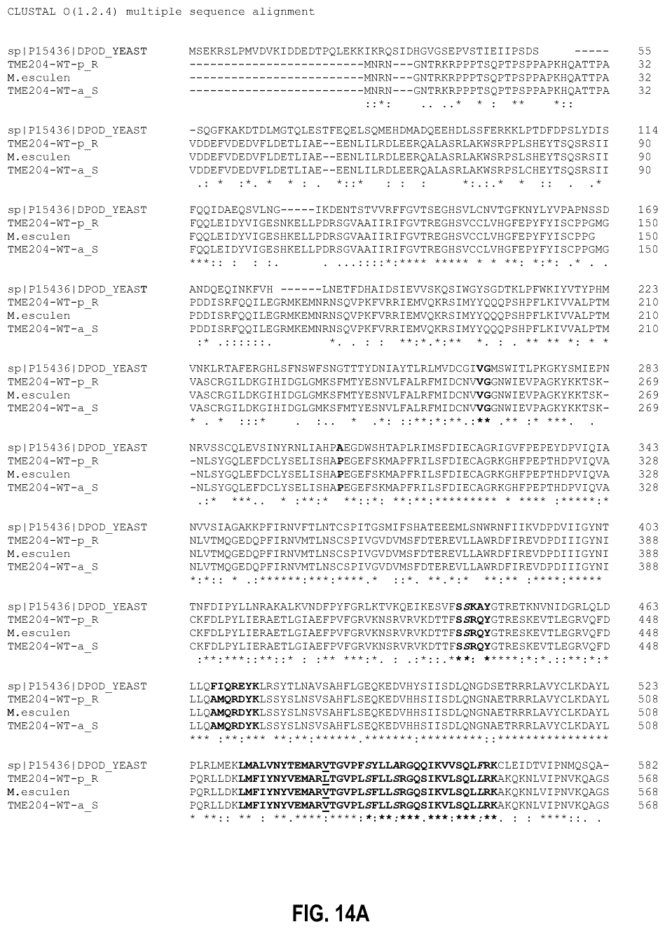

The above data suggest a model wherein MePOLD1 is a susceptibility factor involved in cassava geminivirus replication and that nonsynonymous mutations within MePOLD1 lead to CMD2-type resistance. This model was applied to an outstanding problem; the resistant NASE14 parent from the mapping populations is heterozygous for the G680V mutation and does not lose resistance after passage through culture-induced morphogenesis. However, we previously reported an exception, in which the transgenic line 5001-NASE14- #41 lost functional CMD2 resistance to become susceptible to CMD. Targeted Sanger sequencing confirmed that this line retained the G680V mutation. However, examining the cloned, full length CDS revealed the presence of a heterozygous SNP that introduces a premature stop codon at amino acid position 574 within the resistance allele (FIG. 13). Thus, transgenic event 5001-NASE14- #41 lacks a fully functional resistance allele, which would explain its acquired susceptibility to infection by CMGs.

Collectively, the data indicate that amino acid changes near the active centre of MePOLD1 cause the dominant CMD2-type resistance. Several dominant resistance genes for plant viruses have been reported, most of which belong to the NBS-LRR class of proteins. MePOLD1 represents an unexpected, novel type of resistance protein in plants. Evidence suggests that this has been selected as a chimeric clonal variant multiple times by West African farmers, and due to its monogenic, dominant nature is now favoured in breeding programs in Africa, India, and South-East Asia. Mutations in POLD predispose humans and mice to a range of cancers, especially mutations that specifically affect the proofreading activity or dNTP selectivity of the enzyme. It is possible that the identified mutations in MePOLD1 may similarly introduce replication errors in the geminiviruses, which would impair their replication efficacy and thereby reduce virus load in the host plant. This hypothesis is supported by the co-localization of MePOLD1 mutations to those in yeast and humans known to decrease DNA replication activity, and accuracy. We cannot exclude, however, that the MePOLD1 mutations weaken or block interactions with the virus replication-enhancer protein AC3, which interacts with subunits of POLD. CMD2 resistance has remained robust in farmers' fields over at least two decades. However, some caution for overreliance on CMD2 is presented here with evidence that yields and livelihoods for millions of cassava farmers are being secured by a few SNPs in one gene. The identification of mutations in MePOLD1 as the cause for CMD2-type resistance will facilitate the production of CMD resistant cassava varieties by SNP-assisted breeding or genome editing to introduce the identified SNPs into susceptible cultivars and provides opportunity to further elucidate mechanisms of resistance to geminiviruses.

### Methods

### Plant Lines, Mapping Populations and Disease Scoring

A crossing program was conducted in Uganda during the 2017/2018 cropping season to perform controlled crosses between CMD susceptible cultivar TME204-LCR and three CMD resistant wildtype cassava varieties (TME14, NASE14, NASE19) following the standard procedures described by Kawano (1980) and Hahn et el (1980). During the pollination period, special care was taken to cover mature flowers with pollination bags 2-3 days before and after pollination. A total of 7,200 botanical seeds were harvested from mature fruits within three months after pollination and stored in paper bags for approximately three weeks to break dormancy. All seeds were planted in field-conditioned nursery beds and 4,300 resultant seedlings transplanted to a field at six weeks or age and allowed to grow under natural field conditions for 12 months. The field trials were conducted at Namulonge, central Uganda, which is a hotspot for cassava mosaic disease with high whitefly vector populations. CMD-symptomatic plants of local cultivar Bao were planted as spreader rows to augment field inoculation of CMGs. To achieve phenotyping, monthly CMD severity was scores (starting from 1 month after transplanting seedlings) were recorded on a 1-5 scale where: 1=no symptoms; 2=mild chlorotic pattern over the entire leaf although the leaf appears green and healthy; 3=moderate mosaic pattern throughout the leaf, narrowing and distortion in the lower one-third of leaflets; 4=severe mosaic, distortion in two-thirds of the leaflets and general reduction in leaf size; and 5=severe mosaic distortion in the entire leaf. The final CMD severity data recorded at the crop age of 11 months were used for subsequent analyses.

A similar crossing program was established at Kandara, Kenya in which TME204-LCR was crossed with the two CMD resistant wildtype cassava varieties (TME14 and NASE14). Resulting seed were collected and shipped to DDPSC, St Louis, USA.

### Epigenome-Wide Association Studies (EWAS)

Whole genome methylation of TME7 and TME204 background samples were prepared with Bisulfite Kit (Qiagen, Germantown, Maryland, USA) and enzymatic Methyl-Seq kit (New England BioLabs, Ipswich, Massachusetts, USA), respectively. DNA methylation level at each cytosine was calculated by number of methylated C vs. total C and T count. Differentially Methylated Cytosines (DMCs) were identified by methdiff.py in BSMAP where differences in CG, CHG, and CHH methylation were at least 0.3, 0.2, and 0.1, respectively. Methylation levels of DMCs of each sample versus three TME7 and one TME204 wildtype were merged as a consensus DMCs table. Methylation levels of each sample in DMCs table were subjected to one-way ANOVA test by comparing seven resistant vs. seven susceptible samples to calculate p-value of each DMC. Manhattan plot of p-value were generated by R package qqman. Methylation track files were visualised with Integrative Genomics Viewer (IGV, v3.0).

### Determination of CMD Resistance Across Repeated Cycles of Somatic Embryogenesis

The three CMD resistant F1 progeny lines, NASE14×TME204-LCR.82, NASE14×TME204-LCR.73 and NASE14×TME204-LCR.16 were established and micro propagated in tissue culture. Organized somatic embryos (OES) were induced from leaf explants and plants regenerated to produce Cycle 1 OES-derived plants. This process was repeated with Cycle 1 OES plants to produce Cycle 2 OES plants, and again to generate Cycle 3 OES plants for each of the F1 progeny lines. Regenerated plants were established in the greenhouse and inoculated with East African cassava mosaic virus (EACMV-KE2) isolate K201. Ten plants were inoculated from each cycle of OES-derived plants for all three progeny and assessed for development of CMD leaf symptoms over a period of 90 days using a 0-5 visual scoring method. At 51 days after inoculation plants were ratooned and CMD symptoms scored on leaves produced by shoot regrowth.

### Whole Genome Sequencing and Genomic Variant Analysis

Illumina sequencing: Leaf material was collected from 42 cassava genotypes and FEC material from two cassava genotypes for whole genome Illumina sequencing. DNA libraries were prepared using the Illumina TruSeq Nano DNA High Throughput Library Prep Kit (20015965, Illumina, San Diego, California, USA). Libraries were sequenced using an Illumina NovaSeq system for 2×151 cycles. On average 100× Illumina paired-end (PE) data were collected per sample.

Pre-processing and mapping of reads was performed using ezRun in combination with SUSHI. Technical quality was evaluated using FastQC (v0.11.7). Possible contaminations were screened using FastqScreen (v0.11.1) against customized databases. Reads were pre-processed using fastp (v0.20.0) and aligned to the Manihot esculenta TME204 genome (V1.0, FGCZ) using Bowtie2 (v2.3.2) with the “—very-sensitive” option. PCR-duplicates were marked using Picard (v2.9.0). Frequency-based calls for all variants with allele frequency above 20% were performed with freebayes-paralell (v1.2.0-4-gd15209e). Relatedness analysis of SNPs using identity-by-descent (IBD) measures, was performed using the R/Bioconductor Package SNPRelate (v 3.13).

SNP analysis: To find potential SNPs, a custom python script (available on the World Wide Web at github.com/pascalschlaepferprivate/filter_vcf) parses the VCF file produced by freebayes, computes total coverage of the SNP, and then absolute and relative read coverage of all SNP variants. Samples were organized as ingroup (genotypes that show a SNP variant of interest), outgroup (genotypes that do not show SNP variant of interest), facultative ingroup (genotypes that may show SNP variant of interest), and facultative outgroup (genotypes that may not show SNP variant of interest), and SNPs were filtered according to these groups and additional parameters.

### Genetic Mapping

Genotyping by Sequencing and in silico bulk segregant analysis: Approximately 1,300 individual F₁ progeny and the parental lines from the NASE14×TME204-LCR population generated in Kenya were characterised with genotyping-by-sequencing (GBS) at UW-Madison Biotechnology Center following their standard ApeKI restriction enzyme protocol. Reads of 100 bp were demultiplexed and mapped to the TME204 hap1 assembly. SNPs were called using GATK4 and quality filtered SNPs that were heterozygous in both parents retained using vcftools v0.1.14. Using the field phenotypes, a random subset of the most CMD resistant and most susceptible lines was selected as the resistant and susceptible bulks (n=125 each), respectively, to perform in silico bulk segregant analysis using the QTLseqr package.

Fine-mapping using GBS and KASP markers: To further narrow the CMD2 locus, individual F₁ progeny were analysed for recombination events within the defined locus (˜5-13 Mb). While mapping in outcrossers using F₁ populations is established, mapping in this population is complicated by the TME204-LCR parent in that heterozygous progeny can be either resistant or susceptible. Thus, only recombinants with a genotype-phenotype mismatch were selected as informative. For example, in a phenotypically resistant F₁ line with a recombination that transitions from genetically heterozygous to genetically homozygous susceptible, one can exclude the homozygous susceptible region as not carrying CMD2. Six resistant and six susceptible recombinant individuals were identified with such recombination within the broad CMD2 locus and were used to exclude genomic regions in which at least two lines supported such exclusion. The narrow locus defined by GBS (Chromosome12: 8,976,221-9,314,764) was used to design KASP markers spanning 1.5 Mb bracketing this region. Additional recombinants were sought in a similar manner within a second ˜1000 individual population using highly accurate genotyping and phenotyping assays (KASP-marker-based assay combined with phenotyping with a VIGS based approach). All recombinants were sequenced using Illumina WGS data and nucleotide level comparison was performed by alignment to TME7 and TME204 assemblies and manual inspection using CLC Genomics and IGV.

Phenotyping for fine-mapping: F₁ progeny seeds were germinated in a growth chamber at DDPSC, transferred to the greenhouse and inoculated with a virus-induced-gene-silencing version of East African cassava mosaic virus K201 (SPINDLY-VIGS), as described by Beyene et al. (2017). Plants were assessed over a four-week period. Plants which died were scored as CMD susceptible while those that recovered from initial symptoms and re-established healthy growth were scored as CMD resistant.

### RNAseq Analysis

For differential expression analysis, first a transcriptome fasta of the spliced exons was made from the TME204-hap1 gff file using ‘gffread-w’ from the cufflinks package. This transcriptome was then concatenated to the whole genome to prepare an alignment decoy file and index using the commands available on the World Wide Web at combine-lab.github.io/alevin-tutorial/2019/selective-alignment/. Trimmed RNAseq reads were then pseudo-aligned to the TME204-hap1 transcriptome using Salmon v1.5.2 default settings. Read count data was imported into R using the tximport package. Samples were then defined as resistant or susceptible and differential expression on the integer count values was performed using DESeq2. Genes with a sum of less than 50 reads across all samples were excluded from analysis. Differential expression was performed using “apeglm” as the Log Fold Change Shrinkage method. Genes were defined as being significantly differentially expressed if they had an adjusted p-value of less than 0.05. Normalised counts were plotted using ggplot and tidyverse functions in R.

### VIGS Targeting of MePOLD1

A VIGS approach was designed and performed based on Lentz et al. (2019). A 400 bp coding sequence of MePOLD1 (position 438-837, corresponding to 8905774-8905965 of chr12 in AM560 v8, 9076083-9076741 of chr12 in TME204 hap1) as synthesized (Twist Biosciences, California, USA) and inserted in the multiple cloning site of the ACMV-based VIGS vector using KpnI and SpeI. The 400 bp coding sequence is conserved in MePOLD1 of 60444, TME3, TME204 and AM560. The number of 60444 plants inoculated were n=15 for ACMV, n=40 for MePOLD1-VIGS, n=30 for GUS-VIGS, and n=15 for Mock treatments. Leaf symptom scoring was based on Fauquet and Fargette (1990). ACMV titre and MePOLD1 expression were quantified through qPCR from total DNA and RNA extracted respectively from the top 1-2 leaves harvested at first signs of CMD symptoms. A Mann-Whitney U test was used to analyse the statistical significance.

### Identification of Additional MePOLD1 Variants

A publicly available dataset was accessed containing sequencing data of 241 diverse accessions that identified over 28 million segregating variants. All positions within the MePOLD1 gene (AM560-2 v6.1 coordinates) were extracted from the Chromosome12 VCF file available through the cassavabase.org FTP server (c12.DepthFilt_phasedSNPs.vcf), and effects of the variants on the protein coding sequence determined using snpEff. Additional analysis was done with Sanger sequencing.

### POLD1 Protein Sequence Analyses

The 3D structure of the yeast POLD catalytic subunit and template DNA (PDB ID: 3IAY, Swan et al., 2009), was visualized in ChimeraX. The N-terminal domain, exonuclease domain, and finger, palm, and thumb motifs from Swan et al., 2009 were color-coded and the residues corresponding to the nonsynonymous mutations identified across the cassava varieties are highlighted.

### Analysis of MePOLD1 in 5001-NASE 14- #41

The full-length cDNA of MePOLD1 was amplified from cassava plant line 5001-NASE 14- #41. Primers were designed to be specific for the haplotype carrying the resistance MePOLD1 allele and PCR performed. The PCR product was cloned into the binary vector pCAMBIA1305.1 using the In-Fusion® HD Cloning Kit (Takara Bio USA, Inc.) and the resulting clones sequenced by Sanger sequencing.

### Example 2

In this Example, additional amino acid mutations that cause POLD1 to mediate resistance to geminiviruses were identified in tomato, soybean, and cotton.

In tomato, it was determined that POLD1 is in the region of the QTL for the Ty-6 geminivirus resistance locus. Lines known to contain this locus were requested from the Tomato Genetic Resource Center. By sequencing a targeted region of the POLD1 transcript, differences between resistant and susceptible varieties were identified. Resistant tomato varieties have an aspartic acid (D) at the position corresponding to amino acid residue number 622 of the wild-type POLD1 (SEQ ID NO: 5) protein while susceptible varieties have a glutamic acid (E) at residue number 622 of the wild-type POLD1 (SEQ ID NO: 5) protein. Position 622 of tomato POLD1 (SEQ ID NO: 5) corresponds to position 627 of cassava POLD1 (SEQ ID NO: 1). The amino acid sequence of a resistant POLD1 from a wild tomato species (Solanum chilense) is set forth in SEQ ID NO: 45; genomic, transcript, and coding sequences which encode SEQ ID NO: 45 are set forth in SEQ ID NOs: 46, 47, and 48, respectively.

For cotton, POLD1 was analyzed in a publicly available RNA-seq data set (from doi.org/10.1111/pbi.13236) of a resistant variety. Evidence was found that this cotton variety shares a SNP that was identified in cassava (residue 528). Other geminivirus resistant cotton lines were requested which were then sequenced at a targeted region of the POLD1 transcript to identify differences between resistant and susceptible varieties.

For soybean, a published dataset (from dx.doi.org/10.1111/pbi.13466) describing a haplotype map from whole genome sequencing of 1007 soybean accessions was analyzed. Two non-synonymous SNPs were reported in POLD1.

Table 3 shows a summary of the mutations identified in cassava, tomato, cotton, and soybean and their amino acid position in each species.

TABLE 3

| | Position in | Position in | Position in | Position in |
|---|---|---|---|---|
| | SEQ ID NO: 1 | SEQ ID NO: 5 | SEQ ID NO: 9 | SEQ ID NO: 17 |
| | (MePOLD1) | (SlPOLD1) | (GhPOLD1) | (GmPOLD1) |
| Cassava | 528 | 523 | 534 | 522 |
| | 598 | 593 | 604 | 592 |
| | 680 | 675 | 686 | 674 |
| | 684 | 679 | 690 | 678 |
| | 685 | 680 | 691 | 679 |
| Tomato | 627 | 622 | 633 | 621 |
| Cotton | 606 | 601 | 612 | 600 |
| | 714 | 709 | 720 | 708 |
| | 758 | 753 | 764 | 752 |
| Soybean | 520 | 515 | 526 | 514 |
| | 617 | 612 | 623 | 611 |

### Example 3

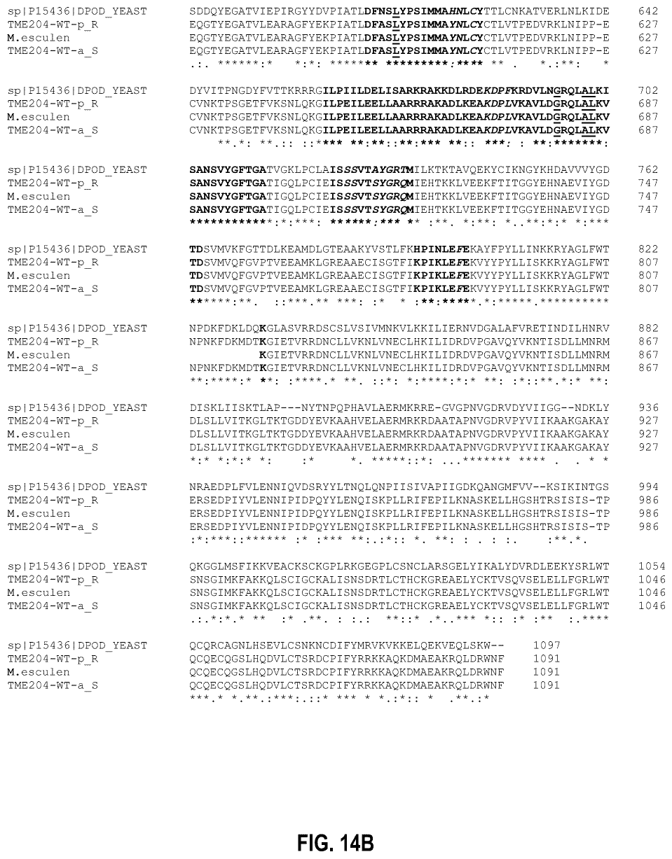

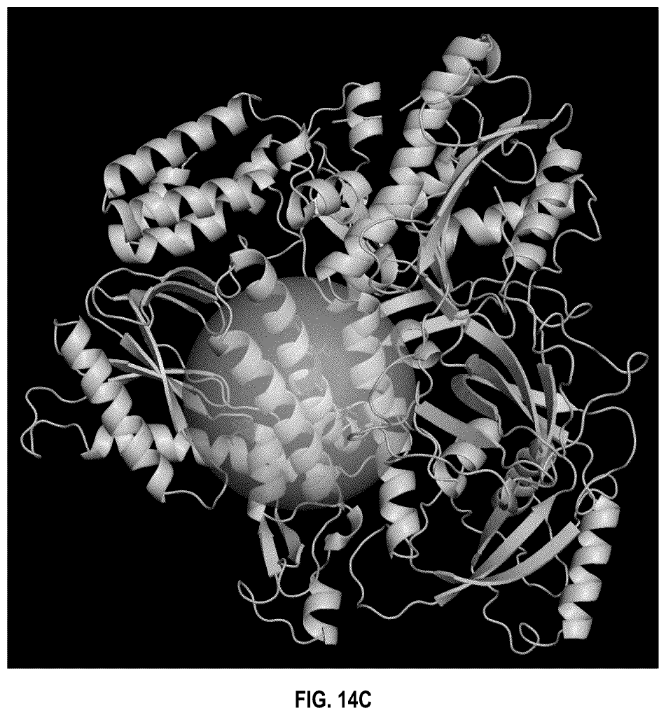

In this Example, amino acid regions of POLD1 predicted to mediate resistance to geminiviruses and were identified. Based on the crystal structure of yeast POLD1 (3IAY), the ClustalW alignment tool (FIG. 14A-B) was used to identify the relative amino acid positions in yeast for which mutations were found in cassava (V543, L612, G695, A699, L700). The arithmetic mid-point of all atoms belonging to the five amino acids was used to define the center of a sphere of influence (FIG. 14C). This sphere then was defined to have a radius to the outermost atom of the five amino acids (H10 of V543), involved in mutations, including its van der Waal radius of 1.2 Å. All amino acids for which the van der Waal radius of one of its atoms either touched or was engulfed by their collective sphere were identified as amino acids of interest. When displayed on the alignment, 21 regions were identified. Regions were merged if two regions were spaced by no more than 5 amino acids. The following 11 regions of amino acids (relative to SEQ ID NO: 1) as relevant in POLD1 resistance to geminiviruses were identified: 252 to 253, 290, 426 to 431, 452 to 458, 516 to 552, 593 to 609, 647 to 698, 708 to 719, 748 to 749, 780 to 787, and 818.

The breadth and scope of the present disclosure should not be limited by any of the above-described examples.

1. A transgenic plant, or a plant cell thereof, with enhanced resistance to at least one geminivirus, the plant comprising a transgene encoding a DNA polymerase delta subunit 1 (POLD1) polypeptide, wherein the POLD1 polypeptide comprises a mutation of at least one amino acid in or near the active center of the POLD1 polypeptide.

2. The transgenic plant of claim 1, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

3. The transgenic plant of claim 1, wherein the POLD1 polypeptide comprises a mutation of at least one amino acid at a position corresponding to position 520, 528, 598, 606, 617, 627, 680, 684, 685, 714, or 758 of SEQ ID NO: 1.

4. The transgenic plant of claim 1, wherein the POLD1 polypeptide comprises an amino acid other than tyrosine at the position corresponding to position 520 of SEQ ID NO: 1, an amino acid other than valine at the position corresponding to position 528 of SEQ ID NO: 1, an amino acid other than leucine at the position corresponding to position 598 of SEQ ID NO: 1, an amino acid other than tyrosine at the position corresponding to position 606 of SEQ ID NO: 1, an amino acid other than glutamic acid or aspartic acid at the position corresponding to position 617 of SEQ ID NO: 1, an amino acid other than glutamic acid at the position corresponding to position 627 of SEQ ID NO: 1, an amino acid other than glycine at the position corresponding to position 680 of SEQ ID NO: 1, an amino acid other than alanine at the position corresponding to position 684 of SEQ ID NO: 1, an amino acid other than leucine at the position corresponding to position 685 of SEQ ID NO: 1, an amino acid other than serine at the position corresponding to position 714 of SEQ ID NO: 1, or an amino acid other than serine or proline at the position corresponding to position 758 of SEQ ID NO: 1.

5. The transgenic plant of claim 1, wherein the POLD1 polypeptide comprises a serine at the position corresponding to position 520 of SEQ ID NO: 1, a leucine at the position corresponding to position 528 of SEQ ID NO: 1, a tryptophan at the position corresponding to position 598 of SEQ ID NO: 1, a cysteine at the position corresponding to position 606 of SEQ ID NO: 1, a lysine at the position corresponding to position 617 of SEQ ID NO: 1, an aspartic acid at the position corresponding to position 627 of SEQ ID NO: 1, a valine or an arginine at the position corresponding to position 680 of SEQ ID NO: 1, a glycine at the position corresponding to position 684 of SEQ ID NO: 1, a phenylalanine at the position corresponding to position 685 of SEQ ID NO: 1, an arginine at the position corresponding to position 714 of SEQ ID NO: 1, or a phenylalanine at the position corresponding to position 758 of SEQ ID NO: 1.

6. The transgenic plant of claim 1, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57, or wherein the transgene encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

7.-11. (canceled)

12. The transgenic plant of claim 1, wherein the plant is a cassava, tomato, cotton, bean, soybean, maize, beet, pepper, grape, sweet potato, cucurbit, or okra plant.

13.-21. (canceled)

22. A plant part obtained from the plant of claim 1, optionally wherein the plant part is a seed.

23. A method of enhancing resistance of a plant to infection by a geminivirus, the method comprising:
modifying at least one plant cell to comprise a polynucleotide encoding a POLD1 polypeptide, wherein the POLD1 polypeptide comprises a mutation of at least one amino acid in or near the active center of the POLD1 polypeptide.

24. The method of claim 23, wherein the POLD1 polypeptide comprises a mutation of at least one amino acid at a position corresponding to positions 252 to 253, 290, 426 to 431, 452 to 458, 516 to 552, 593 to 604 to 609, 647 to 668, 674 to 698, 708 to 719, 748 to 749, 780 to 787, or 818 of SEQ ID NO: 1.

25.-27. (canceled)

28. The method of claim 23, wherein the POLD1 polypeptide has at least 80%, at least 90%, at least 95%, at least 98%, or at least 99% sequence identity to the amino acid sequence set forth in SEQ ID NO: 1, 5, 9, 13, 17, 21, 25, 29, 33, 37, 41, 45, 49, 53, or 57, or wherein the polynucleotide encoding the POLD1 polypeptide has at least 80%, at least 90%, at least 95%, at least 98%, or at least 99% sequence identity to at least one of the nucleotide sequences set forth in SEQ ID NOs: 2-4, 6-8, 10-12, 14-16, 18-20, 22-24, 26-28, 30-32, 34-36, 38-40, 42-44, 46-48, 50-52, 54-56, or 58-60.

29. (canceled)

30. The method of claim 23, wherein the modifying comprises transforming at least one plant cell with a polynucleotide encoding the POLD1 polypeptide, or wherein the modifying comprises using genome editing to modify the nucleotide sequence of a native gene in the genome of the plant cell.

31.-32. (canceled)

33. The method of claim 30, wherein the genome editing comprises using a zinc-finger nuclease (ZFN), a TAL (transcription activator-like) effector nuclease (TALEN), or a Clustered Regularly Interspaced Short Palindromic Repeats/CRISPR-associated nuclease (CRISPR/Cas nuclease) system.

34. The method of claim 23, wherein the plant cell is regenerated into a plant comprising in its genome the polynucleotide.

35. The method of claim 23, further comprising selecting for a plant or a plant cell having enhanced resistance to the geminivirus as compared to a corresponding control plant or plant cell without the polynucleotide.

36.-39. (canceled)

40. A method of limiting a disease caused by a geminivirus in agricultural crop production, the method comprising:
planting a seedling, cutting, tuber, or seed of the plant of claim 1; and growing the seedling, cutting, tuber, or seed under conditions favorable for the growth and development of a plant resulting therefrom, optionally wherein the plant is subjected to geminivirus infection.

41.-52. (canceled)

53. A method for introducing resistance to a disease caused by a geminivirus into a plant, the method comprising:
(a) crossing the plant of claim 1 with a second plant;
(b) genotyping at least one progeny plant for the presence of the mutation of at least one amino acid in or near the active center of the POLD1 polypeptide; and
(c) selecting at least one progeny plant comprising in its genome the polynucleotide encoding the POLD1 polypeptide comprising the mutation.

54.-60. (canceled)

61. A polynucleotide encoding a POLD1 polypeptide, wherein the POLD1 polypeptide comprises a mutation of at least one amino acid in or near the active center of the POLD1 polypeptide, and wherein the polynucleotide is operably linked to a polynucleotide comprising a heterologous promoter.

62-71. (canceled)

72. A method of producing a commodity plant product, the method comprising: (i) processing the plant of claim 1, or a part thereof; and (ii) recovering the commodity plant product from the processed plant or part thereof.

73.-74. (canceled)

75. A biological sample comprising a detectable amount of the polynucleotide of claim 61.

76.-84. (canceled)
